SEQUENCE LISTING (128 sequences)

SEQ ID NO: 1 - protein, 746 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPGGGGSYRYRQGGGGSTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQAGNAQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 2 - protein, 598 aa, synthetic construct
TAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQAGNAQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 3 - protein, 533 aa, synthetic construct
MATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQAGNAQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 4 - protein, 239 aa, Aequorea victoria
MVSKGEELFTGVVPILVELDGDVNGHKFSVRGEGEGDATNGKLTLKFICTTGKLPVPWPTLVTTLGYGVACFARYPDHMKQHDFFKSAMPEGYVQERTISFKDDGTYKTRAEVKFEGDTLVNRIVLKGIDFKEDGNILGHKLEYNFNSHKVYITADKQKNGIKANFKTRHNVEDGGVQLADHYQQNTPIGDGPVLLPDNHYLSHQSKLSKDPNEKRDHMVLKERVTAAGITHDMDELYK

SEQ ID NO: 5 - protein, 550 aa, Luciola italica
MEDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKIAV

SEQ ID NO: 6 - protein, 698 aa, synthetic construct
HHHHHHGLNDIFEAQKIEWHEAPPAPCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 7 - protein, 393 aa, synthetic construct
LEEKKGNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSVDGGSPTPPTPGGGSGLNDIFEAQKIEWHEARAHHHHHH

SEQ ID NO: 8 - protein, 746 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPGGGGSYRYRQGGGGSTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 9 - protein, 598 aa, synthetic construct
TAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 10 - protein, 533 aa, synthetic construct
MATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 11 - protein, 381 aa, synthetic construct
GGGRYESKGGGGGGGSARVDQTPQTITKETGESLTINCVLRDSNCALPSTYWYRKKSGSTNEESISKGGRYVETVNSGSKSFSLRINDLTVEDSGTYRCKVIAQLGWWLRGCNYRKHDVYGDGTAVTVNAGGGGSHHHHHHLEVLFQGPGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 383 aa, synthetic construct
GGGRYESKGGGGGGGSQVQLVESGGGLVQAGGSLRLSCAASGSSISSIAMGWYRQAPGKERELVAAISSGGTINYADSVKGRFTISRDNAKNTVYLQMNSLKPEDTAVYYCNTGRRLQTGSWGQGTQVTVSSGGGGSHHHHHHLEVLFQGPGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 376 aa, synthetic construct
ARVDQTPQTITKETGESLTINCVLRDSNCALPSTYWYRKKSGSTNEESISKGGRYVETVNSGSKSFSLRINDLTVEDSGTYRCKVIAQLGWWLRGCNYRKHDVYGDGTAVTVNAGGGGSHHHHHHGGGRYESKGGGLEVLFQGPGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 378 aa, synthetic construct
QVQLVESGGGLVQAGGSLRLSCAASGSSISSIAMGWYRQAPGKERELVAAISSGGTINYADSVKGRFTISRDNAKNTVYLQMNSLKPEDTAVYYCNTGRRLQTGSWGQGTQVTVSSGGGGSHHHHHHGGGRYESKGGGLEVLFQGPGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 244 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGRYESKGGG

SEQ ID NO: 16 - protein, 253 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSGGGGSGGGRYESKGGG

SEQ ID NO: 17 - protein, 508 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGCGTKLELKGGSGGGGSGGGGSGGGGSGGQVQLKQSGPGLVQPSQSLSITCTVSGFSLTNYGVHWVRQSPGKCLEWLGVIWSGGNTDYNTPFTSRLSINKDNSKSQVFFKMNSLQSNDTAIYYCARALTYYDYEFAYWGQGTLVTVSAGGGGSGGGGSGGGGRYESKGGGLEVLFQGPGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 517 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGCGTKLELKGGSGGGGSGGGGSGGGGSGGQVQLKQSGPGLVQPSQSLSITCTVSGFSLTNYGVHWVRQSPGKCLEWLGVIWSGGNTDYNTPFTSRLSINKDNSKSQVFFKMNSLQSNDTAIYYCARALTYYDYEFAYWGQGTLVTVSAGGGGSGGGGSGGGGSGGGGSGGGRYESKGGGLEVLFQGPGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 243 aa, synthetic construct
EVQLQQSGPELVKPGASMKISCKASGYSFTGYTMNWVKQSHGENLEWIGRINPHNGGTDYNQKFKDKAPLTVDKSSNTAYMELLSLTSEDSAVYYCARGYYYYSLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGRYESKGGG

SEQ ID NO: 20 - protein, 247 aa, synthetic construct
QSMQESGPGLVKPSQTLSLTCTVSGFSLSSYAMSWIRQHPGKGLEWIGYIWSGGSTDYASWAKSRVTISKTSTTVSLKLSSVTAADTAVYYCARRYGTSYPDYGDASGFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGRYESKGGG

SEQ ID NO: 21 - protein, 252 aa, synthetic construct
EVQLQQSGPELVKPGASMKISCKASGYSFTGYTMNWVKQSHGENLEWIGRINPHNGGTDYNQKFKDKAPLTVDKSSNTAYMELLSLTSEDSAVYYCARGYYYYSLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSGGGGSGGGRYESKGGG

SEQ ID NO: 22 - protein, 256 aa, synthetic construct
QSMQESGPGLVKPSQTLSLTCTVSGFSLSSYAMSWIRQHPGKGLEWIGYIWSGGSTDYASWAKSRVTISKTSTTVSLKLSSVTAADTAVYYCARRYGTSYPDYGDASGFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSGGGGSGGGRYESKGGG

SEQ ID NO: 23 - protein, 248 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGRYESKGGG

SEQ ID NO: 24 - protein, 257 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGRYESKGGG

SEQ ID NO: 25 - protein, 245 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGRYESKGGG

SEQ ID NO: 26 - protein, 254 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSGGGGSGGGRYESKGGG

SEQ ID NO: 27 - protein, 509 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGCGTKVEIKGGSGGGGSGGGGSGGGGSGGEVQLVESGGGLVQPGGSLRLSCAASGFNIKDTYIHWVRQAPGKCLEWVARIYPTNGYTRYADSVKGRFTISADTSKNTAYLQMNSLRAEDTAVYYCSRWGGDGFYAMDYWGQGTLVTVSSGGGGSGGGGSGGGGRYESKGGGLEVLFQGPGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 518 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGCGTKVEIKGGSGGGGSGGGGSGGGGSGGEVQLVESGGGLVQPGGSLRLSCAASGFNIKDTYIHWVRQAPGKCLEWVARIYPTNGYTRYADSVKGRFTISADTSKNTAYLQMNSLRAEDTAVYYCSRWGGDGFYAMDYWGQGTLVTVSSGGGGSGGGGSGGGGSGGGGSGGGRYESKGGGLEVLFQGPGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 22 aa, synthetic construct
GGGGSGGGGSGGGGRYESKGGG

SEQ ID NO: 30 - protein, 31 aa, synthetic construct
GGGGSGGGGSGGGGSGGGGSGGGRYESKGGG

SEQ ID NO: 31 - protein, 746 aa, synthetic construct; identical to SEQ ID NO: 1

SEQ ID NO: 32 - protein, 381 aa, synthetic construct; identical to SEQ ID NO: 11

SEQ ID NO: 33 - protein, 383 aa, synthetic construct; identical to SEQ ID NO: 12

SEQ ID NO: 34 - protein, 376 aa, synthetic construct; identical to SEQ ID NO: 13

SEQ ID NO: 35 - protein, 378 aa, synthetic construct; identical to SEQ ID NO: 14

SEQ ID NO: 36 - protein, 244 aa, synthetic construct; identical to SEQ ID NO: 15

SEQ ID NO: 37 - protein, 253 aa, synthetic construct; identical to SEQ ID NO: 16

SEQ ID NO: 38 - protein, 508 aa, synthetic construct; identical to SEQ ID NO: 17

SEQ ID NO: 39 - protein, 517 aa, synthetic construct; identical to SEQ ID NO: 18

SEQ ID NO: 40 - protein, 243 aa, synthetic construct; identical to SEQ ID NO: 19

SEQ ID NO: 41 - protein, 247 aa, synthetic construct; identical to SEQ ID NO: 20

SEQ ID NO: 42 - protein, 252 aa, synthetic construct; identical to SEQ ID NO: 21

SEQ ID NO: 43 - protein, 256 aa, synthetic construct; identical to SEQ ID NO: 22

SEQ ID NO: 44 - protein, 8 aa, synthetic construct
LEVLFQGP

SEQ ID NO: 45 - protein, 887 aa, synthetic construct
RAPRCRELPAQKWWHTGALYRIGDLQAFQGHGAGNLAGLKGRLDYLSSLKVKGLVLGPIHKNQKDDVAQTDLLQIDPNFGSKEDFDSLLQSAKKKSIRVILDLTPNYRGENSWFSTQVDTVATKVKDALEFWLQAGVDGFQVRDIENLKDASSFLAEWQNITKGFSEDRLLIAGTNSSDLQQILSLLESNKDLLLTSSYLSDSGSTGEHTKSLVTQYLNATGNRWCSWSLSQARLLTSFLPAQLLRLYQLMLFTLPGTPVFSYGDEIGLDAAALPGQPMEAPVMLWDESSFPDIPGAVSANMTVKGQSEDPGSLLSLFRRLSDQRSKERSLLHGDFHAFSAGPGLFSYIRHWDQNERFLVVLNFGDVGLSAGLQASDLPASASLPAKADLLLSTQPGREEGSPLELERLKLEPHEGLLLRFPYAAGGSGGGGSGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGDKTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNAYTQKSLSLSPG

SEQ ID NO: 46 - protein, 877 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPGGGGSGGGGSGGGGSGGGGSGGGGSQVQLVESGGGLVQAGGSLRLSCAASGSSISSIAMGWYRQAPGKERELVAAISSGGTINYADSVKGRFTISRDNAKNTVYLQMNSLKPEDTAVYYCNTGRRLQTGSWGQGTQVTVSSGGGGATQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQAGNAQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 47 - protein, 598 aa, synthetic construct; identical to SEQ ID NO: 2

SEQ ID NO: 48 - protein, 214 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 598 aa, synthetic construct
TAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYFLSRTNTPSGTTTQSRLQFSQAGASDIRDQSRNWLPGPCYRQQRVSKTSADNNNSEFSWTGATKYHLNGRDSLVNPGPAMASHKDDEEKFFPQSGVLIFGKQGSEKTNVDIEKVMITDEEEIRTTNPVATEQYGSVSTNLQAGNAQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRFLTRNL

SEQ ID NO: 50 - protein, 867 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPGGGGSGGGGSQVQLVESGGGLVQAGGSLRLSCAASGSSISSIAMGWYRQAPGKERELVAAISSGGTINYADSVKGRFTISRDNAKNTVYLQMNSLKPEDTAVYYCNTGRRLQTGSWGQGTQVTVSSGGGGSGGGGSTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQAGNAQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 51 - protein, 890 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPGGGGSGGGGSGGGGSGGGGSGGGGSQVQLVESGGGLVQTGGSLRLSCAASGLTFSNYAMGWFRQAPGKKREFVAHIGGSGGTWRYADSVKGRFTISRDNAKNMVYLQMNSLNTEDTAIYYCAADQRAGSYSSGWYTRSSDSLYWGQGTQVTVSSGGGGATQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQAGNAQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 52 - protein, 599 aa, Adeno-associated dependoparvovirus A
TAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 53 - protein, 877 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGGGGGSGGGGSGGGGSGGGGSGGGGSQVQLVESGGGLVQAGGSLRLSCAASGSSISSIAMGWYRQAPGKERELVAAISSGGTINYADSVKGRFTISRDNAKNTVYLQMNSLKPEDTAVYYCNTGRRLQTGSWGQGTQVTVSSGGGGAQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 54 - protein, 599 aa, Adeno-associated dependoparvovirus A; identical to SEQ ID NO: 52

SEQ ID NO: 55 - protein, 867 aa, synthetic construct
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTGGGGSGGGGSGGGGSGGGGSGGGGSQVQLVESGGGLVQAGGSLRLSCAASGSSISSIAMGWYRQAPGKERELVAAISSGGTINYADSVKGRFTISRDNAKNTVYLQMNSLKPEDTAVYYCNTGRRLQTGSWGQGTQVTVSSGGGGAGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 56 - protein, 721 aa, Adeno-associated dependoparvovirus B
VDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 57 - protein, 822 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPGGGGSGGGGSGGGGSGGGGSGSREGCASRCTKYNAELEKCEARVMSMSNTEEDCEQELEDLLHCLDHCHSQGGGGSGGGGSGGGGSGGGGSTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQAGNAQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 58 - protein, 822 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPGGGGSGGGGSGGGGSGGGGSGSREGCASRCMKYNDELEKCEARMMSMSNTEEDCEQELEDLLYCLDHCHSQGGGGSGGGGSGGGGSGGGGSTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQAGNAQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 59 - protein, 886 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPGGGGSGGGGSGGGGSGGGGSGGGGSQVQLVESGGGLVQAGGSLRLSCAASGRAFSSNGMGWFRQAPGKEREFVATISCSGTSTYYADSVKGRFTISRDNAKNTVYLQMNSLKPEDTAVYFCAADTRHCMGYTKDEGYAYWGQGTQVTVSSGGGGATQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQAGNAQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 60 - protein, 886 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPGGGGSGGGGSGGGGSGGGGSGGGGSQVHLVESGGGLVQAGGSLRLSCAASGRTISTYGMGWFRQAPGKEREFVGTISCSGTSTYYTDSVKGRFTISRDNAKNTVYLEMNSLKPEDTAVYFCAADTRHCMGYTKHGGYDYWGQGTQVTVSSGGGGATQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQAGNAQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 61 - protein, 25 aa, synthetic construct
GGGGSGGGGSGGGGSGGGGSGGGGS

SEQ ID NO: 62 - protein, 10 aa, synthetic construct
GGGGSGGGGS

SEQ ID NO: 63 - protein, 5 aa, synthetic construct
GGGGA

SEQ ID NO: 64 - protein, 20 aa, synthetic construct
GGGGSGGGGSGGGGSGGGGS

SEQ ID NO: 65 - protein, 735 aa, Adeno-associated dependoparvovirus A [sequence omitted]

SEQ ID NO: 66 - protein, 262 aa, Kutzneria albida
MHKWFLRAAVVAAVGFGLPTLIATTAQAAAVAAPTPRAPLAPPLAEDRSYRTWRVEDYVEAWERYHGREMTEDERENLARGCIGVTVVNLNREDLSNPPLNLSFGSLRTAEAVQAALNKIVDTHPSPAQYEAAVAKDPILKRLKNVVKALPSWIDSAKLKASIFSKRFYSWQNPDWSEERAHTTYRPDRETDQVDMSTYRYRARPGYVNFDYGWFDQDTNTWWHANHEEPRMVVYQSTLRHYSRPLQDFDEQVFTVAFAKKD

SEQ ID NO: 67 - protein, 116 aa, synthetic construct
QVQLVESGGGLVQAGGSLRLSCAASGSSISSIAMGWYRQAPGKERELVAAISSGGTINYADSVKGRFTISRDNAKNTVYLQMNSLKPEDTAVYYCNTGRRLQTGSWGQGTQVTVSS

SEQ ID NO: 68 - protein, 129 aa, synthetic construct
QVQLVESGGGLVQTGGSLRLSCAASGLTFSNYAMGWFRQAPGKKREFVAHIGGSGGTWRYADSVKGRFTISRDNAKNMVYLQMNSLNTEDTAIYYCAADQRAGSYSSGWYTRSSDSLYWGQGTQVTVSS

SEQ ID NO: 69 - protein, 51 aa, synthetic construct
GSREGCASRCTKYNAELEKCEARVMSMSNTEEDCEQELEDLLHCLDHCHSQ

SEQ ID NO: 70 - protein, 51 aa, synthetic construct
GSREGCASRCMKYNDELEKCEARMMSMSNTEEDCEQELEDLLYCLDHCHSQ

SEQ ID NO: 71 - protein, 125 aa, synthetic construct
QVQLVESGGGLVQAGGSLRLSCAASGRAFSSNGMGWFRQAPGKEREFVATISCSGTSTYYADSVKGRFTISRDNAKNTVYLQMNSLKPEDTAVYFCAADTRHCMGYTKDEGYAYWGQGTQVTVSS

SEQ ID NO: 72 - protein, 125 aa, synthetic construct
QVHLVESGGGLVQAGGSLRLSCAASGRTISTYGMGWFRQAPGKEREFVGTISCSGTSTYYTDSVKGRFTISRDNAKNTVYLEMNSLKPEDTAVYFCAADTRHCMGYTKHGGYDYWGQGTQVTVSS

SEQ ID NO: 73 - protein, 5 aa, Kutzneria albida
YRYRQ

SEQ ID NO: 74 - protein, 15 aa, synthetic construct
GGGGSYRYRQGGGGS

SEQ ID NO: 75 - protein, 5 aa, Kutzneria albida
RYESK

SEQ ID NO: 76 - protein, 11 aa, synthetic construct
GGGRYESKGGG

SEQ ID NO: 77 - protein, 214 aa, synthetic construct; identical to SEQ ID NO: 48

SEQ ID NO: 78 - protein, 213 aa, synthetic construct
QIVLTQSPAIMSASPGEKVTMTCSASSSIRYIHWYQQRPGTSPKRWIYDTSNLASGVPARFSGSGSGTSYSLTISSMEAEDAATYYCHQRNSYPWTFGGGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 79 - protein, 217 aa, synthetic construct
AIQLTQSPSSLSASVGDRVTITCRASQSISSYLAWYQQKPGKAPKLLIYRASTLASGVPSRFSGSGSGTDFTLTISSLQPEDFATYYCQQNYASSNVDNTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 80 - protein, 213 aa, synthetic construct; identical to SEQ ID NO: 78

SEQ ID NO: 81 - protein, 217 aa, synthetic construct; identical to SEQ ID NO: 79

SEQ ID NO: 82 - protein, 226 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 83 - protein, 226 aa, synthetic construct; identical to SEQ ID NO: 82

SEQ ID NO: 84 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 85 - protein, 214 aa, synthetic construct; identical to SEQ ID NO: 84

SEQ ID NO: 86 - protein, 254 aa, synthetic construct; identical to SEQ ID NO: 26

SEQ ID NO: 87 - protein, 254 aa, synthetic construct; identical to SEQ ID NO: 26

SEQ ID NO: 88 - protein, 275 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGCGTKVEIKGGSGGGGSGGGGSGGGGSGGEVQLVESGGGLVQPGGSLRLSCAASGFNIKDTYIHWVRQAPGKCLEWVARIYPTNGYTRYADSVKGRFTISADTSKNTAYLQMNSLRAEDTAVYYCSRWGGDGFYAMDYWGQGTLVTVSSGGGGSGGGGSGGGGRYESKGGGLEVLFQ

SEQ ID NO: 89 - protein, 284 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGCGTKVEIKGGSGGGGSGGGGSGGGGSGGEVQLVESGGGLVQPGGSLRLSCAASGFNIKDTYIHWVRQAPGKCLEWVARIYPTNGYTRYADSVKGRFTISADTSKNTAYLQMNSLRAEDTAVYYCSRWGGDGFYAMDYWGQGTLVTVSSGGGGSGGGGSGGGGSGGGGSGGGRYESKGGGLEVLFQ

SEQ ID NO: 90 - protein, 214 aa, synthetic construct; identical to SEQ ID NO: 84

SEQ ID NO: 91 - protein, 214 aa, synthetic construct; identical to SEQ ID NO: 84

SEQ ID NO: 92 - protein, 275 aa, synthetic construct; identical to SEQ ID NO: 88

SEQ ID NO: 93 - protein, 284 aa, synthetic construct; identical to SEQ ID NO: 89

SEQ ID NO: 94 - protein, 625 aa, synthetic construct
QVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEAAA

SEQ ID NO: 95 - protein, 226 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 96 - protein, 226 aa, synthetic construct; identical to SEQ ID NO: 95

SEQ ID NO: 97 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALANHATQKSLSLSPGL

SEQ ID NO: 98 - protein, 422 aa, synthetic construct
AIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSAESHLSLLYHLTAVSSPAPGTPAFWVSGWLGPQQYLSYNSLRGEAEPCGAWVWENQVSWYWEKETTDLRIKEKLFLEAFKALGGKGPYTLQGLLGCELGPDNTSVPTAKFALNGEEFMNFDLKQGTWGGDWPEALAISQRWQQQDKAANKELTFLLFSCPHRLREHLERGRGNLEWKEPPSMRLKARPSSPGFSVLTCSAFSFYPPELQLRFLRNGLAAGTGQGDFGPNSDGSFHASSSLTVKSGDEHHYCCIVQHAGLAQPLRVELESPAKSSLEHHHHHHHHHHIGLNDIFEAQKIEWHE

SEQ ID NO: 99 - protein, 226 aa, synthetic construct; identical to SEQ ID NO: 95

SEQ ID NO: 100 - protein, 226 aa, synthetic construct; identical to SEQ ID NO: 95

SEQ ID NO: 101 - protein, 227 aa, synthetic construct; identical to SEQ ID NO: 97

SEQ ID NO: 102 - protein, 485 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGCGTKVEIKGGSGGGGSGGGGSGGGGSGGEVQLVESGGGLVQPGGSLRLSCAASGFNIKDTYIHWVRQAPGKCLEWVARIYPTNGYTRYADSVKGRFTISADTSKNTAYLQMNSLRAEDTAVYYCSRWGGDGFYAMDYWGQGTLVTVSSGGSGGGGSGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 103 - protein, 485 aa, synthetic construct; identical to SEQ ID NO: 102

SEQ ID NO: 104 - protein, 393 aa, synthetic construct; identical to SEQ ID NO: 7

SEQ ID NO: 105 - protein, 234 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGSGYRYRQEPEA

SEQ ID NO: 106 - protein, 5 aa, synthetic construct
RYESK

SEQ ID NO: 107 - protein, 234 aa, synthetic construct; identical to SEQ ID NO: 105

SEQ ID NO: 108 - protein, 5 aa, synthetic construct
RYESK

SEQ ID NO: 109 - protein, 5 aa, synthetic construct
RYESK

SEQ ID NO: 110 - protein, 736 aa, synthetic construct
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVGGGGSYRYRQGGGGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQVATNNQSSTTAPATGTVNTQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 111 - protein, 736 aa, synthetic construct
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNGGGGSYRYRQGGGGSVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQVATNNQSSTTAPATGTVNTQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 112 - protein, 736 aa, synthetic construct
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQVATNNQSSGGGGSYRYRQGGGGSPATGTVNTQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 113 - protein, 588 aa, Adeno-associated dependoparvovirus A
TAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQVATNNQSSTTAPATGTVNTQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 114 - protein, 532 aa, Adeno-associated dependoparvovirus A
MSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQVATNNQSSTTAPATGTVNTQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 115 - protein, 736 aa, synthetic construct; identical to SEQ ID NO: 110

SEQ ID NO: 116 - protein, 736 aa, synthetic construct; identical to SEQ ID NO: 111

SEQ ID NO: 117 - protein, 736 aa, synthetic construct; identical to SEQ ID NO: 112

SEQ ID NO: 118 - protein, 736 aa, synthetic construct; identical to SEQ ID NO: 111

SEQ ID NO: 119 - protein, 724 aa, Adeno-associated dependoparvovirus A
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 120 - protein, 871 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTGGGGSGGGGSQVQLVESGGGLVQAGGSLRLSCAASGSSISSIAMGWYRQAPGKERELVAAISSGGTINYADSVKGRFTISRDNAKNTVYLQMNSLKPEDTAVYYCNTGRRLQTGSWGQGTQVTVSSGGGGSGGGGSQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQAGNAQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 121 - protein, 874 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGGGGSGGGGSGGGGSGGGGSGGGGSQVQLVESGGGLVQAGGSLRLSCAASGSSISSIAMGWYRQAPGKERELVAAISSGGTINYADSVKGRFTISRDNAKNTVYLQMNSLKPEDTAVYYCNTGRRLQTGSWGQGTQVTVSSGGGGALQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQAGNAQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 122 - protein, 245 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGSGLNDIFEAQKIEWHE

SEQ ID NO: 123 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 124 - protein, 237 aa, synthetic construct
GGGRYESKGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 125 - protein, 237 aa, synthetic construct
GGGRYESKGGGDKTHTCPPCPAPELLGGPSVFLFPPKPKDTLMISRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALANHATQKSLSLSPG

SEQ ID NO: 126 - protein, 237 aa, synthetic construct; identical to SEQ ID NO: 125

SEQ ID NO: 127 - protein, 513 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGCGTKLELKGGSGGGGSGGGGSGGGGSGGQVQLKQSGPGLVQPSQSLSITCTVSGFSLTNYGVHWVRQSPGKCLEWLGVIWSGGNTDYNTPFTSRLSINKDNSKSQVFFKMNSLQSNDTAIYYCARALTYYDYEFAYWGQGTLVTVSAGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGRYESKGGG

SEQ ID NO: 128 - protein, 214 aa, synthetic construct; identical to SEQ ID NO: 48

(19) United States
(12) Patent Application Publication
(10) Pub. No.: US 2026/0200989 A1
(43) Pub. Date: Jul. 16, 2026

(54) MODIFIED AAV PARTICLES

(71) Applicant: Hoffmann-La Roche Inc., Little Falls, NJ (US)

(72) Inventors: Ataurehman ALI, Starnberg (DE); Simon Thomas AUSLAENDER, Wolfratshausen (DE); Ulrich BRINKMANN, Weilheim (DE); Steffen DICKOPF, Penzberg (DE); Julia FAKHIRI, Penzberg (DE); Linda Elise FRANKEN, Basel (CH); Guy GEORGES, Habach (DE); Hubert KETTENBERGER, Muenchen (DE); Nico LAUMANN-LIPP, München (DE); Jens NIEWOEHNER, Neuried (DE); Johannes REUSCH, Taufkirchen (DE); Tilman Sebastian SCHLOTHAUER, Mountain View, CA (US); Tobias STRITTMATTER, Murnau (DE)

(73) Assignee: Hoffmann-La Roche Inc., Little Falls, NJ (US)

(21) Appl. No.: 19/299,013

(22) Filed: Aug. 13, 2025

(30) Foreign Application Priority Data
Aug. 13, 2024 (EP) 24194284.6

(51) Int. Cl. C07K 14/005; C12N 15/86

(52) U.S. Cl. CPC C07K 14/005; C12N 15/86; C07K 2319/30; C12N 2750/14122; C12N 2750/14143; C12N 2750/14151

(57) ABSTRACT

Herein is reported a novel variant adeno-associated virus (AAV) capsid protein comprising a heterologous peptide or polypeptide covalently inserted in the GH-loop of the capsid protein relative to a corresponding parental AAV capsid protein, wherein the heterologous peptide or polypeptide comprises a recognition sequence of a transglutaminase and the amino acids residues at positions 452-455 or 453-459 of VP1 of AAV-2 (SEQ ID NO: 65) or at the corresponding positions in the capsid protein of another AAV serotype are replaced by the heterologous peptide or polypeptide or inserted at amino acid position 456.

### CROSS REFERENCE TO RELATED APPLICATIONS

This application claims benefit of priority to European Application No. 24194284.6, filed Aug. 13, 2024, which is incorporated herein by reference in its entirety.

### SEQUENCE LISTING

This application contains a Sequence Listing which has been submitted electronically in XML format and is hereby incorporated by reference in its entirety. Said XML copy, created on Aug. 13, 2025, is named P39520-US-1_Sequence_Listing.xml and is 160,101 bytes in size.

### FIELD OF REFERENCE

The current invention is in the field of gene therapy. In more detail, herein are reported novel variants of wild-type AAV capsid proteins that have been modified by conjugating either enzymatically or genetically to targeting binding sites in order to change the tissue specificity of the capsid protein.

### BACKGROUND OF THE INVENTION

Adeno-associated viruses (AAVs) have a wide-ranging tropism, allowing them to infect various cell types in different tissues. While this generic specificity is beneficial for some applications, it lacks the precision needed for targeted payload delivery as in therapeutic uses. The inherent challenge lies within the AAV's natural ability to bind to common cellular receptors present on many cell types, leading to parallel infection of target and non-target cells and tissues. This indiscriminate targeting can dilute therapeutic efficacy, increase the potential for off-target effects, and elevate the risk of immune responses against the vector or the transduced cells. Consequently, enhancing the targeting and selectivity of AAV vectors by overcoming their natural tropism is one aim in the development of effective and safe gene therapies.

In the area of gene therapy, the functionalization of AAV capsids to enhance targeting and selectivity represents one strategy for re-engineering AAVs towards a desired target/tropism.

Recent advancements in the engineering of AAV vectors has accentuated their significance in the realm of gene therapy for rare diseases. By refining the specificity, and immune evasion of AAV vectors, as well as optimizing their production, the foundation for their expanded application and therapeutic outcomes could be established.

It has been reported that the tropism of AAVs can be altered by introducing cell surface receptor-binding entities, such as antibodies or fragments thereof, through genetic engineering or chemical ligations to the viral capsid's surface-exposed loops (Eichhoff et al., 2019). It has been shown that the incorporation of peptides, nanobodies, and DARPins into these loops modifies AAVs to bind to particular cell types (Buning et al., 2019; Eichhoff et al., 2019; Hamann et al., 2021; Theuerkauf et al 2023; Hoffmann et al, 2023).

The integration of peptides and nanobodies transferred monospecific binding properties to the AAV particles, whereas the introduction of designed ankyrin repeat proteins (DARPins) that target CD4 and CD32a resulted in the creation of bispecific AAV-2 vectors (Theuerkauf et al 2023).

The modification of AAV tropism through genetic fusion strategies has several limitations that impede its widespread application. Notably, the incorporation of ligands into the AAV capsid is constrained by the finite capacity of the capsid structure to accommodate exogenous sequences. Moreover, the production of ligand-targeted AAVs via genetic fusion precludes a modular strategy and necessitates bespoke re-engineering for each novel target. To date, the genetic integration of single-chain variable fragments (scFvs), antigen-binding fragments (Fabs), or entire antibodies into the surface-exposed loops of the capsid has not been achieved, due to the rather complex nature of these macromolecules.

To address these challenges, chemical ligation methods have been employed. In more detail, Puzzo et al. reported azide modified AAV particles produced by using AMBER Suppression. A stop codon (TAG) was introduced in different positions (N589, D555, A587, T456) in all three VPs. AAV particles were expressed in an amber suppression cell line, carrying a tRNA recognizing the stop codon TAG. This tRNA incorporates an unnatural amino acid (azide-lysine) into the polypeptide chain during expression. Azide modified particles were then conjugated with DBCO-aptamers via click chemistry. (Mol. Ther. Nucl. Acids 31 (2023) 383-397). Puzzo et al 2023; Pearce et al reported the functionalization of surface exposed Arg residues with 4-azidophenyl glyoxal (APGO) to obtain azide modified viral particles. Antibody fragments were functionalized with the enzyme sortase with a BCN ((bicyclo[6.1.0]non-4-yn-9-ol) to enable strain-promoted azide-alkyne cycloaddition (SPAAC) (Mol. Ther. Meth. Clin. Dev. 14 (2019) 261-269).

Click chemistry, for instance, could facilitate the attachment of ligands to the AAV surface either specifically or non-specifically. However, when utilizing non-orthogonal click reactions, precise control over the stoichiometry and site of ligand conjugation is unattainable. The introduction of non-natural amino acids amenable to click chemistry reactions offers a solution, enabling the precise and site-specific attachment of ligands to the capsid (Puzzo et al 2023). Despite the promise of this technique, it has been associated with significantly reduced viral vector yields.

Recently a novel chemical conjugation method utilizing the SpyTag/SpyCatcher system has been reported. This system involves the integration of a 13 amino acid SpyTag peptide into the AAV-9 capsid's surface-exposed loops, and the fusion of a 15 kDa SpyCatcher protein to scFvs or antibodies. Subsequent co-incubation of both components results in the spontaneous formation of an isopeptide bond, thereby covalently attaching the targeting moiety to the AAV (WO 2019/157224). Although this platform underscores the advantages of a modular system in generating retargeted AAV vectors, utilizing a 15 kDa non-human protein (SpyCatcher) as a conjugation module on a virus particle raises safety concerns regarding potential immunogenicity.

Shi and Bartlett (Mol. Ther. 7 (2003) 515-525) reported inclusion of Arg-Gly-Asp (RGD) in VP3 provides adeno-associated virus type 2 (AAV-2)-based vectors with a heparan sulfate-independent cell entry mechanism. The authors explored the modification of AAV-2 vectors to enable them to infect cells independently of heparan sulfate proteoglycan (HSPG), which is the primary attachment receptor for AAV-2. This is achieved by incorporating an RGD-containing peptide into the AAV-2 capsid.

Stachler et al. (Mol. Ther. 16 (2008) reported the site-specific modification of AAV vector particles with biophysical probes and targeting ligands using biotin ligase. This method employs the Escherichia coli enzyme biotin ligase (BirA), which ligates biotin to a 15-mer biotin acceptor peptide (BAP) that is genetically inserted into the AAV capsid proteins.

Muench et al. (Mol. Ther. 21 (2013) 109-118) reported that displaying high-affinity ligands such as DARPins on adeno-associated viral vectors enables tumor cell-specific and safe gene transfer.

Liu et al. (Small 9 (2013) 421-429) reported site-specific modification of adeno-associated viruses via a genetically engineered aldehyde tag. In this document an aldehyde-tag (13 mer, Ald13) was inserted into all 60 VPs of the capsid. During expression the Cys residue of the tag is converted to a formylglycine (FGly), catalyzed by an intra-cellular formylglycine-forming enzyme (FGE). The authors report a conversion rate of 12% (6-9 aldehyde tags have a Cys converted to FGly).

WO 2016/100735 reported a system and method for the identification and characterization of a transglutaminase. Reported are further transglutaminase enzymes for forming isopeptide bonds, methods of forming isopeptide bonds in the presence of transglutaminases, and substrate tags for use with transglutaminases.

Muik et al. (Biomat. 144 (2017) 84-94) reported the covalent coupling of high-affinity ligands to the surface of viral vector particles by protein trans-splicing mediates cell type-specific gene transfer. The authors introduced NpuDnaE Intein C (Npu C, C-terminal part of the split intein) to the N-terminus of VP2.

Kelemen et al. (Meth. Mol. Biol. 1728 (2018) 313-326) reported production and chemoselective modification of adeno-associated virus by site-specifically incorporating an unnatural amino acid residue into its capsid.

Buening and Srivastava (Mol. Ther. Meth. Clin. Dev. 12 (2019) 248-265) reported capsid modifications for targeting and improving the efficacy of AAV vectors.

Eichhoff et al. (Mol. Ther. Meth. Clin. Dev. 15 (2019) 211-220) reported nanobody-enhanced targeting of AAV gene therapy vectors.

WO 2019/157224 reported compositions and methods for delivering a therapeutic protein to the central nervous system (CNS), in order to treat diseases and disorders that impair the CNS, such as treating lysosomal storage diseases are disclosed. Therapeutic proteins delivered via a therapeutically effective amount of a nucleotide composition encoding the therapeutic protein conjugated to a cell surface receptor-binding protein that crosses the blood brain barrier (BBB) are provided.

Mevel et al. (Chem. Sci. 11 (2020) 1122) reported chemical modification of the adeno-associated virus capsid to improve gene delivery.

WO 2020/206189 reported recombinant adeno-associated viruses having capsid proteins engineered to include amino acid sequences that confer and/or enhance desired properties. In particular, the invention provides engineered capsid proteins comprising peptide insertions from heterologous proteins inserted within or near variable region IV (VR-IV) of the virus capsid, such that the insertion is surface exposed on the AAV particle. The invention also provides capsid proteins that direct rAAVps to target tissues, in particular, capsid proteins comprising peptides derived from erythropoietin or dynein that are inserted into surface-exposed variable regions and that target rAAVps to retinal tissue and/or neural tissue, including the central nervous system, and deliver therapeutics for treating neurological and/or eye disorders.

WO 2020/242984 reported modified viral particles and uses thereof. Reported are recombinant AAV viral particles comprising (i) an AAV capsid comprising AAV VP1, VP2, and VP3 capsid proteins, and (ii) packaged within the AAV capsid, a nucleic acid sequence comprising an AAV Inverted Terminal Repeat (ITR) sequence, wherein at least one of the AAV capsid proteins comprises an amino acid sequence having significant sequence identity, e.g., at least 95% identity, to the amino acid sequence of a capsid protein of a non-primate animal AAV or portion thereof, or a remote AAV or a portion thereof, and wherein one viral capsid protein further comprises a modification selected from the group consisting of (a) a first member of a protein:protein binding pair, wherein the protein:protein binding pair directs the tropism of the AAV viral particle, (b) a detectable label, (c) a point mutation, preferably wherein the point mutation reduces the natural tropism of the AAV viral particle and/or creates a detectable label, (d) a chimeric amino acid sequence, and (e) any combination of (a), (b), (c), and (d).

Hamann et al. (PLoS ONE 16 (2021) e0261269) reported improved targeting of human CD4+ T cells by nanobody-modified AAV-2 gene therapy vectors.

Yin et al. (J. Clin. Med. 10 (2021) 1323) reported preclinical characterization of the distribution, catabolismand elimination of a Polatuzumab Vedotin-Piiq (POLIVY®) antibody-drug conjugate in Sprague Dawley rats.

Leray et al. (Biomed. Pharmacother. 171 (2023) 116148) reported that novel chemical tyrosine functionalization of adeno-associated virus improved gene transfer efficiency in liver and retina.

Hoffmann et al. (Mol. Ther. Meth. Clin. Dev. 31 (2023) 101143) reported multiparametric domain insertional profiling of adeno-associated virus VP1.

WO 2023/081850 reported compositions and methods for retargeting viral particles, e.g., adeno-associated viral (AAV) particles, to muscle cells using muscle-specific surface proteins. In some embodiments, a capsid protein described herein comprises a first member comprising SpyTag operably linked to the viral capsid protein, and covalently linked to the SpyTag, a second member comprising SpyCatcher linked to a targeting ligand comprising an antibody variable domain and an IgG heavy chain domain, wherein SpyCatcher and the IgG heavy chain domain are linked via an amino acid linker, e.g., GSGESG.

WO 2023/187728 reported viral vectors comprising an engineered transgene capable of crossing the blood brain barrier and uses thereof in the treatment of diseases presenting with central nervous system manifestations, such as, but not limited to Hunter syndrome, Gaucher disease, and Sanfilippo syndrome.

WO 2023/194796 reported recombinant adeno-associated virus (AAV) capsid proteins, compositions (e.g., rAAV) comprising the capsid proteins, nucleic acids encoding the capsid proteins, and methods of making and using the capsid proteins. The recombinant AAV capsid proteins provided are engineered to comprise a peptide within a parental AAV capsid protein. In an aspect, the recombinant AAV capsid proteins provided herein are engineered to comprise a peptide within loop IV and/or loop VIII of a parental AAV capsid protein. These recombinant AAV capsid proteins demonstrate significantly increased transduction of muscle cells compared to the parental AAV capsid proteins.

Huang et al. (Science (2024) 10.1126) reported an AAV capsid reprogrammed to bind human transferrin receptors and mediating brain-wide gene delivery. (Huang et al 2024, 10.1126/science.adm8386 (2024).)

However, there is still a need for a generally applicable, modular AAV capsid modification method.

### SUMMARY OF THE INVENTION

The current invention is based, at least in part, on the finding that suitable insertion sites for heterologous peptides and polypeptides, such as, e.g., targeting ligands, in adeno-associated virus (AAV) capsid proteins are within the GH2/GH3 loop (VR IV) of AAV-2, AAV-5 and AAV-9 as well as corresponding positions in other serotypes. In more detail, it has been found that i) in AAV-2 amino acid residues S452-T455 or G453-R459 can be replaced by tags for enzymatic conjugation, nanobodies, antibody binding sites or peptide sequences or inserted at T456; ii) in AAV-5 amino acid residues N442-T444, T444-G446 or T557-A579 can be genetically replaced by tags for enzymatic conjugation, nanobodies, antibody binding sites or peptide; and iii) in AAV-9 amino acid residues S454-Q458 can be replaced by tags for enzymatic conjugation, nanobodies, antibody binding sites or peptide sequences.

Herein is reported amongst other things a novel variant AAV capsid protein comprising a heterologous peptide or polypeptide covalently inserted in the GH-loop of the capsid protein relative to a corresponding parental AAV capsid protein, wherein the heterologous peptide or polypeptide comprises a recognition sequence of a transglutaminase and, in one preferred embodiment, the amino acids residues at positions 452-455 or 453-459 of VP1 of AAV-2 (SEQ ID NO: 65) or at the corresponding positions in the respective capsid protein of another AAV serotype are replaced by the heterologous peptide or polypeptide or inserted at 456 of VP1 of AAV-2 (SEQ ID NO: 65) or at the corresponding positions in the respective capsid protein of another AAV serotype.

The current invention is based, at least in part, on the finding that the first peptide tag, a pentapeptide designated as the Q-tag (YRYRQ; SEQ ID NO: 73), can be engineered into the GH2/GH3 loop of an AAV-2 capsid. Preferably, this Q-tag is flanked C- and N-terminally a peptidic by linker sequence (GGGGSYRYRQGGGGS; SEQ ID NO: 74). The second peptide tag, a pentapeptide referred to as the K-tag (RYSEK; SEQ ID NO: 75), is fused to the target-binding moiety (GGGRYESKGGG; SEQ ID NO: 76). The KTG enzyme specifically catalyzes the formation of an isopeptide bond between the glutamine residue within the Q-tag and the lysine residue of the K-tag, resulting in the formation of a stable covalent isopeptide bond.

With the modification according to the current invention, the AAV-tropism can be modified by inserting protein domains with receptor binding abilities.

The current invention encompasses at least the following independent and dependent embodiments:
1. A variant adeno-associated virus (AAV) capsid protein comprising a heterologous peptide or polypeptide covalently inserted into or replacing a part of the GH-loop of the capsid protein relative to a corresponding parental AAV capsid protein, wherein the heterologous peptide or polypeptide comprises a recognition sequence of a transglutaminase.
1a. A variant adeno-associated virus (AAV) capsid protein comprising a heterologous peptide or polypeptide covalently inserted into or replacing a part of loop 4 or loop 8 of the capsid protein relative to a corresponding parental AAV capsid protein, wherein the heterologous peptide or polypeptide comprises a recognition sequence of a transglutaminase.
2. The variant AAV capsid protein according to any one of embodiments 1 to 1a, wherein
   a) the amino acids residues at positions 452-455 or 453-459 of VP1 of AAV-2 (SEQ ID NO: 65) or at the corresponding positions in the capsid protein of another AAV serotype are replaced by the heterologous peptide or polypeptide; or
   b) the heterologous peptide or polypeptide is inserted at amino acid position 456 of VP1 of AAV-2 (SEQ ID NO: 65) or at the corresponding positions in the capsid protein of another AAV serotype; or
   c) the amino acids residues at positions 577-579 of VP1 of AAV-5 (SEQ ID NO: 119) or at the corresponding positions in the capsid protein of another AAV serotype are replaced by the heterologous peptide or polypeptide.
3. A variant adeno-associated virus (AAV) capsid protein comprising a heterologous peptide or polypeptide covalently inserted relative to a corresponding parental AAV capsid protein, wherein
   a) the amino acids residues at positions 452-455 or 453-459 of VP1 of AAV-2 (SEQ ID NO: 65) or at the corresponding positions in the capsid protein of another AAV serotype are replaced by the heterologous peptide or polypeptide; or
   b) the heterologous peptide or polypeptide is inserted at amino acid position 456 of VP1 of AAV-2 (SEQ ID NO: 65) or at the corresponding positions in the capsid protein of another AAV serotype; or
   c) the amino acids residues at positions 577-579 of VP1 of AAV-5 (SEQ ID NO: 119) or at the corresponding positions in the capsid protein of another AAV serotype are replaced by the heterologous peptide or polypeptide.
4. The variant AAV capsid protein according to embodiment 3, wherein the heterologous peptide or polypeptide comprises a recognition sequence of a transglutaminase.
4a. The variant AAV capsid protein according to any one of embodiments 1 to 4, wherein the heterologous peptide or polypeptide has a length of at least 8 amino acid residues.
4b. The variant AAV capsid protein according to any one of embodiments 1 to 4, wherein the heterologous peptide or polypeptide has a length of at least 10 amino acid residues.
4c. The variant AAV capsid protein according to any one of embodiments 1 to 4, wherein the heterologous peptide or polypeptide has a length of at least 12 amino acid residues.
4d. The variant AAV capsid protein according to any one of embodiments 1 to 4, wherein the heterologous peptide or polypeptide has a length of at least 15 amino acid residues.
5. The variant AAV capsid protein according to any one of embodiments 1 to 4d, wherein the heterologous peptide or polypeptide comprises a recognition sequence of the microbial transglutaminase derived from Kutzneria albida (SEQ ID NO: 66).
6. The variant AAV capsid protein according to any one of embodiments 1 to 5, wherein the heterologous peptide or polypeptide comprises the amino acid sequence YRYRQ (SEQ ID NO: 73).
7. The variant AAV capsid protein according to any one of embodiments 1 to 6, wherein the heterologous peptide or polypeptide comprises
   i) the amino acid sequence YRYRQ (SEQ ID NO: 73), and
   ii) at its N-terminus and at its C-terminus independently of each other a peptidic linker of at most 10 amino acid residues.
8. The variant AAV capsid protein according to any one of embodiments 1 to 7, wherein the heterologous peptide or polypeptide comprises the amino acid sequence GGGGSYRYRQGGGGS (SEQ ID NO: 74).
9. The variant AAV capsid protein according to any one of embodiments 1 to 8, wherein the heterologous peptide or polypeptide is covalently conjugated to an antigen binding site or an Fc fragment.
10. The variant AAV capsid protein according to any one of embodiments 1 to 9, wherein the heterologous peptide or polypeptide is covalently conjugated to an antigen binding site or an Fc fragment via an isopeptide bond.
11. The variant AAV capsid protein according to any one of embodiments 1 to 2 and 4 to 8, wherein the heterologous peptide or polypeptide is covalently conjugated to an antigen binding site or an Fc fragment via an isopeptide bond that has been formed between a glutamine residue within the recognition sequence of the transglutaminase and a lysine residue of the antigen binding site or Fc fragment.
12. The variant AAV capsid according to any one of embodiments 1 to 11, wherein the heterologous peptide or polypeptide is covalently conjugated to an antigen binding site or an Fc fragment which comprises the amino acid sequence RYESK (SEQ ID NO: 75).
13. The variant AAV capsid protein according to any one of embodiments 1 to 12, wherein the heterologous peptide or polypeptide is covalently conjugated to an antigen binding site or Fc fragment which comprises the amino acid sequence GGGRYESKGGG (SEQ ID NO: 76).
14. The variant AAV capsid protein according to any one of embodiments 1 to 13, wherein the heterologous peptide or polypeptide is covalently conjugated to an antigen binding site or an Fc fragment which comprises the amino acid sequence RYESK (SEQ ID NO: 75) at its N-terminus or at its C-terminus, preferably at its C-terminus.
15. The variant AAV capsid protein according to any one of embodiments 1 to 14, wherein the heterologous peptide or polypeptide is covalently conjugated to an antigen binding site which comprises the amino acid sequence GGGRYESKGGG (SEQ ID NO: 76) at its C-terminus.
16. The variant AAV capsid protein according to embodiment 3, wherein the heterologous peptide or polypeptide is an antigen binding site.
17. The variant AAV capsid protein according to any one of embodiments 9 to 16, wherein the antigen binding site is a full-length antibody or an antibody fragment.
18. The variant AAV capsid protein according to any one of embodiments 9 to 16, wherein the antigen binding site is a nanobody, a BiTE, a diabody, a DART, a scdiabody, a diabody-CH3, a triple body, a miniantibody, a minibody, a diabody-CH3, ascDiabody-CH3, a Fab, a scFab, a Fab-scFv, a scFv-CH.CL.scFv, a F(ab′) 2, a scFv-Fc or a full-length antibody, preferably a nanobody, a scFv or a Fab.
18a. The variant AAV capsid protein according to any one of embodiments 9 to 16 and 20 to 22, wherein the antigen binding site is bispecific.
19. The variant AAV capsid protein according to any one of embodiments 9 to 18a, wherein the antigen binding site is flanked N-terminally by the amino acid sequence (GGGGS) 5 (SEQ ID NO: 61) or (GGGGS) 4 (SEQ ID NO: 64) or (GGGGS) 2 (SEQ ID NO: 62) and C-terminally by the amino acid sequence GGGGA (SEQ ID NO: 63) or (GGGGS) 2 (SEQ ID NO: 62).
20. The variant AAV capsid protein according to any one of embodiments 9 to 19, wherein the antigen binding site is flanked N-terminally by the amino acid sequence (GGGGS) 5 (SEQ ID NO: 61) and C-terminally by the amino acid sequence GGGGA (SEQ ID NO: 63).
21. The variant AAV capsid protein according to any one of embodiments 9 to 18a, wherein the antigen binding site is flanked C-terminally by the amino acid sequence (GGGGS) 4 (SEQ ID NO: 64) or (GGGGS) 2 (SEQ ID NO: 62).
22. The variant AAV capsid protein according to any one of embodiments 9 to 18a and 21, wherein the antigen binding site is flanked C-terminally by the amino acid sequence (GGGGS) 4 (SEQ ID NO: 64).
23. The variant AAV capsid protein according to any one of embodiments 9 to 18 and 21 to 22, wherein the antigen binding site is flanked C-terminally by the amino acid sequence (GGGGS) 4 (SEQ ID NO: 64) followed by the amino acid sequence RYESK (SEQ ID NO: 75). 23a.
   The variant AAV capsid protein according to any one of embodiments 9 to 14, wherein the heterologous peptide or polypeptide is covalently conjugated to a Fc fragment which comprises the amino acid sequence GGGRYESKGGG (SEQ ID NO: 76) at its N-terminus.
23b. The variant AAV capsid protein according to any one of embodiments 1 to 14 and 23a, wherein the heterologous peptide or polypeptide is covalently conjugated to a Fc fragment wherein
   a) both Fc heavy chains comprise a binding sites for human single chain FcRn;
   b) one Fc heavy chain comprise a binding site for human single chain FcRn and the other Fc heavy chain demonstrates abolished binding to human single chain FcRn; or
   c) both Fc heavy chains demonstrates abolished binding to human single chain FcRn.
23c. The variant AAV capsid protein according to any one of embodiments 1 to 14 and 23a to 23b, wherein the heterologous peptide or polypeptide is covalently conjugated to an Fc fragment wherein
   a) neither Fc heavy chain comprise a mutation abolishing binding to human single chain FcRn;
   b) one Fc heavy chain does not comprise a mutation abolishing binding to human single chain FcRn and the other Fc heavy chain comprises the mutations H310A, H433A and Y436A; or
   c) both Fc heavy chains comprise the mutations H310A, H433A and Y436A.
23d. The variant AAV capsid protein according to any one of embodiments 1 to 14 and 23a to 23c, wherein the heterologous peptide or polypeptide is covalently conjugated to a Fc fragment wherein
   a) the Fc fragment comprises the sequences of SEQ ID NO: 95 and SEQ ID NO: 124;
   b) the Fc fragment comprises the sequences of SEQ ID NO: 96 and SEQ ID NO: 125; or
   c) the Fc fragment comprises the sequences of SEQ ID NO: 97 and SEQ ID NO: 126.
24. The variant AAV capsid protein according to any one of embodiments 1 to 23d, wherein the variant AAV capsid protein is a variant AAV-2 capsid protein or a variant AAV-5 capsid protein or a variant AAV-9 capsid protein or a variant chimeric AAV-2/9 capsid protein.
25. The variant AAV capsid protein according to any one of embodiments 1 to 24, wherein the variant AAV capsid protein is a variant AAV-2 capsid protein wherein
   a) amino acid residues S452-T455 or G453-R459 are replaced by the heterologous peptide or polypeptide; or
   b) the heterologous peptide or polypeptide is inserted at amino acid residue T456.
26. The variant AAV capsid protein according to any one of embodiments 1 to 24, wherein the variant AAV capsid protein is a variant AAV-5 capsid protein wherein
   a) amino acid residues N442-T444 or T444-G446 are replaced by the heterologous peptide or polypeptide; or
   b) amino acid residues T557-A579 are replaced by the heterologous peptide or polypeptide.
27. The variant AAV capsid protein according to any one of embodiments 1 to 24, wherein the variant AAV capsid protein is a variant AAV-9 capsid protein wherein amino acid residues S454-Q458 are replaced by the heterologous peptide or polypeptide.
28. The variant AAV capsid protein according to any one of embodiments 1 to 27, wherein variant AAV capsid protein does not bind to/is silent with respect to binding to HSPG.
29. The variant AAV capsid protein according to any one of embodiments 1 to 28, wherein the amino acids residues at positions 585 and 588 of VP1 of AAV-2 (SEQ ID NO: 65) or at the corresponding positions in the capsid protein of another AAV serotype are alanine.
30. The variant AAV capsid protein according to any one of embodiments 1 to 29, wherein the variant capsid protein is a variant AAV-2 capsid protein with the mutations R585A and R588A.
30a. The variant AAV capsid protein according to embodiment 26, wherein the variant AAV capsid protein does not bind to/is silent with respect to binding to natural sialic acid and sulfoglycan.
30b. The variant AAV capsid protein according to any one of embodiments 26 and 30a, wherein the variant AAV capsid protein comprises the mutations M569V, Y585V and L587T.
30c. The variant AAV capsid protein according to any one of embodiments 1 to 30 and 33 to 35, wherein the variant capsid protein is a variant AAV-2 capsid protein with the mutations Y444F, Y500F and Y730F.
31. The variant AAV capsid protein according to any one of embodiments 9 to 30c, wherein the antigen binding site is conjugated to the heterologous peptide or polypeptide by the amino acid sequence (GGGGS) 4 (SEQ ID NO: 64) or (GGGGS) 2 (SEQ ID NO: 62).
32. The variant AAV capsid protein according to any one of embodiments 9 to 31, wherein the antigen binding site is conjugated to the heterologous peptide or polypeptide by the amino acid sequence (GGGGS) 4 (SEQ ID NO: 64).
33. The variant AAV capsid protein according to any one of embodiments 9 to 32, wherein the antigen binding site specifically binds to human transferrin receptor (huTfR), human epidermal growth factor receptor 2 (HER2), human endothelial growth factor receptor (huEGFR) and/or human CD98 (huCD98).
34. The variant AAV capsid protein according to any one of embodiments 9 to 33, wherein the antigen binding site is a Fab specifically binding to huTfR and is conjugated to the heterologous peptide or polypeptide by the amino acid sequence (GGGGS) 4 (SEQ ID NO: 64).
35. The variant AAV capsid protein according to embodiment 34, wherein the antigen binding site is a Fab specifically binding to huTfR with low affinity and is conjugated to the heterologous peptide or polypeptide by the amino acid sequence (GGGGS) 4 (SEQ ID NO: 64).
36. The variant AAV capsid protein according to any one of embodiments 9 to 33, wherein the antigen binding site is a Fab specifically binding to huEGFR and is conjugated to the heterologous peptide or polypeptide by the amino acid sequence (GGGGS) 4 (SEQ ID NO: 64).
36a. The variant AAV capsid protein according to any one of embodiments 9 to 33, wherein the antigen binding site is a Fab specifically binding to huEGFR and is conjugated to the heterologous peptide or polypeptide by the amino acid sequence (GGGGS) 2 (SEQ ID NO: 62).
36b. The variant AAV capsid protein according to any one of embodiments 9 to 33, wherein the antigen binding site is a scFv specifically binding to huEGFR and is conjugated to the heterologous peptide or polypeptide by the amino acid sequence (GGGGS) 4 (SEQ ID NO: 64).
36c. The variant AAV capsid protein according to any one of embodiments 9 to 33, wherein the antigen binding site is a scFv specifically binding to EGFR and is conjugated to the heterologous peptide or polypeptide by the amino acid sequence (GGGGS) 2 (SEQ ID NO: 62).
36d. The variant AAV capsid protein according to any one of embodiments 9 to 33, wherein the antigen binding site is a Fab specifically binding to huHer2 and is conjugated to the heterologous peptide or polypeptide by the amino acid sequence (GGGGS) 4 (SEQ ID NO: 64).
36e. The variant AAV capsid protein according to any one of embodiments 9 to 33, wherein the antigen binding site is a Fab specifically binding to Her2 and is conjugated to the heterologous peptide or polypeptide by the amino acid sequence (GGGGS) 2 (SEQ ID NO: 62).
36f. The variant AAV capsid protein according to any one of embodiments 9 to 33, wherein the antigen binding site is a scFv specifically binding to Her2 and is conjugated to the heterologous peptide or polypeptide by the amino acid sequence (GGGGS) 4 (SEQ ID NO: 64).
36g. The variant AAV capsid protein according to any one of embodiments 9 to 33, wherein the antigen binding site comprises a scFv specifically binding to Her2 and a scFv specifically binding to EGFR and is conjugated to the heterologous peptide or polypeptide by the amino acid sequence (GGGGS) 2 (SEQ ID NO: 62).
37. The variant AAV capsid protein according to any one of embodiments 9 to 33, wherein the antigen binding site is a nanobody specifically binding to huCD98 and is replacing residues 452-455 of the parental capsid protein.
38. The variant AAV capsid protein according to any one of embodiments 9 to 37, wherein the KD value of the conjugated binding site is about the same as the KD value of the non-conjugated binding site.
39. A recombinant adeno-associated virus particle (rAAVp) comprising the variant AAV capsid protein according to any one of embodiments 1 to 38.
40. The rAAVp according to embodiment 39, wherein the rAAVp comprises about 3 to 8 copies of the variant AAV capsid proteins according to any one of embodiments 1 to 38.
41. A rAAVp comprising the variant AAV capsid protein according to any one of embodiments 1 to 8 and 16-30 and 33-38.
42. A method for producing a rAAVp according to any one of embodiments 39 to 41, comprising the step of
   incubating a mixture comprising the rAAVp according to embodiment 40 with an antigen binding site or a Fc fragment in the presence of the microbial transglutaminase derived from Kutzneria albida (KTG) (SEQ ID NO: 66) at pH 7.4 and about 37° C. for about 15 hours, whereby the rAAVp is present in the mixture at a concentration of about 150 nM to 300 nM, the transglutaminase is present in the mixture at a concentration of about 50 nM to 200 nM and the concentration of the binding site or Fc fragment is about 15 μM.
43. The method according to embodiment 42, wherein the rAAVp is present in the mixture at a concentration of about 300 nM (corresponding to 1.8E+14 vp/mL).
44. The method according to any one of embodiments 42 to 43, wherein the KTG is present in the mixture at a concentration of about 50 nM.
45. The method according to any one of embodiments 42 to 44, wherein the antigen binding site or Fc fragment comprises the amino acid sequence RYESK (SEQ ID NO: 75).
46. The method according to any one of embodiments 42 to 45, wherein the antigen binding site or Fc fragment comprises the amino acid sequence GGGRYESKGGG (SEQ ID NO: 76).
47. The method according to any one of embodiments 42 to 46, wherein the antigen binding site comprises the amino acid sequence RYESK (SEQ ID NO: 75) at its C-terminus.
48. The method according to any one of embodiments 42 to 47, wherein the antigen binding site comprises the amino acid sequence GGGRYESKGGG (SEQ ID NO: 76) at its C-terminus.
48a. The method according to any one of embodiments 42 to 46, wherein the Fc fragment comprises the amino acid sequence RYESK (SEQ ID NO: 75) at its N-terminus.
48b. The method according to any one of embodiments 42 to 46 and 48a, wherein the Fc fragment comprises the amino acid sequence GGGRYESKGGG (SEQ ID NO: 76) at its N-terminus.
48c. The method according to any one of embodiments 42 to 46 and 48a to 48b, wherein the heterologous peptide or polypeptide is covalently conjugated to a Fc fragment wherein
   a) both Fc heavy chains comprise a binding sites for human single chain FcRn;
   b) one Fc heavy chain comprise a binding site for human single chain FcRn and the other Fc heavy chain demonstrates abolished binding to human single chain FcRn; or
   c) both Fc heavy chains demonstrates abolished binding to human single chain FcRn.
48d. The method according to any one of embodiments 42 to 46 and 48a to 48c, wherein the heterologous peptide or polypeptide is covalently conjugated to an Fc fragment wherein
   a) neither Fc heavy chain comprise a mutation abolishing binding to human single chain FcRn;
   b) one Fc heavy chain does not comprise a mutation abolishing binding to human single chain FcRn and the other Fc heavy chain comprises the mutations H310A, H433A and Y436A; or
   c) both Fc heavy chains comprise the mutations H310A, H433A and Y436A.
48e. The method according to any one of embodiments 42 to 46 and 48a to 48d, wherein the heterologous peptide or polypeptide is covalently conjugated to a Fc fragment wherein
   a) the Fc fragment comprises the sequences of SEQ ID NO: 95 and SEQ ID NO: 124;
   b) the Fc fragment comprises the sequences of SEQ ID NO: 96 and SEQ ID NO: 125; or
   c) the Fc fragment comprises the sequences of SEQ ID NO: 97 and SEQ ID NO: 126.
49. The method according to any one of embodiments 42 to 48, wherein the antigen binding site is a full-length antibody or an antibody fragment.
50. The method according to any one of embodiments 42 to 49, wherein the antigen binding site is a nanobody, a BiTE, a diabody, a DART, a scdiabody, a Diabody-CH3, a triple body, a miniantibody, a minibody, a diabody-CH3, ascDiabody-CH3, a Fab, a scFab, a Fab-scFv, a scFv-CH.CL.scFv, a F(ab′)2, a scFv or a full-length antibody, preferably a nanobody, a scFv or a Fab.
51. The method according to any one of embodiments 42 to 50, wherein the antigen binding site is flanked N-terminally by the amino acid sequence (GGGGS) 5 (SEQ ID NO: 61) or (GGGGS) 4 (SEQ ID NO: 64) or (GGGGS) 2 (SEQ ID NO: 62) and C-terminally by the amino acid sequence GGGGA (SEQ ID NO: 63) or (GGGGS) 2 (SEQ ID NO: 62).
52. The method according to any one of embodiments 42 to 51, wherein the antigen binding site is flanked N-terminally by the amino acid sequence (GGGGS) 5 (SEQ ID NO: 61) and C-terminally by the amino acid sequence GGGGA (SEQ ID NO: 63).
53. The method according to any one of embodiments 42 to 52, wherein the rAAVp is a variant recombinant AAV-2 particle or a variant recombinant AAV-5 particle or a variant recombinant AAV-9 particle or a variant chimeric recombinant AAV-2/9 particle.
54. The method according to any one of embodiments 42 to 53, wherein the rAAVp is a variant recombinant AAV-2 particle wherein
   a) amino acid residues S452-T455 or G453-R459 are replaced by the heterologous peptide or polypeptide; or
   b) the heterologous peptide or polypeptide is inserted at amino acid position T456.
55. The method according to any one of embodiments 42 to 53, wherein the rAAVp is a variant recombinant AAV-5 particle wherein
   a) amino acid residues T444-G446 or N442-T444 are replaced by the heterologous peptide or polypeptide; or
   b) amino acid residues T557-A579 are replaced by the heterologous peptide or polypeptide.
56. The method according to any one of embodiments 42 to 53, wherein the rAAVp is a variant recombinant AAV-9 particle wherein amino acid residues S454-Q458 are replaced.
57. The method according to any one of embodiments 42 to 56, wherein the in the VP1 of the rAAVp the amino acids residues at positions 585 and 588 of VP1 of AAV-2 (SEQ ID NO: 65) or at the corresponding positions in the capsid protein of another AAV serotype are alanine.
58. The method according to any one of embodiments 42 to 54, wherein the rAAVp is a variant recombinant AAV-2 particle with the mutations R585A and R588A.
59. The method according to any one of embodiments 47 to 58, wherein the antigen binding site comprises the amino acid sequence (GGGGS) 4 (SEQ ID NO: 64) or (GGGGS) 2 (SEQ ID NO: 62) N-terminal to the C-terminal amino acid sequence.
60. The method according to any one of embodiments 47 to 59, wherein the antigen binding site comprises the amino acid sequence (GGGGS) 4 (SEQ ID NO: 64) N-terminal to the C-terminal amino acid sequence.
61. The method according to any one of embodiments 42 to 60, wherein the antigen binding site specifically binds to the human transferrin receptor (huTfR), human endothelial growth factor receptor (huEGFR), human epidermal growth factor receptor 2 (HER2) and/or human CD98 (huCD98).
62. The method according to any one of embodiments 42 to 61, wherein the antigen binding site is a Fab specifically binding to huTfR and is conjugated to the N- or C-terminal, preferably the C-terminal, amino acid sequence by the amino acid sequence (GGGGS) 4 (SEQ ID NO: 64).
63. The method according to embodiment 62, wherein the antigen binding site is a Fab specifically binding to huTfR with low affinity and is conjugated to the N- or C-terminal, preferably the C-terminal, amino acid sequence by the amino acid sequence (GGGGS) 4 (SEQ ID NO: 64).
64. The method according to any one of embodiments 42 to 61, wherein the antigen binding site is a scFv specifically binding to huTfR and is conjugated to the N- or C-terminal, preferably the C-terminal, amino acid sequence by the amino acid sequence (GGGGS) 4 (SEQ ID NO: 64).
65. The method according to embodiment 64, wherein the antigen binding site is a scFv specifically binding to huTfR with low affinity and is conjugated to the N- or C-terminal, preferably the C-terminal, amino acid sequence by the amino acid sequence (GGGGS) 4 (SEQ ID NO: 64).
66. The method according to any one of embodiments 42 to 61, wherein the antigen binding site is a Fab specifically binding to huEGFR and is conjugated to N- or C-terminal, preferably the C-terminal, amino acid sequence by the amino acid sequence (GGGGS) 4 (SEQ ID NO: 64).
66a. The method according to any one of embodiments 42 to 61, wherein the antigen binding site is a scFv specifically binding to huEGFR and is conjugated to N- or C-terminal, preferably the C-terminal, amino acid sequence by the amino acid sequence (GGGGS) 4 (SEQ ID NO: 64).
67. The method according to any one of embodiments 42 to 61, wherein the antigen binding site is a Fab specifically binding to HER2 and is conjugated to N- or C-terminal, preferably C-terminal, amino acid sequence by the amino acid sequence (GGGGS) 4 (SEQ ID NO: 64).
68. The method according to any one of embodiments 42 to 61, wherein the antigen binding site is a scFv specifically binding to HER2 and is conjugated to N- or C-terminal, preferably C-terminal, amino acid sequence by the amino acid sequence (GGGGS) 4 (SEQ ID NO: 64).
68a. The method according to any one of embodiments 42 to 61, wherein the antigen binding site comprises a scFv specifically binding to HER2 and a scFv specifically binding to EGFR and is conjugated to N- or C-terminal, preferably C-terminal, amino acid sequence by the amino acid sequence (GGGGS) 4 (SEQ ID NO: 64).
69. A nucleic acid encoding the variant AAV capsid protein according to any one of embodiments 1 to 8 and 16-30 and 33-38.
70. A cell comprising the nucleic acid according to embodiment 69.
71. The cell according to embodiment 70, wherein the nucleic acid encoding the variant AAV capsid protein comprises a mutated splice acceptor site (sa; CCGGA→CCAGA).
72. The cell according to any one of embodiments 70 to 71, wherein the cell further comprises a second nucleic acid encoding wild-type AAV capsid proteins VP2 and VP3 with a mutated start codon (sc; ATG→AAG) for the VP1 coding region.
73. A method for producing a rAAVp comprising the variant AAV capsid protein according to any one of embodiments 1 to 8 and 16-30 and 33-38 comprising the steps of
   a) cultivating a cell according to any one of embodiments 70 to 72 in a medium suitable for the production of the rAAVp and under conditions suitable for the production of the rAAVp,
   b) recovering the rAAVp from the cell and the cultivation medium, and
   c) optionally purifying the rAAVp obtained in step b) with one or more chromatography steps,
   and thereby producing the rAAVp.

In addition to the various embodiments depicted and claimed, the disclosed subject matter is also directed to other embodiments having other combinations of the features disclosed or claimed herein. As such, the particular features presented herein can be combined with each other in other manners within the scope of the disclosed subject matter such that the disclosed subject matter includes any suitable combination of the features disclosed herein. The foregoing description of specific embodiments of the disclosed subject matter has been presented for purposes of illustration and description. It is not intended to be exhaustive or to limit the disclosed subject matter to those embodiments disclosed.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1: Crystal structure of the AAV-2-VP1 capsid protein displayed as ribbon structure (blue). Within the GH2/GH3 loop, amino acids S452-G453-T454-T455 (orange) are deleted and replaced with GGGGSYRYRQGGGGS (SEQ ID NO: 74). (PDB ID: 1LP3, https://doi.org/10.1073/pnas.162250899).

FIG. 2: VP1, VP2 VP3 in ratio 1:1:10 build the viral capsid. The Q-tag, a short peptide sequence (YRYRQ; SEQ ID NO: 73), flanked by a G4S linker, was inserted into the GH2/GH3 loop and replaced the residues S452-T455. Additionally, mutation R585A and R588A were introduced to silence the AAV-2 natural HSPG primary receptor binding site. The plasmid that encodes for VP1-Q-tag variant capsid protein is further mutated by inactivating the splice acceptor (sa) site CCGGA→CCAGA that prevents VP2 and VP3 production. VP2 and VP3 expression was guaranteed using a second rep/cap plasmid, where the start codon (sc) for VP1 was mutated thereby inactivated (ATG→AAG). (see, e.g., Eichhoff et al 2019 https://doi.org/10.1016/j.omtm.2019.09.003).

FIG. 3: Western Blot of AAV-2-Q-tag variant particles and different K-tagged nanobody constructs enzymatically conjugated via KTG. Lane 1: Negative control (AAV-2-Q-tag variant particle only, SEQ ID NO: 1, 2, 3), Lane 2: K-tag-VNAR (SEQ ID NO: 11), Lane 3: K-tag-MH12 (SEQ ID NO: 12), Lane 4: VNAR-K-tag (SEQ ID NO: 13), Lane 5: MH12-K-tag (SEQ ID NO: 14).

FIG. 4: Western Blot of the conjugation of AAV-2-Q-tag variant particle with MH12-K-tag using KTG. VP3 appears at MW of 65 kDa, VP2 at 83 kDa, VP1 at 104 kDa, and VP1-Nanobody (SEQ ID NO: 1, 2, 3, 14) at 126 kDa. AAV-2-Q-tag variant particle concentrations: Lane 1:453 nM, Lane 2:400 nM, Lane 3:350 nM, Lane 4:300 nM, Lane 5:250 nM, Lane 6:200 nM, Lane 7:150 nM, Lane 8:100 nM. Expected bands (kDa): VP1: 103/VP2: 83/VP3: 65/VP1-Nanobody: 127.

FIG. 5: Western Blot of the reaction product obtained with different enzyme concentrations visualized with anti-AAV VP1/VP2/VP3 rabbit polyclonal antibody dilution 1:20.

FIG. 6: Western Blot of the purified enzymatic conjugation product of AAV-2-Q-tag variant particle and nanobody MH12-K-tag. Lane 1: AAV-2-Q-tag variant particle (SEQ ID NO: 1, 2, 3)+KTG without nanobody, Lane 2: AAV-2-Q-tag variant particle (SEQ ID NO: 1, 2, 3)+KTG+MH12-K-tag (SEQ ID NO: 14), Lane 3: AAV-2-Q-tag variant particle (SEQ ID NO: 1, 2, 3)+MH12-K-tag (SEQ ID NO: 14) without KTG. Conjugation can only be observed in the presence of KTG, the AAV-Q-tag variant particle and nanobody-K-tag.

FIG. 7: SPR sensorgram of (dimeric) TfR ectodomain injections in five concentrations with highest concentration of 1000 nM applying a two-fold dilution over AAV-2-MH12 conjugate.

FIG. 8: SPR sensorgram of (dimeric) TfR ectodomain injections in five concentrations with highest concentration of 1000 nM applying a two-fold dilution over AAV-2-Q-tag variant particle/nanobody mixture (negative control 1).

FIG. 9: SPR sensorgram of (dimeric) TfR ectodomain injections in five concentrations with highest concentration of 1000 nM applying a two-fold dilution over AAV-2-Q-tag variant particle+KTG mixture (negative control 2).

FIG. 10: HeLa wild-type cells were seeded at a density of 5E+3 cell/well in 100 μL appropriate medium in 96-well format. After 5 h, cells were transduced with MOI series (5E+5-7.81E+3) of AAV-2-MH12 (SEQ ID NO: 1, 2, 3, 14)/AAV-2-Q-tag variant particle (SEQ ID NO: 1, 2, 3) at 37° C. After 72 h cells were detached with Accutase (StemPro™ Accutase™ Cell Dissociation Reagent, Gibco, A1110501) and transgene expression (transgene: mGL) was analyzed via FACS.

FIG. 11: HeLa TfR KO cells were seeded at a density of 5E+4 cell/well in 100 μL appropriate medium in 96-well format. After 5 h, cells were transduced with MOI series (5E+5-7.81E+3) of AAV-2-MH12 (SEQ ID NO: 1, 2, 3, 14)/AAV-2-Q-tag variant particle (SEQ ID NO: 1, 2, 3) at 37° C. After 72 h cells were detached with Accutase (StemPro™ Accutase™ Cell Dissociation Reagent, Gibco, A1110501) and transgene expression (transgene: mGL) was analyzed via FACS.

FIG. 12: Western Blot of AAV-2-Fab conjugation reaction for different conjugation conditions detecting VP1, 2, 3 and VP1-Fab via anti-VP1-2-3 antibody; the lanes correspond to the numbers in Table 3.

FIG. 13: SPR sensorgram of (dimeric) TfR ectodomain injections in five concentrations with highest concentration of 1000 nM applying a three-fold dilution over AAV-2-Q-tag variant particle (negative control).

FIG. 14: SPR sensorgram of (dimeric) TfR ectodomain injections in five concentrations with highest concentration of 1000 nM applying a three-fold dilution over AAV-2-aTfR-Fab-2G4S (high affinity) conjugate.

FIG. 15: SPR sensorgram of (dimeric) TfR ectodomain injections in five concentrations with highest concentration of 1000 nM applying a three-fold dilution over AAV-2-aTfR-Fab-4G4S (high affinity) conjugate.

FIG. 16: SPR sensorgram of (dimeric) TfR ectodomain injections in five concentrations with highest concentration of 1000 nM applying a three-fold dilution over AAV-2-aTfR-Fab-2G4S (low affinity) conjugate.

FIG. 17: SPR sensorgram of (dimeric) TfR ectodomain injections in five concentrations with highest concentration of 1000 nM applying a three-fold dilution over AAV-2-aTfR-Fab-4G4S (low affinity) conjugate.

FIG. 18: HeLa wild-type cells were seeded at a density of 5E+3 cell/well in 100 μL appropriate medium in 96-well format. After 5 h, cells were transduced with MOI series (5E+5-7.81E+3) of AAV-2-Fab with HSPG-mut (silenced). After 72 h, cells were analyzed using FACS.

FIG. 19: HeLa TfR KO cells were seeded at a density of 5E+3 cell/well in 100 μL appropriate medium in 96-well format. After 5 h, cells were transduced with MOI series (5E+5-7.81E+3) of AAV-2-Fab with HSPG-mut (silenced). After 72 h, cells were analyzed using FACS.

FIG. 20: Western Blot of AAV-2-Fab conjugation reaction for different conjugation conditions detecting VP1, 2, 3 and VP1-Fab/scFv via anti-VP1-2-3 antibody; the lanes correspond to the numbers in Table 5.

FIG. 21: SPR sensorgram of recombinant EGFR injections in five concentrations with highest concentration of 1,500 nM applying a three-fold dilution over AAV-2-Q-tag variant particle (negative control).

FIG. 22: SPR sensorgram of recombinant EGFR injections in five concentrations with highest concentration of 1,500 nM applying a three-fold dilution over AAV-2-aEGFR-Fab-2G4S.

FIG. 23: SPR sensorgram of recombinant EGFR injections in five concentrations with highest concentration of 1,500 nM applying a three-fold dilution over AAV-2-aEGFR-Fab-4G4S.

FIG. 24: SPR sensorgram of recombinant EGFR injections in five concentrations with highest concentration of 1,500 nM applying a three-fold dilution over AAV-2-aEGFR-scFv-2G4S.

FIG. 25: SPR sensorgram of recombinant EGFR injections in five concentrations with highest concentration of 1,500 nM applying a three-fold dilution over AAV-2-aEGFR-scFv-4G4S.

FIG. 26: HCC827 (EGFR positive) cells were seeded at a density of 5E+3 cell/well in 100 μL appropriate medium in 96-well format. After 5 h, cells were transduced with MOI series (5E+5-7.81E+3) of AAV-2-Fab/scFv with HSPG-mut (silenced). After 72 h, cells were analyzed using FACS.

FIG. 27: NCI-H446 (EGFR negative) cells were seeded at a density of 5E+3 cell/well in 100 μL appropriate medium in 96-well format. After 5 h, cells were transduced with MOI series (5E+5-7.81E+3) of AAV-2-Fab/scFv with HSPG-mut (silenced). After 72 h, cells were analyzed using FACS.

FIG. 28: Western Blot of AAV2-Qtag and different K-tagged scFv (SEQ ID NO: 25, 84 and SEQ ID NO: 26, 85) and Fab Fragments (SEQ ID NO: 27 and SEQ ID NO: 28) enzymatically conjugated via KTG. VP3 appears at MW of 65 kDa, VP2 at 83 kDa, VP1 at 104 kDa, VP1-Fab (SEQ ID NO: 1, 2, 3, 25, 84, SEQ ID NO: 1, 2, 3, 26, 85) and VP1-ScFv (SEQ ID NO: 1, 2, 3, 27, SEQ ID NO: 1, 2, 3, 28) as additional band. Different Linker lengths 2 (G4S) or 4 (G4S) between Fab or scFv and C-terminal K-Tag were tested.

FIGS. 29-32: SPR Binding Kinetics of human Her2 injections (SEQ ID NO: 94) over captured AAV2-Qtag-aHer2-Fab (SEQ ID NO: 1, 2, 3, 25, 84, SEQ ID NO: 1, 2, 3, 26, 85) or AAV2-Qtag-aHer2-scFv (SEQ ID NO: 1, 2, 3, 27, SEQ ID NO: 1, 2, 3, 28).

FIG. 29: SPR sensorgram of recombinant Her2 injections in five concentrations with highest concentration of 100 nM applying a two-fold dilution series over AAV2-Q-aHER2-Fab-2G4S-Ktag.

FIG. 30: sensorgram of recombinant Her2 injections in five SPR concentrations with highest concentration of 100 nM applying a two-fold dilution series over AAV2-Q-aHER2-Fab-4G4S-Ktag.

FIG. 31: SPR sensorgram of recombinant Her2 injections in five concentrations with highest concentration of 100 nM applying a two-fold dilution series over AAV2-Q-aHER2-scFv-2G4S-Ktag.

FIG. 32: SPR sensorgram of recombinant Her2 injections in five concentrations with highest concentration of 100 nM applying a two-fold dilution series over AAV2-Q-aHER2-scFv-4G4S-Ktag.

FIG. 33: Transduction of Her2 positive cell line with AAV2-Qtag-aHer2-Fab (SEQ ID NO: 1, 2, 3, 25, 84, SEQ ID NO: 1, 2, 3, 26, 85) or AAV2-Qtag-aHer2-scFv (SEQ ID NO: 1, 2, 3, 27, SEQ ID NO: 1, 2, 3, 28) applying different MOIs.

FIG. 34: Transduction of Her2 negative cell line with AAV2-Qtag-aHer2-Fab (SEQ ID NO: 1, 2, 3, 25, 84, SEQ ID NO: 1, 2, 3, 26, 85) or AAV2-Qtag-aHer2-scFv (SEQ ID NO: 1, 2, 3, 27, SEQ ID NO: 1, 2, 3, 28) applying different MOIs.

FIG. 35: Western Blot of AAV2-Qtag and different K-tagged antibody Fc fragments (SEQ ID NO: 1, 2, 3, 95, 124, SEQ ID NO: 1, 2, 3, 96, 125, SEQ ID NO: 1, 2, 3, 97, 126) enzymatically conjugated via KTG. VP3 appears at MW of 65 kDa, VP2 at 83 kDa, VP1 at 104 kDa, VP1-Fc (SEQ ID NO: 1, 2, 3, 95, 124, SEQ ID NO: 1, 2, 3, 96, 125, SEQ ID NO: 1, 2, 3, 97, 126) appears as an additional band.

FIG. 36: AAV2-Qtag-Fc-only-K-WT (SEQ ID NO: 1, 2, 3, 95, 124) capture levels. AAV-binding site conjugate capture levels (shown in Response Units, RU) are adjusted to similar levels for all constructs to approx. 4000 RU.

FIG. 37: AAV2-Qtag-Fc-only-K-WT-AAA (SEQ ID NO: 1, 2, 3, 96, 125) capture level. AAV-binding site conjugate capture levels (shown in Response Units, RU) are adjusted to similar levels for all constructs to approx. 4000 RU.

FIG. 38: AAV2-Qtag-Fc-only-K-AAA (SEQ ID NO: 1, 2, 3, 97, 126) capture level.

FIG. 39: SPR Kinetics hsc-FcRn (Sed ID; 98) injection over captured AAV2-Qtag-Fc-only-K-WT (SEQ ID NO: 1, 2, 3, 95, 124).

FIG. 40: SPR Kinetics hsc-FcRn (Sed ID; 98) injection over captured AAV2-Qtag-Fc-only-K-WT-3A (SEQ ID NO: 1, 2, 3, 96, 125).

FIG. 41: SPR Kinetics hsc-FcRn (Sed ID; 98) injection over captured AAV2-Qtag-Fc-only-K-3A (SEQ ID NO: 1, 2, 3, 97, 126).

FIG. 42: Western Blot of AAV2-Qtag-aEGFR-aHer2-scFv-Fc-K (SEQ ID NO: 1, 2, 3, 102, 127) enzymatically conjugated via KTG. VP3 appears at MW of 65 kDa, VP2 at 83 kDa, VP1 at 104 kDa, VP1-EGFR-aHer2-scFv-Fc (SEQ ID NO: 1, 2, 3, 102, 127103) as an additional band. Unmodified AAV2-Qtag (SEQ ID NO: 1, 2, 3) only shows viral proteins VP1, 2, 3.

FIG. 43: SPR Binding Kinetics AAV2 Qtag (SEQ ID NO: 1, 2, 3) 100 nM EGFR and 300 nM Her2 injection.

FIG. 44: SPR Binding Kinetics AAV2-Qtag-aEGFR-aHer2-scFv-Fc-K (SEQ ID NO: 1, 2, 3, 102, 127) 100 nM EGFR and 300 nM Her2 injection.

FIG. 45: DBCO-Azide click reaction to modify AAV capsids. Two separate reactions are performed.
1) Modification of AAV-Q-tag variant particle particles with K-tag-N₃.
2) Modification of binding site with a DBCO functional group.
In a second step, both modified compounds are incubated to covalently attach the binding site to the capsid.

FIG. 46: Western Blot of click reaction between azide modified AAV-2-Qtag and DBCO modified aEGFR-Fab-GSG. VP1, 2, 3 were detected with anti-VP1-2-3 antibody.

FIG. 47: Transduction assay of AAV-2-aEGFR-Fab-GSG generated via click chemistry. SEQ ID NO: 1, 2, 3, 105, 128 was incubated on EGFR displaying HCC_827 cells at three different MOIs for 72 h at 37° C.

FIG. 48: Transduction assay of AAV-2-aEGFR-Fab-GSG generated via click chemistry. SEQ ID NO: 1, 2, 3, 105, 128 was incubated on EGFR negative NCL_446 cells at three different MOIs for 72 h at 37° C.

FIG. 49: Western blot of AAV-5-Qtag conjugation. Anti-AAV VP1 antibody used to detect VP1 and VP1-Fab.

FIG. 50: Viral genome (vg) titer of genetic AAV-Nb and -peptide variant particles. vg titer was determined by dPCR.

FIG. 51: Total yield of genetic AAV-Nb and -peptide variant particles. Total yield was determined as vg per mL cell culture.

FIG. 52: Recombinant AAV-2^{RA}-Nb and -peptide variant particles were analyzed for incorporation of VP1-Nb and -peptide variants proteins by Jess Western blotting using an antibody that recognizes a common epitope in the capsid proteins.

FIG. 53: Recombinant chAAV-9/2^{RA}-Nb and -peptide variant particles were analyzed for incorporation of VP1-Nb and -peptide variants proteins by Jess Western blotting using an antibody that recognizes a common epitope in the capsid proteins.

FIG. 54: Recombinant AAV-2^{RA}-Nb variant particles were analyzed for incorporation of VP1-Nb and -peptide variants proteins by Jess Western blotting using an antibody that recognizes a common epitope in the capsid proteins.

FIG. 55: Recombinant chAAV-9/2^{RA}-Nb and AAV-9-Nb variant particles were analyzed for incorporation of VP1-Nb and -peptide variants proteins by Jess Western blotting using an antibody that recognizes a common epitope in the capsid proteins.

FIG. 56: Scheme of SPR binding experiment.

FIG. 57: SPR binding kinetics of human TfR and captured wild-type AAV particles.

FIG. 58: SPR binding kinetics of human TfR and captured AAV-2^{RA}-VP2/VP3 particles.

FIG. 59: SPR binding kinetics of human TfR and captured AAV-2^{RA}-aTfR-NB-1 particles.

FIG. 60: SPR binding kinetics of human TfR and captured AAV-2^{RA}-aTfR-Pept-1 particles.

FIG. 61: SPR binding kinetics of human TfR and captured AAV-2^{RA}-aTfR-Pept-2 particles.

FIG. 62: SPR binding kinetics of human TfR with captured AAV-2^{RA}-Nb control particles.

FIG. 63: SPR binding kinetics of human TfR with captured AAV-2^{RA}-aTfR-Nb-1 particles.

FIG. 64: SPR binding kinetics of human TfR with captured AAV-2^{RA}-aTfR-Nb-1-(G4S) 2 particles.

FIG. 65: SPR binding kinetics of human TfR with captured AAV-2^{RA}-aTfR-Nb-2 particles.

FIG. 66: SPR binding kinetics of human CD98 with captured AAV-2^{RA}-Nb-control particles.

FIG. 67: SPR binding kinetics of human CD98 with captured AAV-2^{RA}-aCD98-Nb-1 particles.

FIG. 68: SPR binding kinetics of human CD98 with captured AAV-2^{RA}-aCD98-Nb-2 particles.

FIG. 69: SPR binding kinetics of human TfR and captured wild-type AAV-9 particles.

FIG. 70: SPR binding kinetics of human TfR and captured chimeric AAV-9/2^{RA}-aTfR-Nb-1 particles.

FIG. 71: SPR binding kinetics of human TfR and captured chimeric AAV-9/2^{RA}-aTfR-Pept-1 particles.

FIG. 72: SPR binding kinetics of human TfR and captured chimeric AAV-9/2^{RA}-aTfR-Pept-2 particles.

FIG. 73-80: Binding of AAV-Nb and -peptide variant particles to huTfR-expressing CHO cells. CHO-huTfR or wild-type CHO cells were incubated in presence or absence of holo-transferrin for one hour on ice with AAV variant particles at MOIs ranging from 1E+3 to 1E+5. Bound AAV was labeled using an anti-AAV-2 or anti-AAV-9 antibody followed by flow cytometry to quantify the percentage of AAV-bound cells.

FIG. 73: Binding of AAV-2^{RA}-aTfR-Nb-1 variant particles to huTfR-expressing CHO cells.

FIG. 74: Binding of AAV-2^{RA}-aTfR-Pept-1 variant particles to huTfR-expressing CHO cells.

FIG. 75: Binding of AAV-2^{RA}-aTfR-Pept-2 variant particles to huTfR-expressing CHO cells.

FIG. 76: Binding of wild-type AAV-2 particles to huTfR-expressing CHO cells.

FIG. 77: Binding of chimeric AAV-9/2^{RA}-aTfR-Nb-1 variant particles to huTfR-expressing CHO cells.

FIG. 78: Binding of chimeric AAV-9/2^{RA}-aTfR-Pept-1 variant particles to huTfR-expressing CHO cells.

FIG. 79: Binding of chimeric AAV-9/2^{RA}-aTfR-Pept-2 variant particles to huTfR-expressing CHO cells.

FIG. 80: Binding of wild-type AAV-9 particles to huTfR-expressing CHO cells.

FIG. 81-88: Binding of AAV-Nb and -peptide variant particles to chimeric TfR^{hu/mu}-expressing MDCKII cells. MDCKII-chuTfR or wild-type MDCKII cells were incubated in presence or absence of holo-transferrin for one hour on ice with AAV candidates at MOIs ranging from 1E+3 to 1E+5. Bound AAV was labeled using an anti-AAV-2 or anti-AAV-9 antibody followed by flow cytometry to quantify the percentage of AAV-bound cells.

FIG. 81: Binding of AAV-2^{RA}-aTfR-Nb-1 variant particles to chimeric TfR^{hu/mu}-expressing MDCKII cells.

FIG. 82: Binding of AAV-2^{RA}-aTfR-Pept-1 variant particles to chimeric TfR^{hu/mu}-expressing MDCKII cells.

FIG. 83: Binding of AAV-2^{RA}-aTfR-Pept-2 variant particles to chimeric TfR^{hu/mu}-expressing MDCKII cells.

FIG. 84: Binding of wild-type AAV-2 particles to chimeric TfR^{hu/mu}-expressing MDCKII cells.

FIG. 85: Binding of chimeric AAV-9/2^{RA}-aTfR-Nb-1 variant particles to chimeric TfR^{hu/mu}-expressing MDCKII cells.

FIG. 86: Binding of chimeric AAV-9/2^{RA}-aTfR-Pept-1 variant particles to chimeric TfR^{hu/mu}-expressing MDCKII cells.

FIG. 87: Binding of chimeric AAV-9/2^{RA}-aTfR-Pept-2 variant particles to chimeric TfR^{hu/mu}-expressing MDCKII cells.

FIG. 88: Binding of wild-type AAV-9 particles to chimeric TfR^{hu/mu}-expressing MDCKII cells.

FIG. 89-95: Binding of AAV-2-Nb variant particles to chimeric TfR^{hu/mu}-expressing MDCKII cells. MDCKII-chuTfR or MDCKII-WT cells were incubated for one hour on ice with AAV candidates at MOIs ranging from 1E+3 to 1E+5. Bound AAV was labeled using an anti-AAV-2 antibody followed by flow cytometry to quantify the percentage of AAV-bound cells.

FIG. 89: Binding of AAV-2^{RA}-Nb-control variant particles to chimeric TfR^{hu/mu}-expressing MDCKII cells.

FIG. 90: Binding of AAV-2^{RA}-aTfR-Nb-2 variant particles to chimeric TfR^{hu/mu}-expressing MDCKII cells.

FIG. 91: Binding of AAV-2^{RA-YF}-aTfR-Nb-2 variant particles to chimeric TfR^{hu/mu}-expressing MDCKII cells.

FIG. 92: Binding of AAV-2^{RA}-aTfR-Nb-1 variant particles to chimeric TfR^{hu/mu}-expressing MDCKII cells.

FIG. 93: Binding of AAV-2^{RA-YF}-aTfR-Nb-1 variant particles to chimeric TfR^{hu/mu}-expressing MDCKII cells.

FIG. 94: Binding of AAV-2^{RA}-aTfR-Nb-1-(G4S) 2 variant particles to chimeric TfR^{hu/mu}-expressing MDCKII cells.

FIG. 95: Binding of AAV-2^{RA-YF}-aTfR-Nb-1-(G4S) 2 variant particles to chimeric TfR^{hu/mu}-expressing MDCKII cells.

FIG. 96-101: Binding of AAV-9-Nb variant particles to chimeric TfR^{hu/mu}-expressing MDCKII cells. MDCKII-chuTfR or MDCKII-WT cells were incubated for one hour on ice with AAV candidates at MOIs ranging from 1E+3 to 1E+5. Bound AAV was labeled using an anti-AAV-9 antibody followed by flow cytometry to quantify the percentage of AAV-bound cells.

FIG. 96: Binding of chimeric AAV-9/2^{RA}-Nb-control variant particles to chimeric TfR^{hu/mu}-expressing MDCKII cells.

FIG. 97: Binding of wild-type AAV-9 particles to chimeric TfR^{hu/mu}-expressing MDCKII cells.

FIG. 98: Binding of AAV-9.CPP.16 particles to chimeric TfR^{hu/mu}-expressing MDCKII cells.

FIG. 99: Binding of chimeric AAV-9/2^{RA}-aTfR-Nb-1 variant particles to chimeric TfR^{hu/mu}-expressing MDCKII cells.

FIG. 100: Binding of AAV-9-aTfR-Nb-1 variant particles to chimeric TfR^{hu/mu} expressing MDCKII cells.

FIG. 101: Binding of AAV-9-aTfR-Nb-1_v2 variant particles to chimeric TfR^{hu/mu}-expressing MDCKII cells.

FIG. 102-104: Binding of AAV-2-Nb variant particles to huCD98 expressing MDCKII cells. MDCKII-CD98 or MDCKII-WT cells were incubated for one hour on ice with AAV candidates at MOIs ranging from 1E+3 to 1E+5. Bound AAV was labeled using an anti-AAV-2 antibody followed by flow cytometry to quantify the percentage of AAV-bound cells.

FIG. 102: Binding of AAV-2^{RA}-Nb-control variant particles to huCD98-expressing MDCKII cells.

FIG. 103: Binding of AAV-2^{RA}-aCD98-Nb-1 variant particles to huCD98-expressing MDCKII cells.

FIG. 104: Binding of AAV-2^{RA}-aCD98-Nb-2 variant particles to huCD98-expressing MDCKII cells.

FIG. 105: Transduction kinetics of Hela cells with genetic AAV-Nb variant particles. 5,000 HeLa cells were transduced at a MOI of 1E+5 followed by live-cell imaging every 6 h to quantify the percentage of mGL positive cells.

FIG. 106-92: Transduction of HeLa cells with AAV-Nb variant particles. 5,000 HeLa cells were transduced at MOIs ranging from 1E+3 to 1E+5 followed by flow cytometry after 3 days to quantify the percentage of mGL positive cells.

FIG. 106: Transduction of Hela cells with AAV-2^{RA}-Nb-control variant particles.

FIG. 107: Transduction of HeLa cells with AAV-2.7m8 particles.

FIG. 108: Transduction of HeLa cells with AAV-2^{RA}-aTfR-Nb-2 variant particles.

FIG. 109: Transduction of HeLa cells with AAV-2^{RA-YF}-aTfR-Nb-2 variant particles.

FIG. 110: Transduction of Hela cells with AAV-2^{RA}-aTfR-Nb-1 variant particles.

FIG. 111: Transduction of HeLa cells with AAV-2^{RA-YF}-aTfR-Nb-1 variant particles.

FIG. 112: Transduction of Hela cells with AAV-2^{RA}-aTfR-Nb-1-(G4S) 2 variant particles.

FIG. 113: Transduction of Hela cells with AAV-2^{RA-YF}-aTfR-Nb-1-(G4S) 2 variant particles.

FIG. 114: Transduction kinetics of HeLa cells with AAV-9-aTfR-Nb and AAV-9/2-aTfR-Nb variant particles. 5,000 HeLa cells were transduced at a MOI of 1E+5 followed by live-cell imaging every 6 h to quantify the percentage of mGL positive cells.

FIG. 115-120: Transduction of HeLa cells with AAV-Nb variant particles. 5,000 HeLa cells were transduced at MOIs ranging from 1E+3 to 1E+5 followed by flow cytometry after 3 days to quantify the percentage of mGL positive cells. Endpoint measurements of FIG. 93.

FIG. 115: Transduction of HeLa cells with chimeric AAV-9/2^{RA}-Nb-control variant particles.

FIG. 116: Transduction of HeLa cells with wild-type AAV-9 particles.

FIG. 117: Transduction of HeLa cells with chimeric AAV-9/2^{RA}-aTfR-Nb-1 variant particles.

FIG. 118: Transduction of HeLa cells with AAV-2^{RA}-aTfR-Nb-1 variant particles.

FIG. 119: Transduction of HeLa cells with AAV-9-aTfR-Nb-1 variant particles.

FIG. 120: Transduction of Hela cells with chimeric AAV-9/2^{RA}-Nb-control variant particles.

FIG. 121-109: AAV-2-Nb variant particles transduce glutamatergic neurons with high efficiency. 65,000 iCell GlutaNeurons were transduced with MOI 1E+5 and transduction was assessed via live-cell imaging. Representative images are shown at day 7 of transduction.

FIG. 121: AAV-2.7m8 particles transduce glutamatergic neurons with high efficiency.

FIG. 122: AAV-2^{RA}-Nb-control variant particles transduce glutamatergic neurons with low efficiency.

FIG. 123: AAV-2^{RA}-aTfR-Nb-2 variant particles transduce glutamatergic neurons with high efficiency.

FIG. 124: AAV-2^{RA-YF}-aTfR-Nb-2 variant particles transduce glutamatergic neurons with high efficiency.

FIG. 125: AAV-2^{RA}-aTfR-Nb-1 variant particles transduce glutamatergic neurons with high efficiency.

FIG. 126: AAV-2^{RA-YF}-aTfR-Nb-1 variant particles transduce glutamatergic neurons with high efficiency.

FIG. 127: AAV-2^{RA}-aTfR-Nb-1-(G4S) 2 variant particles transduce glutamatergic neurons with high efficiency.

FIG. 128: AAV-2^{RA-YF}-aTfR-Nb-1-(G4S) 2 variant particles transduce glutamatergic neurons with high efficiency.

FIG. 129: AAV-2^{RA}-aCD98-Nb-1 variant particles transduce glutamatergic neurons with high efficiency.

FIG. 130: AAV-2^{RA}-aCD98-Nb-2 variant particles transduce glutamatergic neurons with high efficiency.

FIG. 131: AAV-2-Nb variant particles transduce glutamatergic neurons with high efficiency. 65,000 iCell GlutaNeurons were transduced with MOI 1E+5 and transduction was assessed via live-cell imaging. Quantification of mGreenLantern (mGL) positive cells. After nuclear staining, the ratio of mGL positive to total nuclei was calculated.

FIG. 132: AV-2.7m8 particles transduce glutamatergic neurons with high efficiency.

FIG. 133: AAV-9 particles transduce glutamatergic neurons with low efficiency.

FIG. 134 chAAV-9/2^{RA}aTfR-Nb-1 particles transduce glutamatergic neurons with high efficiency.

FIG. 135: AAV-9-aTfR-Nb-1 particles transduce glutamatergic neurons with high efficiency.

FIG. 136: AAV-9-aTfR-Nb-1_v2 particles transduce glutamatergic neurons with high efficiency.

FIG. 137: chAAV-9/2^{RA}aCD98-Nb-2 particles transduce glutamatergic neurons with high efficiency.

FIG. 138: chAAV-9/2^{RA}-Nb-Ctrl particles transduce glutamatergic neurons with low efficiency.

FIG. 139: AAV-9-Nb variant particles transduce glutamatergic neurons with high efficiency. 65,000 iCell GlutaNeurons were transduced with MOI 5E+4 and transduction was assessed via live-cell imaging. Quantification of mGreenLantern (mGL) positive cells and ratio of mGL positive to total cells was calculated.

FIG. 140: AAV-2.7m8 particles transduce NGN+ neurons with high efficiency.

FIG. 141: AAV-9 particles transduce NGN+ neurons with low efficiency.

FIG. 142: chAAV-9/2^{RA}aTfR-Nb-1 particles transduce NGN2+ neurons with high efficiency.

FIG. 143: AAV-9-aTfR-Nb-1 particles transduce NGN+ neurons with high efficiency.

FIG. 144: AAV-9-aTfR-Nb-1 particles transduce NGN+ neurons with high efficiency.

FIG. 145: chAAV-9/2^{RA}-aCD98-Nb-2 particles transduce NGN2+ neurons with high efficiency.

FIG. 146: chAAV-9/2^{RA}-Nb-Ctrl particles transduce NGN2+ neurons with low efficiency.

FIG. 147: AAV-9-Nb variant particles transduce NGN2+ neurons with high efficiency. 75,000 NGN2+ neurons were transduced with MOI 1E+5 and transduction was assessed via live-cell imaging. Quantification of mGreenLantern (mGL) intensity per image was calculated.

FIG. 148: Distribution of AAV variant particles in different regions of the brain reported as viral genomes (vg) per diploid genome (dg).

FIG. 149: Distribution of AAV variant particles in different other organs reported as viral genomes (vg) per diploid genome (dg).

FIG. 150: Transgene RNA expression in different regions of the brain reported as copies per nanogram (ng) RNA.

FIG. 151: Transgene RNA expression in different other organs reported as copies per nanogram (ng) RNA.

FIG. 152: Representative images of different regions of the brain and transgene expression in chAAV-9/2^{RA}-aTfR-Nb-1 transduced cells. Brain sections were stained for neuronal marker NeuN.

FIG. 153: Representative images of different regions of the brain and transgene expression in AAV-9 transduced cells. Brain sections were stained for neuronal marker NeuN.

FIG. 154: Representative images of different regions of the brain and transgene expression in AAV-9.PHP.eB transduced cells. Brain sections were stained for neuronal marker NeuN.

FIG. 155: Representative images of different regions of the brain and transgene expression in AAV-2^{RA}-aTfR-Nb-1 transduced cells. Brain sections were stained for neuronal marker NeuN.

FIG. 156: Representative images of different regions of the brain and transgene expression in AAV-2^{RA} transduced cells. Brain sections were stained for neuronal marker NeuN.

FIG. 157: Transduction of HeLa cells with AAV-2^{RA}-aTfR-Nb-1_T456.

FIG. 158: Transduction of HeLa cells with AAV-2^{RA}-aTfR-Nb-1_del453-459.

FIG. 159: Transduction of HeLa cells with chAAV-9/2^{RA}-aTfR-Nb-1_T456.

FIG. 160: Transduction of HeLa cells with chAAV-9/2^{RA}-aTfR-Nb-1_del453-459.

FIG. 161: Formula 2.

### DETAILED DESCRIPTION OF EMBODIMENTS OF THE INVENTION

Herein is reported amongst other things a novel method for the generation of retargeted adeno-associated viruses (AAVs) that circumvents the limitations inherent to prior methodologies through the application of an enzymatic conjugation technology. This novel retargeting strategy mitigates the drawbacks of previously known methods, such as, amongst other things, immunogenicity concerns.

The method according to the current invention employs a microbial transglutaminase derived from Kutzneria albida (KTG), which catalyzes the formation of an isopeptide bond between two distinct peptide tags, for the conjugation of a specific binder, i.e. a target-binding moiety, to a modified AAV particle. The method is generally applicable to any binding or targeting moiety as long as the respective peptide tag can be introduced therein. Due to this enzymatic process, the modified AAV particle can be combined with any binding specificity obviating the need for repeated capsid modifications.

In more detail, the current invention is based, at least in part, on the finding that the first peptide tag, a pentapeptide designated as the Q-tag (YRYRQ; SEQ ID NO: 73), can be engineered into the GH2/GH3 loop of an AAV-2 capsid. Preferably, this Q-tag is flanked C- and N-terminally by a peptidic linker sequence (e.g., such as in GGGGSYRYRQGGGGS; SEQ ID NO: 74). It has to be pointed out that the peptidic linker at the N-terminus and the peptidic linker at the C-terminus do not need to be identical in length and composition. Each of the peptidic linkers can be selected independently of the other peptidic linker with respect to amino acid sequence and length. The second peptide tag, a pentapeptide referred to as the K-tag (RYSEK; SEQ ID NO: 75), is fused to the target-binding moiety (e.g., such as in GGGRYESKGGG; SEQ ID NO: 76). The KTG enzyme specifically catalyzes the formation of an isopeptide bond between the glutamine residue within the Q-tag and the lysine residue of the K-tag, resulting in the formation of a stable covalent isopeptide bond.

The enzymatic conjugation strategy according to the current invention represents a significant advancement in the retargeting of AAV particles, expanding the addressable target space by enhancing their specificity and reducing potential immunogenicity.

Further, the current invention is based, at least in part, on the finding that for the conjugation of a binding site, e.g. a nanobody, a scFv or a Fab, by KTG to an AAV particle the K-tag has to be at the C-terminus of the binding site. Thereby higher enzymatic conjugation yields can be achieved.

Moreover, Fc domains can be conjugated to AAV particles allowing for the interaction with FcRn.

### Definitions

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. The methods and techniques of the present disclosure are generally performed according to conventional methods well known in the art. Generally, nomenclatures used in connection with, and techniques of biochemistry, enzymology, molecular, and cellular biology, microbiology, genetics and protein and nucleic acid chemistry and hybridization described herein are those well-known and commonly used in the art.

General information regarding the nucleotide sequences of human immunoglobulins light and heavy chains is given in: Kabat, E. A., et al., Sequences of Proteins of Immunological Interest, 5th ed., Public Health Service, National Institutes of Health, Bethesda, MD (1991).

As used herein, the amino acid positions of all constant regions and domains of the heavy and light chain are numbered according to the Kabat numbering system described in Kabat, et al., Sequences of Proteins of Immunological Interest, 5th ed., Public Health Service, National Institutes of Health, Bethesda, MD (1991) and is referred to as “numbering according to Kabat” herein. Specifically, the Kabat numbering system (see pages 647-660) of Kabat, et al., Sequences of Proteins of Immunological Interest, 5th ed., Public Health Service, National Institutes of Health, Bethesda, MD (1991) is used for the light chain constant domain CL of kappa and lambda isotype, and the Kabat EU index numbering system (see pages 661-723) is used for the constant heavy chain domains (CH1, Hinge, CH2 and CH3, which is herein further clarified by referring to “numbering according to Kabat EU index” in this case).

Likewise the hypervariable regions (HVRs) in the heavy and light chain variable domains of non-human and human antibodies are determined following Kabat, E. A., et al., Sequences of Proteins of Immunological Interest, 5th ed., Public Health Service, National Institutes of Health, Bethesda, MD (1991) and are denoted as “according to Kabat”.

It must be noted that as used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art, and so forth. As well, the terms “a” (or “an”), “one or more” and “at least one” can be used interchangeably herein. It is also to be noted that the terms “comprising”, “including”, and “having” can be used interchangeably.

The terms “comprise(s),” “include(s),” “having,” “has,” “can,” “contain(s)” and variants thereof, as used herein, are intended to be open-ended transitional phrases, terms or words that do not preclude the possibility of additional acts or structures. The term “comprising” also encompasses the term “consisting of”. The present disclosure also contemplates other embodiments “comprising”, “consisting of” and “consisting essentially of” the embodiments or elements presented herein, whether explicitly set forth or not.

The term “about” as used herein in connection with a specific value (e.g. temperature, concentration, time and others) shall refer to a variation of +/−1% of the specific value that the term “about” refers to.

Useful methods and techniques for carrying out the current invention are described in e.g. Ausubel, F. M. (ed.), Current Protocols in Molecular Biology, Volumes I to III (1997); Glover, N. D., and Hames, B. D., ed., DNA Cloning: A Practical Approach, Volumes I and II (1985), Oxford University Press; Freshney, R.I. (ed.), Animal Cell Culture—a practical approach, IRL Press Limited (1986); Watson, J. D., et al., Recombinant DNA, Second Edition, CHSL Press (1992); Winnacker, E. L., From Genes to Clones; N.Y., VCH Publishers (1987); Celis, J., ed., Cell Biology, Second Edition, Academic Press (1998); Freshney, R.I., Culture of Animal Cells: A Manual of Basic Technique, second edition, Alan R. Liss, Inc., N.Y. (1987).

The use of recombinant DNA technology enables the generation of derivatives of a nucleic acid. Such derivatives can, for example, be modified in individual or several nucleotide positions by substitution, alteration, exchange, deletion or insertion. The modification or derivatization can, for example, be carried out by means of site directed mutagenesis. Such modifications can easily be carried out by a person skilled in the art (see e.g. Sambrook, J., et al., Molecular Cloning: A laboratory manual (1999) Cold Spring Harbor Laboratory Press, New York, USA; Hames, B. D., and Higgins, S. G., Nucleic acid hybridization—a practical approach (1985) IRL Press, Oxford, England).

An “isolated” nucleic acid refers to a nucleic acid molecule that has been separated from a component of its natural environment.

In more detail, the term “isolated” refers to material, which is substantially or essentially free from components that normally accompany the material as it is found in its native state. Thus, an “isolated plasmid” or “isolated plasmid DNA” does not contain materials normally associated with the plasmid or plasmid DNA in their in situ environment. For example, a nucleic acid or polynucleotide is said to be “isolated” when it is substantially separated from contaminant polynucleotides that correspond or are complementary to genes other than the target genes or that encode polypeptides other than the target gene product or fragments thereof. A skilled artisan can readily employ nucleic acid isolation procedures to obtain an isolated polynucleotide.

An “individual” or “subject” is a mammal. Mammals include, but are not limited to, domesticated animals (e.g. cows, sheep, cats, dogs, and horses), primates (e.g., humans and non-human primates such as monkeys), rabbits, and rodents (e.g., mice and rats). In certain embodiments, the individual or subject is a human.

The term “package insert” is used to refer to instructions customarily included in commercial packages of therapeutic products, that contain information about the indications, usage, dosage, administration, combination therapy, contraindications and/or warnings concerning the use of such therapeutic products.

The term “pharmaceutical formulation” refers to a preparation which is in such form as to permit the biological activity of an active ingredient contained therein, e.g. of a therapeutic antibody, to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

The term “polynucleotide” means a polymeric form of nucleotides of at least 10 bases or base pairs in length, either ribonucleotides or deoxynucleotides or a modified form of either type of nucleotide, and is meant to include single and double stranded forms of DNA and/or RNA. In the art, this term is often used interchangeably with “oligonucleotide”.

As used herein, the term “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In certain embodiments, an antibody according to the current invention is used to delay or prevent development of a disease or to slow the progression of a disease.

When a range of values is listed herein, it is intended to encompass the boundaries as well as each value and sub-range within that range. For example, “2 mg/kg to 6 mg/kg” is intended to encompass, for example, 2.0 mg/kg, 2.5 mg/kg, 3 mg/kg, 3.5 mg/kg, 4 mg/kg, 4.5 mg/kg, 5 mg/kg, 5.5 mg/kg, 6 mg/kg, 2.5 mg/kg to 3 mg/kg, 2.5 mg/kg to 4.5 mg/kg, 3 mg/kg to 4.5 mg/kg, 4.5 mg/kg to 6 mg/kg, 2.5 mg/kg to 4 mg/kg, and so forth.

The term “transgene” denotes a nucleic acid derived from a wild-type genome of an adeno-associated virus, wherein except for the ITR (adeno-associated virus Inverted Terminal Repeat) sequences all endogenous AAV nucleic acids are replaced by one or more exogenous nucleic acid(s). For example, such an exogenous nucleic acid can be a nucleic acid transcribed into a transcript of interest or that encodes a therapeutic protein or a therapeutic nucleic acid. Typically, for a transgene one or both ITR sequences of a wild-type AAV genome are retained. Thus, a transgene can be distinguished from a wild-type AAV genome, since all or at least a part of the viral genome has been replaced with a non-native (i.e. exogenous) nucleic acid with respect to the virus. Incorporation of a non-native nucleic acid therefore defines the transgene as a “recombinant”. It has to be pointed out that the serotype of the ITRs in the transgene does not need to be the same as the serotype of the adeno-associated capsid polypeptides forming the shell of the rAAVp comprising said transgene. Neither does one or do both ITRs have to be wild-type, i.e. are an engineered ITR variant.

Thus, the term “transgene” also denotes the portion of a larger nucleic acid, e.g. of a recombinant plasmid, that is ultimately packaged or encapsulated or encapsidated either directly or in form of a single strand or in form of RNA into a protein shell composed of adeno-associated virus capsid polypeptides to form a rAAVp. In cases where recombinant plasmids are used to construct or manufacture rAAVps, the viral particle does not include the portion of the “plasmid” that does not correspond to the transgene part of the recombinant plasmid. For example, in case of a rAAVp the encapsidated nucleic acid comprises that part of the recombinant plasmid that is interspaced between two AAV ITRs. The non-transgene portion of the recombinant plasmid is referred to as the “plasmid backbone”. The plasmid backbone is important for cloning and amplification of the plasmid, a process that is needed for propagation and recombinant virus production, but is not itself packaged or encapsulated or encapsidated into the rAAVp. Thus, a “transgene” refers to the nucleic acid that is packaged or encapsulated or encapsidated by a protein shell composed of adeno-associated virus capsid polypeptides, i.e. in a rAAVp.

In principle, any non-AAV nucleic acid can be packaged into a shell composed of adeno-associated capsid polypeptides resulting in a rAAVp, e.g. for subsequent infection (transduction) of a cell, ex vivo, in vitro or in vivo.

The term “serotype” is used herein to denote the origin of the elements forming a rAAVp as well as the origin of elements used for the production of a rAAVp. Thus, the serotype can be used to denote the origin of the amino acid sequence of the polypeptides forming the protein shell (capsid) of the rAAVp as well as the origin of, e.g., the rep gene or its fragments used for the production/packaging of the rAAVp.

General Methods for Producing rAAVp

Piras et al. employed adherent HEK293T/17 cells cultured in Dulbecco's Modified Eagle's Medium with 10% fetal bovine serum supplemented with 2 mmol/l GlutaMAX (Life Technologies, Grand Island, NY). AAV was produced by two-plasmid transfection using PEIpro™.

Powers, A. D., et al. (Hum. Gene Ther. Meth. 27 (2016) 112-121) reported the development and optimization of AAV hFIX particle production by transient transfection in an iCELLis® fixed-bed bioreactor. On day 3 after inoculation with HEK293T/17 cells, the vessel was transfected with plasmid scAAV-LP1-hFIXco-helpv3 and plasmid CR21+LTAAV help 2-8 using polyethylenimine.

Poulain, A., et al. (J. Biotechnol. 255 (2017) 16-27) reported rapid protein production from stable CHO cell pools using plasmid vector and the cumate gene-switch. Cells were transfected using linear polyethylenimine.

WO 2017/096039 reported scalable methods for producing recombinant AAV vectors in serum-free suspension cell culture systems suitable for clinical use. Production of rAAV vectors was performed in bioreactors with HEK293F cells using triple transfection with a plasmid ratio of 1:1:1 and a PEI-based transfection reagent.

Koo, T., et al. (Nat. Commun. 9 (2018) 1855) reported that CRISPR-LbCpf1 prevents choroidal neovascularization in a mouse model of age-related macular degeneration. To produce AAV vectors, they were pseudotyped in AAV-9 capsids. HEK293T cells (ATCC, CRL-3216) were transfected with pAAV-ITR-LbCpf1-crRNA, pAAV-2/9 encoding for AAV-2rep and AAV-9cap, and helper plasmid. Recombinant pseudotyped AAV vector stocks were generated using PEI co-precipitation and triple-transfection with plasmids at a molar ratio of 1:1:1 in HEK293T cells. After 72 h of incubation, cells were lysed and particles were purified by iodixanol step-gradient ultracentrifugation.

The Rep proteins from AAV-2 are commonly and nearly exclusively used in the production of rAAVps derived from the serotypes AAV-1 to AAV-13 (Daya, S., and Berns, K. I., Clin. Microbiol. Rev. 21 (2008) 583-593; Zincarelli, C., et al., Mol. Ther. 16 (2008) 1073-1080).

WO 2019/094253 reported means and methods for preparing viral vectors and uses thereof. Adherent HEK293 cells were cultivated in bioreactors and triple transfected (plasmid ratio 1:1:1) with PEI/DNA at a PEI-plasmid ratio of about 1:1 by weight.

Collaud, F. et al. (Mol. Ther. Meth. Clin. Dev. 12 (2019) 157-174) reported a fully scalable method based on triple transfection of HEK293 cells cultured in suspension. AAV vectors were recovered from both supernatant and cells by mild detergent lysis followed by AVB Sepharose affinity column purification. Purified vectors were then concentrated and tested for quality and potency.

Nyamay'antu, A., et al. (Cell Gen. Ther. Ins. 6 (2020) 655-661) reported that the efficiency of the delivery process is essential to obtain a high number of producing cells. Of the existing transfection methods, the use of PEI-based transfection reagent is predominant in gene therapy as it combines affordability and compatibility for transfection of adherent and suspension cells. In more detail, suspension HEK293T cells were transfected using the respective transfection reagent under the recommended conditions. rAAV-2-GFP were harvested 72 hours post transfection. The results are almost independent of the employed cultivation medium.

In a blog article entitled “Optimization of AAV production for high-yielding and scalable GMP processes with Catalent” (www.polyplus-transfection.com) different transfection reagent to DNA ratios were tested with the two serotypes AAV-9 (1:1 and 2:1) and AAV-2 (3:1.5 and 5:2.5). In a further study comparing additional AAV-2 and AAV-5 vectors (different from the previous AAV-2 and AAV-5 vectors) and using a DoE approach to optimization, experiments were conducted varying transfection reagent to DNA ratios (3:2, 3:1.5) and plasmid DNA molar ratios (1:1:1, 2:1:2, 1:2:1) were performed.

WO 2018/192983 reported an adeno-associated virus (AAV) producer cell comprising nucleic acid sequences encoding rep/cap gene; helper virus genes; and the DNA genome of the AAV vector particle, wherein said nucleic acid sequences are all integrated together at a single locus within the AAV producer cell genome. Reported are also nucleic acid vectors comprising a non-mammalian origin of replication and the ability to hold at least 25 kilo bases (kb) of DNA, characterized in that said nucleic acid vector comprises nucleic acid sequences encoding: rep/cap gene, and helper virus genes as well as uses and methods using said nucleic acid vector in order to produce stable AAV packaging and producer cell lines.

WO 2020/132059 reported a mammalian cell for producing an adeno-associated virus (AAV), comprising (a) a nucleic acid molecule encoding a viral helper gene under control of a first derepressible promoter; (b) a nucleic acid molecule encoding an AAV gene under control of a second derepressible promoter; and (c) a nucleic acid molecule encoding a repressor element of the first and the second derepressible promoters.

EP 3 822 346 reported the use of an engineered mammalian packaging cell line for producing recombinant virus particles, wherein the cell line is engineered to lack cell surface expression of heparan sulfate. Further disclosed are a method for producing recombinant virus particles and a recombinant virus particle obtainable by the method. Further disclosed is a mammalian packaging cell line deposited under number DSM ACC3355 or DSM ACC3356.

WO 2022/112218 reported methods for the production of Adeno-associated virus (AAV), comprising steps of providing a stable AAV producer cell line in which at least some or all genes encoding the components necessary for the production of AAV are stably integrated into the cell genome, and culturing said cells in perfusion culture during the AAV production step (i.e., during the N step), wherein said perfusion culture encompasses continuous replacement of spent media with fresh media, and wherein said continuous replacement of spent media with fresh media continues after the induction of AAV production. In the cell at least (a) a gene encoding the AAV Rep protein Rep78 or Rep68, (b) a gene encoding the AAV Rep protein Rep52 or Rep40, (c) the genes encoding the adenoviral helper functions E4orf6 and E2A stably integrated into the host cell genome. Further at least the following genes are stably integrated into the host cell genome (a) the genes encoding the AAV Cap proteins VP1, VP2, VP3; (b) a gene encoding the AAV Rep protein Rep78 or Rep68; (c) a gene encoding the AAV Rep protein Rep52 or Rep40; (d) the genes encoding the adenoviral helper functions E4orf6 E2A; (e) the gene of interest flanked by AAV ITRs.

WO 2022/173944 reported methods for producing an adeno-associated virus (AAV) in an E1 complementary producer cell. Especially is reported a method of producing an adeno-associated virus (AAV) in an E1 complementary producer cell, comprising (a) transfecting the E1 complementary producer cell with one or more vectors comprising (1) an E1A adenovirus helper gene; (2) an adenovirus helper gene selected from E2A, E4, or both; (3) a viral-associated, non-coding RNA (VA RNA); and (4) an AAV gene selected from Rep, Cap, or both; (b) culturing the transfected E1 complementary producer cell under conditions suitable for producing the AAV; and (c) purifying the AAV from the cultured E1 complementary producer cell, thereby obtaining the AAV.

WO 2022/192261 reported compositions and methods for producing and characterizing stable viral vector producer cell lines that enable industrial scale production of viral vectors. Novel viral vector genome constructs, in which the constructs can be precisely mapped and viral vector genome constructs precisely quantified, are also disclosed for efficient production and characterization of viral vectors in mammalian cells.

WO 2023/077078 reported recombinant adeno-associated virus (rAAV) packaging and/or producer cell lines, which have been engineered to reduce expression and/or activity of one or more genes and/or proteins to increase rAAV titers.

WO 2023/102549 reported systems for increasing AAV particle production. These systems comprise producer cell lines adapted for the production of AAV particles, as well as methods of producing AAV particles using said producer cell lines. Also provided are AAV particles produced by said production systems, producer cell lines and methods.

WO 2023/114897 reported methods for the production of recombinant adeno-associated virus (rAAV) particles. These methods are particularly useful for the large-scale production of AAV particles. Especially it is reported A method for producing recombinant AAV (rAAV) particles, comprising (a) introducing into a mammalian cell a first polynucleotide comprising an rAAV genome, to generate an AAV producer cell; (b) culturing the AAV producer cell in a first culture medium at a first temperature for a first period of time; (c) culturing the AAV producer cell in a second culture medium at a second temperature for a second period of time, wherein the second temperature is about 38° C. to about 42° C., such that rAAV particles are produced by the AAV producer cell, wherein the rAAV particles comprise an rAAV genome comprising a transgene, and an AAV capsid comprising an AAV capsid protein.

The content of all documents outlined in this section are expressly incorporated by reference herein.

### Recombinant Cell

Generally, for efficient as well as large-scale production of a rAAVp a cell expressing and, if possible, also secreting said rAAVp is used. Such a cell is termed “recombinant producer cell” or short “producer cell”.

For the generation of a recombinant producer cell a suitable mammalian cell is transfected with the nucleic acids required for producing said rAAVp, including the required AAV helper functions.

Generally, for expression of a coding sequence, i.e. of an open reading frame, additional regulatory elements, such as a promoter and a polyadenylation signal (sequence), are necessary. Thus, for functional transcription an open reading frame has to be and is operably linked to said additional regulatory elements. This can be achieved by combining these parts into a so-called expression cassette. The minimal regulatory elements required for an expression cassette to be functional in a mammalian cell are a promoter functional in said mammalian cell, which is located upstream, i.e. 5′, to the open reading frame, and a polyadenylation signal (sequence) functional in said mammalian cell, which is located downstream, i.e. 3′, to the open reading frame. Additionally a terminator sequence may be present 3′ to the polyadenylation signal (sequence). For expression, the promoter, the open reading frame/coding region and the polyadenylation signal sequence have to be arranged in an operably linked form.

Likewise, a nucleic acid that is transcribed into a non-protein coding RNA is called “RNA gene”. Also for expression of an RNA gene, additional regulatory elements, such as a promoter and a transcription termination signal or polyadenylation signal (sequence), are necessary. The nature and localization of such elements depends on the RNA polymerase that is intended to drive the expression of the RNA gene. Thus, an RNA gene is normally also integrated into an expression cassette.

In case of an rAAVp, which is composed of different (monomeric) capsid polypeptides and a therein encapsidated single stranded DNA molecule and which in addition requires other viral helper functions for production and encapsidation, a multitude of expression cassettes differing in the contained open reading frames/coding sequences are required. In this case, at least an expression cassette for each of the transgene, for the polypeptides forming the capsid of the rAAVp, for the required viral helper functions are required. Thus, individual expression cassettes at least for each of the helper functions E1A, E1B, E2A, E4orf6, the rep and cap genes are required. HEK293 cells express the E1A and E1B helper functions constitutively.

### Adeno-Associated Virus (AAV)

For a general review of AAVs and of the adenovirus or herpes helper functions see, Berns and Bohensky, Advances in Virus Research, Academic Press., 32 (1987) 243-306. The genome of AAV is described in Srivastava et al., J. Virol., 45 (1983) 555-564. In U.S. Pat. No. 4,797,368 design considerations for constructing recombinant AAV particles are described (see also WO 93/24641). Additional references describing AAV vectors are West et al., Virol. 160 (1987) 38-47; Kotin, Hum. Gene Ther. 5 (1994) 793-801; and Muzyczka J. Clin. Invest. 94 (1994) 1351. Construction of recombinant AAV vectors is described in U.S. Pat. No. 5,173,414; Lebkowski et al., Mol. Cell. Biol. 8 (1988) 3988-3996; Tratschin et al., Mol. Cell. Biol. 5 (1985) 3251-3260; Tratschin et al., Mol. Cell. Biol., 4 (1994) 2072-2081; Hermonat and Muzyczka Proc. Natl. Acad. Sci. USA 81 (1984) 6466-6470; Samulski et al. J. Virol. 63 (1989) 3822-3828.

An AAV is a replication-deficient parvovirus. It can replicate only in cells, in which certain viral functions are provided by a co-infecting helper virus, such as adenoviruses, herpesviruses and, in some cases, poxviruses such as vaccinia. Nevertheless, an AAV can replicate in virtually any cell line of human, simian or rodent origin provided that the appropriate helper viral functions are present.

Without helper viral genes being present, an AAV establishes latency in its host cell. Its genome integrates into a specific site in chromosome 19 [(Chr) 19 (q13.4)], which is termed the adeno-associated virus integration site 1 (AAVS1). For specific serotypes, such as AAV-2 other integration sites have been found, such as, e.g., on chromosome 5 [(Chr) 5 (p13.3)], termed AAVS2, and on chromosome 3 [(Chr) 3 (p24.3)], termed AAVS3.

AAVs are categorized into different serotypes. These have been allocated based on parameters, such as hemagglutination, tumorigenicity and DNA sequence homology. Up to now, more than 12 different serotypes and more than a hundred sequences corresponding to different clades of AAV have been identified.

The capsid protein type and symmetry determines the tissue tropism of the respective AAV. For example, AAV-2, AAV-4 and AAV-5 are specific to retina, AAV-2, AAV-5, AAV-8, AAV-9 and AAV-rh.10 are specific for brain, AAV-1, AAV-2, AAV-6, AAV-8 and AAV-9 are specific for cardiac tissue, AAV-1, AAV-2, AAV-5, AAV-6, AAV-7, AAV-8, AAV-9 and AAV-10 are specific for liver, AAV-1, AAV-2, AAV-5 and AAV-9 are specific for lung.

Pseudotyping denotes a process comprising the cross packaging of the AAV genome between various serotypes, i.e. the genome is packaged with differently originating capsid proteins.

The wild-type AAV genome has a size of about 4.7 kb. The AAV genome further comprises two overlapping genes named rep and cap, which comprise multiple open reading frames (see, e.g., Srivastava et al., J. Viral., 45 (1983) 555-564; Hermonat et al., J. Viral. 51 (1984) 329-339; Tratschin et al., J. Virol., 51 (1984) 611-619). The Rep protein encoding open reading frame provides for four proteins of different size, which are termed Rep78, Rep68, Rep52 and Rep40. These are involved in replication, rescue and integration of the AAV. The Cap protein encoding open reading frame provides four proteins, which are termed VP1, VP2, VP3, and AAP. VP1, VP2 and VP3 are part of the proteinaceous capsid of the AAV particles. The combined rep and cap open reading frames are flanked at their 5′- and 3′-ends by so-called inverted terminal repeats (ITRs). For replication, an AAV requires in addition to the Rep and Cap proteins the products of the genes E1A, E1B, E4orf6, E2A and VA of an adenovirus or corresponding factors of another helper virus.

In the case of an AAV of the serotype 2 (AAV-2), for example, the ITRs each have a length of 145 nucleotides and flank a coding sequence region of about 4470 nucleotides. Of the ITR's 145 nucleotides 125 nucleotides have a palindromic sequence and can form a T-shaped hairpin structure. This structure has the function of a primer during viral replication. The remaining 20, non-paired, nucleotides are denoted as D-sequence.

The wild-type AAV genome harbors three transcription promoters P5, P19, and P40 (Laughlin et al., Proc. Natl. Acad. Sci. USA 76 (1979) 5567-5571) for the expression of the rep and cap genes.

The ITR sequences have to be present in cis to the coding region. The ITRs provide a functional origin of replication (ori), signals required for integration into the target cell's genome, and efficient excision and rescue from host cell chromosomes or recombinant plasmids. The ITRs further comprise origin of replication like-elements, such as a Rep-protein binding site (RBS) and a terminal resolution site (TRS). It has been found that the ITRs themselves can have the function of a transcription promoter (Flotte et al., J. Biol. Chem. 268 (1993) 3781-3790; Flotte et al., Proc. Natl. Acad. Sci. USA 93 (1993) 10163-10167).

For replication and encapsidation, respectively, of the viral single-stranded DNA genome an in trans organization of the rep and cap gene products is required.

The rep gene locus comprises two internal promoters, termed P5 and P19. It comprises open reading frames for four proteins. Promoter P5 is operably linked to a nucleic acid sequence providing for non-spliced 4.2 kb mRNA encoding the Rep protein Rep78 (chromatin nickase to arrest cell cycle), and a spliced 3.9 kb mRNA encoding the Rep protein Rep68 (site-specific endonuclease). Promoter P19 is operably linked to a nucleic acid sequence providing for a non-spliced mRNA encoding the Rep protein Rep52 and a spliced 3.3 kb mRNA encoding the Rep protein Rep40 (DNA helicases for accumulation and packaging).

The two larger Rep proteins, Rep78 and Rep68, are essential for AAV duplex DNA replication, whereas the smaller Rep proteins, Rep52 and Rep40, seem to be essential for progeny and single-strand DNA accumulation (Chejanovsky & Carter, Virology 173 (1989) 120-128).

The larger Rep proteins, Rep68 and Rep78, can specifically bind to the hairpin conformation of the AAV ITR. They exhibit defined enzyme activities, which are required for resolving replication at the AAV termini. Expression of Rep78 or Rep68 could be sufficient for infectious particle formation (Holscher, C., et al. J. Virol. 68 (1994) 7169-7177 and 69 (1995) 6880-6885).

It is deemed that all Rep proteins, primarily Rep78 and Rep68, exhibit regulatory activities, such as induction and suppression of AAV genes as well as inhibitory effects on cell growth (Tratschin et al., Mol. Cell. Biol. 6 (1986) 2884-2894; Labow et al., Mol. Cell. Biol., 7 (1987) 1320-1325; Khleif et al., Virology, 181 (1991) 738-741).

Recombinant overexpression of Rep78 results in phenotype with reduced cell growth due to the induction of DNA damage. Thereby the host cell is arrested in the S phase, whereby latent infection by the virus is facilitated (Berthet, C., et al., Proc. Natl. Acad. Sci. USA 102 (2005) 13634-13639).

Tratschin et al. reported that the P5 promoter is negatively auto-regulated by Rep78 or Rep68 (Tratschin et al., Mol. Cell. Biol. 6 (1986) 2884-2894). Due to the toxic effects of expression of the Rep protein, only very low expression has been reported for certain cell lines after stable integration of AAV (see, e.g., Mendelson et al., Virol. 166 (1988) 154-165).

The cap gene locus comprises one promoter, termed P40. Promoter P40 is operably linked to a nucleic acid sequence providing for 2.6 kb mRNA, which, by alternative splicing and use of alternative start codons, encodes the Cap proteins VP1 (87 kDa, non-spliced mRNA transcript), VP2 (72 kDa, from the spliced mRNA transcript), and VP3 (61 kDa, from alternative start codon). VP1 to VP3 constitute the building blocks of the viral capsid. The capsid has the function to bind to a cell surface receptor and allow for intracellular trafficking of the virus. VP3 accounts for about 90% of total viral particle protein. Nevertheless, all three proteins are essential for effective capsid production.

It has been reported that inactivation of all three capsid proteins VP1 to VP3 prevents accumulation of single-strand progeny AAV DNA. Mutations in the VP1 amino-terminus (“Lip-negative” or “Inf-negative”) still allows for assembly of single-stranded DNA into viral particles whereby the infectious titer is greatly reduced.

The AAP open reading frame is encoding the assembly activating protein (AAP). It has a size of about 22 kDa and transports the native VP proteins into the nucleolar region for capsid assembly. This open reading frame is located upstream of the VP3 protein encoding sequence.

In individual AAV particles, only one single-stranded DNA molecule is contained. This may be either the “plus” or “minus” strand. AAV particles containing a DNA molecule are infectious. Inside the infected cell, the parental infecting single stranded DNA is converted into a double stranded DNA, which is subsequently amplified. The amplification results in a large pool of double stranded DNA molecules from which single strands are displaced and packaged into capsids.

Adeno-associated viral (AAV) vectors can transduce dividing cells as well as resting cells. It can be assumed that a transgene introduced using an AAV vector into a target cell will be expressed for a long period. One drawback of using an AAV vector is the limitation of the size of the transgene that can be introduced into cells.

Parvovirus particles, including AAV serotypes and variants thereof, provide a means for ex vivo, in vitro and in vivo delivery of nucleic acid, which excert an effect by themselves or encode proteins, into cells such that the infected cells express the encoded protein. AAVs are viruses useful as gene therapy vectors as they can penetrate cells and introduce nucleic acid/genetic material so that the nucleic acid/genetic material may be stably maintained in the infected cells. Because AAV are not associated with pathogenic disease in humans, AAVs are able to deliver heterologous polynucleotide sequences (e.g., therapeutic proteins and agents) to human patients without causing substantial AAV-related pathogenesis or disease.

AAV particles used as vehicles for effective gene delivery possess a number of desirable features for such applications, including tropism for dividing and non-dividing cells. Early clinical experience with these vectors also demonstrated no sustained toxicity and immune responses were minimal or undetectable. AAV are known to infect a wide variety of cell types in vivo and in vitro by receptor-mediated endocytosis or by transcytosis. These vector systems have been tested in humans targeting retinal epithelium, liver, skeletal muscle, airways, brain, joints and hematopoietic stem cells.

Recombinant AAV particles do not typically include viral genes associated with pathogenesis. Such particles typically comprise a genome, wherein one or more of the wild-type AAV genes have been deleted in whole or in part, for example, rep and/or cap genes, but retain at least one functional flanking ITR sequence, as necessary for the rescue, replication, and packaging of the recombinant vector into an rAAV. Thus, an AAV vector includes sequences required in cis for replication and packaging (i.e. functional ITR sequences).

Recombinant AAV particles, as well as methods and uses thereof, can be based on any wild-type AAV genome or serotype or combination thereof. As a non-limiting example, a rAAV can be based upon any wild-type AAV genome, i.e. comprise the respective ITR sequences, such as AAV-1, -2, -3, -4, -5, -6, -7, -8, -9, -10, -11, -12, 2i8, rh.74, rh.10 or 7m8 for example. Such particles can be based on the same strain or serotype (or subgroup or variant), or be different from each other. As a non-limiting example, a rAAV based upon one wild-type genome can be identical or different to one or more of the capsid proteins that package the vector. In addition, a recombinant AAV vector can be based upon an AAV (e.g., AAV-2) wild-type serotype genome distinct from one or more of the AAV capsid proteins that package the vector. For example, the AAV vector can be based upon AAV-2, whereas at least one of the three capsid proteins could be an AAV-1, AAV-3, AAV-4, AAV-5, AAV-6, AAV-7, AAV-8, AAV-9, AAV-10, AAV-11, AAV-12, AAV-218, AAV-rh.74, AAV-rh.10 or AAV-7m8 or a variant thereof, for example. AAV variant particles include variants and chimeras of AAV-1, AAV-2, AAV-3, AAV-4, AAV-5, AAV-6, AAV-7, AAV-8, AAV-9, AAV-10, AAV-11, AAV-12, AAV-218, AAV-rh.74, AAV-rh.10 and AAV-7m8 capsids.

In certain embodiments of all aspects and embodiments of the invention, the rAAVp is derived from a wild-type AAV particle selected from the group consisting of AAV-1, AAV-2, AAV-3, AAV-4, AAV-5, AAV-6, AAV-7, AAV-8, AAV-9, AAV-10, AAV-11, AAV-12, AAV-218, AAV-rh.74, AAV-rh.10 and AAV-7m8, as well as variant particles (e.g., capsid variants, such as amino acid insertions, additions, substitutions and deletions) thereof, for example, as set forth in WO 2013/158879, WO 2015/013313 and US 2013/0059732 (disclosing LK01, LK02, LK03, etc.).

In certain embodiments of all aspects and embodiments of the invention, the rAAVp comprises a capsid polypeptides with an amino acid sequence having 70% or more sequence identity to an wild-type AAV-1, AAV-2, AAV-3, AAV-4, AAV-5, AAV-6, AAV-7, AAV-8, AAV-9, AAV-10, AAV-11, AAV-12, AAV-218, AAV-rh.10, AAV-rh.74, or AAV-7m8 capsid sequence.

In certain embodiments of all aspects and embodiments of the invention, the rAAVp comprises one or two ITR sequence having 70% or more sequence identity to a wild-type AAV-1, AAV-2, AAV-3, AAV-4, AAV-5, AAV-6, AAV-7, AAV-8, AAV-9, AAV-10, AAV-11 or AAV-12 ITR sequence.

Recombinant AAV particles can be incorporated into pharmaceutical compositions. Such pharmaceutical compositions are useful for, among other things, administration and delivery to a subject in vivo or ex vivo. In certain embodiments, the pharmaceutical composition contains a pharmaceutically acceptable carrier or excipient. Such excipients include any pharmaceutical agent that does not itself induce an immune response harmful to the individual receiving the composition, and which may be administered without undue toxicity.

Protocols for the generation of adenoviral vectors have been described in U.S. Pat. Nos. 5,998,205; 6,228,646; 6,093,699; 6,100,242; WO 94/17810 and WO 94/23744, which are incorporated herein by reference in their entirety.

Recombinant Adeno-Associated Virus Particles (rAAVp)

Different methods are known in the art for generating recombinant AAV particles. For example, transfection with a transgene comprising plasmid and a plasmid comprising AAV helper sequences (rep and cap) in conjunction with co-infection with one AAV helper virus (e.g., adenovirus, herpesvirus, or vaccinia virus) or transfection with a recombinant transgene comprising plasmid, an AAV helper plasmid (comprising rep and cap), and an helper function plasmid. Non-limiting methods for generating rAAVp are described, for example, in U.S. Pat. Nos. 6,001,650, 6,004,797, WO 2017/096039, and WO 2018/226887. Following rAAVp production (i.e. particle generation in cell culture systems), rAAVp can be recovered from the host cells and/or cell culture supernatant and purified.

For the generation of recombinant AAV particles, expression of the Rep and Cap proteins, the helper proteins E1A, E1B, E2A and E4orf6 as well as optionally the adenoviral VA RNA in a single mammalian cell is required. The helper proteins E1A, E1B, E2A and E4orf6 can be expressed using any promoter as shown by Matsushita et al. (Gene Ther. 5 (1998) 938-945), especially the CMV IE promoter. Thus, any promoter can be operably linked to said genes for functional expression.

Generally, to produce rAAVp, different, complementing plasmids are co-transfected into a host cell. One of the plasmids comprises the transgene sandwiched between the two cis acting AAV ITRs. The missing AAV elements required for replication and subsequent packaging of progeny recombinant genomes, i.e. the open reading frames for the Rep and Cap proteins, are contained in trans on a second plasmid. The overexpression of the Rep proteins results in inhibitory effects on cell growth (Li, J., et al., J. Virol. 71 (1997) 5236-5243). Additionally, a third plasmid comprising the genes of a helper virus, i.e. E1, E4orf6, E2A and VA from adenovirus, is required for rAAV production.

To reduce the number of required plasmids, rep, cap and the adenovirus helper genes may be combined on a single plasmid.

Alternatively, the host cell may already stably express the E1 gene products. Such a cell is a HEK293 cell. The human embryonic kidney clone denoted as 293 was generated back in 1977 by integrating adenoviral DNA into human embryonic kidney cells (HEK cells) (Graham, F. L., et al., J. Gen. Virol. 36 (1977) 59-74). The HEK293 cell line comprises base pair 1 to 4344 of the adenovirus serotype 5 genome. This encompasses the E1A and E1B genes as well as the adenoviral packaging signals (Louis, N., et al., Virology 233 (1997) 423-429).

When using HEK293 cells the missing E2A, E4orf6 and VA genes can be introduced either by co-infection with an adenovirus or by co-transfection with an E2A-, E4orf6- and VA-expressing plasmid (see, e.g., Samulski, R. J., et al., J. Virol. 63 (1989) 3822-3828; Allen, J. M., et al., J. Virol. 71 (1997) 6816-6822; Tamayose, K., et al., Hum. Gene Ther. 7 (1996) 507-513; Flotte, T. R., et al., Gene Ther. 2 (1995) 29-37; Conway, J. E., et al., J. Virol. 71 (1997) 8780-8789; Chiorini, J. A., et al., Hum. Gene Ther. 6 (1995) 1531-1541; Ferrari, F. K., et al., J. Virol. 70 (1996) 3227-3234; Salvetti, A., et al., Hum. Gene Ther. 9 (1998) 695-706; Xiao, X., et al., J. Virol. 72 (1998) 2224-2232; Grimm, D., et al., Hum. Gene Ther. 9 (1998) 2745-2760; Zhang, X., et al., Hum. Gene Ther. 10 (1999) 2527-2537). Alternatively, adenovirus/AAV or herpes simplex virus/AAV hybrid vectors can be used (see, e.g., Conway, J. E., et al., J. Virol. 71 (1997) 8780-8789; Johnston, K. M., et al., Hum. Gene Ther. 8 (1997) 359-370; Thrasher, A. J., et al., Gene Ther. 2 (1995) 481-485; Fisher, J. K., et al., Hum. Gene Ther. 7 (1996) 2079-2087; Johnston, K. M., et al., Hum. Gene Ther. 8 (1997) 359-370).

In order to limit the transgene activity to specific tissues, i.e. to limit the site of action, the transgene can be operably linked to an inducible or tissue specific promoter (see, e.g., Yang, Y., et al. Hum. Gene. Ther. 6 (1995) 1203-1213).

The coding sequences of E1A and E1B (open reading frames) can be derived from a human adenovirus, such as, e.g., in particular of human adenovirus serotype 2 or serotype 5. An exemplary sequence of human Ad5 (adenovirus serotype 5) is found in GenBank entries X02996, AC_000008 and that of an exemplary human Ad2 in GenBank entry AC_000007. Nucleotides 505 to 3522 comprise the nucleic acid sequences encoding E1A and E1B of human adenovirus serotype 5. Plasmid pSTK146 as reported in EP 1 230 354, as well as plasmids pGS119 and pGS122 as reported in WO 2007/056994, can also be used as a source for the E1A and E1B open reading frames.

E1A is the first viral helper gene that is expressed after adenoviral DNA enters the cell nucleus. The ELA gene encodes the 12S and 13S proteins, which are based on the same E1A mRNA by alternative splicing. Expression of the 12S and 13S proteins results in the activation of the other viral functions E1B, E2, E3 and E4. Additionally, expression of the 12S and 13S proteins force the cell into the S phase of the cell cycle. If only the E1A-derived proteins are expressed, the cell will die (apoptosis).

E1B is the second viral helper gene that is expressed. It is activated by the E1A-derived proteins 12S and 13S. The E1B gene derived mRNA can be spliced in two different ways resulting in a first 55 kDa transcript and a second 19 kDa transcript. The E1B 55 kDa protein is involved in the modulation of the cell cycle, the prevention of the transport of cellular mRNA in the late phase of the infection, and the prevention of E1A-induced apoptosis of cells.

The E2 gene encodes different proteins. The E2A transcript codes for the single strand-binding protein (SSBP), which is essential for AAV replication

In addition, the E4 gene encodes several proteins. The E4 gene derived 34 kDa protein (E4orf6) prevents the accumulation of cellular mRNAs in the cytoplasm together with the E1B 55 kDa protein, but also promotes the transport of viral RNAs from the cell nucleus into the cytoplasm.

The viral associated RNA (VA RNA) is a non-coding RNA of adenovirus (Ad), regulating translation. The adenoviral genome comprises two independent copies: VAI (VA RNAI) and VAII (VA RNAII). Both are transcribed by RNA polymerase III (see, e.g., Machitani, M., et al., J. Contr. Rel. 154 (2011) 285-289) from a type 2 polymerases III promoter. For recombinant AAV particle production, the adenoviral VA RNA gene can be driven by any promoter.

The structure, function, and evolution of adenovirus-associated RNA using a phylogenetic approach was investigated by Ma, Y. and Mathews, M. B. (J. Virol. 70 (1996) 5083-5099). They provided alignments as well as consensus VA RNA sequences based on 47 known human adenovirus serotypes. Said disclosure is herewith incorporated by reference in its entirety into the current application.

VA RNAs, VAI and VAII, are composed of 157-160 nucleotides (nt).

Depending on the serotype, adenoviruses contain one or two VA RNA genes. VA RNAI is believed to play the dominant pro-viral role, while VA RNAII can partially compensate for the absence of VA RNAI (Vachon, V. K. and Conn, G. L., Virus Res. 212 (2016) 39-52).

The VA RNAs are not essential, but play an important role in efficient viral growth by overcoming cellular antiviral machinery. That is, although VA RNAs are not essential for viral growth, VA RNA-deleted adenovirus cannot grow during the initial step of vector generation, where only a few copies of the viral genome are present per cell, possibly because viral genes other than VA RNAs that block the cellular antiviral machinery may not be sufficiently expressed (see Maekawa, A., et al. Nature Sci. Rep. 3 (2013) 1136).

Maekawa, A., et al. (Nature Sci. Rep. 3 (2013) 1136) reported efficient production of adenovirus vector lacking genes of virus-associated RNAs that disturb cellular RNAi machinery, wherein HEK293 cells that constitutively and highly express flippase recombinase were infected to obtain VA RNA-deleted adenovirus by FLP recombinase-mediated excision of the VA RNA locus.

The human adenovirus 2 VA RNAI corresponds to nucleotides 10586-10810 of GenBank entry AC_000007 sequence. The human adenovirus 5 VA RNAI corresponds to nucleotides 10579-10820 of GenBank entry AC_000008 sequence.

### General Description of Recombinant AAV Particle Production

After entry into the host cell nucleus, AAV can follow either one of two distinct and interchangeable pathways of its life cycle: the lytic or the lysogenic. The former develops in cells infected with a helper virus such as Ad or herpes simplex virus (HSV) whereas the latter is established in host cells in the absence of a helper virus.

When a latently infected cell is super-infected with a helper virus, the AAV gene expression program is activated leading to the AAV Rep-mediated rescue (i.e., excision) of the provirus DNA from the host cell chromosome followed by replication and packaging of the viral genome. Finally, upon helper virus-induced cell lysis, the newly assembled virions (particles) are released. Thus, the lytic phase of the AAV life cycle is induced.

Therefore, in the presence of Ad helper functions, the transgene is subjected to the wild-type AAV lytic processes by being rescued from the plasmid backbone, replicated and packaged into preformed AAV capsids as single-stranded molecules (Gonçalves, M.A.F.V., Virol. J., 2 (2005) 43).

Generation of a recombinant AAV particle involves replacing a majority of the AAV's wild-type genome with a desired transgene and providing the viral genes that are essential for virus packaging in-trans on a separate plasmid. Once all components are transfected together into a packaging cell line, recombinant AAV particles are assembled using the cell's cellular machineries. The process of viral assembly and encapsulation takes roughly two days, after which the cells are lysed to release the rAAVps for further purification and concentration (https://old.abmgood.com/marketing/knowledge_base/Adeno_Associated_Virus_P roduction_and_Modification_of_AAV.php).

rAAVps are not released very efficiently from the cells, although major differences have been observed between serotypes (see, e.g., Strobel, B., et al., Lamla T. Comparative Analysis of Cesium Chloride- and Iodixanol-Based Purification of Recombinant Adeno-Associated Viral Vectors for Preclinical Applications. Hum. Gene Ther. Methods 26 (2015) 147-157). When harvesting the culture, a cell disruption method is usually applied to recover the vectors entrapped in the cells.

Historically, manufacturing of rAAVps was performed by double transfection of a plasmid containing the rep and the cap ORFs and a plasmid with the gene of interest flanked by ITRs. Then, a helper virus, typically Adenovirus, was co-infected (see, e.g., Aponte-Ubillus, J. J., et al., Appl. Microbiol. Biotechnol. 102 (2018) 1045-1054; Muzyczka, N., Curr. Top. Microbiol. Immunol. 158 (1992) 97-129). In this setting, the separation of the helper virus from the final product was difficult, but a critical element to avoid induction of inflammatory responses after injection into patients (see, e.g., Schnell, M. A., et al., Mol. Ther. 3 (2001) 708-722). Therefore, production of rAAVps nowadays moved towards an adenovirus-free approach by utilizing triple transfection (see, e.g., Large, E. E., et al., Viruses 13 (2021) 1336). To this end, three components are needed: one plasmid encoding the genes for Rep and Cap without the ITRs, a second plasmid with the transgene of interest flanked by ITRs, and a helper plasmid to provide the helper genes of the helper virus (see, e.g., Aponte-Ubillus, J. J., et al., Appl. Microbiol. Biotechnol. 102 (2018) 1045-1054; Farris, K. D. and Pintel, D. J., Hum. Gene Ther. 19 (2008) 1421-1427; Grimm, D., et al., Hum. Gene Ther. 9 (1998) 2745-2760; Ferrari, F. K., et al., Nat. Med. 3 (1997) 1295-1297). For example, the Adenovirus helper bears the minimal required adenoviral genes E2A, E4 and VA. It is important to note, that the Human Embryonic Kidney cells 293 (HEK293) constitutively express the adenoviral genes E1A/B, which are also required for production of rAAVps. Therefore, HEK293 cells are classic producer cells for rAAVps and for manufacturing. Other cell types require a supplementation of E1A/B.

Carter et al. have shown that the entire rep and cap open reading frames in the wild-type AAV genome can be deleted and replaced with a transgene (Carter, B. J., in “Handbook of Parvoviruses”, ed. by P. Tijssen, CRC Press, pp. 155-168 (1990)). Further, it has been reported that the ITRs have to be maintained to retain the function of replication, rescue, packaging, and integration of the transgene into the genome of the target cell.

When cells comprising the respective viral helper genes are transduced by a transgene, or, vice versa, when cells comprising an integrated transgene are transduced by a suitable helper virus, then the AAV provirus is activated and enters a lytic infection cycle again (Clark, K. R., et al., Hum. Gene Ther. 6 (1995) 1329-1341; Samulski, R. J., Curr. Opin. Genet. Dev. 3 (1993) 74-80).

Producer cells contain the rep and cap gene sequences, as well as the transgene cassette flanked by ITR sequences on one or more plasmids that are retained, e.g., via drug selection. Production of rAAVps in these cell lines generally occurs after their infection with the required helper functions. Therefore, cells are infected either with replication-competent adenovirus (usually wild-type Ad5) or with a plasmid comprising the respective helper genes to supply helper virus proteins and initiate rAAVp production. A packaging cell line differs from a producer cell line as it only contains the rep and cap genes.

More generally, cells transfected or transduced with DNA for the recombinant production of AAV particles can be referred to as a “recombinant cell”. Such a cell can be any mammalian cell that has been used as recipient of a nucleic acid (plasmid) encoding packaging proteins, such as AAV packaging proteins, a nucleic acid (plasmid) encoding helper proteins, and a nucleic acid (plasmid) that encodes a protein or is transcribed into a transcript of interest, i.e. a transgene placed between two AAV ITRs. The term includes the progeny of the original cell, which has been transduced or transfected. It is understood that the progeny of a single parental cell may not necessarily be completely identical in morphology or in genomic or total nucleic acid complement as the original parent, due to natural, accidental, or deliberate mutation.

Numerous cell growth media appropriate for sustaining cell viability or providing cell growth and/or proliferation are commercially available. Examples of such medium include serum free eukaryotic growth mediums, such as medium for sustaining viability or providing for the growth of mammalian (e.g., human) cells. Non-limiting examples include Ham's F12 or F12K medium (Sigma-Aldrich), FreeStyle (FS) F17 medium (Thermo-Fisher Scientific), MEM, DMEM, RPMI-1640 (Thermo-Fisher Scientific) and mixtures thereof. Such media can be supplemented with vitamins and/or trace minerals and/or salts and/or amino acids, such as essential amino acids for mammalian (e.g., human) cells.

For producing rAAVps, three plasmids are co-transfected into a mammalian cell. The transgene plasmid encodes the expression cassette, which is cloned between the AAV ITRs, whereas rep and cap genes are provided in trans by co-transfecting a second, packaging plasmid (rep/cap plasmid) to ensure AAV replication and packaging. The third plasmid, also referred to as helper plasmid, contains the minimal helper virus factors, commonly adenoviral E2A, E4orf6 and VA genes, but lacking AAV ITRs.

Diverse methods for the DNA transfer into mammalian cells have been reported in the art. These are all useful in the methods according to the current invention. In certain embodiments of all aspects and embodiments, electroporation, nucleofection, or microinjection for nucleic acid transfer/transfection is used. In certain embodiments of all aspects and embodiments, an inorganic substance (such as, e.g., calcium phosphate/DNA co-precipitation), a cationic polymer (such as, e.g., polyethylenimine, DEAE-dextran), or a cationic lipid (lipofection) is used for nucleic acid transfer/transfection. Calcium phosphate and polyethylenimine are the most commonly used reagents for transfection for nucleic acid transfer in larger scales (see, e.g., Baldi et al., Biotechnol. Lett. 29 (2007) 677-684), whereof polyethylenimine is preferred.

The growth in serum-free suspension culture and improvement of efficiency and reproducibility of transfection conditions using PEI as a transfection reagent permits ready scale-up the AAV production using shake-flasks, wave, or stirred-tank bioreactors.

The composition may comprise further plasmids or/and cells. Such plasmids and cells may be in contact with free PEI.

In addition to PEI, valproic acid (VPA) can be used to improve transfection efficiency. VPA, a branched short-chain fatty acid and inhibits histone deacetylase activity. Due to this reason, it is commonly added to mammalian cell culture as an enhancer of recombinant protein production.

Encoded AAV packaging proteins include, in certain embodiments of all aspects and embodiments, AAV rep and/or AAV cap. Such AAV packaging proteins include, in certain embodiments of all aspects and embodiments, AAV rep and/or AAV cap proteins of any AAV serotype.

Encoded helper proteins include, in certain embodiments of all aspects and embodiments, adenovirus E1A and E1B, adenovirus E2 and/or E4, VA RNA, and/or non-AAV helper proteins.

The cultivation can be performed using the generally used conditions for the cultivation of eukaryotic cells of about 37° C., 95% humidity and 5 vol.-% CO2. The cultivation can be performed in serum containing or serum free medium, in adherent culture or in suspension culture. The suspension cultivation can be performed in any fermentation vessel, such as, e.g., in stirred tank reactors, wave reactors, rocking bioreactors, shaker vessels or spinner vessels or so called roller bottles. Transfection can be performed in high throughput format and screening, respectively, e.g. in a 96 or 384 well format.

Methods according to the current invention can include AAV particles of any serotype, or a variant thereof. In certain embodiments of all aspects and embodiments, a recombinant AAV particle comprises any of AAV serotypes 1-12, an AAV VP1, VP2 and/or VP3 capsid protein, or a modified or variant AAV VP1, VP2 and/or VP3 capsid protein, or wild-type AAV VP1, VP2 and/or VP3 capsid protein. In certain embodiments of all aspects and embodiments, an AAV particle comprises an AAV serotype or an AAV pseudotype, where the AAV pseudotype comprises an AAV capsid serotype different from an ITR serotype.

Expression control elements include constitutive or regulatable control elements, such as a tissue-specific expression control element or promoter.

ITRs can be any of AAV-2 or AAV-6 or AAV-8 or AAV-9 serotypes, or a combination thereof. AAV particles can include any VP1, VP2 and/or VP3 capsid protein having 75% or more sequence identity to any of AAV-1, AAV-2, AAV-3, AAV-4, AAV-5, AAV-6, AAV-10, AAV-11, AAV-12, AAV 218, AAV rh. 10, AAV rh.74 or AAV 7m8 VP1, VP2 and/or VP3 capsid proteins, or comprises a modified or variant VP1, VP2 and/or VP3 capsid protein selected from any of AAV-1, AAV-2, AAV-3, AAV-4, AAV-5, AAV-6, AAV-8, AAV-9, AAV-218, AAV-rh.10, AAV-rh.74 and AAV-7m8 AAV serotypes.

Following production of recombinant viral (e.g., AAV) particles, if desired, the viral (e.g., rAAV) particles can be purified and/or isolated from host cells using a variety of conventional methods. Such methods include column chromatography, CsCl gradients, iodixanol gradient and the like.

For example, a plurality of column purification steps such as purification over an anion exchange column, an affinity column and/or a cation exchange column can be used. (See, e.g., WO 02/12455 and US 2003/0207439). Alternatively, or in addition, an iodixanol or CsCl gradient steps can be used (see, e.g., US 2012/0135515; and US 2013/0072548). Further, if the use of infectious virus is employed to express the packaging and/or helper proteins, residual virus can be inactivated, using various methods. For example, adenovirus can be inactivated by heating to temperatures of approximately 60° C. for, e.g., 20 minutes or more. This treatment effectively inactivates the helper virus since AAV is heat stable while the helper adenovirus is heat labile.

An objective in the rAAVp production and purification systems is to implement strategies to minimize/control the generation of production related impurities such as proteins, nucleic acids, and vector-related impurities, including wild-type/pseudo wild-type AAV species (wtAAV) and AAV-encapsulated residual DNA impurities.

Considering that the rAAVps represents only a minor fraction of the biomass, rAAVps need to be purified to a level of purity, which can be used as a clinical human gene therapy product (see, e.g., Smith P. H., et al., Mo. Therapy 7 (2003) 8348; Chadeuf G., et al, Mo. Therapy 12 (2005) 744; report from the CHMP gene therapy expert group meeting, European Medicines Agency EMEA/CHMP 2005, 183989/2004).

In certain embodiments of all aspects and embodiments of the method according to the current invention, as an initial step, typically the cultivated cells that produce the rAAVps are harvested, optionally in combination with harvesting cell culture supernatant (medium) in which the cells (suspension or adherent) producing recombinant AAV particles have been cultured. The harvested cells and optionally cell culture supernatant may be used as is, as appropriate, lysed or concentrated. Further, if infection is employed to express helper functions, residual helper virus can be inactivated. For example, adenovirus can be inactivated by heating to temperatures of approximately 60° C. for, e.g., 20 minutes or more, which inactivates only the helper virus since rAAVps are heat stable while the helper adenovirus is heat labile.

The cells in the harvested cultivation broth can be lysed using methods now in the art, such as, e.g., detergent lysis or freeze-thaw cycles, to release the rAAVps. Concurrently during cell lysis or subsequently after cell lysis, a nuclease, such as, e.g., benzonase, is added to degrade contaminating DNA. Typically, the resulting lysate is clarified to remove cell debris, e.g. by filtering or centrifuging, to render a clarified cell lysate. In a particular example, the lysate is filtered with a micron diameter pore size filter (such as a 0.1-10.0 μm pore size filter, for example, a 0.45 μm and/or pore size 0.2 μm filter), to produce a clarified lysate.

The lysate (optionally clarified) contains recombinant AAV particles (comprising full as well as empty rAAVps) and production/process related impurities, such as soluble cellular components from the host cells that can include, inter alia, cellular proteins, lipids, and/or nucleic acids, and cell culture medium components. The optionally clarified lysate is then subjected to purification steps to purify the rAAVps (comprising (rAAV) transgenes) from impurities using chromatography. The clarified lysate may be diluted or concentrated with an appropriate buffer prior to the first chromatography step.

After cell lysis, optional clarifying, and optional dilution or concentration, a plurality of subsequent and sequential chromatography steps can be used to purify the rAAVps.

The first chromatography step is preferably an affinity chromatography step using an AAV affinity chromatography ligand.

If the first chromatography step is affinity chromatography the second chromatography step can be anion exchange chromatography. Thus, in certain embodiments of all aspects and embodiments, rAAVp purification is via affinity chromatography, followed by purification via anion exchange chromatography or/and cation exchange chromatography or/and size exclusion chromatography, in any order or sequence or combination.

The removal of empty capsids from full ones, for example, during downstream processing is based on their different isoelectric points (pI) in anion exchange chromatography. The average calculated pI across all serotypes is 5.9 for full capsids and 6.3 for empty capsids (Venkatakrishnan, B., et al., J. Virol. 87 (2013) 4974-4984).

Alternatively, gradient centrifugation is known for being able to separate empty and full particles based on their distinctive densities.

Cation exchange chromatography functions to separate the rAAVps from cellular and other components present in the clarified lysate and/or column eluate from an affinity or size exclusion chromatography. Examples of strong cation exchange resins capable of binding rAAVps over a wide pH range include, without limitation, any sulfonic acid based resin as indicated by the presence of the sulfonate functional group, including aryl and alkyl substituted sulfonates, such as sulfopropyl or sulfoethyl resins. Representative matrices include but are not limited to POROS HS, POROS HS 50, POROS XS, POROS SP, and POROS S (strong cation exchangers available from Thermo Fisher Scientific, Inc., Waltham, MA, USA). Additional examples include Capto S, Capto S ImpAct, Capto S ImpRes (strong cation exchangers available from GE Healthcare, Marlborough, MA, USA), and commercial DOWEX®, AMBERLITE®, and AMBERLYST® families of resins available from Aldrich Chemical Company (Milliwaukee, WI, USA). Weak cation exchange resins include, without limitation, any carboxylic acid based resin. Exemplary cation exchange resins include carboxymethyl (CM), phospho (based on the phosphate functional group), methyl sulfonate(S) and sulfopropyl (SP) resins.

Anion exchange chromatography functions to separate rAAVps from proteins, cellular and other components present in the clarified lysate and/or column eluate from an affinity or cation exchange or size exclusion chromatography. Anion exchange chromatography can also be used to reduce and thereby control the amount of empty rAAVps in the eluate. For example, the anion exchange column having full and empty rAAVps bound thereto can be washed with a solution comprising NaCl at a modest concentration (e.g., about 100-125 mM, such as 110-115 mM) and a portion of the empty rAAVps can be eluted in the flow through without substantial elution of the full rAAVps. Subsequently, full rAAVps bound to the anion exchange column can be eluted using a solution comprising NaCl at a higher concentration (e.g., about 130-300 mM NaCl), thereby producing a column eluate with reduced or depleted amounts of empty rAAVps and proportionally increased amounts of full rAAVps comprising a transgene.

Exemplary anion exchange resins include, without limitation, those based on polyamine resins and other resins. Examples of strong anion exchange resins include those based generally on the quaternized nitrogen atom including, without limitation, quaternary ammonium salt resins such as trialkylbenzyl ammonium resins. Suitable exchange chromatography materials include, without limitation, MACRO PREP Q (strong anion-exchanger available from BioRad, Hercules, CA, USA); UNOSPHERE Q (strong anion-exchanger available from BioRad, Hercules, CA, USA); POROS 50HQ (strong anion-exchanger available from Applied Biosystems, Foster City, CA, USA); POROS XQ (strong anion-exchanger available from Applied Biosystems, Foster City, CA, USA); POROS SOD (weak anion-exchanger available from Applied Biosystems, Foster City, CA, USA); POROS 50PI (weak anion-exchanger available from Applied Biosystems, Foster City, CA, USA); Capto Q, Capto XQ, Capto Q ImpRes, and SOURCE 30Q (strong anion-exchanger available from GE healthcare, Marlborough, MA, USA); DEAE SEPHAROSE (weak anion-exchanger available from Amersham Biosciences, Piscataway, NJ, USA); Q SEPHAROSE (strong anion-exchanger available from Amersham Biosciences, Piscataway, NJ, USA). Additional exemplary anion exchange resins include aminoethyl (AE), diethylaminoethyl (DEAE), diethylaminopropyl (DEPE) and quaternary amino ethyl (QAE).

A commercial manufacturing process to purify recombinant AAV particles intended as a product to treat human disease should achieve the following objectives: 1) consistent particle purity, potency and safety; 2) manufacturing process scalability; and 3) acceptable cost of manufacturing.

Exemplary processes for recombinant AAV particle purification are reported in WO 2019/006390.

Methods to determine infectious titer of rAAV particles containing a transgene are known in the art (see, e.g., Zhen et al., Hum. Gene Ther. 15 (2004) 709). Methods for assaying for empty rAAV and full rAAV with packaged transgenes are known (see, e.g., Grimm et al., Gene Therapy 6 (1999) 1322-1330; Sommer et al., Malec. Ther. 7 (2003) 122-128).

To determine the presence or amount of degraded/denatured capsid, purified rAAVps can be subjected to SDS-polyacrylamide gel electrophoresis, consisting of any gel capable of separating the three capsid proteins, for example, a gradient gel, then running the gel until sample is separated, and blotting the gel onto nylon or nitrocellulose membranes. Anti-AAV capsid antibodies are then used as primary antibodies that bind to denatured capsid proteins (see, e.g., Wobus et al., J. Viral. 74 (2000) 9281-9293). A secondary antibody that binds to the primary antibody contains a means for detecting the primary antibody. Binding between the primary and secondary antibodies is detected semi-quantitatively to determine the amount of capsids. Another method would be analytical HPLC with a SEC column or analytical ultracentrifuge.

### Exemplary Embodiments of the Current Invention

To modify the natural tropism of AAV particles, capsid proteins can be modified by either the genetic insertion of or the enzymatic conjugation to binding sites as targeting domains, such as, e.g., peptides and nanobodies. The modified AAV particles, i.e. AAV particles genetically or enzymatically conjugated to a binding site, have the potential to efficiently transduce cells expressing the target of the binding site and thereby change the natural tropism of an AAV particle.

AAV serotypes differ in their capsid composition and structure, which determines virus binding to cell receptors and the overall tropism. Many non-conserved capsid domains, so called variable regions (VRs), map to exposed surface loops, which mediate specific receptor binding and cell entry. Those surface loops can be manipulated to prevent natural binding of the virus to (co-) receptors and change the tropism of the respective AAV particle by inserting or conjugating protein domains with target binding abilities.

The current invention is based, at least in part, on the finding that suitable insertion sites for binding sites as targeting ligands can be found within the GH2/GH3 loop (VR IV, loop 4) or within VRVIII (loop 8) of AAV-2, AAV-5 and AAV-9. In more detail, it has been found that i) in an AAV-2 VP1 capsid protein amino acid residues S452-T455 or G453-R459 can be replaced by tags for enzymatic conjugation, nanobodies, binding sites or peptide sequences or they can be inserted at amino acid residue T456; ii) in an AAV-5 VP1 capsid protein amino acid residues N442-T444 or T444-G446 or T557-A579 can be replaced by tags for enzymatic conjugation, nanobodies, binding sites or peptide; and iii) in an AAV-9 VP1 capsid protein amino acid residues S454-Q458 can be replaced by tags for enzymatic conjugation, nanobodies, binding sites or peptide sequences.

The current invention is exemplified in the following with certain constructs. These are presented merely to exemplify the invention and shall not be construed as limitation. The true scope of the invention is set forth in the appended claims.

As one example for a change of the tropism of an AAV particle the blood-brain barrier (BBB) was chosen. The BBB represents a major obstacle for efficiently delivering drugs into the brain. Towards this end, a change of the tropism of AAV particles was achieved according to the current invention by enzymatic conjugation of TfR binding sites or by genetic fusions of TfR or CD98 binding sites to allow for receptor-mediated uptake at the BBB and thereby enhanced delivery to the brain.

Other examples include targeting of Her2 and/or EGFR positive cells.

### Enzymatic Conjugation

Herein is reported a novel method for the generation of retargeted recombinant adeno-associated virus particles (rAAVps) that circumvents the limitations inherent to prior art methodologies by the use of an enzymatic conjugation technology. This novel strategy for changing the tropism of AAV particles mitigates the drawbacks of previously known methods, such as, amongst other things, immunogenicity concerns.

AAV serotypes differ in their capsid protein amino acid sequence and structure, which determines the binding to cell receptors and the overall AAV tropism. Many non-conserved capsid domains, so called variable regions (VRs), map to exposed surface loops, which mediate specific receptor binding and cell entry. Those surface loops can be manipulated to prevent natural binding of the virus to (co-) receptors and re-direct AAV-tropism by using them as conjugation sites.

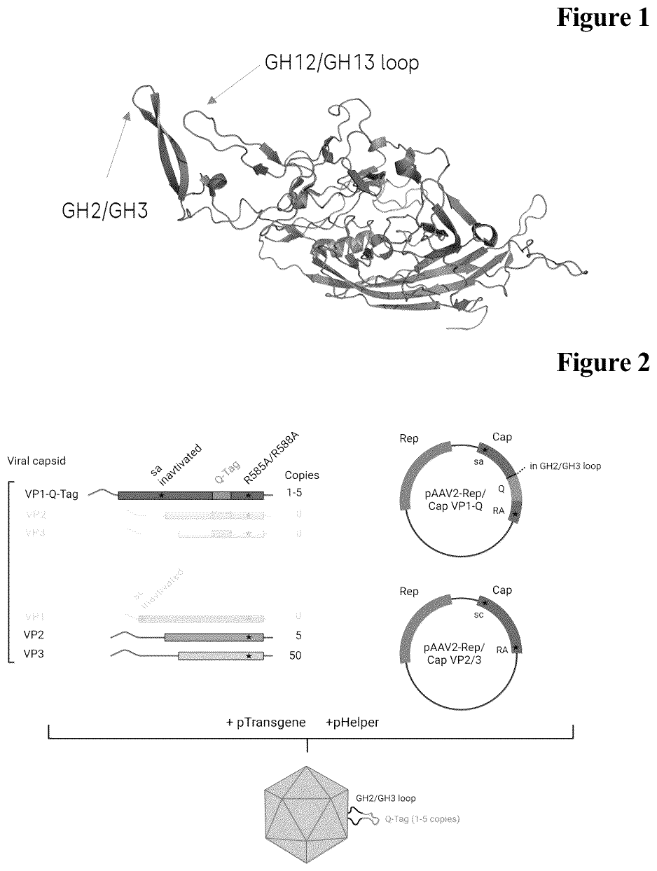

In the current invention, the GH2/GH3 loop (VR IV, loop 4) of AAV-2 has been found to be a particularly suitable insertion site. The inserted peptide tag was flanked by G4S-linker. This construct replaced residues S452-T455 of a wild-type AAV-2 VP1 of SEQ ID NO: 01 (see FIG. 1).

To remove the wild-type tropism of AAV-2, mutations R585A and R588A were introduced to silence HSPG binding (SEQ ID NO: 1, 2, 3).

Viral capsid proteins VP1, VP2, and VP3 are encoded by one open reading frame (ORF) and assemble in a ratio of 1:1:10. In order to target only VP1 as a conjugation site, two distinct expression plasmids for viral capsid proteins VP1 and VP2/3 were used. The first expression plasmid encodes AAV VP1 with a Q-tag inserted at a site according to the current invention and carries a mutated splice acceptor site (sa), which prevents expression of VP2 and VP3 with a Q-tag. Thereby the number of conjugation sites is controlled. Non-modified AAV VP2 and VP3 are encoded by the second expression plasmid, in which a mutated start codon (sc) for the VP1 coding region prevents expression of non-modified AAV VP1. Schemes of the plasmids are shown in FIG. 2.

It is known from the art that the yield of enzymatic conjugations using KTG is depending on the conjugation site. However, a preference for a specific conjugation site cannot be predicted (see, e.g., WO 2023/118398).

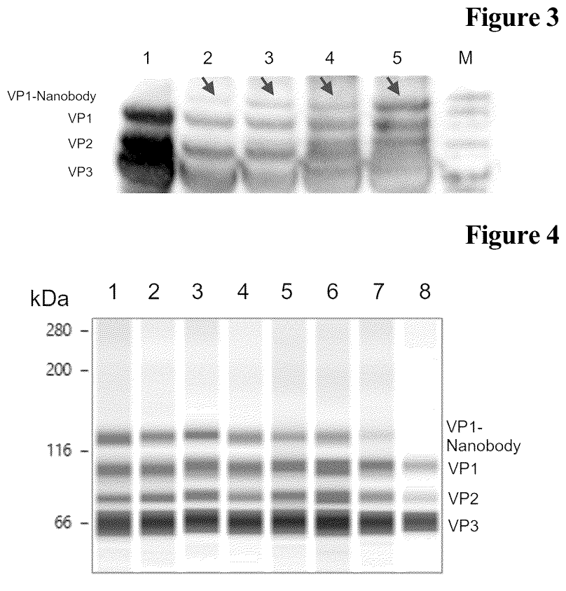

In a first set of experiments, two nanobodies specifically binding to the human transferrin receptor (TfR) were modified by introducing a K-tag at either the N- or the C-terminus (SEQ ID NO: 11, 12, 13 and 14). KTG-mediated conjugation of the respective K-tagged nanobodies was carried out at 37° C. in PBS at pH 7.4. About 170 nM AAV-2-Q-tag variant particle (1E+14 vp/mL) was incubated with 1.7 μM K-tagged nanobody and 500 nM KTG. After overnight incubation, the reaction mixtures were analyzed by Western Blot using an antibody against all three capsid proteins (anti-AAV VP1/VP2/VP3 rabbit polyclonal VP51, 61084, Progen, dilution 1:20). The results are shown in FIG. 3.

It can be seen that a new band appeared when the viral particles were incubated with the K-tagged nanobody in the presence of the enzyme (see FIG. 3, marked with an arrow). Further, for both nanobodies, the coupling efficiency was significantly higher when the K-tag was located at the C-terminus (FIG. 3, lanes 3 and 5).

Thus, it has unexpectedly been found that for the conjugation of a nanobody by KTG to an AAV particle comprising a Q-tag the location of the K-tag at the C-terminus resulted in higher enzymatic conjugation yields.

In a second set of experiments, in the conjugation reaction different concentrations of AAV-2-Q-tag variant particles were used. Concentrations in the range from 100 nM to 453 nM were tested. Nanobody MH12-K-tag (SEQ ID NO: 14) was used in this experiment. The concentration of the nanobody was fixed at 4.53 μM and 500 nM KTG was used. Reactions were carried out at 37° C. overnight (15 hours). Samples were analyzed via Western Blot. The results are shown in FIG. 4.

It has been found that the minimum concentration needed for a successful coupling reaction is 150 nM of the AAV-Q-tag variant particle whereas maximum conjugation occurred at a concentration of 300 nM of the AAV-Q-tag variant particle. Unexpectedly, above said concentration no significant increase in coupling efficiency could be achieved. Therefore, the conjugation of AAV-Q-tag variant particles with nanobody-K-tag conjugates is preferably conducted with a concentration of 150 nM to 300 nM of the AAV-Q-tag variant particle whereby the concentration of about 300 nM (corresponding to 1.8E+14 vp/mL) is most preferred for coupling reactions with a nanobody-K-tag fusion by KTG.

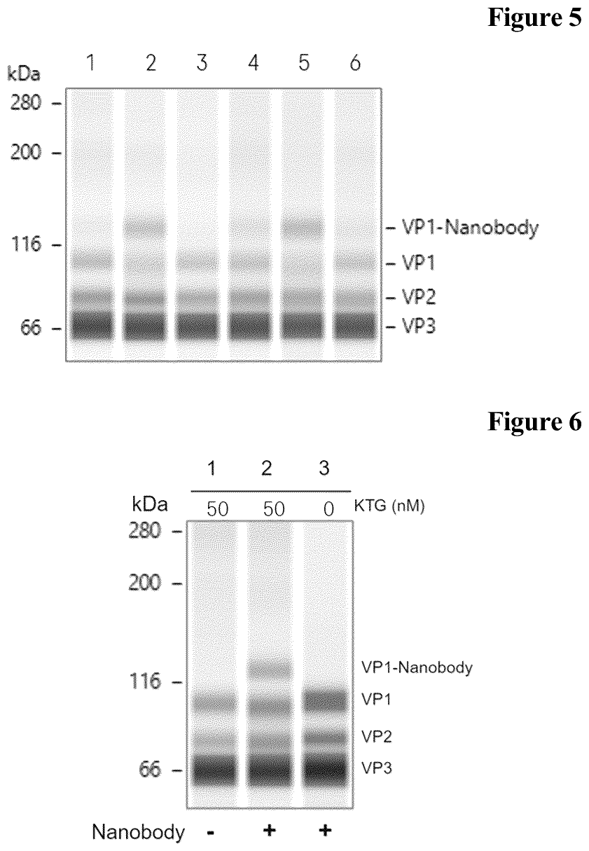

In a third set of experiments, different concentrations of the transglutaminase were tested. Therefore enzymatic conjugation reactions were carried out with 300 nM AAV-Q-tag variant particles (AAV-2), 15 μM nanobody-K-tag conjugate (SEQ ID NO: 14) and different concentrations of the enzyme in PBS at a pH of 7.4 at 37° C. overnight (15 hours). The conditions are shown in Table 1 and the results are shown in FIG. 5.

TABLE 1
Summary of conditions for conjugation reaction.
All reactions were incubated for 15 h at 37° C.

| | AAV-2-Q-tag variant | KTG | MH12-K-tag [nM] |
|---|---|---|---|
| Lane | particle [nM] | [nM] | (SEQ ID NO: 14) |
| 1 | 300 | 362 | 15,000 |
| 2 | | 50 | 15,000 |
| 3 | | 362 | 4,500 |
| 4 | | 362 | 15,000 |
| 5 | | 100 | 15,000 |
| 6 | | 500 | 15,000 |

It has been found that using too much of the enzyme is actually decreasing the conjugation yield. Lanes 2 and 5 of FIG. 5 obtained with 50 nM and 100 nM KTG, respectively, show better conjugation yields than lanes 1, 3, 4 and 6 with 362 nM and 500 nM KTG, respectively. Unexpectedly, when only 50 nM KTG was added to the reaction mixture, the best conjugation efficiency was achieved. Therefore, the conjugation of AAV-Q-tag variant particles with nanobody-K-tag conjugates is preferably conducted with a concentration of 50 nM to 200 nM of the transglutaminase whereby the concentration of about 50 nM to 100 nM is most preferred.

In a confirmatory experiment, an enzymatic conjugation reaction was carried out with the preferred conditions identified in the previous experiments and the AAV-2-nanobody conjugated particle was thereafter purified via AAVx affinity chromatography, yielding pure AAV-2-MH12-nanobody conjugates. The results are shown in FIG. 6.

Thus, in one preferred embodiment, the conjugation of AAV-Q-tag variant particles with nanobody-K-tag conjugates is conducted with a concentration of about 300 nM of the AAV-Q-tag variant particle (corresponding to 1.8E+14 vp/mL), a concentration of about 50 nM of the transglutaminase, preferably of KTG, at a temperature of 37° C. for about 15 hours. The nanobody-K-tag conjugate is preferably used at a concentration of about 15 μM.

The AAV-nanobody conjugates have been analyzed with respect to their binding properties via surface plasmon resonance (SPR). Therefore, recombinant human transferrin receptor (TfR) as the antigen for the nanobody in the conjugate was used as an analyte for determining the binding kinetics. Conjugated AAV-nanobody constructs served as capture ligands.

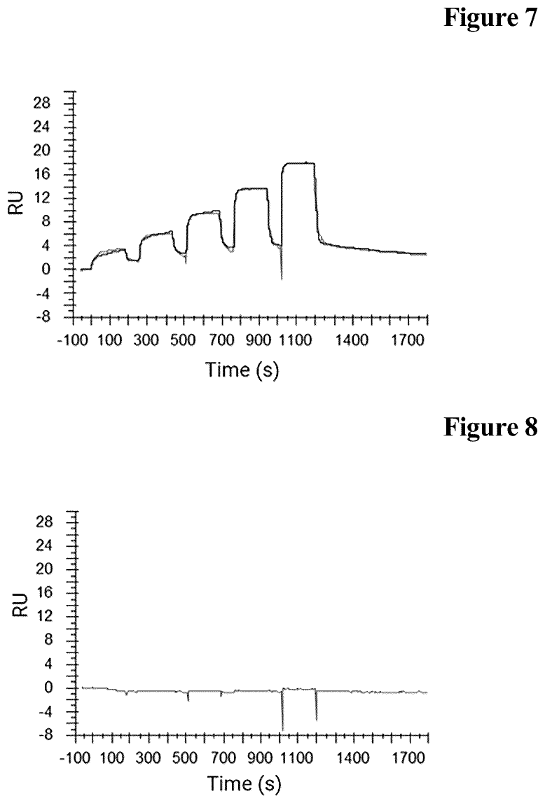

Three samples were applied to the AAVx immobilized chips: i) AAV-2-MH12 conjugate (SEQ ID NO: 1, 2, 3, 14), ii) mixture of non-conjugated AAV-2-Q-tag variant particles (SEQ ID NO: 1, 2, 3) incubated with nanobody (SEQ ID NO: 14) without KTG, and iii) mixture of AAV-2-Q-tag variant particles (SEQ ID NO: 1, 2, 3) incubated with KTG without nanobody. The respective sensorgrams are shown in FIG. 7 to FIG. 9.

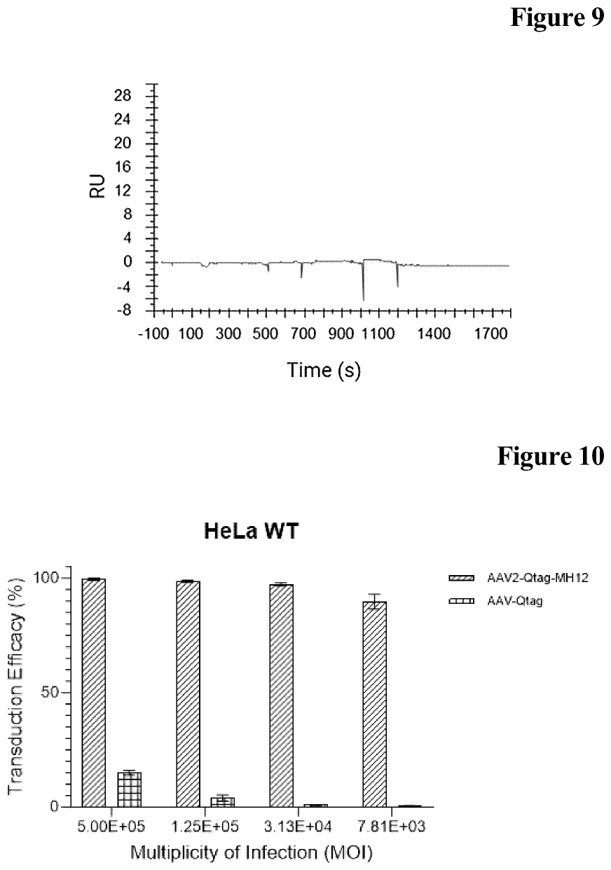

The results show that binding can only be detected in the presence of AAV-nanobody conjugates (FIG. 7). Binding cannot be detected in case of non-conjugated material (FIG. 8 and FIG. 9).

The respective KD values are summarized in Table 2.

TABLE 2
Kinetic rate parameters determined from FIG. 7
for affinity and avidity of the AAV-2-MH12 conjugate
(SEQ ID NO: 1, 2, 3, 14) to dimeric TfR.

| affinity | | | avidity | | |
|---|---|---|---|---|---|
| ka1 | kd1 | KD1 | ka2 | kd2 | KD2 |
| [1/Ms] | [1/s] | [M] | [1/Ms] | [1/s] | [M] |
| 1.70E+05 | 0.122 | 7.17E−07 | 3.96E+04 | 9.97E−04 | 2.52E−08 |

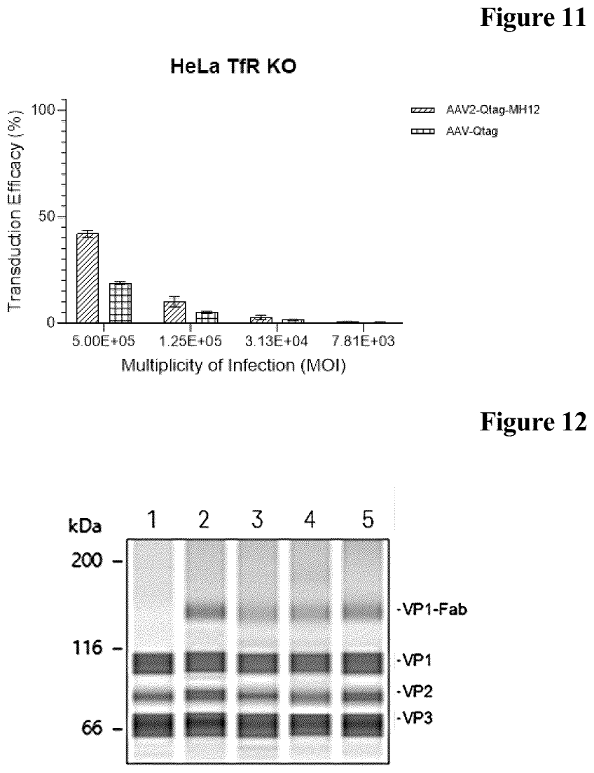

Cell transduction assays were performed to demonstrate the biological activity of the generated AAV-nanobody conjugates. Therefore, conjugated and non-conjugated AAV-2-Q-tag variant particles carrying mGreenLantern (mGL) as transgene were incubated on TfR expressing wild-type HeLa (WT) cells and HeLa TfR knockout (KO) cells. For the assessment of transgene expression, a FACS analysis was conducted. This allowed for the measurement of green fluorescent activity, which is indicative of the level of mGL transgene expression. The data from these assays were analyzed to determine the efficiency of AAV-mediated gene delivery across the different MOIs tested. The results are shown in FIG. 10 and FIG. 11.

AAV-nanobody conjugates showed MOI-dependent specific transduction of WT cells, while non-conjugated AAV-Q-tag variant particles did not transduce the cells efficiently (FIG. 10). This demonstrates that AAV particles with abolished natural tropism can be modified to bind to a specific target by enzymatic conjugation with a target specific binding site.

The assay provided a comprehensive evaluation of the transduction capabilities of the AAV particles and was designed to be adaptable for high-throughput applications, suitable for a variety of cell types as well as AAV-based constructs.

To show the general applicability of the approach according to the current invention, additional binding sites were conjugated to the AAV-2-Q-tag variant particle.

First, a TfR targeting Fragment antigen-binding region (Fab) was used.

As described above, it has been found that KTG preferred the K-tag at the C-terminus of the conjugation partner for conjugation to AAV-Q-tag variant particles. Hence, the K-tag was conjugated to the C-terminus of the Fab. Since these molecules have a higher molecular weight than nanobodies (48 kDa vs. 17 kDa), two different linker lengths were employed (SEQ ID NO: 29 and SEQ ID NO: 30). Additionally, two different Fabs (high affinity TfR binding site (SEQ ID NO: 19/78 and 21/80); low affinity TfR binding site (SEQ ID NO: 20/79 and 22/81)) were used. The constructs are shown in Table 3.

TABLE 3
Summary of constructs for conjugation reaction.

| number | AAV variant particle | SEQ ID NO: |
|---|---|---|
| 1 | negative control (AAV-2-Q-tag) | 1, 2, 3 |
| 2 | AAV-2-aTFR-Fab-2G4S (low affinity) | 1, 2, 3, 20, 79 |
| 3 | AAV-2-aTFR-Fab-2G4S (high affinity) | 1, 2, 3, 19, 78 |
| 4 | AAV-2-aTFR-Fab-4G4S (low affinity) | 1, 2, 3, 22, 81 |
| 5 | AAV-2-aTFR-Fab-4G4S (high affinity) | 1, 2, 3, 21, 80 |

The results of the conjugation reaction are shown in FIG. 12. All reactions were performed with 50 nM KTG and 15 μM Fab-K-tag for 15 hours at 37° C.

It can be seen that the linker length does not influence the conjugation efficiency.

Thus, it has been shown that the method according to the current invention is also applicable to the conjugation of K-tag Fabs to AAV-Q-tag variant particles, enabling the generation of bispecific AAV-2 particles.

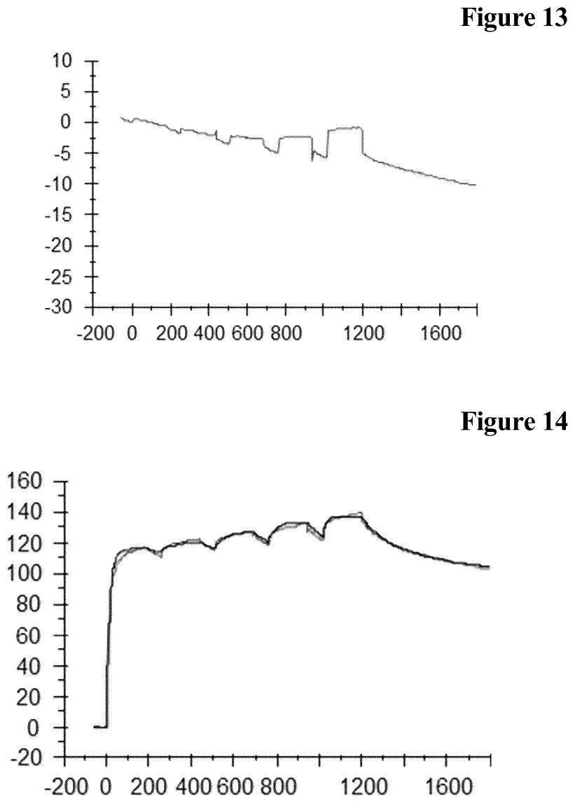

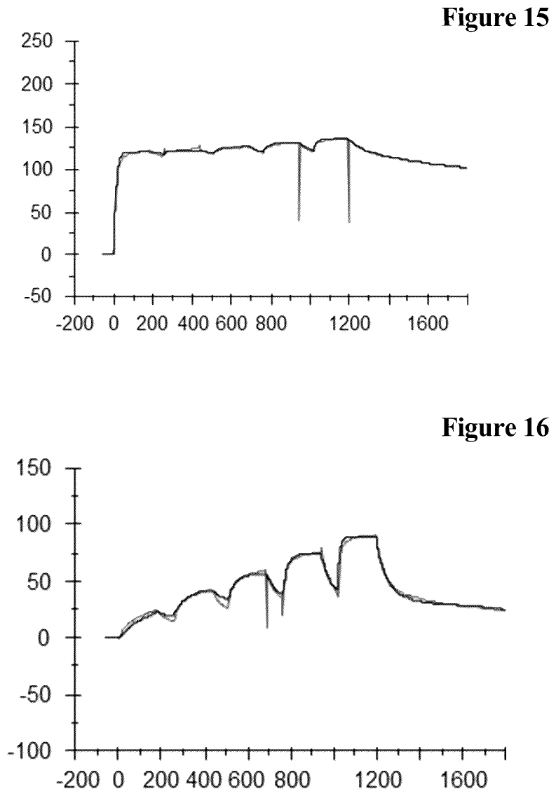

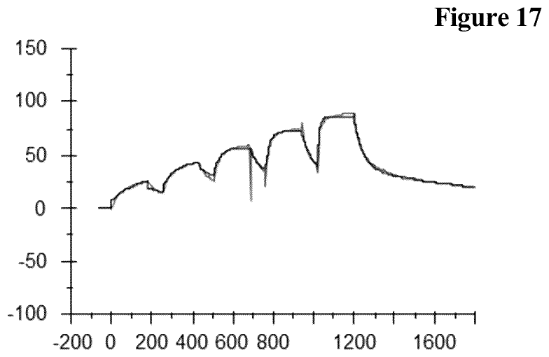

The binding properties of the AAV-Fab conjugates towards the biological target of the Fab were analyzed via surface plasmon resonance. The results are shown in FIG. 13 to FIG. 17.

The sensorgrams showed that AAV-2-aTfR-Fab conjugate binding towards the target of the Fab, i.e. the human transferrin receptor, was specifically mediated by the covalently conjugated Fab binding site, while the negative controls did not show any binding at all.

The respective KD values are summarized in Table 4.

TABLE 4
Kinetic rate parameters determined from FIGS. 13 to 17.

| Ligand | ka1 | SE[ka1] | kd1 | SE[kd1] | KD1 | SE |
|---|---|---|---|---|---|---|
| (SEQ ID NO:) | [1/Ms] | [1/Ms] | [1/s] | [1/s] | [M] | [KD1] |
| 19/78 | 5.78E+04 | 4.58E+02 | 7.82E−03 | 4.51E−05 | 1.35E−07 | 1.33E−09 |
| 20/79 | 5.83E+04 | 5.51E+02 | 2.07E−02 | 1.84E−04 | 3.55E−07 | 4.61E−09 |
| 21/80 | 4.71E+04 | 1.20E+03 | 1.24E−02 | 2.19E−04 | 2.63E−07 | 8.16E−09 |
| 22/81 | 4.36E+04 | 7.11E+02 | 2.23E−02 | 2.83E−04 | 5.11E−07 | 1.06E−08 |
| Ligand | ka2 | SE[ka2] | kd2 | SE[kd2] | KD2 | SE |
| (SEQ ID NO:) | [1/Ms] | [1/Ms] | [1/s] | [1/s] | [M] | [KD2] |
| 19/78 | 3.87E+06 | 1.06E+04 | 1.56E−04 | 1.04E−06 | 4.03E−11 | 2.91E−13 |
| 20/79 | 2.65E+05 | 2.16E+03 | 5.25E−04 | 1.40E−05 | 1.98E−09 | 5.52E−11 |
| 21/80 | 4.82E+06 | 2.80E+04 | 2.75E−04 | 1.91E−06 | 5.71E−11 | 5.17E−13 |
| 22/81 | 1.95E+05 | 2.13E+03 | 1.04E−03 | 1.65E−05 | 5.33E−09 | 1.03E−10 |

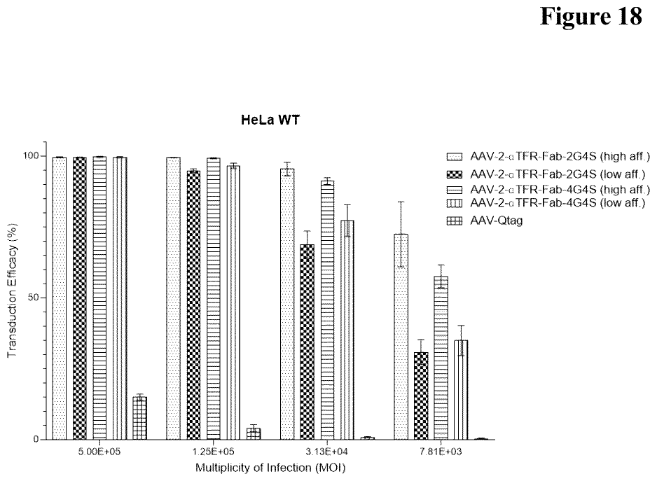

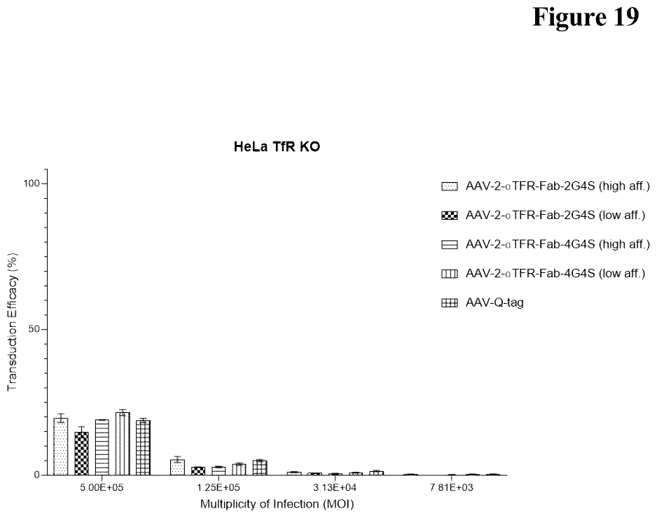

To demonstrate the biological activity of the AAV-Fab conjugates, transduction assays were performed. Therefore, conjugated and non-conjugated AAV-2 particles carrying mGreenLantern (mGL) as transgene were incubated with TfR displaying wild-type HeLa (WT) cells and HeLa TfR knockout (KO) cells. The results are shown in FIG. 18 and FIG. 19.

A MOI-dependent transduction was observed for all TfR targeting AAV-Fab variant particles. Furthermore, none of the variant particles nor the negative control transduced the TfR KO cell line efficiently, showing the advantage of the Fab-mediated targeting.

Second, Epidermal Growth Factor Receptor (EGFR) targeting fragment antigen-binding regions (Fab) and single-chain fragment variable domains (scFv) were used. As conjugation by KTG is preferably done with the K-tag conjugated to the C-terminus of the binding site as shown above, all Fab and scFv constructs were produced with the K-tag at the respective C-terminus. As these more complex molecules have a significantly higher molecular weight compared to nanobodies (48/28 kDa vs. 17 kDa), two different linker lengths were tested (SEQ ID NO: 29 and SEQ ID NO: 30). The used constructs are shown in Table 5.

TABLE 5
Summary of constructs for conjugation reaction.

| number | AAV variant particle | SEQ ID NO: |
|---|---|---|
| 1 | negative control | 1, 2, 3 |
| | (AAV-2-Q-tag) | |
| 2 | AAV-2-aEGFR-Fab-2G4S | 1, 2, 3, 15, 48 |
| 3 | AAV-2-aEGFR-Fab-4G4S | 1, 2, 3, 16, 77 |
| 4 | AAV-2-aEGFR-scFv-2G4S | 1, 2, 3, 17 |
| 5 | AAV-2-aEGFR-scFv-4G4S | 1, 2, 3, 18 |

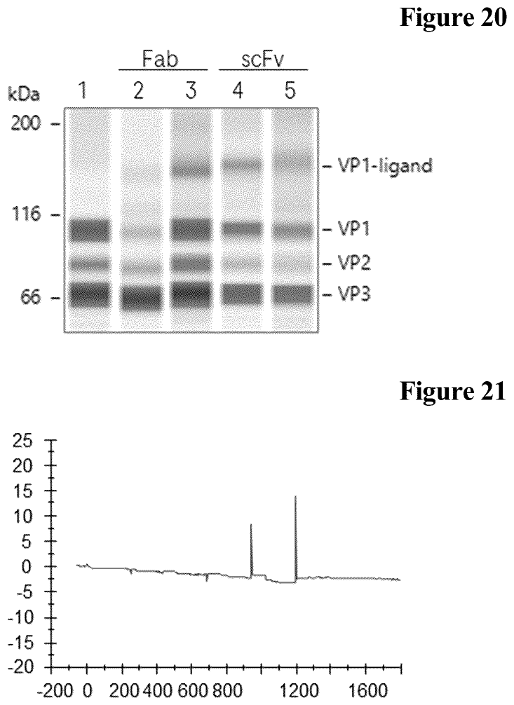

The results of the conjugation reaction are shown in FIG. 20. All reactions were performed with 50 nM KTG and 15 μM Fab-K-tag or scFv-K-tag with an incubation for 15 hours at 37° C.

It can be seen that the Fab construct with the long linker (SEQ ID NO: 1, 2, 3, 16, 77) was conjugated better compared to the construct with the short linker (SEQ ID NO: 1, 2, 3, 15, 48). Nonetheless, these results demonstrated the advantage and general applicability of the use of KTG to specifically conjugate different antibody formats to the viral capsid to generate targeted AAVps, i.e. change the tropism of the AAV particle.

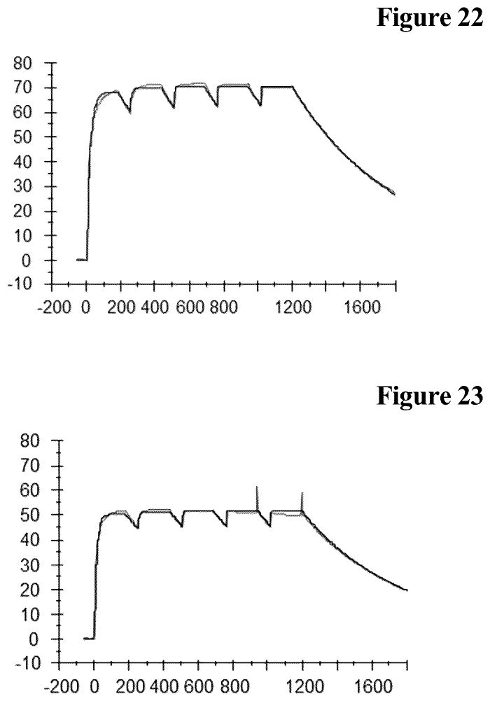

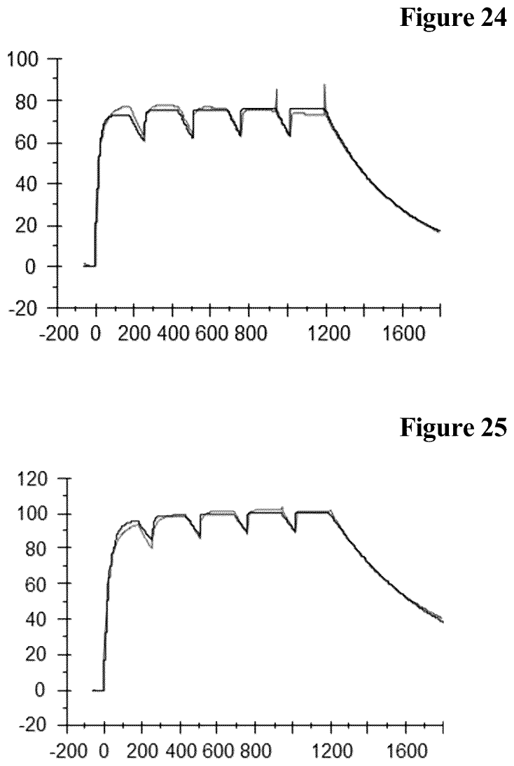

The binding properties of the AAV-Fab and AAV-scFv conjugates, i.e. of the AAV variant particles generated with the method according to the current invention, towards the biological target of the Fab/scFv were analyzed via surface plasmon resonance. The results are shown in FIG. 21 to FIG. 25.

The results showed that AAV-2-Fab/scFv binding towards EGFR was specifically mediated by the covalently conjugated binding sites, while the negative controls did not show any binding at all.

The respective KD values are summarized in Table 6.

TABLE 6
Kinetic rate parameters determined from FIGS. 21 to 25.

| Sample | | | | | | |
|---|---|---|---|---|---|---|
| (SEQ | ka | SE[ka] | kd | SE[kd] | KD | SE |
| ID NO:) | [1/Ms] | [1/Ms] | [1/s] | [1/s] | [M] | [KD] |
| 1, 2, 3, | 2.57E+06 | 4.92E+03 | 1.63E−03 | 1.12E−06 | 6.34E−10 | 1.29E−12 |
| 15, 48 | | | | | | |
| 1, 2, 3, | 3.87E+06 | 2.03E+04 | 1.71E−03 | 4.24E−06 | 4.42E−10 | 2.56E−12 |
| 16, 77 | | | | | | |
| 1, 2, 3, | 3.30E+06 | 1.85E+04 | 2.62E−03 | 8.13E−06 | 7.94E−10 | 5.09E−12 |
| 17 | | | | | | |
| 1, 2, 3, | 1.78E+06 | 3.98E+03 | 1.60E−03 | 1.62E−06 | 8.99E−10 | 2.21E−12 |
| 18 | | | | | | |

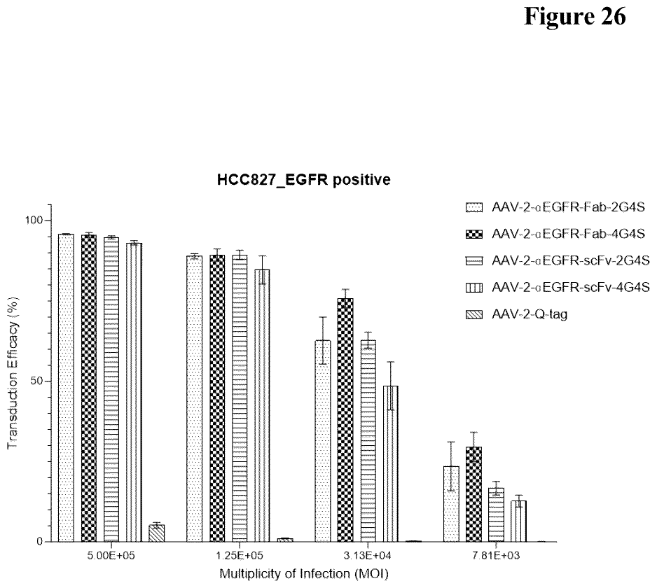

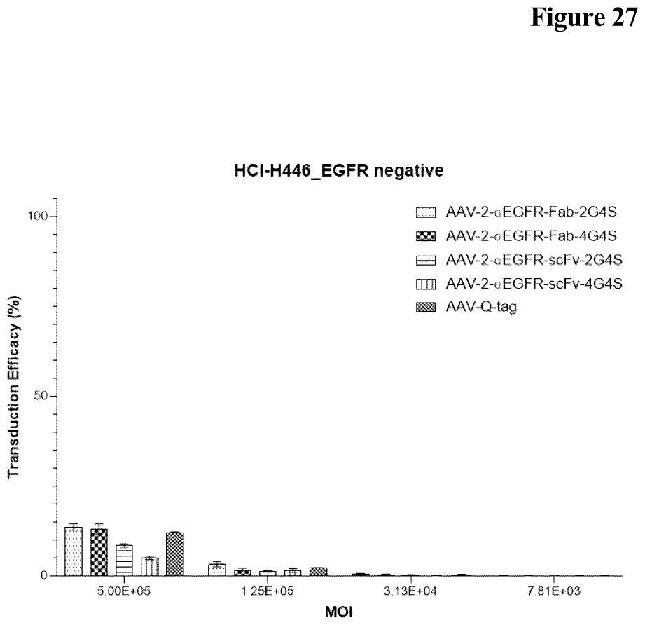

To demonstrate the biological activity of the AAV-Fab/scFv conjugates, cell transduction assays were performed. Therefore, conjugated and non-conjugated AAV-2 particles carrying mGreenLantern (mGL) as transgene were incubated with EGFR displaying HCC827 cells and the negative, not EGFR displaying cell line NCI-H446. The results are shown in FIG. 26 and FIG. 27.

MOI-dependent transduction was observed for all EGFR targeting AAV conjugates. All scFv and Fab conjugates showed specific targeting, underlining the versatility of this approach.

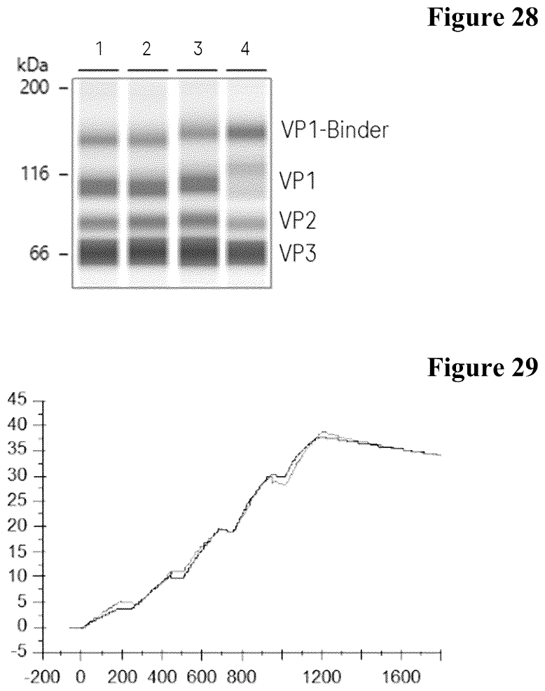

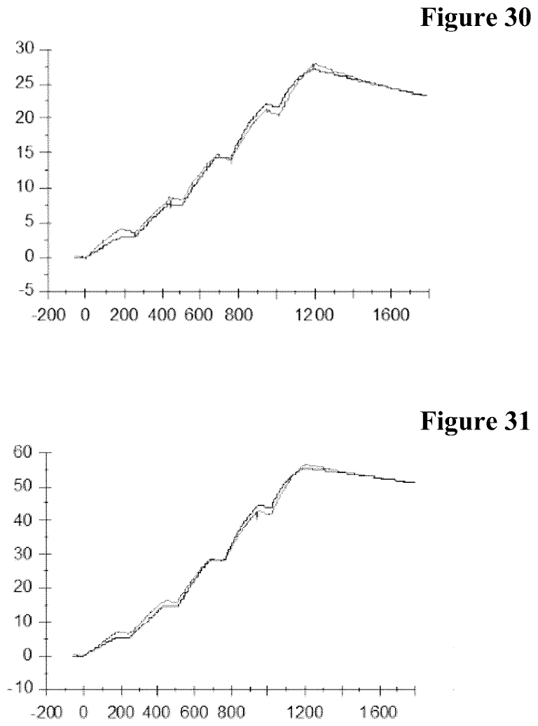

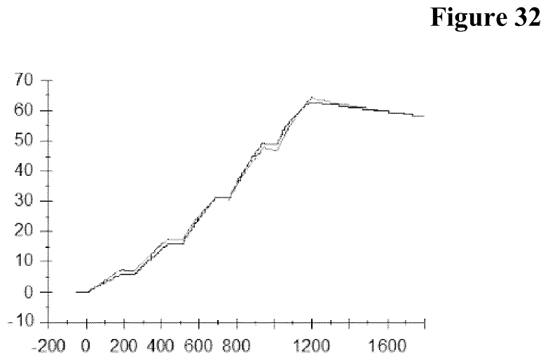

Further, conjugation of AAV-2-Q-tag (SEQ ID NO: 1, 2, 3, 4) to various anti-Her2 binder formats, specifically scFvs (SEQ ID NO: 25, 84, SEQ ID NO: 26, 85) and Fab Fragments (SEQ ID NO: 27 and SEQ ID NO: 28) was tested. Successful conjugation was determined via Western blot analysis, Surface Plasmon Resonance (SPR) Binding Kinetics Assay and Transduction Assay. The results are shown in FIGS. 28-34.

TABLE 7
Summary of conjugates used in Western blot shown in FIG. 28.

| Lane | SEQ ID NO | Construct Name |
|---|---|---|
| 1 | 25, 84 | aHER2-Fab-2G4S-Ktag |
| 2 | 26, 85 | aHER2-Fab-4G4S-Ktag |
| 3 | 27 | aHER2-scFv-2G4S-Ktag |
| 4 | 28 | aHER2-scFv-4G4S-Ktag |

TABLE 8
Kinetic rate parameters determined from FIGS. 29 to 32.

| Sample | ka | kd | KD |
|---|---|---|---|
| (SEQ ID NO) | (1/Ms) | (1/s) | (M) |
| 1, 2, 3, 25, | 8.63E+04 | 1.70E−04 | 1.97E−09 |
| 84 + 94 | | | |
| 1, 2, 3, 26, | 9.43E+04 | 2.60E−04 | 2.75E−09 |
| 85 + 94 | | | |
| 1, 2, 3, | 2.85E+04 | 1.36E−04 | 4.76E−09 |
| 27 + 94 | | | |
| 1, 2, 3, | 2.67E+04 | 1.23E−04 | 4.58E−09 |
| 28 + 94 | | | |

SPR data shows similar affinities for all constructs, independent of antibody format conjugated to AAV2-Q or linker length (2xG4S vs 4xG4S) between antibody format and AAV2-Q.

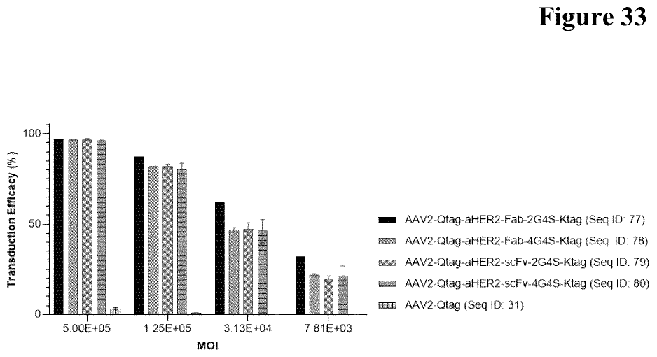

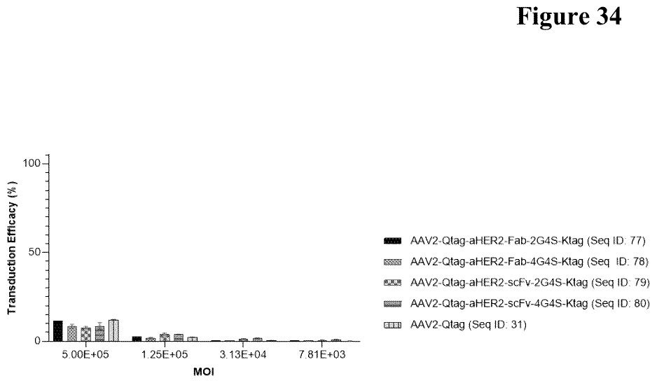

To show specific transduction of rAAVs, AAV2-Qtag-aHer2-Fab (SEQ ID NO: 1, 2, 3, 25, 84, SEQ ID NO: 1, 2, 3, 26, 85) or AAV2-Qtag-aHer2-scFv (SEQ ID NO: 1, 2, 3, 27, SEQ ID NO: 1, 2, 3, 28) were applied on a Her2 positive cell line and Her2 negative cell line. AAV2-Qtag-aHer2-Fab and AAV2-Qtag-aHer2-scFv show superior transduction efficiency on Her2 positive cells compared to Her2 negative cells (FIGS. 33 and 34).

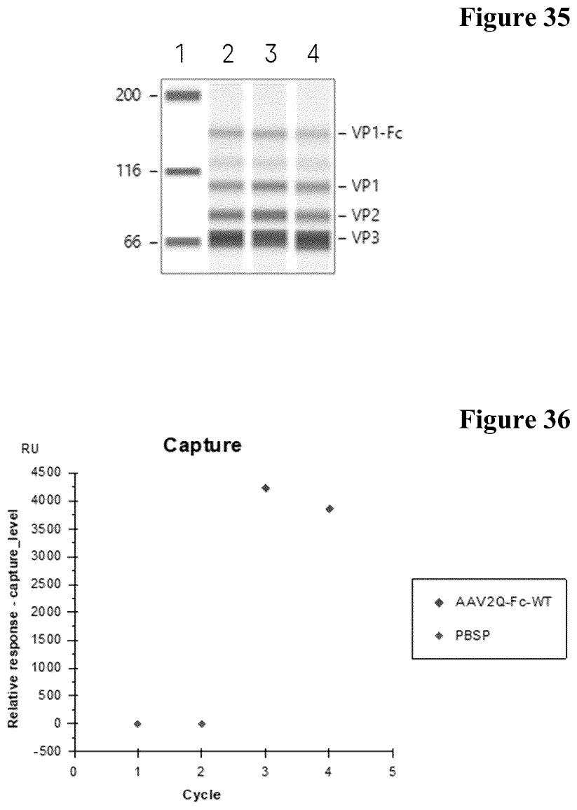

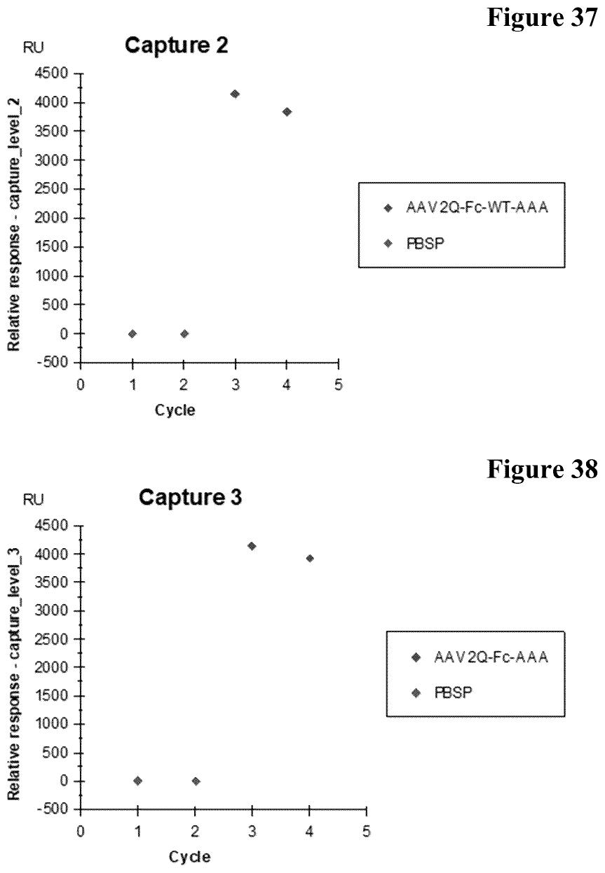

In order to show that AAV-2 can be equipped with an Fc functionality allowing the interaction with FcRn, antibody Fc fragments were conjugated to AAV-2-Qtag (SEQ ID NO: 1, 2, 3). Specifically, three different antibody Fc fragments were conjugated: i) Fc-only-K-WT (SEQ ID NO: 95, 124) has two binding sites for human single chain FcRn (hsc-FcRn, SEQ ID NO: 98), ii) Fc-only-K-WT-3A (SEQ ID NO: 96, 125) features only one binding site for FcRn as it is an asymmetrical Fc design where one heavy chain is wildtype while the other one has a AAA (H310A, H433A, Y436A) mutation abolishing FcRn binding, iii) Fc-only-K-3A (SEQ ID NO: 97, 126) has symmetrical AAA mutation in both heavy chains abolishing FcRn binding. Successful conjugation is shown via Western Blot Analysis and FcRn binding kinetics via SPR. Results are shown in FIGS. 35 to 41.

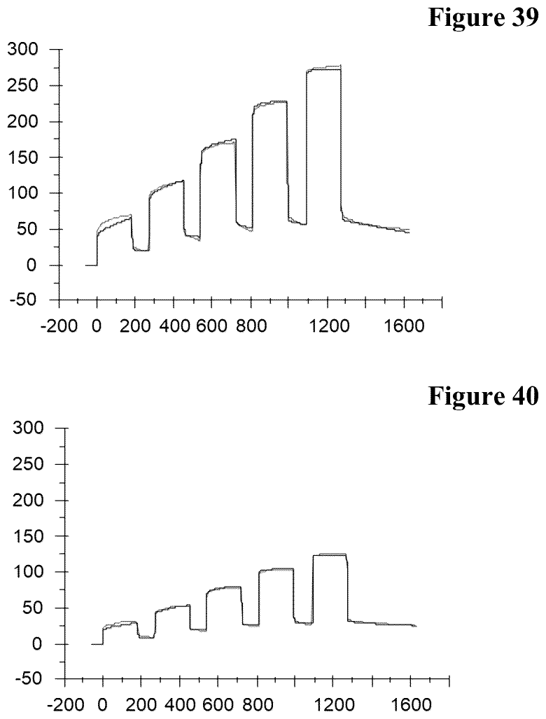

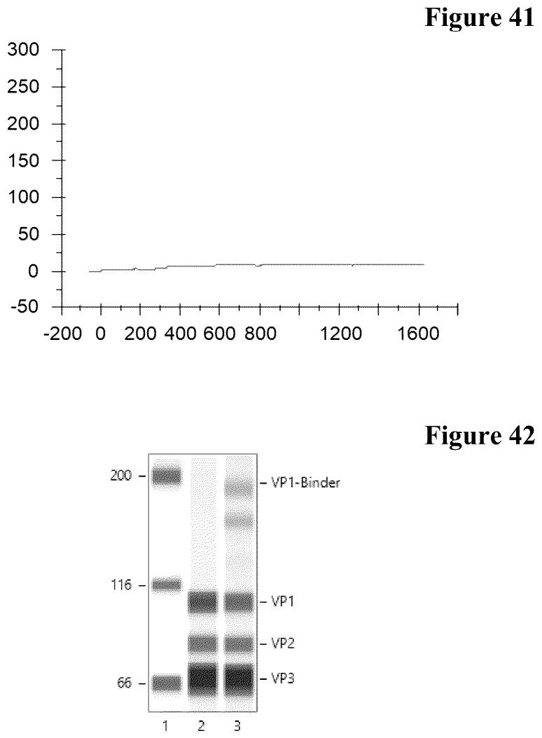

Comparison of FIGS. 39 and 40 reveals that AAV2-Qtag-Fc-only-K-WT-3A (SEQ ID NO: 1, 2, 3, 95, 126), modified with an asymmetrical Fc composed of WT and AAA heavy chain, produces only half the signal change observed with AAV2-Qtag-Fc-only-K-WT, which contains the symmetrical Fc WT heavy chains (SEQ ID NO: 1, 2, 3, 95, 124). This observation is based on equal capture levels of the modified AAV2-Qtag when hsc-FcRn is injected. These results indicate that the Fc WT-AAA asymmetrical variant allows only half the amount of hsc-FcRn to bind compared to the Fc WT, while the Fc AAA variant completely abolishes binding (FIG. 41).

TABLE 9
Kinetic rate parameters determined from FIGS. 39 to 41.

| Sample | ka1 | kd1 | KD1 | ka2 | kd2 | KD2 |
|---|---|---|---|---|---|---|
| Seq ID | (1/Ms) | (1/s) | (M) | (1/Ms) | (1/s) | (M) |
| 1, 2, | 5.15E+05 | 5.04E−01 | 9.79E−07 | 1.13E+04 | 5.36E−04 | 4.74E−08 |
| 3, 95, | | | | | | |
| 124 + 98 | | | | | | |
| 1, 2, | 3.84E+05 | 3.90E−01 | 1.02E−06 | 1.31E+04 | 9.13E−04 | 6.97E−08 |
| 3, 96, | | | | | | |
| 125 + 98 | | | | | | |
| 1, 2, | NA | NA | NA | NA | NA | NA |
| 3, 97, | | | | | | |
| 126 + 98 | | | | | | |

In order to allow dual targeting of AAV-2, a bispecific construct aEGFR-aHer2-Fc-K, comprising two scFv fused to an antibody Fc fragment (SEQ ID NO: 102, 127) was conjugated to AAV-2-Qtag (SEQ ID NO: 1, 2, 3). The resulting AAV-2-aEGFR-aHer2-scFv-Fc-K (SEQ ID NO: 1, 2, 3, 102, 127) is equipped with two binding moieties targeting human EGFR (SEQ ID NO: 104) and human Her2 (SEQ ID NO: 94) simultaneously. To demonstrate activity of both scFvs, Western Blot analysis and SPR binding kinetics were performed. The results are shown in FIGS. 42 to 44.

TABLE 10
Summary of conjugates used in Western blot shown in FIG. 42.

| Lane | SEQ ID NO | Construct Name |
|---|---|---|
| 1 | | Ladder/Marker |
| 2 | 1, 2, 3 | AAV2-Qtag |
| 3 | 1, 2, 3, | AAV2-Qtag-aEGFR-aHer2- |
| | 102, 127 | scFv-Fc-Ktag |

Unmodified AAV2-Qtag does not exhibit any signal change upon injection of EGFR or HER2. In contrast, AAV2-Qtag conjugated to an anti-EGFR-HER2 bispecific antibody format demonstrates specific interactions with the recombinant receptors upon injection.

Insertion of Qtag within the VP1 protein of the AAV5 capsid was investigated at variable regions (VR) 1, 4, and 8. This strategy involved mutating key residues to negate natural sialic acid binding (Y585V, L587T) and sulfoglycan binding (M569V). The Qtag was inserted as a G4S-YRYRQ-G4S (SEQ ID NO: 74) motif, replacing three amino acids in VR1 (residues D256, G257, S258) (SEQ ID NO: 110), VR4 (residues T444, G445, G446) (SEQ ID NO: 111), or VR8 (residues T577, T578, A579) (SEQ ID NO: 112). This approach aimed to create a versatile AAV platform for targeted delivery.

AAV particles with SEQ ID NO: 110, 113, 114 were not producable. AAV particles with SEQ ID NO: 112, 113, 114 yielded low capsid titers not sufficient for a conjugation reaction. Therefore, the conjugation of binders to the capsid was only performed with SEQ ID NO: 111, 113, 114, 300 nM AAV-5-Qtag (1.5E+14 pt/mL) were incubated with 50 nM KTG and 15 μM ligand (SEQ ID NO: 25) at 37° C. overnight. Samples were analyzed via Western Blot (Simple Western™ Automated Western Blot Systems, Biotechne). Bands were visualized with an anti-AAV VP1 mouse monoclonal (690056, Progen) primary antibody and an anti-Mouse Detection Kit (Biotechne). Samples were diluted to a titer of 2.5E+11 pt/ml and 4 μL was applied. A conjugation of 10% was observed in the Western blot (FIG. 49).

TABLE 11
Summary of constructs used in Western blot shown in FIG. 49.

| Lane | AAV variant particle | SEQ ID NO: |
|---|---|---|
| 1 | AAV-5-Qtag-VR4 | 111, 113, 114 |
| 2 | AAV-5-aHER2-Fab- | 111, 113, 114, |
| | 2G4S-Ktag | 25, 84 |

### Click Chemistry Approach

In order to show the general applicability of covalent attachment of binding sites to viral particles, a KTG mediated click chemistry approach was used (see FIG. 45). Therein, in a first step, two separate reactions are carried out. Functionalized moieties are generated by KTG mediated conjugation of azide or DBCO modified K- or Q-tags to the AAV-2 and binding site comprising the corresponding K- or Q-tag. In a second step the click reaction is performed by incubating the functionalized moieties and thus allowing for the conjugation of the binding site to the AAV-2.

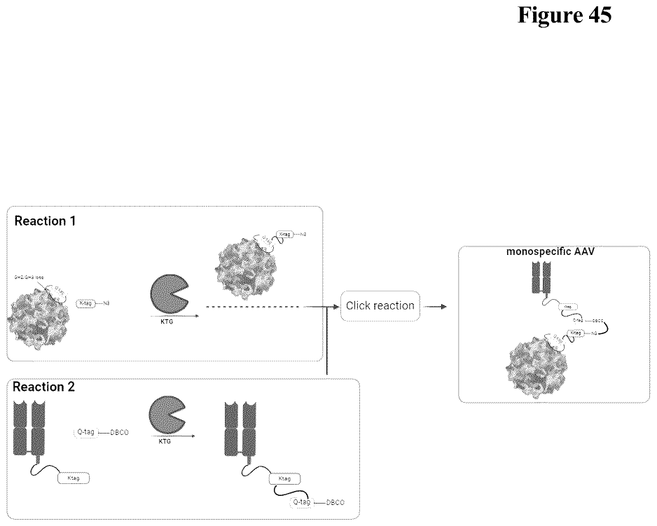

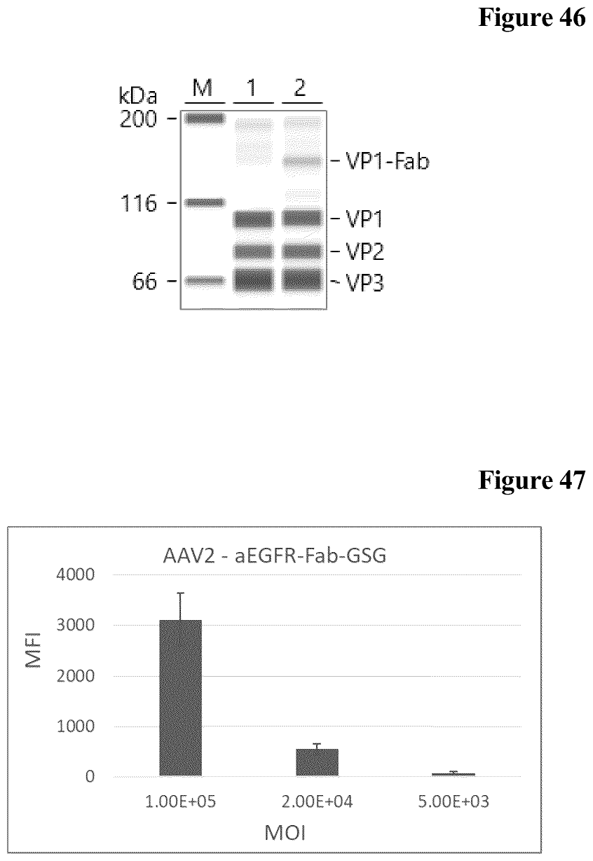

First AAV modification with an azide group. Therefore, about 300 nM AAV-2-Q-tag variant particles (SEQ ID NO: 1, 2, 3) were incubated with KTG (50 nM) and 30 μM of K-tag-PEG6-N3 (Formula 1) at 4° C. for 2-3 hours. Afterwards, the reaction mixture was purified using a desalting column to remove excess amounts of the peptide-azide (Zeba™ Spin Desalting Columns, 7K MWCO, 0.5 mL). Second, binding sites modified with a functional dibenzocyclooctyne (DBCO) group were generated. Therefore, about 10 μM aEGFR-Fab-Qtag-GSG (SEQ ID NO: 105, 128) with a C-terminal Q-tag was incubated with 50 nM KTG and 50 μM K-tag-PEG12-DBCO (Formula 2) at 25° C. for 3 hours. Subsequently, the reaction mixture was purified using protein A spin columns (NAb™ Protein A Plus Spin Columns, 1 mL). The functionalized moieties were incubated overnight at 37° C. with a 100-fold excess of the Fab-DBCO conjugate. Efficiency of the Click reaction was analyzed using western blot (Simple Western™ Automated Western Blot Systems, Biotechne) (see FIG. 46). This resulted in a conjugation efficiency of 33%.

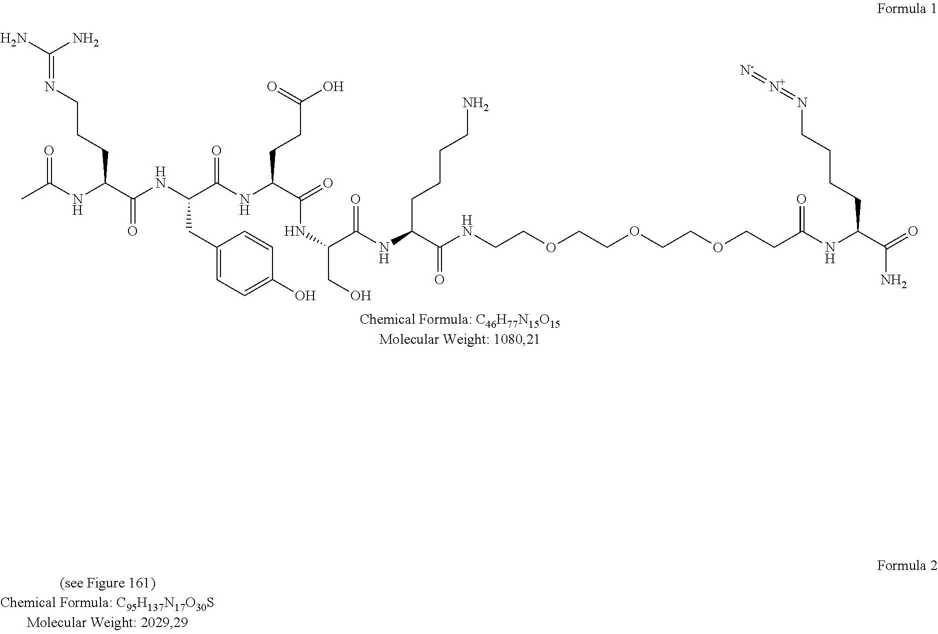 [SMILES: CC(=O)N[C@@H](CCCN=C(N)N)C(=O)N[C@@H](Cc1ccc(O)cc1)C(=O)N[C@@H](CCC(=O)O)C(=O)N[C@@H](CO)C(=O)N[C@@H](CCCCN)C(=O)NCCOCCOCCOCCC(=O)N[C@@H](CCCCN=[N+]=[N-])C(N)=O]

TABLE 12
Summary of constructs used in Western blot shown in FIG. 46.

| Lane | AAV variant particle | SEQ ID NO: |
|---|---|---|
| 1 | Negative control; azide modified | 1, 2, 3 |
| | AAV-2-Q-tag (AAV-2-N3) | |
| 2 | AAV-2-aEGFR-Fab-GSG | 1, 2, 3, |
| | | 105, 128 |

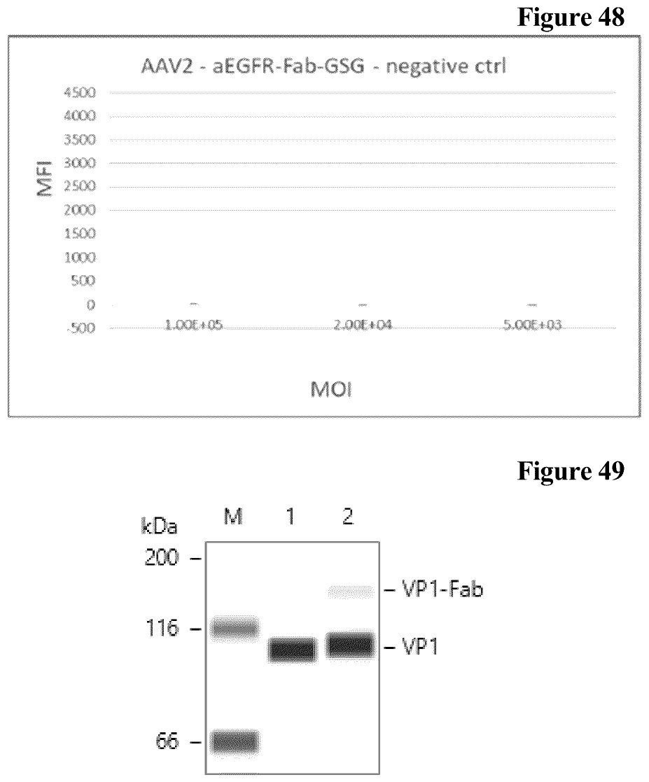

To verify biological activity of the modified viral particle by click chemistry a transduction assay on EGFR positive and negative cells was performed. Specific binding was observed at MOI 1E+5 on EGFR positive cells, while no signal was detected on EGFR negative cells (FIGS. 47 and 48).

This approach can likewise be used to track AAV trafficking in vivo. For this purpose, azide modified viral particles are incubated with DBCO modified tracking molecules (fluorogenic or radioactive probes).

The method for producing the recombinant AAV comprises the following steps:
a) incubating a mixture comprising the rAAVp according to the invention with an azide modified peptide tag in the presence of the microbial transglutaminase derived from Kutzneria albida (KTG) (SEQ ID NO: 66)
b) incubating a mixture comprising a binding site or Fc fragment with a DBCO modified peptide tag in the presence of the microbial transglutaminase derived from Kutzneria albida (KTG) (SEQ ID NO: 66)
c) incubating a mixture comprising the resulting azide-modified AAV and DBCO-modified binding site or Fc fragment from steps a) and b).

The rAAVp according to one embodiment of the invention comprises the amino acid sequence YRYRQ (SEQ ID NO: 73), i.e. the Q-tag. Thus in the current method the rAAVp is incubated with the corresponding azide modified K-tag comprising the amino acid sequence RYESK (SEQ ID NO: 75). The binding site or Fc fragment is incubated with either a DBCO-modified Q-tag or K-tag, depending on which tag is attached to the binding site or Fc fragment. If the binding site or Fc fragment comprises a Q-tag it will be incubated with a DBCO-modified K-tag. While if the binding site or Fc fragment comprises a K-tag it will be incubated with a DBCO-modified Q-tag.

Accordingly, in one embodiment of the invention the heterologous peptide or polypeptide of the variant AAV capsid protein is covalently conjugated to an antigen binding site or an Fc fragment via a dibenzocyclooctyne (DBCO) group and an azide group. The heterologous peptide or polypeptide may be covalently conjugated to an azide modified peptide comprising the amino acid sequence of SEQ ID NO: 75. The heterologous peptide or polypeptide may be covalently conjugated to the molecule of Formula 1. The antigen binding site or Fc fragment conjugated to the AAV comprises a DBCO group. Further the binding site or Fc fragment comprise the amino acid sequence YRYRQ (SEQ ID NO: 73), i.e. a Q-tag, and the amino acid sequence RYESK (SEQ ID NO: 75), i.e. a K-tag. The DBCO group may be attached to the Q- or the K-tag. In one embodiment, the antigen binding site or Fc fragment is covalently conjugated to the molecule of Formula 2.

### Genetic Fusion to Generate AAV Capsid Variants

Genetic fusion of transferrin- and CD98 receptor-binding nanobody or peptide sequences to the exposed GH2/GH3 loop in VR IV of AAV capsids has been found to be advantageous for the modification of AAV particles. It has been shown, both by binding and by transduction of the modified AAV particles, that the cellular uptake of the modified AAV variant particles according to the current invention is receptor-mediated.

The current invention is based, at least in part, on the finding that both nanobody- and peptide-presenting AAV variant particles can be produced at a titer comparable to the non-modified AAV particles.

Further, the current invention is based, at least in part, on the finding that the production of genetically fused AAV-nanobody or -peptide variant particles does not require additional purification steps, which could eventually lead to loss of modified AAV particles.

Nanobodies that bind huTfR (SEQ ID NO: 67, 68) were genetically inserted into the GH2/GH3 loop of the AAV-2 VP1 capsid protein by replacing amino acid residues S452-T455, G453-R459 or by opening the loop at residue T456. The anti-transferrin receptor (aTfR) nanobody was flanked N-terminally by a peptidic linker with the amino acid sequence (GGGGS) 5 (SEQ ID NO: 61) or (GGGGS) 2 (SEQ ID NO: 62) and C-terminally by a peptidic linker with the amino acid sequence GGGGA (SEQ ID NO: 63) or (GGGGS) 2 (SEQ ID NO: 62). Arginine residues R585 and R588 of the AAV-2 capsid protein were mutated to alanine (denoted as superscript RA) in VP1, VP2 and VP3 to prevent binding of the AAV-2 variant particles to heparan sulfate proteoglycan (HSPG) (see, e.g., Kern et al., J. Virol 77 (2003) 11072-11081). Some AAV-2^{RA}-aTfR-Nb (Nb denotes nanobody) variant particles included mutations of conserved exposed tyrosine residues (Y444F, Y500F, Y730F) (denoted as superscript RA-YF) of the viral capsid proteins VP2 and VP3 (see, e.g., Li et al., Hum. Gene Ther. 21 (2010) 1527-1543).

Nanobodies that bind huCD98 (SEQ ID NO: 71 and SEQ ID NO: 72) were genetically inserted into the GH2/GH3 loop of AAV-2 VP1 capsid protein by replacing amino acid residues S452-T455. The anti-human CD98 (aCD98)-nanobody sequence was flanked N-terminally by a peptidic linker with the amino acid sequence (GGGGS) 5 (SEQ ID NO: 61) and C-terminally by a peptidic linker with the amino acid sequence GGGGA (SEQ ID NO: 63). Arginine residues R585 and R588 were mutated to alanine in VP1, VP2 and VP3 to prevent binding of AAV-2 variant particles to heparan sulfate proteoglycan (HSPG) (see, e.g., Kern et al., J. Virol. 77 (2003) 11072-11081).

To show that genetic VP1-nanobody fusion according to the current invention can be applied to different serotypes, the respective AAV capsid proteins were assembled to chimeric AAV-9/2^{RA}-aTfR-Nb variant particles. For the recombinant production, a mutated start codon and/or a premature stop codon was introduced into the VP1 coding sequence of AAV-9 allowing expression of non-modified VP2 and VP3 only. In the second expression plasmid, a TfR-binding nanobody flanked N-terminally by a peptidic linker with the amino acid sequence (GGGGS) 5 (SEQ ID NO: 61) and C-terminally by a peptidic linker with the amino acid sequence GGGGA (SEQ ID NO: 63) was inserted into the GH2/GH3 loop of AAV-2-VP1^{RA} variant capsid protein by replacing amino acid residues S452-T455.

To show that the nanobody-based targeting strategy according to the current invention can be applied to different AAV serotypes, the respective insertion sites identified above in the corresponding GH2/GH3 loop of the AAV-5 serotype and the AAV-9 serotype were used. Nanobodies that bind huTfR were genetically inserted into the GH2/GH3 loop by replacing amino acid residues N442-T444 and S454-Q458 for the AAV-5 serotype and the AAV-9 serotype, respectively. The aTfR-nanobody sequence was flanked N-terminally by a peptidic linker with the amino acid sequence (GGGGS) 5 (SEQ ID NO: 61) and C-terminally by a peptidic linker with the amino acid sequence GGGGA (SEQ ID NO: 63).

Peptides that bind huTfR (SEQ ID NO: 69 and SEQ ID NO: 70) were genetically inserted into the GH2/GH3 loop of the AAV-2-VP1 capsid protein by replacing amino acid residues S452-T455. The aTfR peptides were interspaced between peptidic linkers each with the amino acid sequence (GGGGS) 4 (SEQ ID NO: 64).

The variant particles are summarized in Table 13.

TABLE 13
Summary of the exemplary genetically engineered AAV-Nb and -peptide variant particles.

| target | variant particle | insertion site | inserted sequence |
|---|---|---|---|
| huTfR | AAV-2^{RA}-aTfR-Nb-1 | VP1 ΔS452-T455 | (G4S)5-aTfR-Nb-1-(G4A) |
| | AAV-2^{RA/YF}-aTfR-Nb-1 | VP1 ΔS452-T455 | (G4S)5-aTfR-Nb-1-(G4A) |
| | AAV-2^{RA}-aTfR-Nb-1-(G4S)2 | VP1 ΔS452-T455 | (G4S)2-aTfR-Nb-1-(G4S)2 |
| | AAV-2^{RA/YF}-aTfR-Nb-1-(G4S)2 | VP1 ΔS452-T455 | (G4S)2-aTfR-Nb-1-(G4S)2 |
| | AAV-2^{RA}-aTfR-Nb-2 | VP1 ΔS452-T455 | (G4S)5-aTfR-Nb-2-(G4A) |
| | AAV-2^{RA/YF}-aTfR-Nb-2 | VP1 ΔS452-T455 | (G4S)5-aTfR-Nb-2-(G4A) |
| | chAAV-9/2^{RA}-aTfR-Nb-1 | VP1 ΔS452-T455 | (G4S)5-aTfR-Nb-1-(G4A) |
| | AAV-9-aTfR-Nb-1 | VP1 ΔS454-Q458 | (G4S)5-aTfR-Nb-1-(G4A) |
| | AAV-9-aTfR-Nb-1_v2 | VP1 ΔS454-Q458 | (G4S)5-aTfR-Nb-1-(G4A) |
| | AAV-5-aTfR-Nb-1 | VP1 ΔN442-T444 | (G4S)5-aTfR-Nb-1-(G4A) |
| | AAV-2^{RA}-aTfR-Pept-1 | VP1 ΔS452-T455 | (G4S)4-aTfR-Pept-1-(G4S)4 |
| | AAV-2^{RA}-aTfR-Pept-2 | VP1 ΔS452-T455 | (G4S)4-aTfR-Pept-2-(G4S)4 |
| | chAAV-9/2^{RA}-aTfR-Pept-1 | VP1 ΔS452-T455 | (G4S)4-aTfR-Pept-1-(G4S)4 |
| | chAAV-9/2^{RA}-aTfR-Pept-2 | VP1 ΔS452-T455 | (G4S)4-aTfR-Pept-2-(G4S)4 |
| | AAV-2^{RA}-aTfR-Nb-1_T456 | VP1 insert T456 | (G4S)2-aTfR-Nb-1-(G4S)2 |
| | AAV-2^{RA}-aTfR-Nb-1_del453-459 | VP1 ΔG453-R459 | (G4S)5-aTfR-Nb-1-(G4A) |
| | chAAV-9/2^{RA}-aTfR-Nb-1_T456 | VP1 insert T456 | (G4S)2-aTfR-Nb-1-(G4S)2 |
| | chAAV-9/2^{RA}-aTfR-Nb-1_del453-459 | VP1 ΔG453-R459 | (G4S)5-aTfR-Nb-1-(G4A) |
| huCD98 | AAV-2^{RA}-aCD98-Nb-1 | VP1 ΔS452-T455 | (G4S)5-aCD98-Nb-1-(G4A) |
| | AAV-2^{RA}-aCD98-Nb-2 | VP1 ΔS452-T455 | (G4S)5-aCD98-Nb-2-(G4A) |

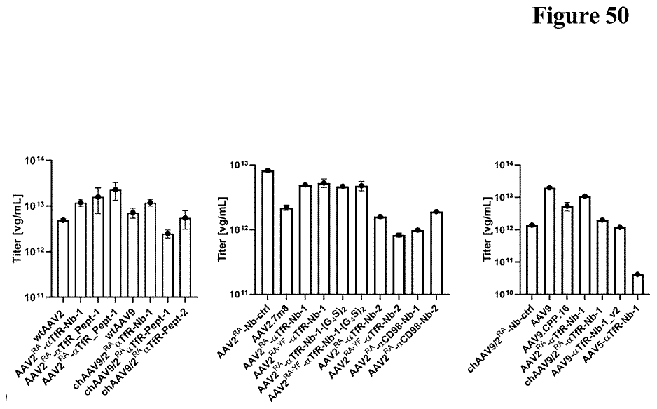

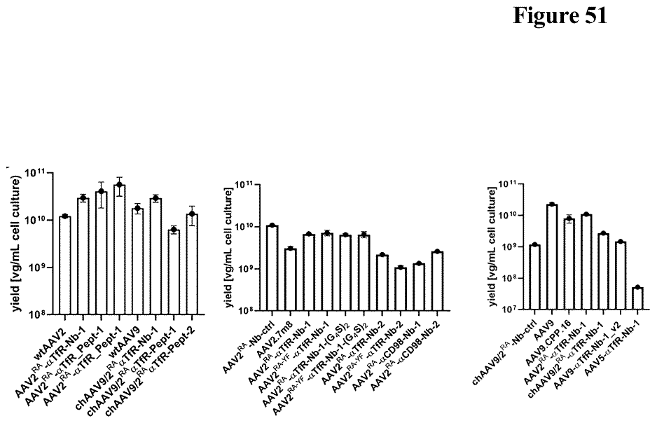

To assess whether the insertion of the aTfR nanobody had an effect on capsid packaging and assembly, titers (viral genomes in vg/mL) were determined using dPCR. The determined titers showed that all variant particles were generally produced with titers comparable to unmodified rAAVps, however AAV-5-aTfR-Nb-1 variant particle showed approximately a 100-fold lower titer (see FIG. 50 and FIG. 51).

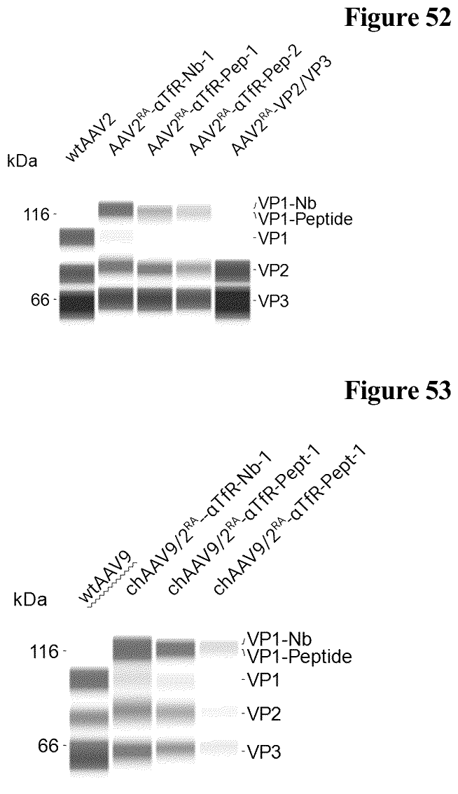

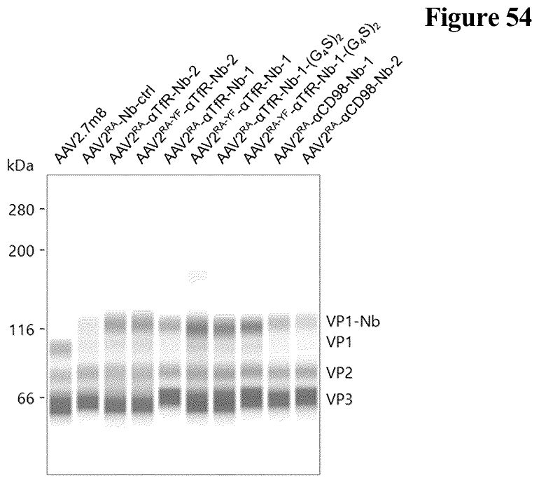

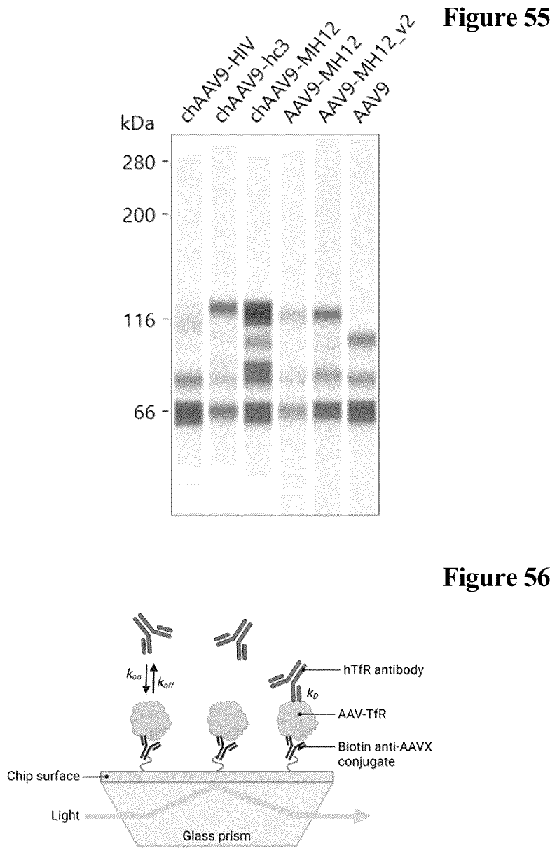

Moreover, recombinant AAV-nanobody and -peptide variant particles were analyzed for nanobody and peptide incorporation by Jess Western blotting. Using an antibody that recognizes a common epitope in the capsid proteins, the fusion of the VP1 capsid protein to the nanobody and peptide, respectively, was confirmed by an increase of the size by approximately 12-15 kDa compared to unmodified VP1 capsid protein (see FIG. 52 to FIG. 55).

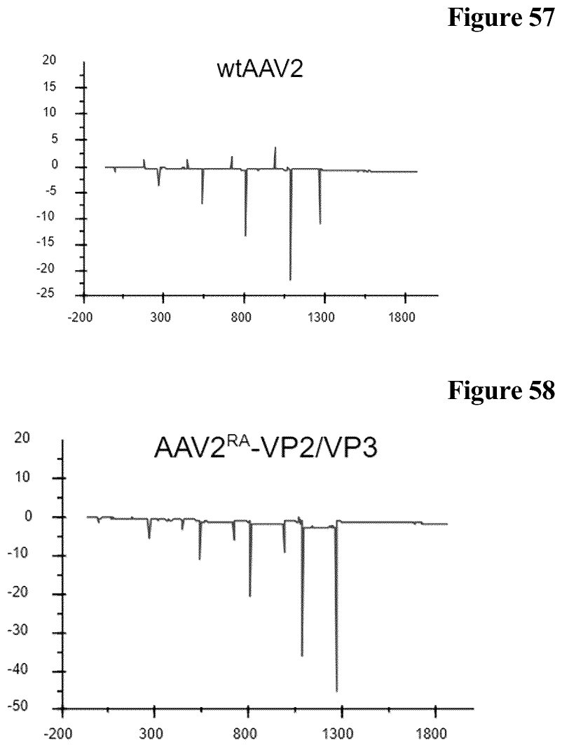

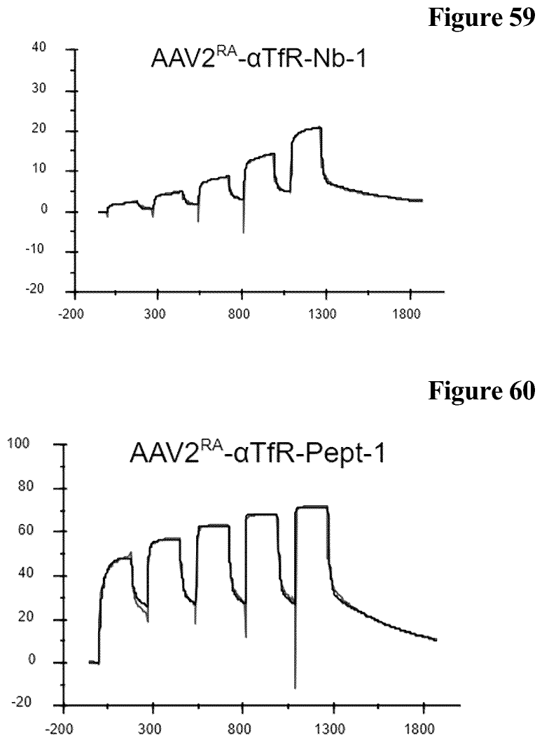

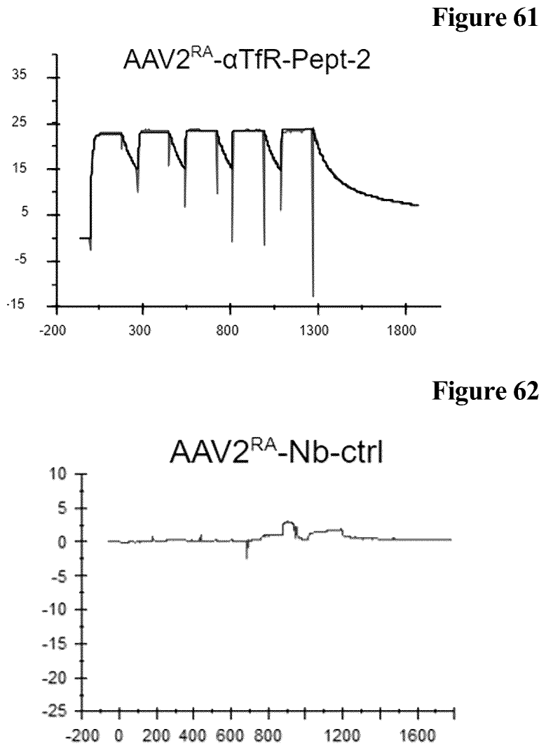

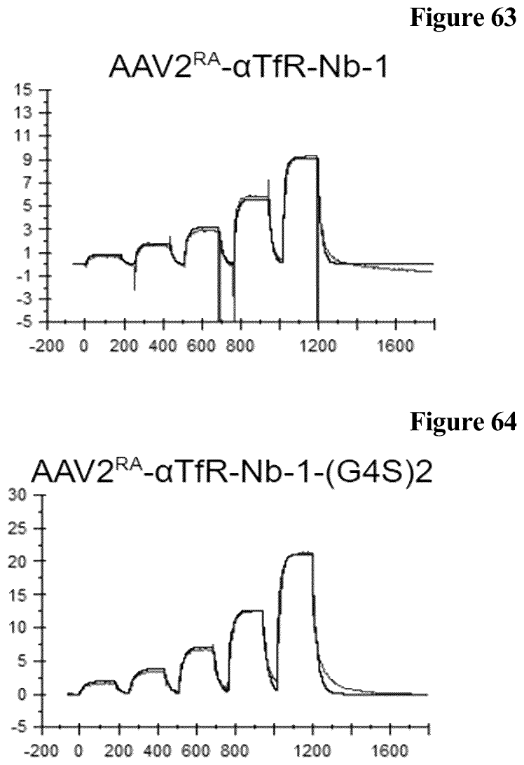

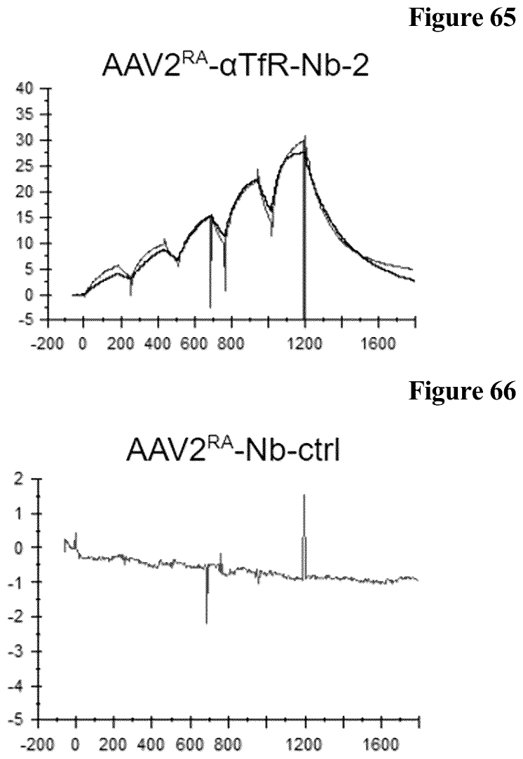

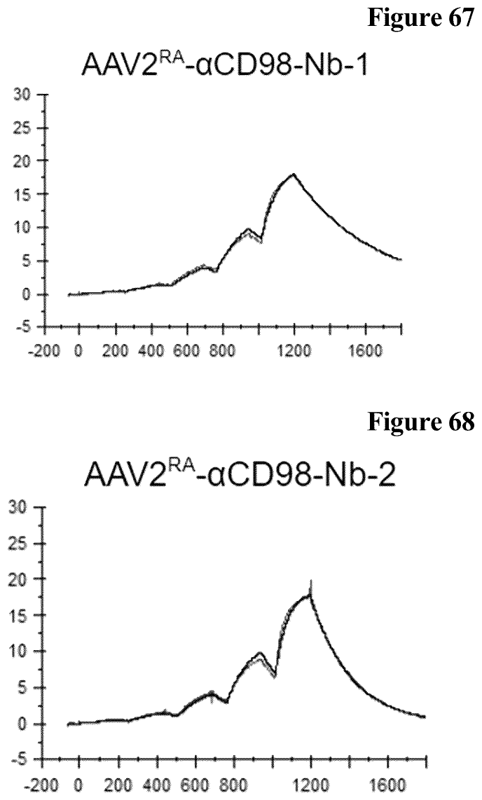

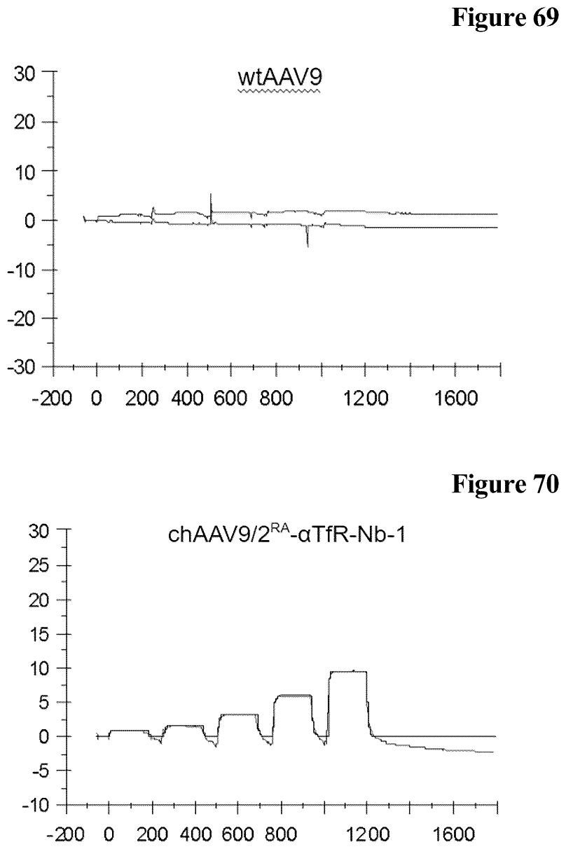

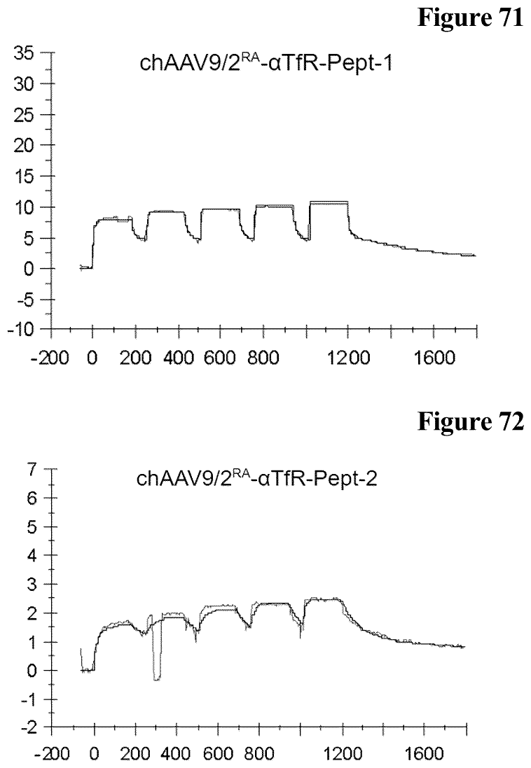

The binding properties of the AAV-Nb and -peptide variant particles towards the biological target of the Nb/peptide were analyzed via surface plasmon resonance (see FIG. 56 for SPR setup). The results are shown in FIG. 57 to FIG. 72.

The respective KD values are summarized in Table 14.

TABLE 14
Kinetic rate parameters determined from FIGS. 57 to 72.

| variant particle | ka [1/Ms] | kd [1/s] | KD [M] |
|---|---|---|---|
| AAV-2^{RA}-aTfR-Nb-1 | 3.18E+04 | 5.32E−02 | 1.67E−06 |
| AAV-2^{RA}-aTfR-Nb-1-(G4S)2 | 2.1E+04 | 4.21E−02 | 1.99E−06 |
| AAV-2^{RA}-aTfR-Nb-2 | 2.27E+04 | 6.3E−03 | 2.28E−07 |
| chAAV-9/2^{RA}-aTfR-Nb-1 | 8.47E+04 | 1.51E−01 | 1.78E−06 |
| AAV-2^{RA}-aTfR-Pept-1 | 5.63E+05 | 1.90E−03 | 3.38E−09 |
| AAV-2^{RA}-aTfR-Pep-t2 | 1.74E+06 | 1.09E−02 | 6.26E−09 |
| chAAV-9/2^{RA}-aTfR-Pept-1 | 1.87E+06 | 1.23E−01 | 6.61E−08 |
| chAAV-9/2^{RA}-aTfR-Pept-2 | 7.09E+05 | 1.18E−02 | 1.66E−08 |
| AAV-2^{RA}-aCD98-Nb-1 | 3.26E+04 | 2.28E−03 | 6.98E−08 |
| AAV-2^{RA}-aCD98-Nb-2 | 3.95E+04 | 4.88E−03 | 1.234E−07 |

To determine the binding specificity of AAV-aTfR-Nb and -peptide variant particles, CHO cells expressing a recombinant human transferrin receptor were used. To assess whether AAV-Nb and -peptide variant particles and transferrin bind competitively to huTfR, cells were pre-incubated with holo-transferrin and AAV-Nb and -peptide variant particles were added thereafter at three different MOIs (1E+3 to 1E+5) to the cells. After incubation, the cells were fixed and stained using monoclonal mouse anti-AAV-2 or anti-AAV-9 antibodies.

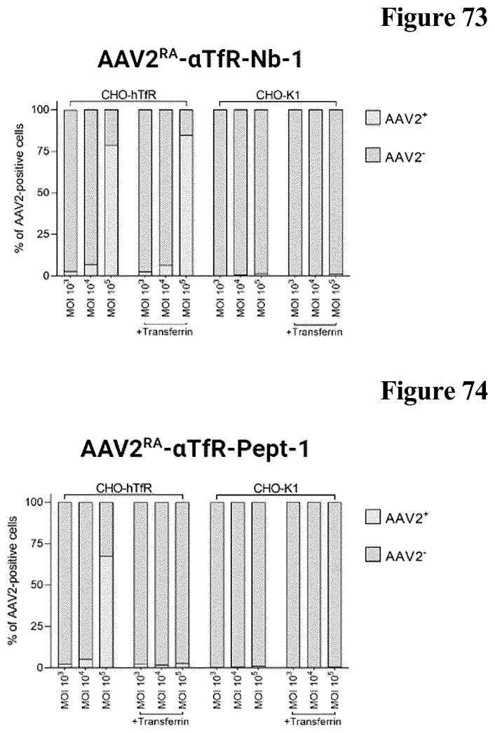

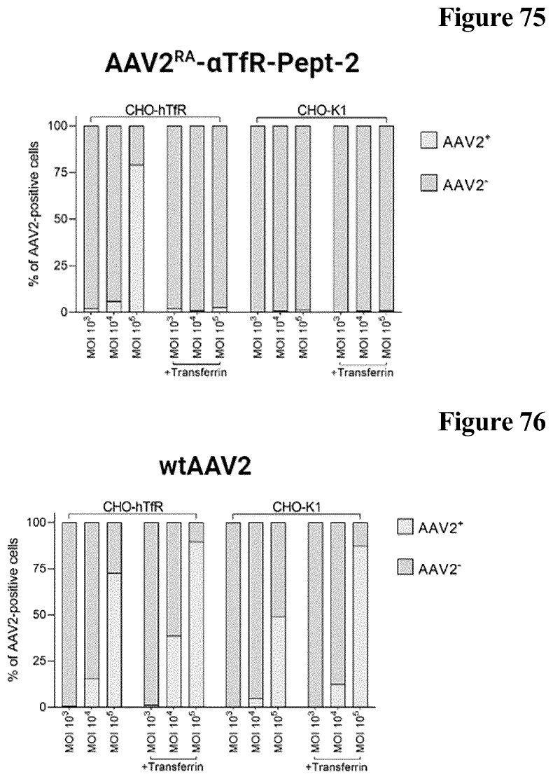

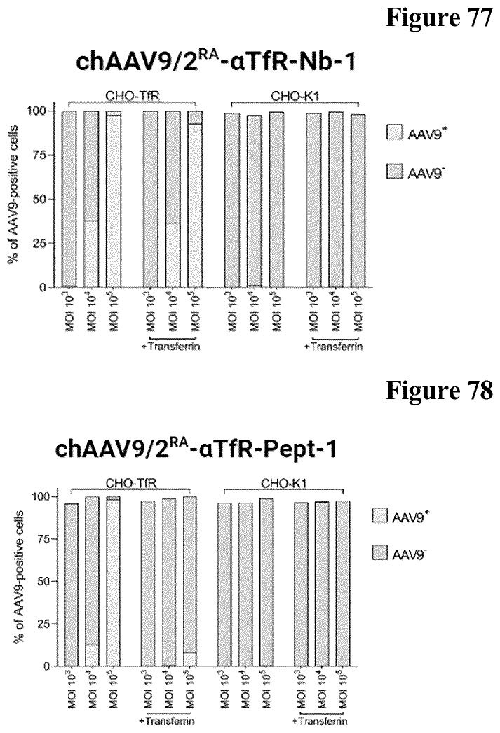

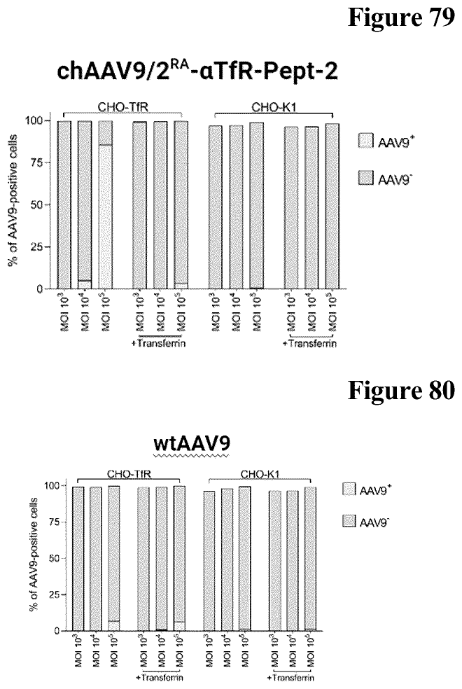

All nanobody or peptide conjugated AAV variant particles showed specific binding to CHO cells expressing huTfR receptor (see FIG. 73 to FIG. 80).

Compared to the reference, non-modified AAV-2 particles, no or only little binding of AAV-Nb and -peptide variant particles was observed in wild-type CHO cells, i.e. cells not expressing human TfR. To assess whether AAV-Nb and -peptide variant particles bind competitively with transferrin to the human TfR, cells were cultured in presence of transferrin. While binding of AAV-Nb variant particles was not competitive with transferrin, all AAV-peptide variant particles bound competitively with transferrin to the human TfR as expected.

To determine the binding specificity of AAV-aTfR-Nb and -peptide variant particles, MDCK cells expressing a chimeric human/mouse transferrin receptor (TfR^{hu/mu}) were used. These were pre-incubated with holo-transferrin. AAV-Nb and -peptide variant particles were added at three different MOIs (1E+3 to 1E+5) to the cells and incubated with or without holo-transferrin. After incubation, cells were fixed and stained using monoclonal mouse anti-AAV-2 or anti-AAV-9 antibodies.

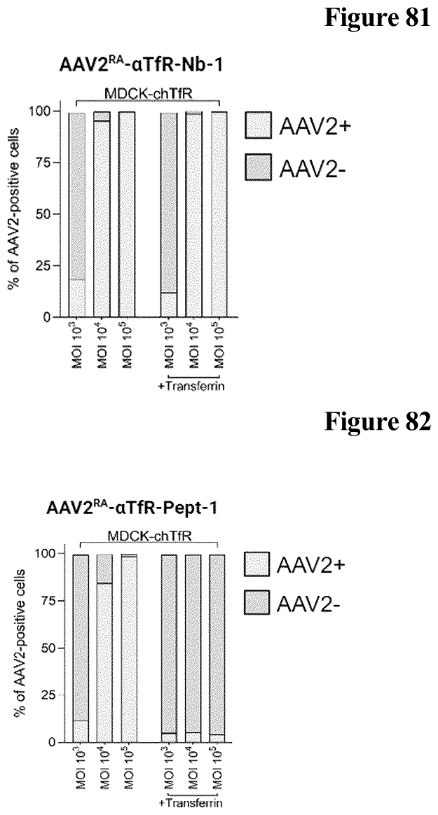

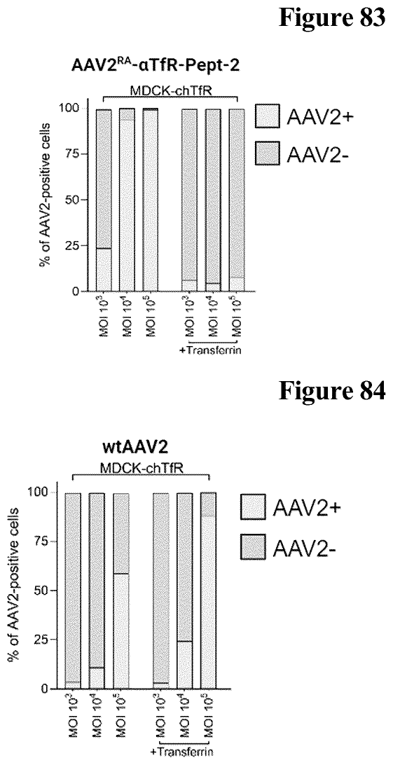

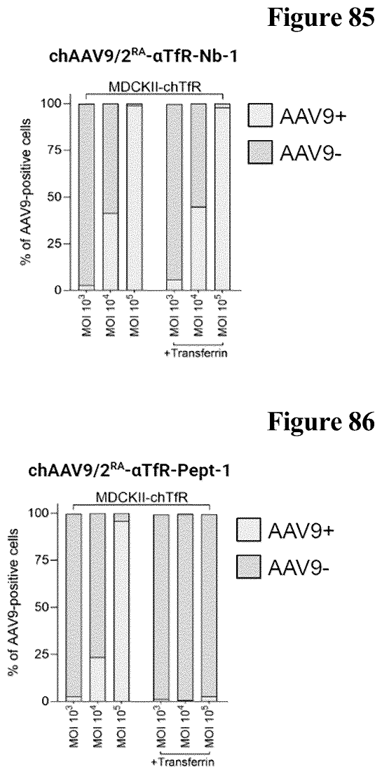

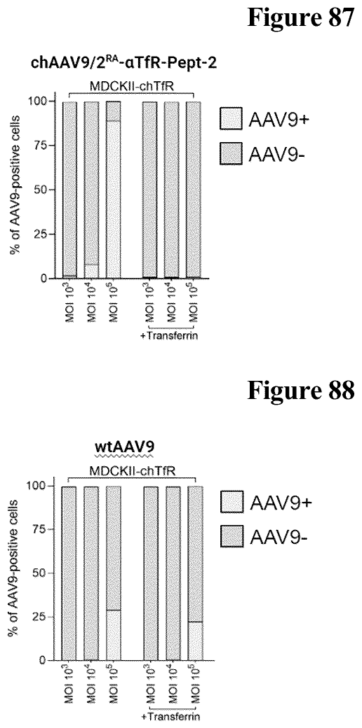

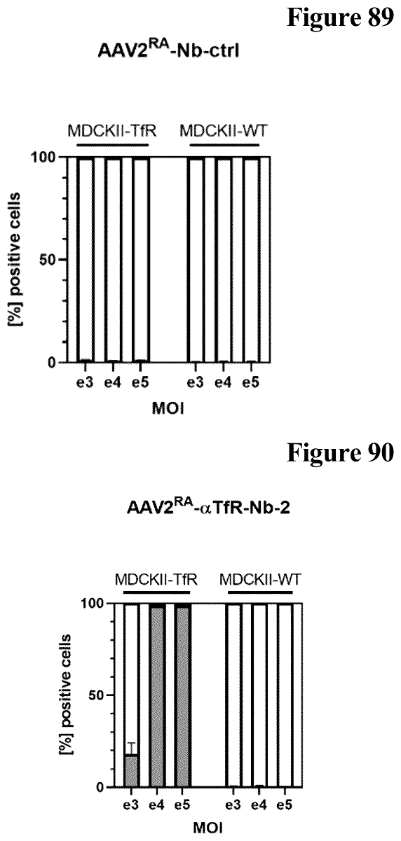

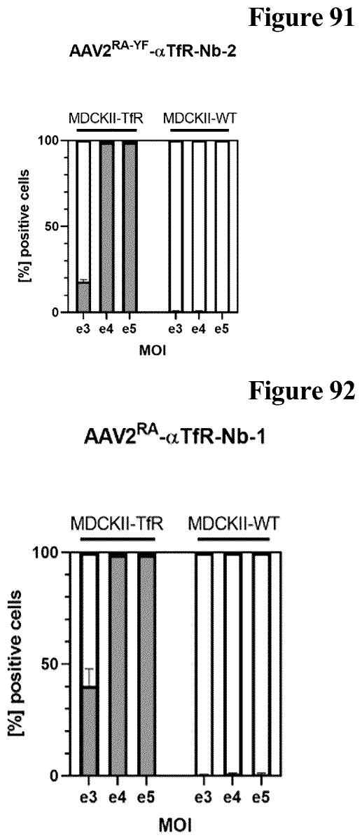

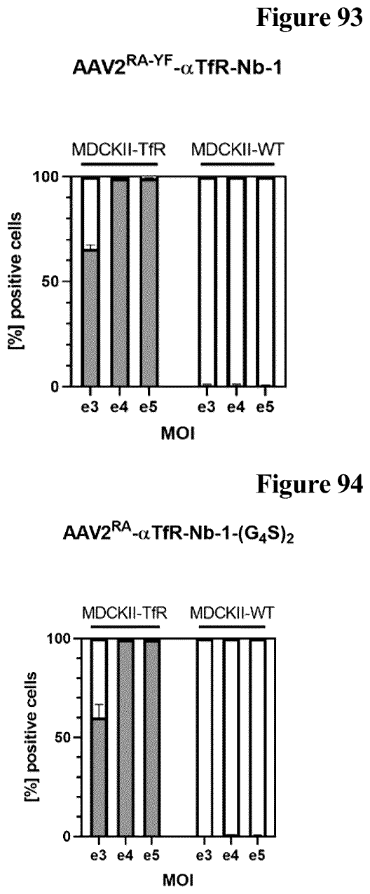

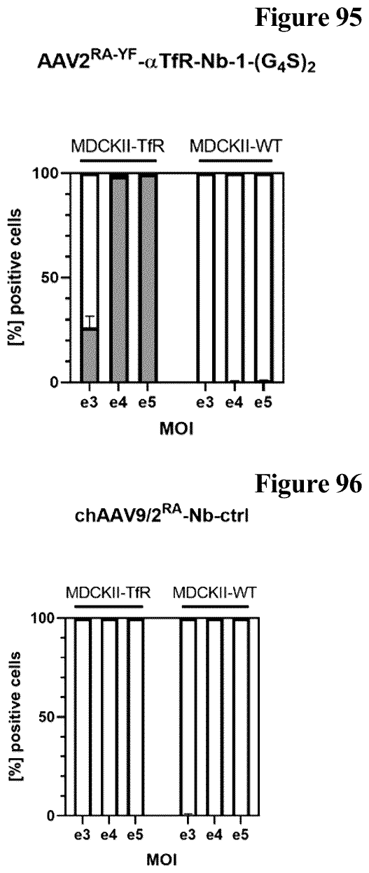

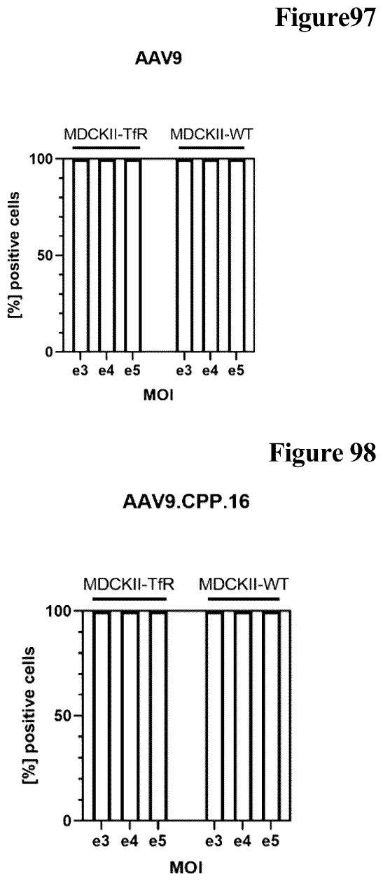

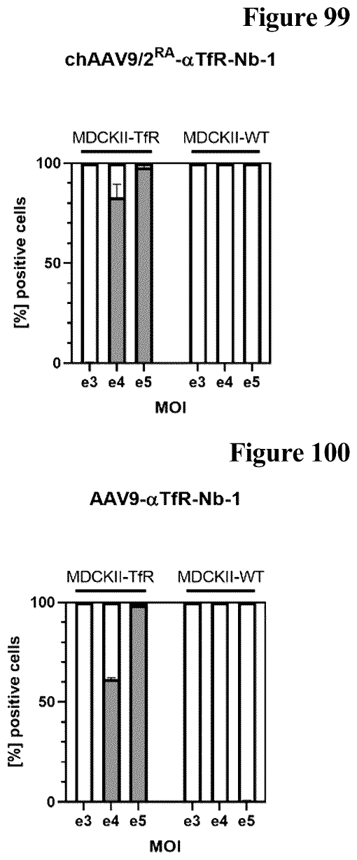

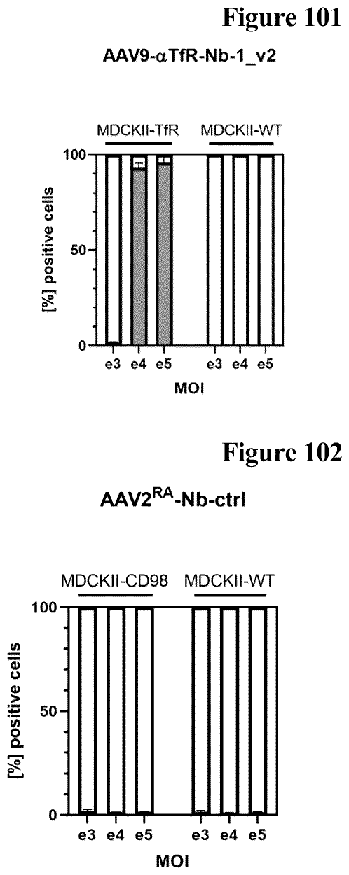

All aTfR-nanobody or -peptide conjugated AAV variant particles showed specific binding in MDCK cells expressing TfR^{hu/mu} (see FIG. 81 to FIG. 101).

Compared to wild-type AAV-2 particles, no or only little binding of AAV-aTfR-Nb and -peptide variant particles was observed in wild-type MDCKII cells, which do not express TfR^{hu/mu}. To assess whether AAV-aTfR-Nb and -peptide variant particles bind competitively with transferrin to TfR, cells were incubated in the presence of transferrin. While binding of AAV-aTfR-Nb variant particles was not competitive with transferrin, all AAV-aTfR-peptide variant particles bound competitively with transferrin to TfR^{hu/mu} as expected.

To determine the binding specificity of AAV-2-aCD98-Nb variant particles, MDCK cells expressing the human CD98 were incubated at three different MOIs (1E+3 to 1E+5) with the AAV-2-aCD98-Nb variant particles. After incubation, cells were fixed and stained using a monoclonal mouse anti-AAV-2 antibody.

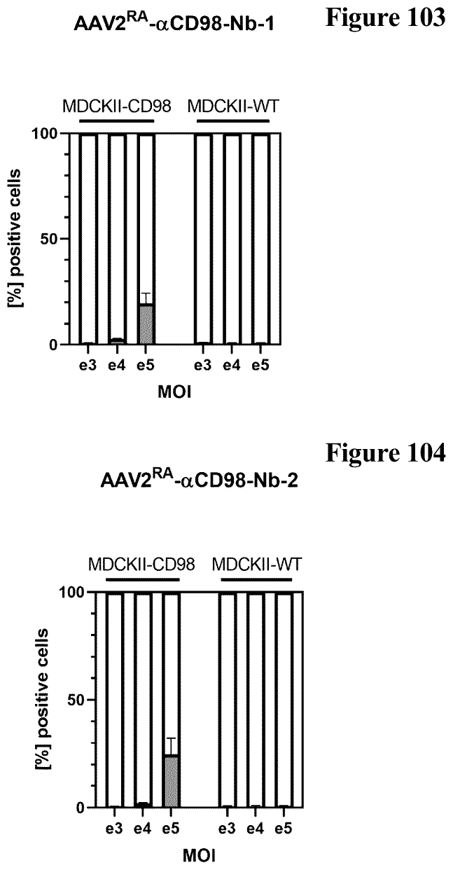

AAV-2-aCD98-Nb variant particles specifically bound to huCD98-expressing MDCKII cells and no binding was observed in wild-type MDCKII cells, which do not express huCD98 (see FIG. 102 to FIG. 104).

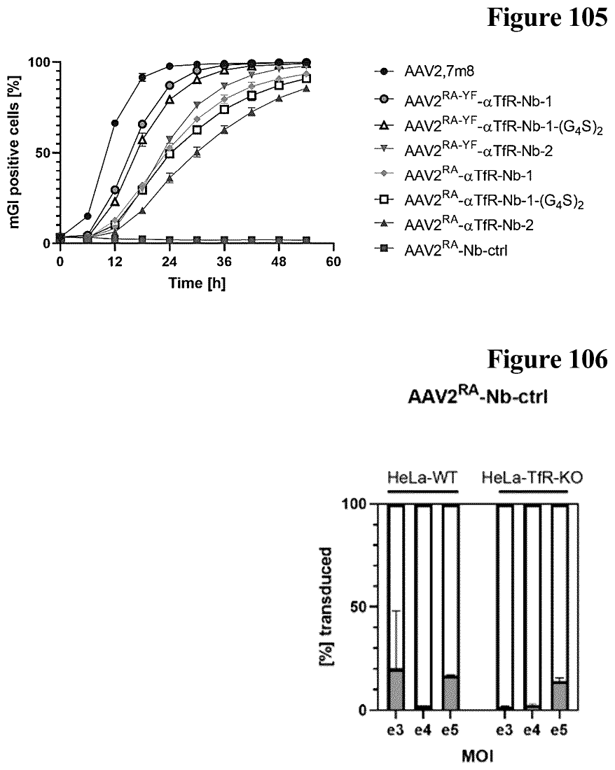

A transduction assay was performed with the genetic fusion AAV-2-aTfR-Nb variant particles. Therefore, an mGreenLantern-encoding transgene was used. The AAV-2 variant particles were added to wild-type HeLa or HeLa TfR knockout cells at MOIs ranging from 1E+3 to 1E+5. The results are shown in FIG. 105.

While differences in mGL expression kinetics were monitored for the different AAV-2-aTfR-Nb variant particles, strong transduction with more than 85% mGL-positive cells was observed for all AAV-2-aTfR-Nb variant particles after 54 hours. Capsid variants including mutations of conserved exposed tyrosine residues (Y444F, Y500F, Y730F) showed faster mGL expression kinetics.

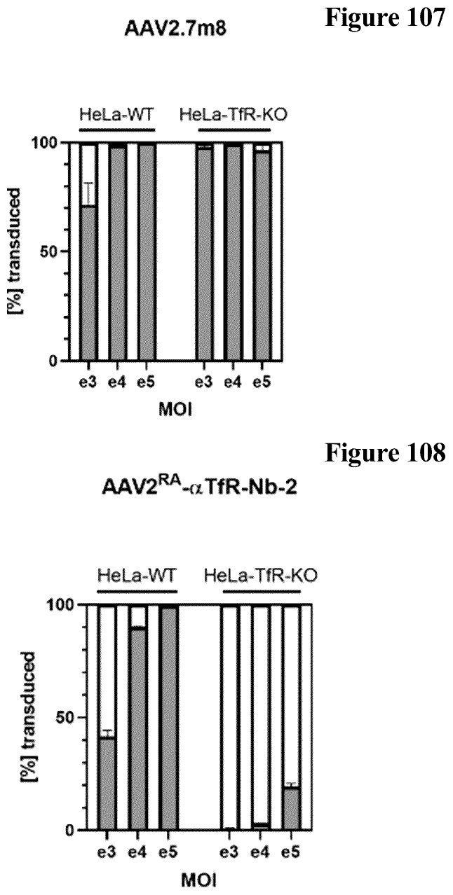

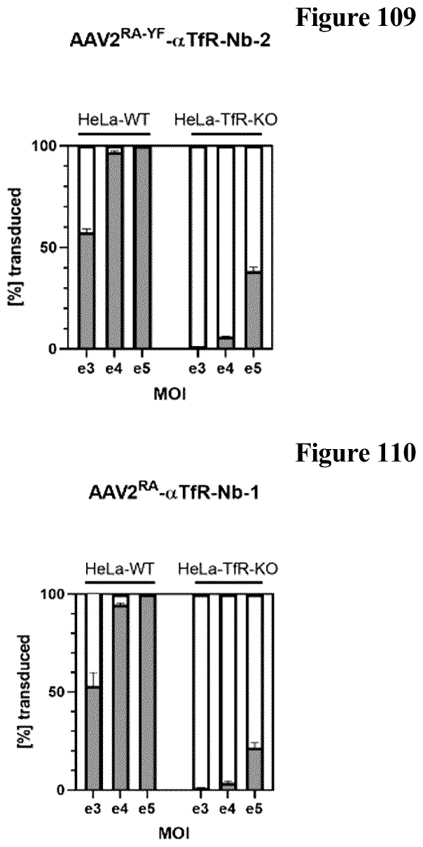

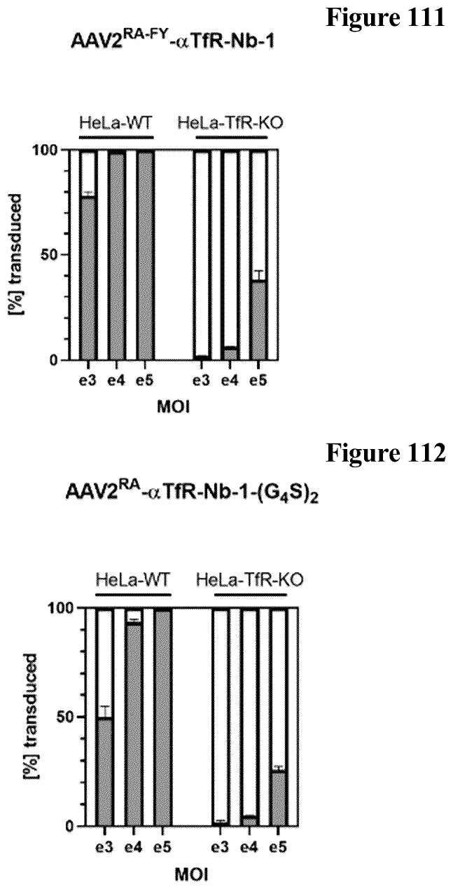

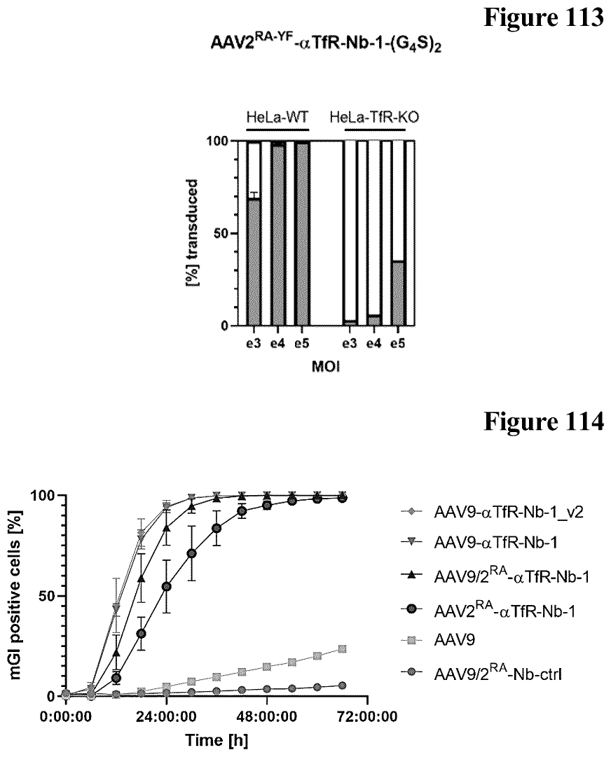

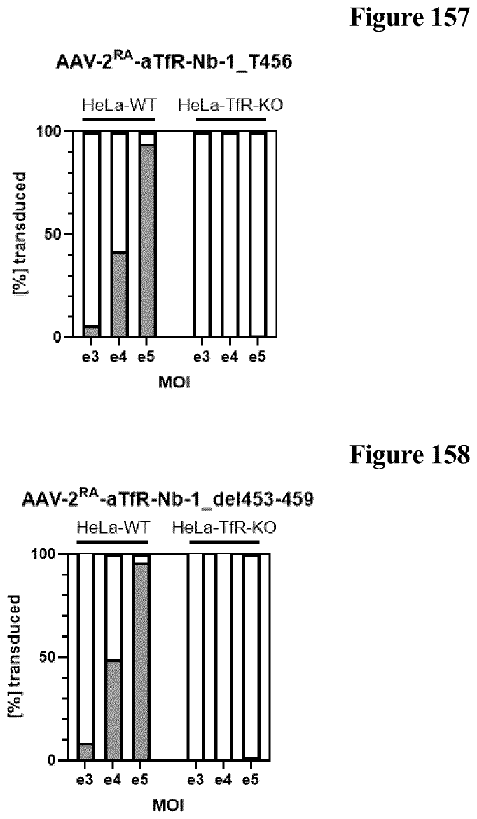

Without being bound by this theory, since no mGL expression was observed in HeLa cells transduced with control AAV-2^{RA}-Nb-Ctrl variant particles displaying a non-binding nanobody on the capsid surface, transduction of all AAV-aTfR-Nb variant particles was therefore mediated by TfR binding and receptor-mediated uptake. The percentage of mGL-positive cells in HeLa TfR KO cells transduced with AAV-2-aTfR-Nb variant particles was significantly lower compared to wild-type HeLa cells indicating that cell entry is mediated by TfR-uptake (see FIG. 106 to FIG. 113 and FIG. 157 to FIG. 158).

A transduction assay was performed with the genetic fusion AAV-9-aTfR-Nb and AAV-9/2-TfR-Nb variant particles. An mGreenLantern-encoding transgene was used. The AAV-9 variant particles were added to wild-type HeLa or HeLa TfR knockout cells at MOIs ranging from 1E+3 to 1E+5. The results are shown in FIG. 114.

Differences in mGL expression kinetics were observed between chimeric (AAV-9/2) non-chimeric (AAV-2, AAV-9) aTfR-displaying variant particles. Non-chimeric AAV-9-aTfR-Nb variant particles showed faster transduction kinetics compared to chimeric AAV-9/2-aTfR-Nb and non-chimeric AAV-2-aTfR-Nb variant particles. Since no or only little mGL expression was observed in HeLa cells transduced with control AAV-9/2RA-Nb-ctrl variant particle or wild-type AAV-9 particles, respectively, transduction for chimeric and non-chimeric AAV-9-aTfR-Nb variant particles was mediated by TfR binding and receptor uptake.

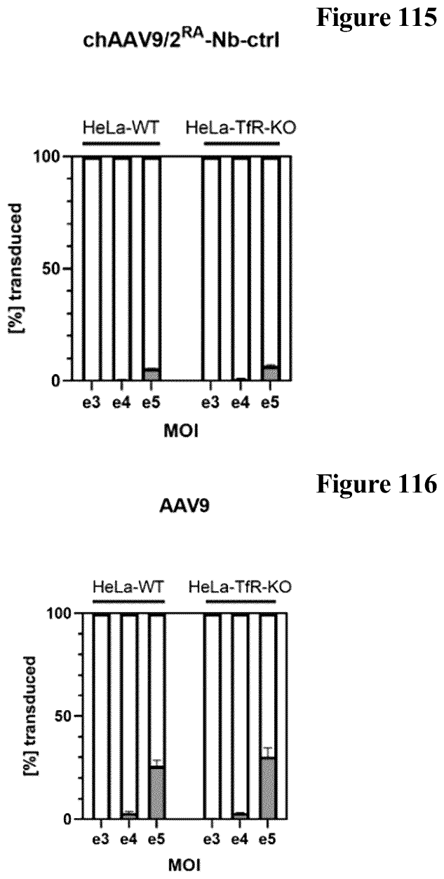

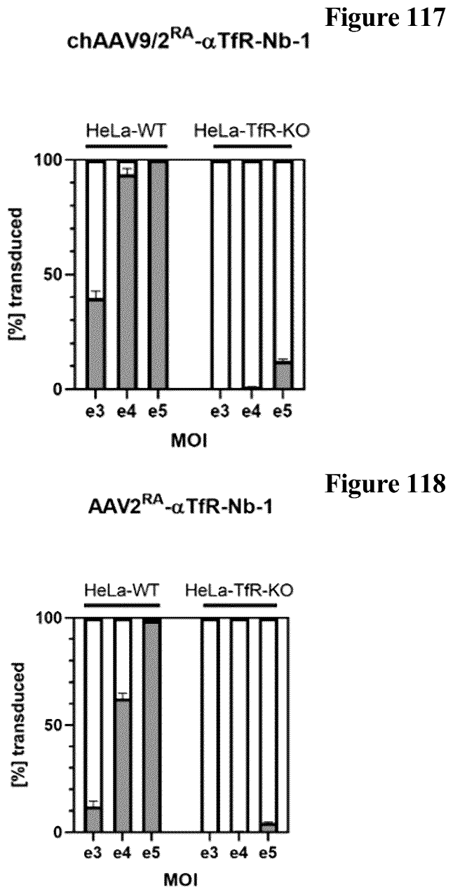

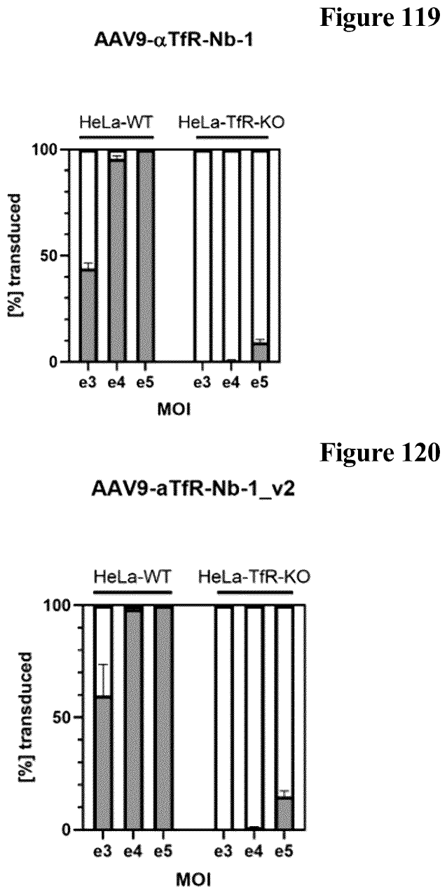

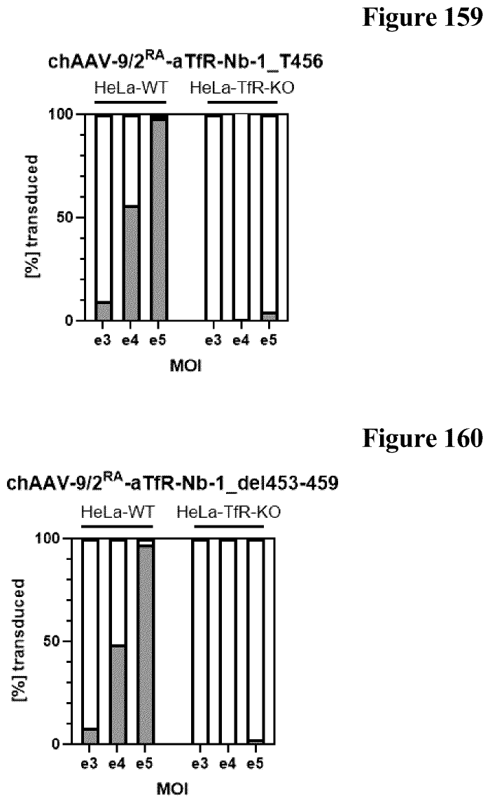

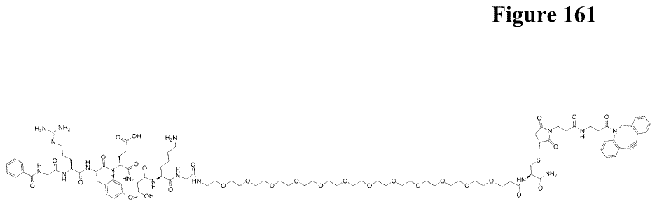

The percentage of mGL-positive cells in HeLa TfR KO cells transduced with chimeric or non-chimeric AAV-9-aTfR-Nb variant particles was significantly lower compared to wild-type HeLa cells indicating that cell entry is mediated by TfR-uptake (see FIG. 115 to FIG. 120 and FIG. 159 to FIG. 160).

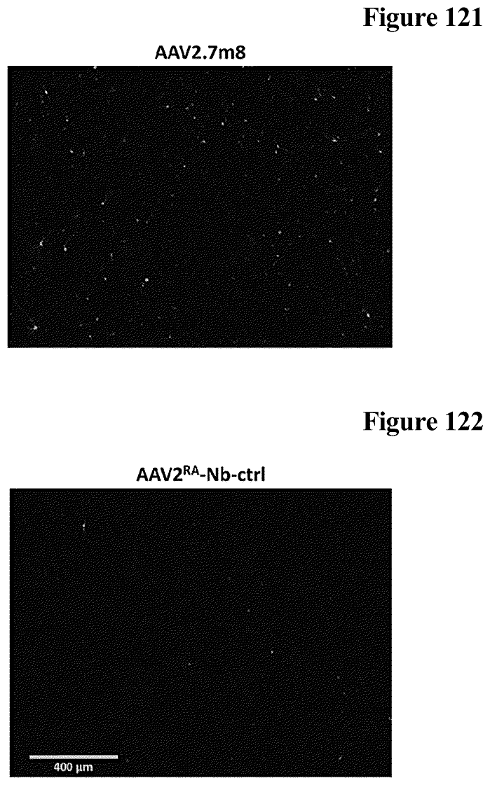

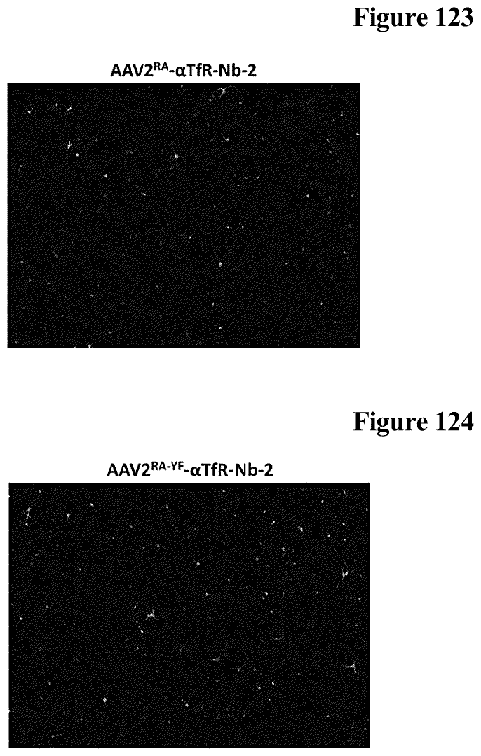

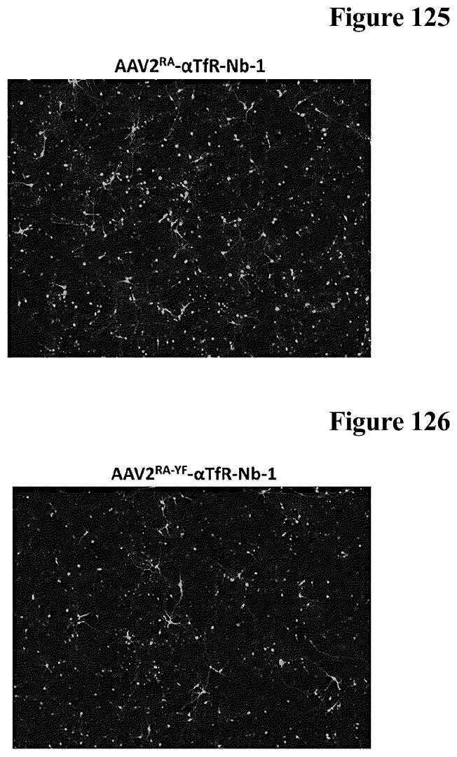

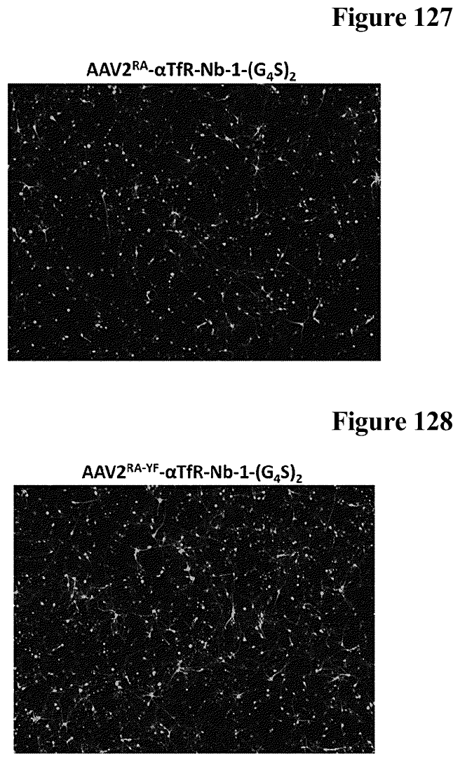

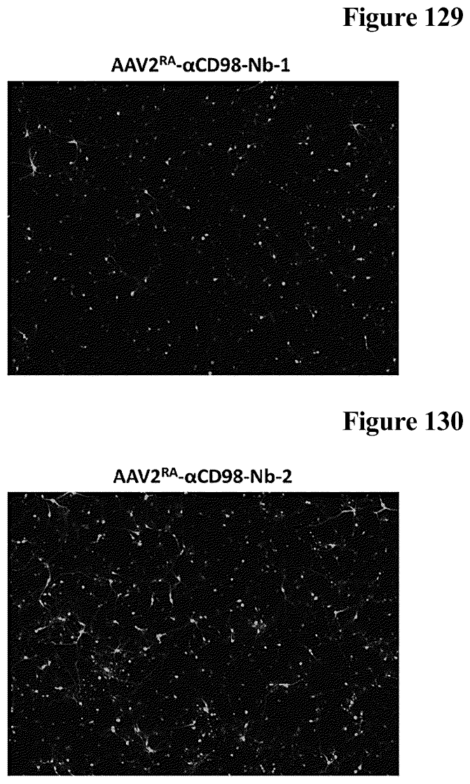

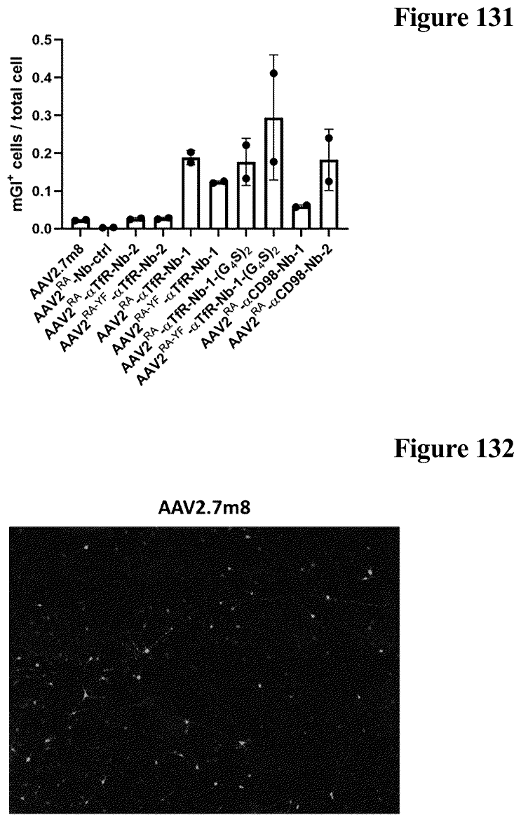

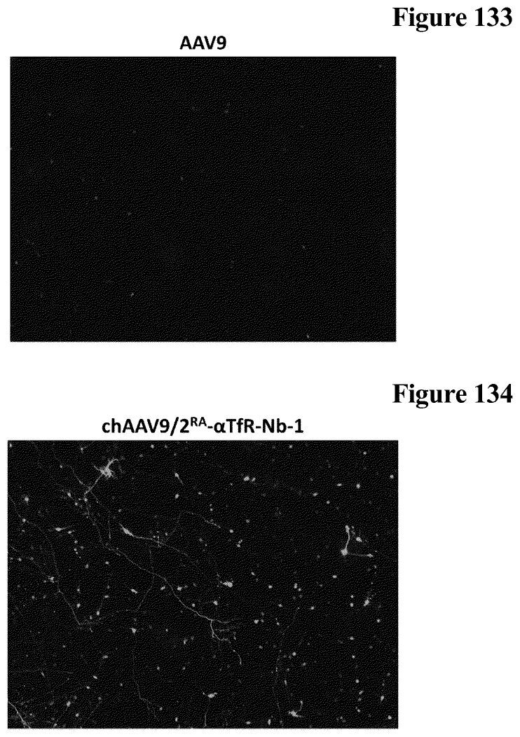

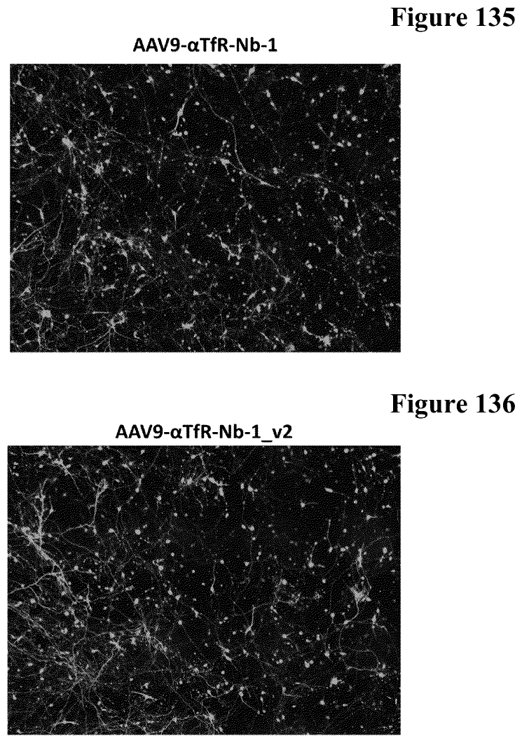

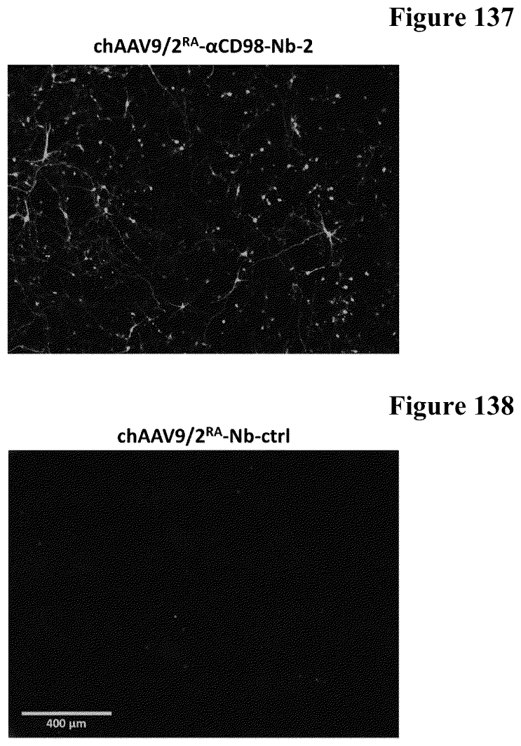

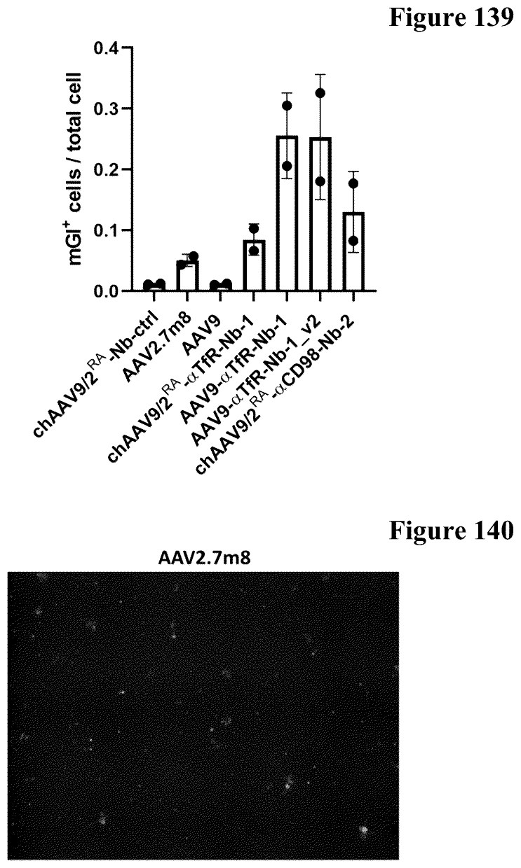

Transduction efficiencies of AAV-2-Nb variant particles compared to non-modified AAV-2 particles as well as AAV-9-Nb variant particles compared to non modified AAV-9 particles were determined using glutamatergic neurons differentiated from human iPSCs. Particles packaged with an mGreenLantern-encoding transgene were added to iCell GlutaNeurons at MOIs ranging from 1E+3 to 1E+5. Transduction kinetics were followed by live imaging every six hours for one week. Seven days post-infection, cells were stained with the nuclear dye Draq5 and transduction efficiencies were determined by quantifying the percentage of mGL-/Draq5-positive cells. The results are shown in FIG. 121 to FIG. 139.

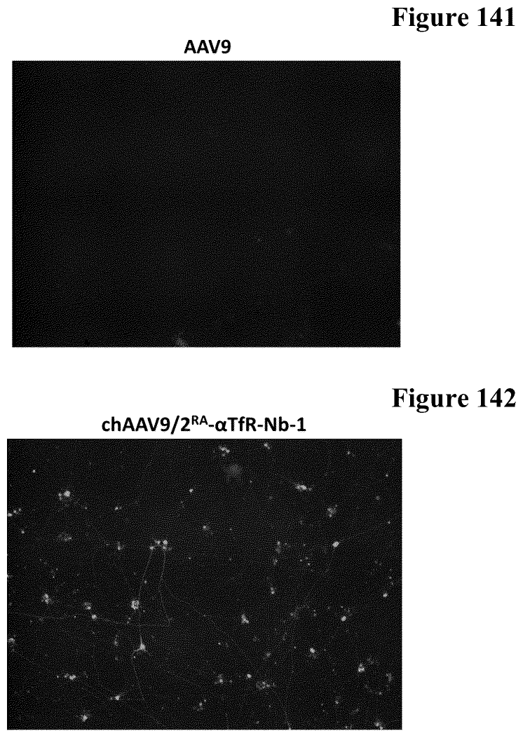

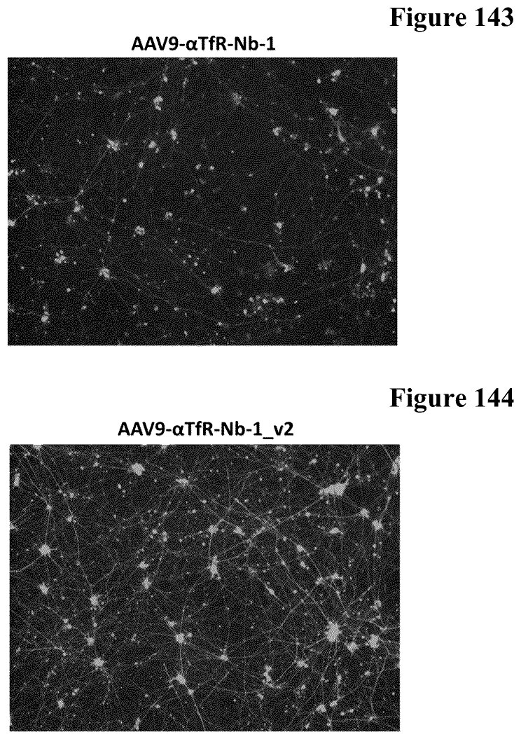

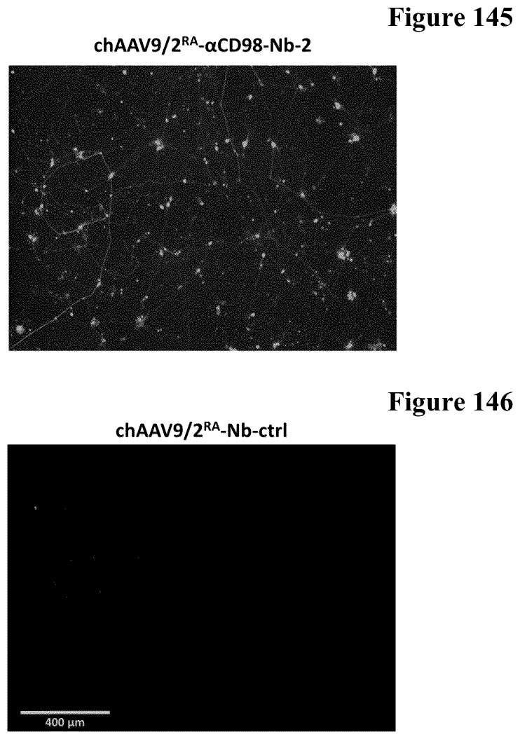

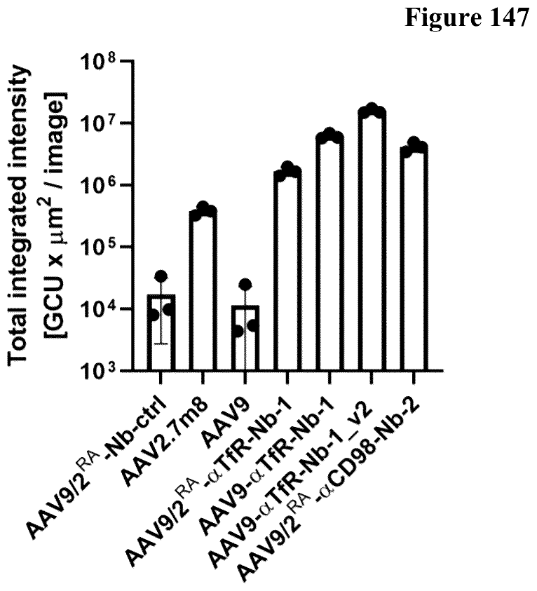

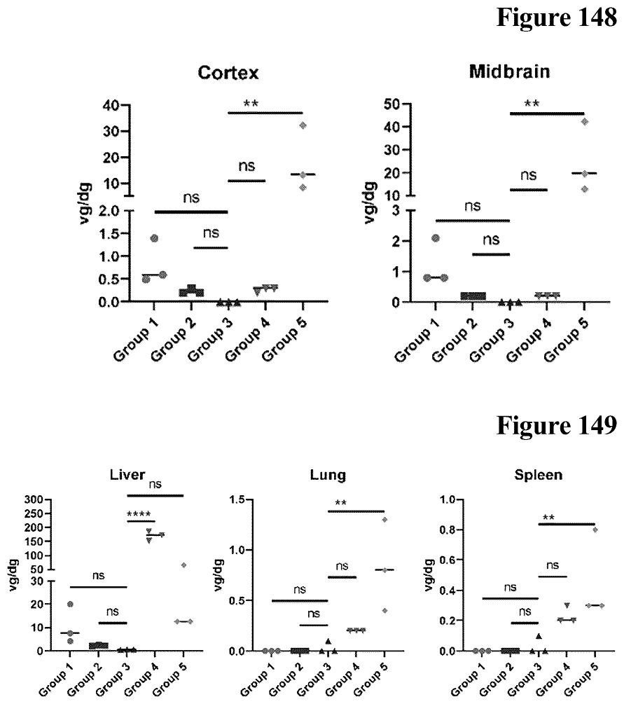

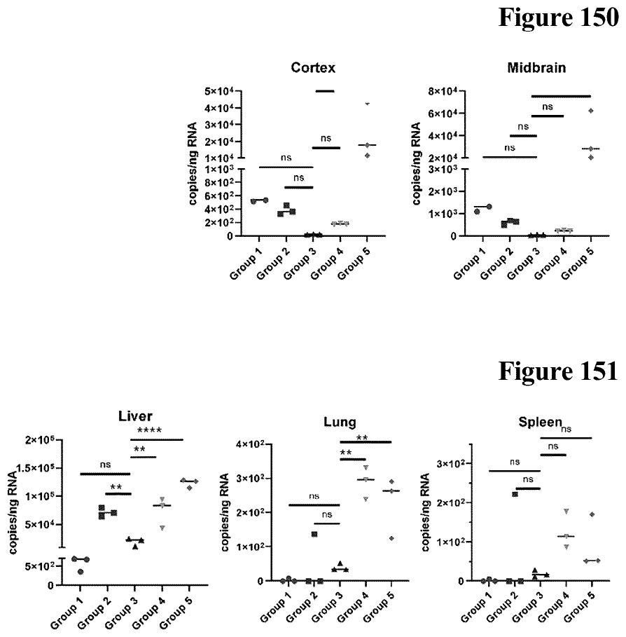

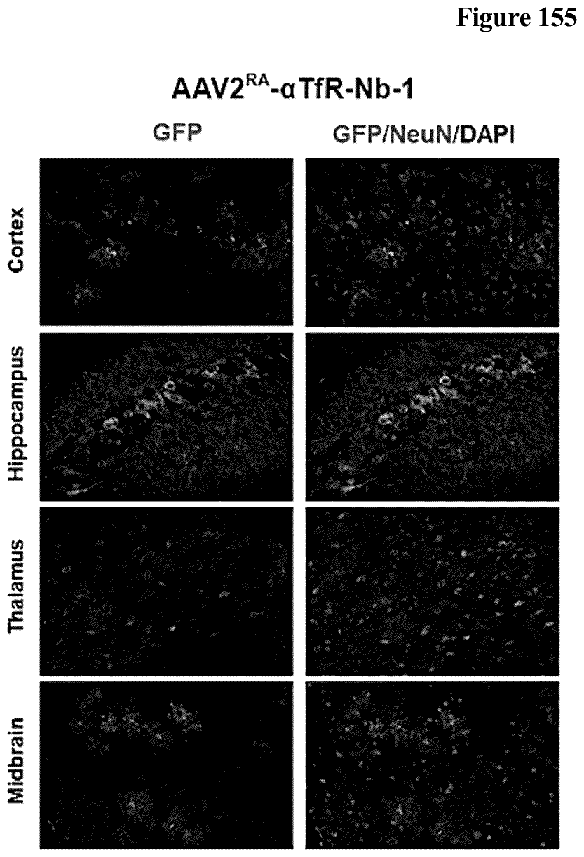

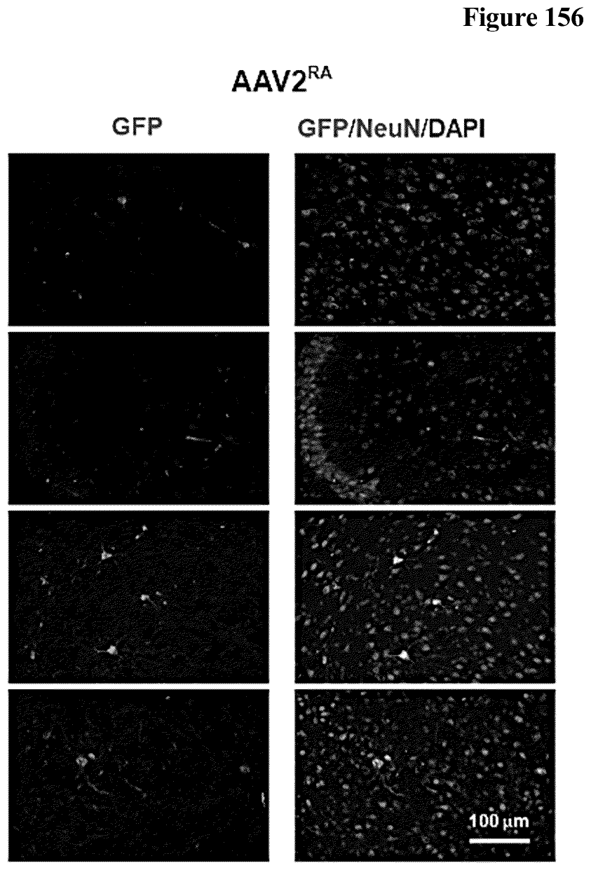

Transduction efficiencies of AAV-9-Nb variant particles and non-modified AAV-9 wild-type particles with NGN2-positive neurons differentiated from human iPSCs were determined. To infect cells, particles packaged with an mGreenLantern reporter encoding transgene were added to NGN2-positive neurons at MOIs ranging from 1E+3 to 1E+5. Transduction kinetics were followed by live imaging every six hours for one week. Seven days post-infection, transduction efficiencies were determined by quantifying mGL intensity per image using the Incucyte® Live-Cell Analysis software. The results are shown in FIG. 140 to FIG. 147. The ability of genetically fused AAV-aTfR-Nb variant particles for transport across the blood-brain barrier by engaging with TfR was assessed in an in vivo mouse study. Biodistribution of chAAV-9/2^{RA}-aTfR-Nb-1 variant particle (group 1), AAV-2^{RA}-aTfR-Nb-1 variant particle (group 2), AAV-2^{RA} variant particle (group 3), wild-type AAV-9 particle (group 4) and AAV-9-PHP.eB variant particle (group 5) was assessed after a single intravenous injection to male mice expressing chimeric human/mouse TfR (TfR^{hu/mu} knock-in mice). Twenty-eight days after application, tissue samples were dissected for DNA and RNA isolation. Both viral genomes (vg) and RNA expression of the viral vector transgene (mGreenLantern) was determined by dPCR in different regions of the brain (on-target) as well as in liver, lung and spleen (off-target). The results are shown in FIG. 148 to FIG. 156. The applied doses are shown in Table 15.

TABLE 15
Doses applied for biodistribution study.

| group | AAV particle | dose [vg/kg/mouse] |
|---|---|---|
| 1 | chAAV-9/2^{RA}-aTfR-Nb-1 | 2.5E+13 |
| 2 | AAV-2^{RA}-aTfR-Nb-1 | 5E+13 |
| 3 | AAV-2^{RA} | 4.4E+13 |
| 4 | wild-type AAV-9 | 4.8E+13 |
| 5 | AAV-9-PHP.eB | 4.2E+13 |

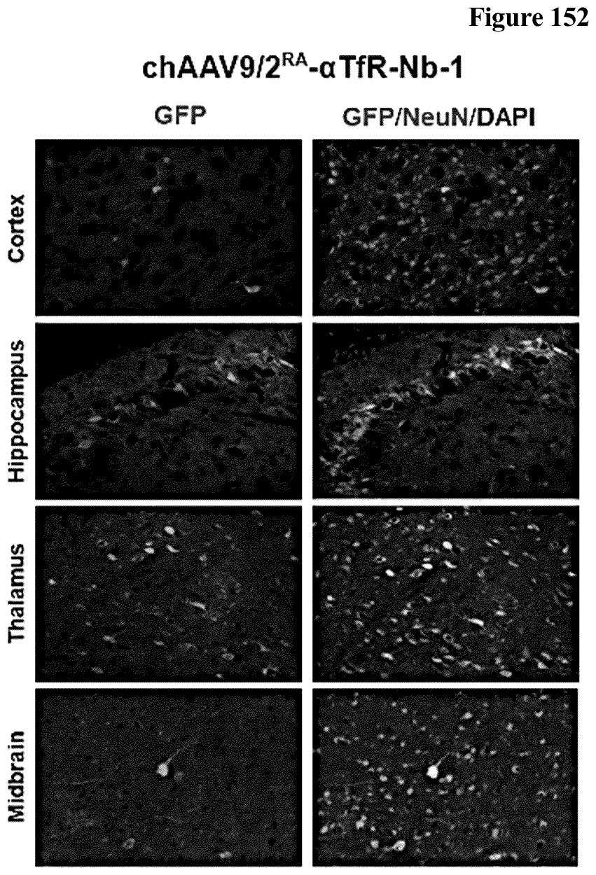

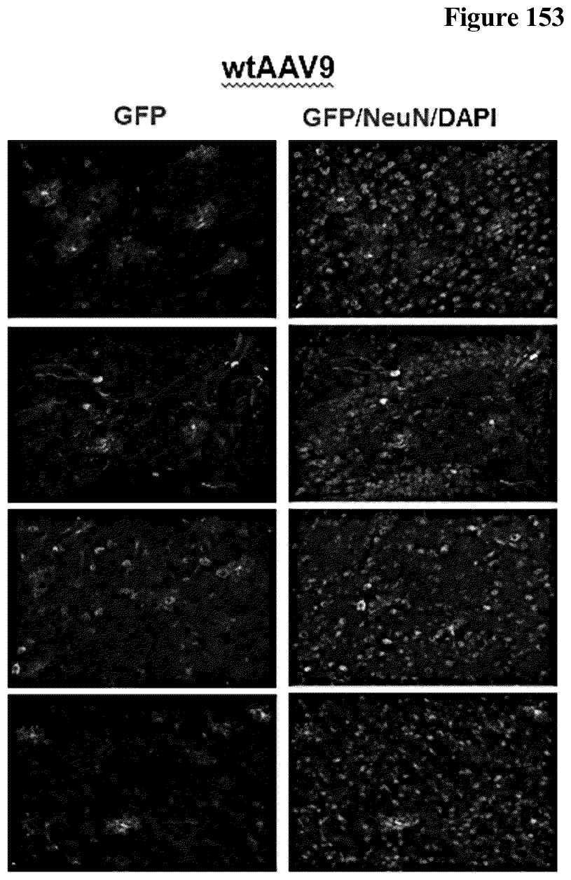

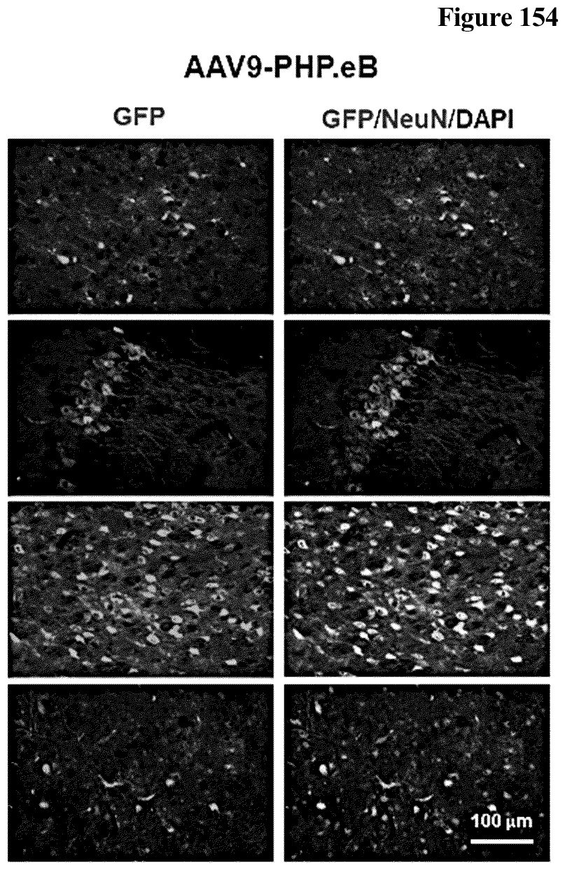

Comparing TfR^{hu/mu} knock-in mice that received chAAV-9/2^{RA}-aTfR-Nb-1 variant particles (group 1) to TfR^{hu/mu} knock-in mice that received wild-type AAV-9 particles (group 4), 2-4-fold more viral genomes per cell and 3-6 fold more RNA were detected in the brain (see FIG. 151 and FIG. 153). Unexpectedly, viral genomes and RNA expression were strongly reduced in mice that received chAAV-9/2^{RA}-aTfR-Nb-1 variant particles (group 1) compared to wild-type AAV-9 particles (group 4) (see FIG. 152 and FIG. 154).

Summarizing the above, the method according to the current invention, i.e. the enzymatic conjugation as well as the direct genetic fusion, have been shown to be robust and reproducible and provide for AAV variant particles with new and advantageous properties.

All publications, patents, and patent applications cited herein are hereby incorporated by reference herein in their entirety for all purposes to the same extent as if each individual publication, patent, and patent application were specifically and individually indicated to be so incorporated by reference. In the event that one or more of the incorporated literature and similar materials differs from or contradicts this application, including but not limited to defined terms, term usage, described techniques, or the like, this application controls.

The following examples and figures as well as the sequences are provided to aid the understanding of the present invention, the true scope of which is set forth in the appended claims. It is understood that modifications can be made in the procedures set forth without departing from the spirit of the invention.

That is, although the disclosed teachings have been described with reference to various applications, methods, and compositions, it will be appreciated that various changes and modifications can be made without departing from the teachings herein and the claimed invention below. The examples are provided to better illustrate the disclosed teachings and are not intended to limit the scope of the teachings presented herein. While the present teachings have been described in terms of these exemplary embodiments, the skilled artisan will readily understand that numerous variations and modifications of these exemplary embodiments are possible without undue experimentation. All such variations and modifications are within the scope of the current teachings.

Sequences

| SEQ | | |
|---|---|---|
| ID NO | description | sequence |
| | VP1-Q- | MAADGYLPDWLEDTLSEGIRQWWKLK |
| 1 | tag_mutHSPG, | PGPPPPKPAERHKDDSRGLVLPGYKYLG |
| | Q-tag YRYRQ, | PFNGLDKGEPVNEADAAALEHDKAYDR |
| | with N- and C- | QLDSGDNPYLKYNHADAEFQERLKEDT |
| | terminal 1xG4S, R | SFGGNLGRAVFQAKKRVLEPLGLVEEP |
| | -> A mutations at | VKTAPGKKRPVEHSPVEPDSSSGTGKAG |
| | amino residues 585 | QQPARKRLNFGQTGDADSVPDPQPLGQ |
| | and 588 | PPAAPSGLGTNTMATGSGAPMADNNEG |
| | (mutHSPG). | ADGVGNSSGNWHCDSTWMGDRVITTS |
| | | TRTWALPTYNNHLYKQISSQSGASNDN |
| | | HYFGYSTPWGYFDFNRFHCHFSPRDWQ |
| | | RLINNNWGFRPKRLNFKLFNIQVKEVTQ |
| | | NDGTTTIANNLTSTVQVFTDSEYQLPYV |
| | | LGSAHQGCLPPFPADVFMVPQYGYLTL |
| | | NNGSQAVGRSSFYCLEYFPSQMLRTGN |
| | | NFTFSYTFEDVPFHSSYAHSQSLDRLMN |
| | | PLIDQYLYYLSRTNTPGGGGSYRYRQG |
| | | GGGSTQSRLQFSQAGASDIRDQSRNWLP |
| | | GPCYRQQRVSKTSADNNNSEYSWTGAT |
| | | KYHLNGRDSLVNPGPAMASHKDDEEKF |
| | | FPQSGVLIFGKQGSEKTNVDIEKVMITD |
| | | EEEIRTTNPVATEQYGSVSTNLQAGNAQ |
| | | AATADVNTQGVLPGMVWQDRDVYLQ |
| | | GPIWAKIPHTDGHFHPSPLMGGFGLKHP |
| | | PPQILIKNTPVPANPSTTFSAAKFASFITQ |
| | | YSTGQVSVEIEWELQKENSKRWNPEIQY |
| | | TSNYNKSVNVDFTVDTNGVYSEPRPIGT |
| | | RYLTRNL |
| 2 | VP2_mutHSPG, R | TAPGKKRPVEHSPVEPDSSSGTGKAGQQ |
| | -> A mutations at | PARKRLNFGQTGDADSVPDPQPLGQPP |
| | amino residues 585 | AAPSGLGTNTMATGSGAPMADNNEGA |
| | and 588 | DGVGNSSGNWHCDSTWMGDRVITTST |
| | (mutHSPG) | RTWALPTYNNHLYKQISSQSGASNDNH |
| | | YFGYSTPWGYFDFNRFHCHFSPRDWQR |
| | | LINNNWGFRPKRLNFKLFNIQVKEVTQN |
| | | DGTTTIANNLTSTVQVFTDSEYQLPYVL |
| | | GSAHQGCLPPFPADVFMVPQYGYLTLN |
| | | NGSQAVGRSSFYCLEYFPSQMLRTGNN |
| | | FTFSYTFEDVPFHSSYAHSQSLDRLMNP |
| | | LIDQYLYYLSRTNTPSGTTTQSRLQFSQ |
| | | AGASDIRDQSRNWLPGPCYRQQRVSKT |
| | | SADNNNSEYSWTGATKYHLNGRDSLV |
| | | NPGPAMASHKDDEEKFFPQSGVLIFGKQ |
| | | GSEKTNVDIEKVMITDEEEIRTTNPVATE |
| | | QYGSVSTNLQAGNAQAATADVNTQGV |
| | | LPGMVWQDRDVYLQGPIWAKIPHTDG |
| | | HFHPSPLMGGFGLKHPPPQILIKNTPVPA |
| | | NPSTTFSAAKFASFITQYSTGQVSVEIEW |
| | | ELQKENSKRWNPEIQYTSNYNKSVNVD |
| | | FTVDTNGVYSEPRPIGTRYLTRNL |
| 3 | VP3_mutHSP, R -> | MATGSGAPMADNNEGADGVGNSSGN |
| | A mutations at | WHCDSTWMGDRVITTSTRTWALPTYN |
| | amino residues 585 | NHLYKQISSQSGASNDNHYFGYSTPWG |
| | and 588 | YFDFNRFHCHFSPRDWQRLINNNWGFR |
| | (mutHSPG) | PKRLNFKLFNIQVKEVTQNDGTTTIANN |
| | | LTSTVQVFTDSEYQLPYVLGSAHQGCLP |
| | | PFPADVFMVPQYGYLTLNNGSQAVGRS |
| | | SFYCLEYFPSQMLRTGNNFTFSYTFEDV |
| | | PFHSSYAHSQSLDRLMNPLIDQYLYYLS |
| | | RTNTPSGTTTQSRLQFSQAGASDIRDQS |
| | | RNWLPGPCYRQQRVSKTSADNNNSEYS |
| | | WTGATKYHLNGRDSLVNPGPAMASHK |
| | | DDEEKFFPQSGVLIFGKQGSEKTNVDIE |
| | | KVMITDEEEIRTTNPVATEQYGSVSTNL |
| | | QAGNAQAATADVNTQGVLPGMVWQD |
| | | RDVYLQGPIWAKIPHTDGHFHPSPLMG |
| | | GFGLKHPPPQILIKNTPVPANPSTTESAA |
| | | KFASFITQYSTGQVSVEIEWELQKENSK |
| | | RWNPEIQYTSNYNKSVNVDFTVDTNGV |
| | | YSEPRPIGTRYLTRNL |
| 4 | mGreenLantern | MVSKGEELFTGVVPILVELDGDVNGHK |
| | | FSVRGEGEGDATNGKLTLKFICTTGKLP |
| | | VPWPTLVTTLGYGVACFARYPDHMKQ |
| | | HDFFKSAMPEGYVQERTISFKDDGTYKT |
| | | RAEVKFEGDTLVNRIVLKGIDFKEDGNI |
| | | LGHKLEYNFNSHKVYITADKQKNGIKA |
| | | NFKTRHNVEDGGVQLADHYQQNTPIGD |
| | | GPVLLPDNHYLSHQSKLSKDPNEKRDH |
| | | MVLKERVTAAGITHDMDELYK |
| 5 | Firefly | MEDAKNIKKGPAPFYPLEDGTAGEQLH |
| | Luciferase | KAMKRYALVPGTIAFTDAHIEVDITYAE |
| | | YFEMSVRLAEAMKRYGLNTNHRIVVCS |
| | | ENSLQFFMPVLGALFIGVAVAPANDIYN |
| | | ERELLNSMGISQPTVVFVSKKGLQKILN |
| | | VQKKLPIIQKIIIMDSKTDYQGFQSMYTF |
| | | VTSHLPPGFNEYDFVPESFDRDKTIALIM |
| | | NSSGSTGLPKGVALPHRTACVRFSHARD |
| | | PIFGNQIIPDTAILSVVPFHHGFGMFTTLG |
| | | YLICGFRVVLMYRFEEELFLRSLODYKI |
| | | QSALLVPTLFSFFAKSTLIDKYDLSNLHE |
| | | IASGGAPLSKEVGEAVAKRFHLPGIRQG |
| | | YGLTETTSAILITPEGDDKPGAVGKVVP |
| | | FFEAKVVDLDTGKTLGVNQRGELCVRG |
| | | PMIMSGYVNNPEATNALIDKDGWLHSG |
| | | DIAYWDEDEHFFIVDRLKSLIKYKGYQV |
| | | APAELESILLQHPNIFDAGVAGLPDDDA |
| | | GELPAAVVVLEHGKTMTEKEIVDYVAS |
| | | QVTTAKKLRGGVVFVDEVPKGLTGKLD |
| | | ARKIREILIKAKKGGKIAV |
| 6 | TfR | HHHHHHGLNDIFEAQKIEWHEAPPAPC |
| | human TFR ab C89 | KGVEPKTECERLAGTESPVREEPGEDFP |
| | with N-terminal | AARRLYWDDLKRKLSEKLDSTDFTGTI |
| | His6-Avitaq- | KLLNENSYVPREAGSQKDENLALYVEN |
| | IgAProtease site | QFREFKLSKVWRDQHFVKIQVKDSAQN |
| | | SVIIVDKNGRLVYLVENPGGYVAYSKA |
| | | ATVTGKLVHANFGTKKDFEDLYTPVNG |
| | | SIVIVRAGKITFAEKVANAESLNAIGVLI |
| | | YMDQTKFPIVNAELSFFGHAHLGTGDP |
| | | YTPGFPSFNHTQFPPSRSSGLPNIPVQTIS |
| | | RAAAEKLFGNMEGDCPSDWKTDSTCR |
| | | MVTSESKNVKLTVSNVLKEIKILNIFGVI |
| | | KGFVEPDHYVVVGAQRDAWGPGAAKS |
| | | GVGTALLLKLAQMFSDMVLKDGFQPSR |
| | | SIIFASWSAGDFGSVGATEWLEGYLSSL |
| | | HLKAFTYINLDKAVLGTSNFKVSASPLL |
| | | YTLIEKTMQNVKHPVTGQFLYQDSNWA |
| | | SKVEKLTLDNAAFPFLAYSGIPAVSFCFC |
| | | EDTDYPYLGTTMDTYKELIERIPELNKV |
| | | ARAAAEVAGQFVIKLTHDVELNLDYER |
| | | YNSQLLSFVRDLNQYRADIKEMGLSLQ |
| | | WLYSARGDFFRATSRLTTDFGNAEKTD |
| | | RFVMKKLNDRVMRVEYHFLSPYVSPKE |
| | | SPFRHVFWGSGSHTLPALLENLKLRKQN |
| | | NGAFNETLFRNQLALATWTIQGAANAL |
| | | SGDVWDIDNEF |
| 7 | human EGFR (hu | LEEKKGNYVVTDHGSCVRACGADSYE |
| | EGFRVIII ECD Avi | MEEDGVRKCKKCEGPCRKVCNGIGIGE |
| | His) | FKDSLSINATNIKHFKNCTSISGDLHILPV |
| | | AFRGDSFTHTPPLDPQELDILKTVKEITG |
| | | FLLIQAWPENRTDLHAFENLEIIRGRTKQ |
| | | HGQFSLAVVSLNITSLGLRSLKEISDGDV |
| | | IISGNKNLCYANTINWKKLFGTSGQKTK |
| | | IISNRGENSCKATGQVCHALCSPEGCWG |
| | | PEPRDCVSCRNVSRGRECVDKCNLLEG |
| | | EPREFVENSECIQCHPECLPQAMNITCTG |
| | | RGPDNCIQCAHYIDGPHCVKTCPAGVM |
| | | GENNTLVWKYADAGHVCHLCHPNCTY |
| | | GCTGPGLEGCPTNGPKIPSVDGGSPTPPT |
| | | PGGGSGLNDIFEAQKIEWHEARAHHHH |
| | | HH |
| 8 | VP1-Q-tag_HSPG | MAADGYLPDWLEDTLSEGIRQWWKLK |
| | intact, Q-Tag | PGPPPPKPAERHKDDSRGLVLPGYKYLG |
| | YRYRQ, with N- | PFNGLDKGEPVNEADAAALEHDKAYDR |
| | and C-terminal | QLDSGDNPYLKYNHADAEFQERLKEDT |
| | 1xG4S Linker | SFGGNLGRAVFQAKKRVLEPLGLVEEP |
| | sequence in VP1 | VKTAPGKKRPVEHSPVEPDSSSGTGKAG |
| | CDS | QQPARKRLNFGQTGDADSVPDPQPLGQ |
| | | PPAAPSGLGTNTMATGSGAPMADNNEG |
| | | ADGVGNSSGNWHCDSTWMGDRVITTS |
| | | TRTWALPTYNNHLYKQISSQSGASNDN |
| | | HYFGYSTPWGYFDFNRFHCHFSPRDWQ |
| | | RLINNNWGFRPKRLNFKLFNIQVKEVTQ |
| | | NDGTTTIANNLTSTVQVFTDSEYQLPYV |
| | | LGSAHQGCLPPFPADVFMVPQYGYLTL |
| | | NNGSQAVGRSSFYCLEYFPSQMLRTGN |
| | | NFTFSYTFEDVPFHSSYAHSQSLDRLMN |
| | | PLIDQYLYYLSRTNTPGGGGSYRYRQG |
| | | GGGSTQSRLQFSQAGASDIRDQSRNWLP |
| | | GPCYRQQRVSKTSADNNNSEYSWTGAT |
| | | KYHLNGRDSLVNPGPAMASHKDDEEKF |
| | | FPQSGVLIFGKQGSEKTNVDIEKVMITD |
| | | EEEIRTTNPVATEQYGSVSTNLQRGNRQ |
| | | AATADVNTQGVLPGMVWQDRDVYLQ |
| | | GPIWAKIPHTDGHFHPSPLMGGFGLKHP |
| | | PPQILIKNTPVPANPSTTFSAAKFASFITQ |
| | | YSTGQVSVEIEWELQKENSKRWNPEIQY |
| | | TSNYNKSVNVDFTVDTNGVYSEPRPIGT |
| | | RYLTRNL |
| 9 | VP2_HSPG intact | TAPGKKRPVEHSPVEPDSSSGTGKAGQQ |
| | | PARKRLNFGQTGDADSVPDPQPLGQPP |
| | | AAPSGLGTNTMATGSGAPMADNNEGA |
| | | DGVGNSSGNWHCDSTWMGDRVITTST |
| | | RTWALPTYNNHLYKQISSQSGASNDNH |
| | | YFGYSTPWGYFDFNRFHCHFSPRDWQR |
| | | LINNNWGFRPKRLNFKLFNIQVKEVTQN |
| | | DGTTTIANNLTSTVQVFTDSEYQLPYVL |
| | | GSAHQGCLPPFPADVFMVPQYGYLTLN |
| | | NGSQAVGRSSFYCLEYFPSQMLRTGNN |
| | | FTFSYTFEDVPFHSSYAHSQSLDRLMNP |
| | | LIDQYLYYLSRTNTPSGTTTQSRLQFSQ |
| | | AGASDIRDQSRNWLPGPCYRQQRVSKT |
| | | SADNNNSEYSWTGATKYHLNGRDSLV |
| | | NPGPAMASHKDDEEKFFPQSGVLIFGKQ |
| | | GSEKTNVDIEKVMITDEEEIRTTNPVATE |
| | | QYGSVSTNLORGNRQAATADVNTQGV |
| | | LPGMVWQDRDVYLQGPIWAKIPHTDG |
| | | HFHPSPLMGGFGLKHPPPQILIKNTPVPA |
| | | NPSTTFSAAKFASFITQYSTGQVSVEIEW |
| | | ELQKENSKRWNPEIQYTSNYNKSVNVD |
| | | FTVDTNGVYSEPRPIGTRYLTRNL |
| 10 | VP3_HSPG intact | MATGSGAPMADNNEGADGVGNSSGN |
| | | WHCDSTWMGDRVITTSTRTWALPTYN |
| | | NHLYKQISSQSGASNDNHYFGYSTPWG |
| | | YFDFNRFHCHFSPRDWQRLINNNWGFR |
| | | PKRLNFKLFNIQVKEVTQNDGTTTIANN |
| | | LTSTVQVFTDSEYQLPYVLGSAHQGCLP |
| | | PFPADVFMVPQYGYLTLNNGSQAVGRS |
| | | SFYCLEYFPSQMLRTGNNFTFSYTFEDV |
| | | PFHSSYAHSQSLDRLMNPLIDQYLYYLS |
| | | RTNTPSGTTTQSRLQFSQAGASDIRDQS |
| | | RNWLPGPCYRQQRVSKTSADNNNSEYS |
| | | WTGATKYHLNGRDSLVNPGPAMASHK |
| | | DDEEKFFPQSGVLIFGKQGSEKTNVDIE |
| | | KVMITDEEEIRTTNPVATEQYGSVSTNL |
| | | QRGNRQAATADVNTQGVLPGMVWQD |
| | | RDVYLQGPIWAKIPHTDGHFHPSPLMG |
| | | GFGLKHPPPQILIKNTPVPANPSTTFSAA |
| | | KFASFITQYSTGQVSVEIEWELQKENSK |
| | | RWNPEIQYTSNYNKSVNVDFTVDTNGV |
| | | YSEPRPIGTRYLTRNL |

| SEQ | | | | |
|---|---|---|---|---|
| ID | | Tar- | | |
| NO | description | get | Format | Sequence |
| 11 | K-tag- | TfR | VNAR- | GGGRYESKGGGGGGGSARVDQTPQTITKE |
| | G4S- | | Fc | TGESLTINCVLRDSNCALPSTYWYRKKSG |
| | VNAR | | | STNEESISKGGRYVETVNSGSKSFSLRINDL |
| | 8a- | | | TVEDSGTYRCKVIAQLGWWLRGCNYRKH |
| | G4S- | | | DVYGDGTAVTVNAGGGGSHHHHHHLEV |
| | His6- | | | LFQGPGGGGSDKTHTCPPCPAPEAAGGPS |
| | 3C-Fc | | | VFLFPPKPKDTLMISRTPEVTCVVVDVSHE |
| | | | | DPEVKFNWYVDGVEVHNAKTKPREEQYN |
| | | | | STYRVVSVLTVLHQDWLNGKEYKCKVSN |
| | | | | KALGAPIEKTISKAKGQPREPQVYTLPPSR |
| | | | | DELTKNQVSLTCLVKGFYPSDIAVEWESN |
| | | | | GQPENNYKTTPPVLDSDGSFFLYSKLTVD |
| | | | | KSRWQQGNVFSCSVMHEALHNHYTQKSL |
| | | | | SLSPGK |
| 12 | K-tag- | TfR | VHH- | GGGRYESKGGGGGGGSQVQLVESGGGLV |
| | G4S- | | Fc | QAGGSLRLSCAASGSSISSIAMGWYRQAP |
| | MH12- | | | GKERELVAAISSGGTINYADSVKGRFTISR |
| | G4S- | | | DNAKNTVYLQMNSLKPEDTAVYYCNTGR |
| | His6- | | | RLQTGSWGQGTQVTVSSGGGGSHHHHHH |
| | 3C-Fc | | | LEVLFQGPGGGGSDKTHTCPPCPAPEAAG |
| | | | | GPSVFLFPPKPKDTLMISRTPEVTCVVVDV |
| | | | | SHEDPEVKFNWYVDGVEVHNAKTKPREE |
| | | | | QYNSTYRVVSVLTVLHQDWLNGKEYKCK |
| | | | | VSNKALGAPIEKTISKAKGQPREPQVYTLP |
| | | | | PSRDELTKNQVSLTCLVKGFYPSDIAVEW |
| | | | | ESNGQPENNYKTTPPVLDSDGSFFLYSKLT |
| | | | | VDKSRWQQGNVFSCSVMHEALHNHYTQ |
| | | | | KSLSLSPGK |
| 13 | VNAR | TfR | VNAR- | ARVDQTPQTITKETGESLTINCVLRDSNCA |
| | 8a- | | Fc | LPSTYWYRKKSGSTNEESISKGGRYVETV |
| | G4S- | | | NSGSKSFSLRINDLTVEDSGTYRCKVIAQL |
| | His6-K- | | | GWWLRGCNYRKHDVYGDGTAVTVNAGG |
| | tag-3C- | | | GGSHHHHHHGGGRYESKGGGLEVLFQGP |
| | Fc | | | GGGGSDKTHTCPPCPAPEAAGGPSVFLFPP |
| | | | | KPKDTLMISRTPEVTCVVVDVSHEDPEVK |
| | | | | FNWYVDGVEVHNAKTKPREEQYNSTYRV |
| | | | | VSVLTVLHQDWLNGKEYKCKVSNKALGA |
| | | | | PIEKTISKAKGQPREPQVYTLPPSRDELTKN |
| | | | | QVSLTCLVKGFYPSDIAVEWESNGQPENN |
| | | | | YKTTPPVLDSDGSFFLYSKLTVDKSRWQQ |
| | | | | GNVFSCSVMHEALHNHYTQKSLSLSPGK |
| 14 | MH12- | TfR | VHH- | QVQLVESGGGLVQAGGSLRLSCAASGSSIS |
| | G4S- | | Fc | SIAMGWYRQAPGKERELVAAISSGGTINY |
| | His6-K- | | | ADSVKGRFTISRDNAKNTVYLQMNSLKPE |
| | tag-3C- | | | DTAVYYCNTGRRLQTGSWGQGTQVTVSS |
| | Fc | | | GGGGSHHHHHHGGGRYESKGGGLEVLFQ |
| | | | | GPGGGGSDKTHTCPPCPAPEAAGGPSVFLF |
| | | | | PPKPKDTLMISRTPEVTCVVVDVSHEDPEV |
| | | | | KFNWYVDGVEVHNAKTKPREEQYNSTYR |
| | | | | VVSVLTVLHQDWLNGKEYKCKVSNKALG |
| | | | | APIEKTISKAKGQPREPQVYTLPPSRDELTK |
| | | | | NQVSLTCLVKGFYPSDIAVEWESNGQPEN |
| | | | | NYKTTPPVLDSDGSFFLYSKLTVDKSRWQ |
| | | | | QGNVFSCSVMHEALHNHYTQKSLSLSPGK |
| 15 | aEGFR | EGFR | Fab | HC: |
| | Fab- | | | QVQLKQSGPGLVQPSQSLSITCTVSGFSLT |
| | | | | NYGVHWVRQSPGKGLEWLGVIWSGGNT |
| | | | | DYNTPFTSRLSINKDNSKSQVFFKMNSLQS |
| | | | | NDTAIYYCARALTYYDYEFAYWGQGTLV |
| | | | | TVSAASTKGPSVFPLAPSSKSTSGGTAALG |
| | | | | CLVKDYFPEPVTVSWNSGALTSGVHTFPA |
| | | | | VLQSSGLYSLSSVVTVPSSSLGTQTYICNV |
| | | | | NHKPSNTKVDKKVEPKSCGGGGSGGGGS |
| | | | | GGGGRYESKGGG |
| 48 | 2G4S- | | | LC (kappa): |
| | K-tag | | | DILLTQSPVILSVSPGERVSFSCRASQSIGTN |
| | | | | IHWYQQRTNGSPRLLIKYASESISGIPSRES |
| | | | | GSGSGTDFTLSINSVESEDIADYYCQQNNN |
| | | | | WPTTFGAGTKLELKRTVAAPSVFIFPPSDE |
| | | | | QLKSGTASVVCLLNNFYPREAKVQWKVD |
| | | | | NALQSGNSQESVTEQDSKDSTYSLSSTLTL |
| | | | | SKADYEKHKVYACEVTHQGLSSPVTKSFN |
| | | | | RGEC |
| 16 | aEGFR | EGFR | Fab | HC: |
| | Fab- | | | QVQLKQSGPGLVQPSQSLSITCTVSGFSLT |
| | 4G4S- | | | NYGVHWVRQSPGKGLEWLGVIWSGGNT |
| | K-tag | | | DYNTPFTSRLSINKDNSKSQVFFKMNSLOS |
| | | | | NDTAIYYCARALTYYDYEFAYWGQGTLV |
| | | | | TVSAASTKGPSVFPLAPSSKSTSGGTAALG |
| | | | | CLVKDYFPEPVTVSWNSGALTSGVHTFPA |
| | | | | VLQSSGLYSLSSVVTVPSSSLGTQTYICNV |
| | | | | NHKPSNTKVDKKVEPKSCGGGGSGGGGS |
| | | | | GGGGSGGGGSGGGRYESKGGG |
| 77 | | | | LC (kappa): |
| | | | | DILLTQSPVILSVSPGERVSFSCRASQSIGTN |
| | | | | IHWYQQRTNGSPRLLIKYASESISGIPSRFS |
| | | | | GSGSGTDFTLSINSVESEDIADYYCQQNNN |
| | | | | WPTTFGAGTKLELKRTVAAPSVFIFPPSDE |
| | | | | QLKSGTASVVCLLNNFYPREAKVQWKVD |
| | | | | NALQSGNSQESVTEQDSKDSTYSLSSTLTL |
| | | | | SKADYEKHKVYACEVTHQGLSSPVTKSEN |
| | | | | RGEC |
| 17 | aEGFR | EGFR | scFv- | DILLTQSPVILSVSPGERVSFSCRASQSIGTN |
| | scFv- | | Fc | IHWYQQRTNGSPRLLIKYASESISGIPSRES |
| | 2G4S- | | | GSGSGTDFTLSINSVESEDIADYYCQQNNN |
| | K-tag- | | | WPTTFGCGTKLELKGGSGGGGSGGGGSG |
| | 3C-Fc | | | GGGSGGQVQLKQSGPGLVQPSQSLSITCT |
| | | | | VSGFSLTNYGVHWVRQSPGKCLEWLGVI |
| | | | | WSGGNTDYNTPFTSRLSINKDNSKSQVFF |
| | | | | KMNSLQSNDTAIYYCARALTYYDYEFAY |
| | | | | WGQGTLVTVSAGGGGSGGGGSGGGGRYE |
| | | | | SKGGGLEVLFQGPGGGGSDKTHTCPPCPA |
| | | | | PEAAGGPSVFLFPPKPKDTLMISRTPEVTC |
| | | | | VVVDVSHEDPEVKFNWYVDGVEVHNAK |
| | | | | TKPREEQYNSTYRVVSVLTVLHQDWLNG |
| | | | | KEYKCKVSNKALGAPIEKTISKAKGQPREP |
| | | | | QVYTLPPSRDELTKNQVSLTCLVKGFYPS |
| | | | | DIAVEWESNGQPENNYKTTPPVLDSDGSF |
| | | | | FLYSKLTVDKSRWQQGNVFSCSVMHEAL |
| | | | | HNHYTQKSLSLSPGK |
| 18 | aEGFR | EGFR | scFv- | DILLTQSPVILSVSPGERVSFSCRASQSIGTN |
| | _scFv- | | Fc | IHWYQQRTNGSPRLLIKYASESISGIPSRFS |
| | 4G4S- | | | GSGSGTDFTLSINSVESEDIADYYCQQNNN |
| | K-tag | | | WPTTFGCGTKLELKGGSGGGGSGGGGSG |
| | | | | GGGSGGQVQLKQSGPGLVQPSQSLSITCT |
| | | | | VSGFSLTNYGVHWVRQSPGKCLEWLGVI |
| | | | | WSGGNTDYNTPFTSRLSINKDNSKSQVFF |
| | | | | KMNSLQSNDTAIYYCARALTYYDYEFAY |
| | | | | WGQGTLVTVSAGGGGSGGGGSGGGGSGG |
| | | | | GGSGGGRYESKGGGLEVLFQGPGGGGSD |
| | | | | KTHTCPPCPAPEAAGGPSVFLFPPKPKDTL |
| | | | | MISRTPEVTCVVVDVSHEDPEVKFNWYVD |
| | | | | GVEVHNAKTKPREEQYNSTYRVVSVLTVL |
| | | | | HQDWLNGKEYKCKVSNKALGAPIEKTISK |
| | | | | AKGQPREPQVYTLPPSRDELTKNQVSLTC |
| | | | | LVKGFYPSDIAVEWESNGQPENNYKTTPP |
| | | | | VLDSDGSFFLYSKLTVDKSRWQQGNVFSC |
| | | | | SVMHEALHNHYTQKSLSLSPGK |
| 19 | aTFR | TfR | Fab | HC: |
| | Fab- | | | EVQLQQSGPELVKPGASMKISCKASGYSF |
| | 2G4S- | | | TGYTMNWVKQSHGENLEWIGRINPHNGG |
| | | | | TDYNQKFKDKAPLTVDKSSNTAYMELLSL |
| | | | | TSEDSAVYYCARGYYYYSLDYWGQGTSV |
| | | | | TVSSASTKGPSVFPLAPSSKSTSGGTAALG |
| | | | | CLVKDYFPEPVTVSWNSGALTSGVHTFPA |
| | | | | VLQSSGLYSLSSVVTVPSSSLGTQTYICNV |
| | | | | NHKPSNTKVDKKVEPKSCGGGGSGGGGS |
| | | | | GGGGRYESKGGG |
| 78 | K-tag | | | LC (kappa): |
| | (high | | | QIVLTQSPAIMSASPGEKVTMTCSASSSIRY |
| | affinity) | | | IHWYQQRPGTSPKRWIYDTSNLASGVPAR |
| | | | | FSGSGSGTSYSLTISSMEAEDAATYYCHQR |
| | | | | NSYPWTFGGGTRLEIKRTVAAPSVFIFPPS |
| | | | | DEQLKSGTASVVCLLNNFYPREAKVQWK |
| | | | | VDNALQSGNSQESVTEQDSKDSTYSLSSTL |
| | | | | TLSKADYEKHKVYACEVTHQGLSSPVTKS |
| | | | | FNRGEC |
| 20 | aTFR | TfR | Fab | HC: |
| | Fab- | | | QSMQESGPGLVKPSQTLSLTCTVSGFSLSS |
| | 2G4S- | | | YAMSWIRQHPGKGLEWIGYIWSGGSTDY |
| | K-tag | | | ASWAKSRVTISKTSTTVSLKLSSVTAADTA |
| | (low | | | VYYCARRYGTSYPDYGDASGFDPWGQGT |
| | affinity) | | | LVTVSSASTKGPSVFPLAPSSKSTSGGTAA |
| | | | | LGCLVKDYFPEPVTVSWNSGALTSGVHTF |
| | | | | PAVLQSSGLYSLSSVVTVPSSSLGTQTYIC |
| | | | | NVNHKPSNTKVDKKVEPKSCGGGGSGGG |
| | | | | GSGGGGRYESKGGG |
| 79 | | | | LC (kappa): |
| | | | | AIQLTQSPSSLSASVGDRVTITCRASQSISS |
| | | | | YLAWYQQKPGKAPKLLIYRASTLASGVPS |
| | | | | RFSGSGSGTDFTLTISSLQPEDFATYYCQQ |
| | | | | NYASSNVDNTFGGGTKVEIKRTVAAPSVFI |
| | | | | FPPSDEQLKSGTASVVCLLNNFYPREAKV |
| | | | | QWKVDNALQSGNSQESVTEQDSKDSTYS |
| | | | | LSSTLTLSKADYEKHKVYACEVTHQGLSS |
| | | | | PVTKSFNRGEC |
| 21 | aTFR | TfR | Fab | HC: |
| | Fab- | | | EVQLQQSGPELVKPGASMKISCKASGYSF |
| | 4G4S- | | | TGYTMNWVKQSHGENLEWIGRINPHNGG |
| | K-tag | | | TDYNQKFKDKAPLTVDKSSNTAYMELLSL |
| | (high | | | TSEDSAVYYCARGYYYYSLDYWGQGTSV |
| | affinity) | | | TVSSASTKGPSVFPLAPSSKSTSGGTAALG |
| | | | | CLVKDYFPEPVTVSWNSGALTSGVHTFPA |
| | | | | VLQSSGLYSLSSVVTVPSSSLGTQTYICNV |
| | | | | NHKPSNTKVDKKVEPKSCGGGGSGGGGS |
| | | | | GGGGSGGGGSGGGRYESKGGG |
| 80 | | | | LC (kappa): |
| | | | | QIVLTQSPAIMSASPGEKVTMTCSASSSIRY |
| | | | | IHWYQQRPGTSPKRWIYDTSNLASGVPAR |
| | | | | FSGSGSGTSYSLTISSMEAEDAATYYCHQR |
| | | | | NSYPWTFGGGTRLEIKRTVAAPSVFIFPPS |
| | | | | DEQLKSGTASVVCLLNNFYPREAKVQWK |
| | | | | VDNALQSGNSQESVTEQDSKDSTYSLSSTL |
| | | | | TLSKADYEKHKVYACEVTHQGLSSPVTKS |
| | | | | FNRGEC |
| 22 | aTFR | TfR | Fab | HC: |
| | Fab- | | | QSMQESGPGLVKPSQTLSLTCTVSGFSLSS |
| | 4G4S- | | | YAMSWIRQHPGKGLEWIGYIWSGGSTDY |
| | K-tag | | | ASWAKSRVTISKTSTTVSLKLSSVTAADTA |
| | | | | VYYCARRYGTSYPDYGDASGFDPWGQGT |
| | | | | LVTVSSASTKGPSVFPLAPSSKSTSGGTAA |
| | | | | LGCLVKDYFPEPVTVSWNSGALTSGVHTF |
| | | | | PAVLQSSGLYSLSSVVTVPSSSLGTQTYIC |
| | | | | NVNHKPSNTKVDKKVEPKSCGGGGSGGG |
| | | | | GSGGGGSGGGGSGGGRYESKGGG |
| 81 | (low | | | LC (kappa): |
| | affinity) | | | AIQLTQSPSSLSASVGDRVTITCRASQSISS |
| | | | | YLAWYQQKPGKAPKLLIYRASTLASGVPS |
| | | | | RFSGSGSGTDFTLTISSLQPEDFATYYCQQ |
| | | | | NYASSNVDNTFGGGTKVEIKRTVAAPSVFI |
| | | | | FPPSDEQLKSGTASVVCLLNNFYPREAKV |
| | | | | QWKVDNALQSGNSQESVTEQDSKDSTYS |
| | | | | LSSTLTLSKADYEKHKVYACEVTHQGLSS |
| | | | | PVTKSFNRGEC |
| 23 | Fc- | | Fc | Fc knob: |
| | CH2(P | | (knob- | DKTHTCPPCPAPEAAGGPSVFLFPPKPKDT |
| | GLAL | | into- | LMISRTPEVTCVVVDVSHEDPEVKFNWYV |
| | A)-CH3 | | hole) | DGVEVHNAKTKPREEQYNSTYRVVSVLT |
| | (K/H)- | | | VLHQDWLNGKEYKCKVSNKALGAPIEKTI |
| | 2G4S- | | | SKAKGQPREPQVYTLPPCRDELTKNQVSL |
| | K-tag | | | WCLVKGFYPSDIAVEWESNGQPENNYKTT |
| | | | | PPVLDSDGSFFLYSKLTVDKSRWQQGNVF |
| | | | | SCSVMHEALHNHYTQKSLSLSPGGGGGSG |
| | | | | GGGSGGGGRYESKGGG |
| 82 | | | | Fc hole: |
| | | | | DKTHTCPPCPAPEAAGGPSVFLFPPKPKDT |
| | | | | LMISRTPEVTCVVVDVSHEDPEVKFNWYV |
| | | | | DGVEVHNAKTKPREEQYNSTYRVVSVLT |
| | | | | VLHQDWLNGKEYKCKVSNKALGAPIEKTI |
| | | | | SKAKGQPREPQVCTLPPSRDELTKNQVSLS |
| | | | | CAVKGFYPSDIAVEWESNGQPENNYKTTP |
| | | | | PVLDSDGSFFLVSKLTVDKSRWQQGNVFS |
| | | | | CSVMHEALHNRFTQKSLSLSPG |
| 24 | Fc- | | Fc | Fc knob: |
| | CH2(P | | (knob- | DKTHTCPPCPAPEAAGGPSVFLFPPKPKDT |
| | GLAL | | | LMISRTPEVTCVVVDVSHEDPEVKFNWYV |
| | A)-CH3 | | | DGVEVHNAKTKPREEQYNSTYRVVSVLT |
| | | | | VLHQDWLNGKEYKCKVSNKALGAPIEKTI |
| | | | | SKAKGQPREPQVYTLPPCRDELTKNQVSL |
| | | | | WCLVKGFYPSDIAVEWESNGQPENNYKTT |
| | | | | PPVLDSDGSFFLYSKLTVDKSRWQQGNVF |
| | | | | SCSVMHEALHNHYTQKSLSLSPGGGGGSG |
| | | | | GGGSGGGGSGGGGSGGGRYESKGGG |
| 83 | (K/H)- | | into- | Fc hole: |
| | 4G4S- | | hole) | DKTHTCPPCPAPEAAGGPSVFLFPPKPKDT |
| | K-tag | | | LMISRTPEVTCVVVDVSHEDPEVKFNWYV |
| | | | | DGVEVHNAKTKPREEQYNSTYRVVSVLT |
| | | | | VLHQDWLNGKEYKCKVSNKALGAPIEKTI |
| | | | | SKAKGQPREPQVCTLPPSRDELTKNQVSLS |
| | | | | CAVKGFYPSDIAVEWESNGQPENNYKTTP |
| | | | | PVLDSDGSFFLVSKLTVDKSRWQQGNVFS |
| | | | | CSVMHEALHNRFTQKSLSLSPG |
| 25 | aHER2- | HER2 | Fab | HC: |
| | Fab- | | | EVQLVESGGGLVQPGGSLRLSCAASGFNI |
| | | | | KDTYIHWVRQAPGKGLEWVARIYPTNGY |
| | | | | TRYADSVKGRFTISADTSKNTAYLQMNSL |
| | | | | RAEDTAVYYCSRWGGDGFYAMDYWGQG |
| | | | | TLVTVSSASTKGPSVFPLAPSSKSTSGGTA |
| | | | | ALGCLVKDYFPEPVTVSWNSGALTSGVHT |
| | | | | FPAVLQSSGLYSLSSVVTVPSSSLGTQTYIC |
| | | | | NVNHKPSNTKVDKKVEPKSCGGGGSGGG |
| | | | | GSGGGGRYESKGGG |
| 84 | 2G4S- | | | LC (kappa): |
| | K-tag | | | DIQMTQSPSSLSASVGDRVTITCRASQDVN |
| | | | | TAVAWYQQKPGKAPKLLIYSASFLYSGVP |
| | | | | SRESGSRSGTDFTLTISSLQPEDFATYYCQQ |
| | | | | HYTTPPTFGQGTKVEIKRTVAAPSVFIFPPS |
| | | | | DEQLKSGTASVVCLLNNFYPREAKVQWK |
| | | | | VDNALQSGNSQESVTEQDSKDSTYSLSSTL |
| | | | | TLSKADYEKHKVYACEVTHQGLSSPVTKS |
| | | | | FNRGEC |
| 26 | aHER2- | HER2 | Fab | HC: |
| | Fab- | | | EVQLVESGGGLVQPGGSLRLSCAASGFNI |
| | 4G4S- | | | KDTYIHWVRQAPGKGLEWVARIYPTNGY |
| | | | | TRYADSVKGRFTISADTSKNTAYLQMNSL |
| | | | | RAEDTAVYYCSRWGGDGFYAMDYWGQG |
| | | | | TLVTVSSASTKGPSVFPLAPSSKSTSGGTA |
| | | | | ALGCLVKDYFPEPVTVSWNSGALTSGVHT |
| | | | | FPAVLQSSGLYSLSSVVTVPSSSLGTQTYIC |
| | | | | NVNHKPSNTKVDKKVEPKSCGGGGSGGG |
| | | | | GSGGGGSGGGGSGGGRYESKGGG |
| 85 | K-tag | | | LC (kappa): |
| | | | | DIQMTQSPSSLSASVGDRVTITCRASQDVN |
| | | | | TAVAWYQQKPGKAPKLLIYSASFLYSGVP |
| | | | | SRFSGSRSGTDFTLTISSLQPEDFATYYCQQ |
| | | | | HYTTPPTFGQGTKVEIKRTVAAPSVFIFPPS |
| | | | | DEQLKSGTASVVCLLNNFYPREAKVQWK |
| | | | | VDNALQSGNSQESVTEQDSKDSTYSLSSTL |
| | | | | TLSKADYEKHKVYACEVTHQGLSSPVTKS |
| | | | | FNRGEC |
| 27 | aHER2- | HER2 | scFv- | DIQMTQSPSSLSASVGDRVTITCRASQDVN |
| | scFv- | | Fc | TAVAWYQQKPGKAPKLLIYSASFLYSGVP |
| | 2G4S- | | | SRFSGSRSGTDFTLTISSLOPEDFATYYCQQ |
| | K-tag- | | | HYTTPPTFGCGTKVEIKGGSGGGGSGGGG |
| | 3C-Fc | | | SGGGGSGGEVQLVESGGGLVQPGGSLRLS |
| | | | | CAASGFNIKDTYIHWVRQAPGKCLEWVA |
| | | | | RIYPTNGYTRYADSVKGRFTISADTSKNTA |
| | | | | YLQMNSLRAEDTAVYYCSRWGGDGFYA |
| | | | | MDYWGQGTLVTVSSGGGGSGGGGSGGG |
| | | | | GRYESKGGGLEVLFQGPGGGGSDKTHTCP |
| | | | | PCPAPEAAGGPSVFLFPPKPKDTLMISRTPE |
| | | | | VTCVVVDVSHEDPEVKFNWYVDGVEVHN |
| | | | | AKTKPREEQYNSTYRVVSVLTVLHQDWL |
| | | | | NGKEYKCKVSNKALGAPIEKTISKAKGQP |
| | | | | REPQVYTLPPSRDELTKNQVSLTCLVKGF |
| | | | | YPSDIAVEWESNGQPENNYKTTPPVLDSD |
| | | | | GSFFLYSKLTVDKSRWQQGNVFSCSVMHE |
| | | | | ALHNHYTQKSLSLSPGK |
| 28 | aHER2- | HER2 | scFv- | DIQMTQSPSSLSASVGDRVTITCRASQDVN |
| | scFv- | | Fc | TAVAWYQQKPGKAPKLLIYSASFLYSGVP |
| | 4G4S- | | | SRFSGSRSGTDFTLTISSLQPEDFATYYCQQ |
| | K-tag- | | | HYTTPPTFGCGTKVEIKGGSGGGGSGGGG |
| | 3C-Fc | | | SGGGGSGGEVQLVESGGGLVQPGGSLRLS |
| | | | | CAASGFNIKDTYIHWVRQAPGKCLEWVA |
| | | | | RIYPTNGYTRYADSVKGRFTISADTSKNTA |
| | | | | YLQMNSLRAEDTAVYYCSRWGGDGFYA |
| | | | | MDYWGQGTLVTVSSGGGGSGGGGSGGG |
| | | | | GSGGGGSGGGRYESKGGGLEVLFQGPGG |
| | | | | GGSDKTHTCPPCPAPEAAGGPSVFLFPPKP |
| | | | | KDTLMISRTPEVTCVVVDVSHEDPEVKFN |
| | | | | WYVDGVEVHNAKTKPREEQYNSTYRVVS |
| | | | | VLTVLHQDWLNGKEYKCKVSNKALGAPI |
| | | | | EKTISKAKGQPREPQVYTLPPSRDELTKNQ |
| | | | | VSLTCLVKGFYPSDIAVEWESNGQPENNY |
| | | | | KTTPPVLDSDGSFFLYSKLTVDKSRWQQG |
| | | | | NVFSCSVMHEALHNHYTQKSLSLSPGK |
| 73 | Q-tag | - | peptide | YRYRQ |
| 74 | Q-tag | | peptide | GGGGSYRYRQGGGGS |
| | with | | | |
| | linker | | | |
| 75 | K-tag | - | peptide | RYESK |
| 76 | GGG- | - | peptide | GGGRYESKGGG |
| | K-tag- | | | |
| | GGG | | | |
| 29 | 2G4S- | | peptide | GGGGSGGGGSGGGGRYESKGGG |
| | K-tag | | | |
| 30 | 4G4S- | - | peptide | GGGGSGGGGSGGGGSGGGGSGGGRYESK |
| | K-tag | | | GGG |
| | AAV- | | AAV-2 | VP1-Q-tag SEQ ID NO: 01 |
| | 2-Q-tag | | | VP2 SEQ ID NO: 02 |
| | (mutHS | | | VP3 SEQ ID NO: 03 |
| | PG) | | | All three Proteins with R585A and R587A |
| | AAV- | TfR | AAV-2 | AAV-2-Q-tag SEQ ID NO: 1, 2, 3 |
| | 2-Q- | | + | K-tag-VNAR8a SEQ ID NO: 11 |
| | tag- | | VNAR | |
| | VNAR | | 8a | |
| | 8a (N- | | nanobo | |
| | terminal | | dy | |
| | K-tag) | | | |
| | AAV- | TfR | AAV-2 | AAV-2-Q-tag SEQ ID NO: 1, 2, 3 |
| | 2-Q- | | + | K-tag-MH12 SEQ ID NO: 12 |
| | tag- | | MH12 | |
| | MH12 | | nanobo | |
| | (N- | | dy | |
| | terminal | | | |
| | K-tag) | | | |
| | AAV- | TfR | AAV-2 | AAV-2-Q-tag SEQ ID NO: 1, 2, 3 |
| | 2-Q- | | + | VNAR8a-K-tag SEQ ID NO: 13 |
| | tag- | | VNAR | |
| | Vnar8a | | 8a | |
| | (C- | | nanobo | |
| | terminal | | dy | |
| | K-tag) | | | |
| | AAV- | TfR | AAV-2 | AAV-2-Q-tag SEQ ID NO: 1, 2, 3 |
| | 2-Q- | | + | MH12-K-tag SEQ ID NO: 14 |
| | tag- | | MH12 | |
| | MH12 | | nanobo | |
| | (C- | | dy | |
| | terminal | | | |
| | K-tag) | | | |
| | AAV- | EGFR | AAV-2 | AAV-2-Q-tag SEQ ID NO: 1, 2, 3 |
| | 2-Q- | | + Fab | aEGFR_Fab-2G4S-K-tag SEQ ID NO: 15 + 48 |
| | tag- | | | |
| | aEGFR_ | | | |
| | Fab- | | | |
| | 2G4S- | | | |
| | K-tag | | | |
| | AAV- | EGFR | AAV-2 | AAV-2-Q-tag SEQ ID NO: 1, 2, 3 |
| | 2-Q- | | + Fab | aEGFR_Fab-4G4S-K-tag SEQ ID NO: 16 + 77 |
| | tag- | | | |
| | aEGFR_ | | | |
| | Fab- | | | |
| | 4G4S- | | | |
| | K-tag | | | |
| | AAV- | EGFR | AAV-2 | AAV-2-Q-tag SEQ ID NO: 1, 2, 3 |
| | 2-Q- | | + scFv | aEGFR_scFv-2G4S-K-tag SEQ ID NO: 17 |
| | tag- | | | |
| | aEGFR_ | | | |
| | scFv- | | | |
| | 2G4S- | | | |
| | K-tag | | | |
| | AAV- | EGFR | AAV-2 | AAV-2-Q-tag SEQ ID NO: 1, 2, 3 |
| | 2-Q- | | + scFv | aEGFR_scFv-4G4S-K-tag SEQ ID NO: 18 |
| | tag- | | | |
| | aEGFR_ | | | |
| | scFv- | | | |
| | 4G4S- | | | |
| | K-tag | | | |
| | AAV- | TfR | AAV-2 | AAV-2-Q-tag SEQ ID NO: 1, 2, 3 |
| | 2-Q- | | + Fab | aTfR_Fab-2G4S-K-tag SEQ ID NO: 19 + 78 |
| | tag- | | | |
| | aTfR_F | | | |
| | ab- | | | |
| | 2G4S- | | | |
| | K-tag | | | |
| | (high | | | |
| | affinity) | | | |
| | AAV- | TfR | AAV-2 | AAV-2-Q-tag SEQ ID NO: 1, 2, 3 |
| | 2-Q- | | + Fab | aTfR_Fab-2G4S-K-tag SEQ ID NO: 20 + 79 |
| | tag- | | | |
| | aTfR_F | | | |
| | ab- | | | |
| | 2G4S- | | | |
| | K-tag | | | |
| | (low | | | |
| | affinity) | | | |
| | AAV- | TfR | AAV-2 | AAV-2-Q-tag SEQ ID NO: 1, 2, 3 |
| | 2-Q- | | + Fab | aTfR_Fab-4G4S-K-tag SEQ ID NO: 21 + 80 |
| | tag- | | | |
| | aTfR_F | | | |
| | ab- | | | |
| | 4G4S- | | | |
| | K-tag | | | |
| | (high | | | |
| | affinity) | | | |
| | AAV- | TfR | AAV-2 | AAV-2-Q-tag SEQ ID NO: 1, 2, 3 |
| | 2-Q- | | + Fab | aTfR_Fab-4G4S-K-tag SEQ ID NO: 22 + 81 |
| | tag- | | | |
| | aTfR_F | | | |
| | ab- | | | |
| | 4G4S- | | | |
| | K-tag | | | |
| | (low | | | |
| | affinity) | | | |
| 44 | 3C | | peptide | LEVLFQGP |
| | cleavage | | | |
| | site | | | |
| 45 | human | | | RAPRCRELPAQKWWHTGALYRIGDLQAF |
| | CD98 | | | QGHGAGNLAGLKGRLDYLSSLKVKGLVL |
| | ECD- | | | GPIHKNQKDDVAQTDLLQIDPNFGSKEDF |
| | Fc | | | DSLLQSAKKKSIRVILDLTPNYRGENSWFS |
| | Fusion | | | TQVDTVATKVKDALEFWLQAGVDGFQVR |
| | | | | DIENLKDASSFLAEWQNITKGFSEDRLLIA |
| | | | | GTNSSDLQQILSLLESNKDLLLTSSYLSDSG |
| | | | | STGEHTKSLVTQYLNATGNRWCSWSLSQ |
| | | | | ARLLTSFLPAQLLRLYQLMLFTLPGTPVFS |
| | | | | YGDEIGLDAAALPGQPMEAPVMLWDESSF |
| | | | | PDIPGAVSANMTVKGQSEDPGSLLSLFRRL |
| | | | | SDQRSKERSLLHGDFHAFSAGPGLFSYIRH |
| | | | | WDQNERFLVVLNFGDVGLSAGLQASDLP |
| | | | | ASASLPAKADLLLSTQPGREEGSPLELERL |
| | | | | KLEPHEGLLLRFPYAAGGSGGGGSGGDKT |
| | | | | HTCPPCPAPEAAGGPSVFLFPPKPKDTLMI |
| | | | | SRTPEVTCVVVDVSHEDPEVKFNWYVDG |
| | | | | VEVHNAKTKPREEQYNSTYRVVSVLTVLH |
| | | | | QDWLNGKEYKCKVSNKALGAPIEKTISKA |
| | | | | KGQPREPQVYTLPPCRDELTKNQVSLWCL |
| | | | | VKGFYPSDIAVEWESNGQPENNYKTTPPV |
| | | | | LDSDGSFFLYSKLTVDKSRWQQGNVFSCS |
| | | | | VMHEALHNHYTQKSLSLSPGDKTHTCPPC |
| | | | | PAPEAAGGPSVFLFPPKPKDTLMASRTPEV |
| | | | | TCVVVDVSHEDPEVKFNWYVDGVEVHNA |
| | | | | KTKPREEQYNSTYRVVSVLTVLAQDWLN |
| | | | | GKEYKCKVSNKALGAPIEKTISKAKGQPR |
| | | | | EPQVCTLPPSRDELTKNQVSLSCAVKGFYP |
| | | | | SDIAVEWESNGQPENNYKTTPPVLDSDGS |
| | | | | FFLVSKLTVDKSRWQQGNVFSCSVMHEA |
| | | | | LHNAYTQKSLSLSPG |

| variant particle | capsid | transgene |
|---|---|---|
| AAV-2^{RA}-aTfR-Nb-1 | 46, 47 | 4 |
| AAV-2^{RA/YF}-aTfR-Nb-1 | 46, 49 | 4 |
| AAV-2^{RA}-aTfR-Nb-1-(G4S)2 | 50, 47 | 4 |
| AAV-2^{RA/YF}-aTfR-Nb-1-(G4S)2 | 50, 49 | 4 |
| AAV-2^{RA}-aTfR-Nb-2 | 47, 51 | 4 |
| AAV-2^{RA/YF}-aTfR-Nb-2 | 49, 51 | 4 |
| chAAV-9/2^{RA}-aTfR-Nb-1 | 46, 52 | 4 |
| AAV-9-aTfR-Nb-1 | 52, 53 | 4 |
| AAV-9-aTfR-Nb-1_v2 | 53, 54 | 4 |
| AAV-5-aTfR-Nb-1 | 55, 56 | 4 |
| AAV-2^{RA}-aTfR-Pept-1 | 47, 57 | 4 |
| AAV-2^{RA}-aTfR-Pept-2 | 47, 58 | 4 |
| chAAV-9/2^{RA}-aTfR-Pept-1 | 52, 57 | 4 |
| chAAV-9/2^{RA}-aTfR-Pept-2 | 52, 58 | 4 |
| AAV-2^{RA}-aCD98-Nb-1 | 47, 59 | 4 |
| AAV-2^{RA}-aCD98-Nb-2 | 47, 60 | 4 |
| AAV-2^{RA}-aTfR-Nb-1_T456 | 47, 220 | 4 |
| AAV-2^{RA}-aTfR-Nb-1_del453-459 | 47, 221 | 4 |
| chAAV-9/2^{RA}-aTfR-Nb-1_T456 | 52, 220 | 4 |
| chAAV-9/2^{RA}-aTfR-Nb-1_del453-459 | 52, 221 | 4 |

| SEQ ID | | |
|---|---|---|
| NO: | rAAVp | sequence |
| 46 | AAV-2-VP1^{RA}_ | MAADGYLPDWLEDTLSEGIRQWWKLKPGPPPP |
| | aTfR-Nb-1 | KPAERHKDDSRGLVLPGYKYLGPFNGLDKGEPV |
| | | NEADAAALEHDKAYDRQLDSGDNPYLKYNHAD |
| | | AEFQERLKEDTSFGGNLGRAVFQAKKRVLEPLG |
| | | LVEEPVKTAPGKKRPVEHSPVEPDSSSGTGKAG |
| | | QQPARKRLNFGQTGDADSVPDPQPLGQPPAAPS |
| | | GLGTNTMATGSGAPMADNNEGADGVGNSSGN |
| | | WHCDSTWMGDRVITTSTRTWALPTYNNHLYKQ |
| | | ISSQSGASNDNHYFGYSTPWGYFDFNRFHCHFSP |
| | | RDWQRLINNNWGFRPKRLNFKLFNIQVKEVTQN |
| | | DGTTTIANNLTSTVQVFTDSEYQLPYVLGSAHQ |
| | | GCLPPFPADVFMVPQYGYLTLNNGSQAVGRSSF |
| | | YCLEYFPSQMLRTGNNFTFSYTFEDVPFHSSYAH |
| | | SQSLDRLMNPLIDQYLYYLSRTNTPGGGGSGGG |
| | | GSGGGGSGGGGSGGGGSQVQLVESGGGLVQAG |
| | | GSLRLSCAASGSSISSIAMGWYRQAPGKERELVA |
| | | AISSGGTINYADSVKGRFTISRDNAKNTVYLQMN |
| | | SLKPEDTAVYYCNTGRRLQTGSWGQGTQVTVSS |
| | | GGGGATQSRLQFSQAGASDIRDQSRNWLPGPCY |
| | | RQQRVSKTSADNNNSEYSWTGATKYHLNGRDS |
| | | LVNPGPAMASHKDDEEKFFPQSGVLIFGKQGSE |
| | | KTNVDIEKVMITDEEEIRTTNPVATEQYGSVSTN |
| | | LQAGNAQAATADVNTQGVLPGMVWQDRDVYL |
| | | QGPIWAKIPHTDGHFHPSPLMGGFGLKHPPPQILI |
| | | KNTPVPANPSTTFSAAKFASFITQYSTGQVSVEIE |
| | | WELQKENSKRWNPEIQYTSNYNKSVNVDFTVD |
| | | TNGVYSEPRPIGTRYLTRNL |
| 47 | AAV-2^{RA}_ | TAPGKKRPVEHSPVEPDSSSGTGKAGQQPARKR |
| | VP2/3 | LNFGQTGDADSVPDPQPLGQPPAAPSGLGTNTM |
| | | ATGSGAPMADNNEGADGVGNSSGNWHCDSTW |
| | | MGDRVITTSTRTWALPTYNNHLYKQISSQSGAS |
| | | NDNHYFGYSTPWGYFDFNRFHCHFSPRDWQRLI |
| | | NNNWGFRPKRLNFKLFNIQVKEVTQNDGTTTIA |
| | | NNLTSTVQVFTDSEYQLPYVLGSAHQGCLPPFPA |
| | | DVFMVPQYGYLTLNNGSQAVGRSSFYCLEYFPS |
| | | QMLRTGNNFTFSYTFEDVPFHSSYAHSQSLDRL |
| | | MNPLIDQYLYYLSRTNTPSGTTTQSRLQFSQAGA |
| | | SDIRDQSRNWLPGPCYRQQRVSKTSADNNNSEY |
| | | SWTGATKYHLNGRDSLVNPGPAMASHKDDEEK |
| | | FFPQSGVLIFGKQGSEKTNVDIEKVMITDEEEIRT |
| | | TNPVATEQYGSVSTNLQAGNAQAATADVNTQG |
| | | VLPGMVWQDRDVYLQGPIWAKIPHTDGHFHPSP |
| | | LMGGFGLKHPPPQILIKNTPVPANPSTTFSAAKFA |
| | | SFITQYSTGQVSVEIEWELQKENSKRWNPEIQYT |
| | | SNYNKSVNVDFTVDTNGVYSEPRPIGTRYLTRN |
| | | L |
| 49 | AAV-2^{RA-YF}- | TAPGKKRPVEHSPVEPDSSSGTGKAGQQPARKR |
| | VP2/3 | LNFGQTGDADSVPDPQPLGQPPAAPSGLGTNTM |
| | | ATGSGAPMADNNEGADGVGNSSGNWHCDSTW |
| | | MGDRVITTSTRTWALPTYNNHLYKQISSQSGAS |
| | | NDNHYFGYSTPWGYFDFNRFHCHFSPRDWQRLI |
| | | NNNWGFRPKRLNFKLFNIQVKEVTQNDGTTTIA |
| | | NNLTSTVQVFTDSEYQLPYVLGSAHQGCLPPFPA |
| | | DVFMVPQYGYLTLNNGSQAVGRSSFYCLEYFPS |
| | | QMLRTGNNFTFSYTFEDVPFHSSYAHSQSLDRL |
| | | MNPLIDQYLYFLSRTNTPSGTTTQSRLQFSQAGA |
| | | SDIRDQSRNWLPGPCYRQQRVSKTSADNNNSEF |
| | | SWTGATKYHLNGRDSLVNPGPAMASHKDDEEK |
| | | FFPQSGVLIFGKQGSEKTNVDIEKVMITDEEEIRT |
| | | TNPVATEQYGSVSTNLQAGNAQAATADVNTQG |
| | | VLPGMVWQDRDVYLQGPIWAKIPHTDGHFHPSP |
| | | LMGGFGLKHPPPQILIKNTPVPANPSTTFSAAKFA |
| | | SFITQYSTGQVSVEIEWELQKENSKRWNPEIQYT |
| | | SNYNKSVNVDFTVDTNGVYSEPRPIGTRFLTRNL |
| 50 | AAV-2-VP1^{RA}_ | MAADGYLPDWLEDTLSEGIRQWWKLKPGPPPP |
| | aTfR-Nb-1- | KPAERHKDDSRGLVLPGYKYLGPFNGLDKGEPV |
| | (G4S)2 | NEADAAALEHDKAYDRQLDSGDNPYLKYNHAD |
| | | AEFQERLKEDTSFGGNLGRAVFQAKKRVLEPLG |
| | | LVEEPVKTAPGKKRPVEHSPVEPDSSSGTGKAG |
| | | QQPARKRLNFGQTGDADSVPDPQPLGQPPAAPS |
| | | GLGTNTMATGSGAPMADNNEGADGVGNSSGN |
| | | WHCDSTWMGDRVITTSTRTWALPTYNNHLYKQ |
| | | ISSQSGASNDNHYFGYSTPWGYFDFNRFHCHFSP |
| | | RDWQRLINNNWGFRPKRLNFKLFNIQVKEVTQN |
| | | DGTTTIANNLTSTVQVFTDSEYQLPYVLGSAHQ |
| | | GCLPPFPADVFMVPQYGYLTLNNGSQAVGRSSF |
| | | YCLEYFPSQMLRTGNNFTFSYTFEDVPFHSSYAH |
| | | SQSLDRLMNPLIDQYLYYLSRTNTPGGGGSGGG |
| | | GSQVQLVESGGGLVQAGGSLRLSCAASGSSISSI |
| | | AMGWYRQAPGKERELVAAISSGGTINYADSVK |
| | | GRFTISRDNAKNTVYLQMNSLKPEDTAVYYCNT |
| | | GRRLQTGSWGQGTQVTVSSGGGGSGGGGSTQS |
| | | RLQFSQAGASDIRDQSRNWLPGPCYRQQRVSKT |
| | | SADNNNSEYSWTGATKYHLNGRDSLVNPGPAM |
| | | ASHKDDEEKFFPQSGVLIFGKQGSEKTNVDIEKV |
| | | MITDEEEIRTTNPVATEQYGSVSTNLQAGNAQA |
| | | ATADVNTQGVLPGMVWQDRDVYLQGPIWAKIP |
| | | HTDGHFHPSPLMGGFGLKHPPPQILIKNTPVPAN |
| | | PSTTFSAAKFASFITQYSTGQVSVEIEWELQKENS |
| | | KRWNPEIQYTSNYNKSVNVDFTVDTNGVYSEPR |
| | | PIGTRYLTRNL |
| 51 | AAV-2-VP1^{RA}_ | MAADGYLPDWLEDTLSEGIRQWWKLKPGPPPP |
| | aTfR-Nb-2 | KPAERHKDDSRGLVLPGYKYLGPFNGLDKGEPV |
| | | NEADAAALEHDKAYDRQLDSGDNPYLKYNHAD |
| | | AEFQERLKEDTSFGGNLGRAVFQAKKRVLEPLG |
| | | LVEEPVKTAPGKKRPVEHSPVEPDSSSGTGKAG |
| | | QQPARKRLNFGQTGDADSVPDPQPLGQPPAAPS |
| | | GLGTNTMATGSGAPMADNNEGADGVGNSSGN |
| | | WHCDSTWMGDRVITTSTRTWALPTYNNHLYKQ |
| | | ISSQSGASNDNHYFGYSTPWGYFDFNRFHCHFSP |
| | | RDWQRLINNNWGFRPKRLNFKLFNIQVKEVTQN |
| | | DGTTTIANNLTSTVQVFTDSEYQLPYVLGSAHQ |
| | | GCLPPFPADVFMVPQYGYLTLNNGSQAVGRSSF |
| | | YCLEYFPSQMLRTGNNFTFSYTFEDVPFHSSYAH |
| | | SQSLDRLMNPLIDQYLYYLSRTNTPGGGGSGGG |
| | | GSGGGGSGGGGSGGGGSQVQLVESGGGLVQTG |
| | | GSLRLSCAASGLTFSNYAMGWFRQAPGKKREFV |
| | | AHIGGSGGTWRYADSVKGRFTISRDNAKNMVY |
| | | LQMNSLNTEDTAIYYCAADQRAGSYSSGWYTRS |
| | | SDSLYWGQGTQVTVSSGGGGATQSRLQFSQAG |
| | | ASDIRDQSRNWLPGPCYRQQRVSKTSADNNNSE |
| | | YSWTGATKYHLNGRDSLVNPGPAMASHKDDEE |
| | | KFFPQSGVLIFGKQGSEKTNVDIEKVMITDEEEIR |
| | | TTNPVATEQYGSVSTNLQAGNAQAATADVNTQ |
| | | GVLPGMVWQDRDVYLQGPIWAKIPHTDGHFHP |
| | | SPLMGGFGLKHPPPQILIKNTPVPANPSTTFSAAK |
| | | FASFITQYSTGQVSVEIEWELQKENSKRWNPEIQ |
| | | YTSNYNKSVNVDFTVDTNGVYSEPRPIGTRYLT |
| | | RNL |
| 52 | AAV-9-VP2/3 | TAPGKKRPVEQSPQEPDSSAGIGKSGAQPAKKRL |
| | | NFGQTGDTESVPDPQPIGEPPAAPSGVGSLTMAS |
| | | GGGAPVADNNEGADGVGSSSGNWHCDSQWLG |
| | | DRVITTSTRTWALPTYNNHLYKQISNSTSGGSSN |
| | | DNAYFGYSTPWGYFDFNRFHCHFSPRDWQRLIN |
| | | NNWGFRPKRLNFKLFNIQVKEVTDNNGVKTIAN |
| | | NLTSTVQVFTDSDYQLPYVLGSAHEGCLPPFPAD |
| | | VFMIPQYGYLTLNDGSQAVGRSSFYCLEYFPSQ |
| | | MLRTGNNFQFSYEFENVPFHSSYAHSQSLDRLM |
| | | NPLIDQYLYYLSKTINGSGQNQQTLKFSVAGPSN |
| | | MAVQGRNYIPGPSYRQQRVSTTVTQNNNSEFA |
| | | WPGASSWALNGRNSLMNPGPAMASHKEGEDRF |
| | | FPLSGSLIFGKQGTGRDNVDADKVMITNEEEIKT |
| | | TNPVATESYGQVATNHQSAQAQAQTGWVQNQ |
| | | GILPGMVWQDRDVYLQGPIWAKIPHTDGNFHPS |
| | | PLMGGFGMKHPPPQILIKNTPVPADPPTAFNKDK |
| | | LNSFITQYSTGQVSVEIEWELQKENSKRWNPEIQ |
| | | YTSNYYKSNNVEFAVNTEGVYSEPRPIGTRYLTR |
| | | NL |
| 53 | AAV-9-VP1- | MAADGYLPDWLEDNLSEGIREWWALKPGAPQP |
| | aTfR-Nb-1 | KANQQHQDNARGLVLPGYKYLGPGNGLDKGEP |
| | | VNAADAAALEHDKAYDQQLKAGDNPYLKYNH |
| | | ADAEFQERLKEDTSFGGNLGRAVFQAKKRLLEP |
| | | LGLVEEAAKTAPGKKRPVEQSPQEPDSSAGIGKS |
| | | GAQPAKKRLNFGQTGDTESVPDPQPIGEPPAAPS |
| | | GVGSLTMASGGGAPVADNNEGADGVGSSSGNW |
| | | HCDSQWLGDRVITTSTRTWALPTYNNHLYKQIS |
| | | NSTSGGSSNDNAYFGYSTPWGYFDFNRFHCHFS |
| | | PRDWQRLINNNWGFRPKRLNFKLFNIQVKEVTD |
| | | NNGVKTIANNLTSTVQVFTDSDYQLPYVLGSAH |
| | | EGCLPPFPADVFMIPQYGYLTLNDGSQAVGRSSF |
| | | YCLEYFPSQMLRTGNNFQFSYEFENVPFHSSYAH |
| | | SQSLDRLMNPLIDQYLYYLSKTINGGGGGSGGG |
| | | GSGGGGSGGGGSGGGGSQVQLVESGGGLVQAG |
| | | GSLRLSCAASGSSISSIAMGWYRQAPGKERELVA |
| | | AISSGGTINYADSVKGRFTISRDNAKNTVYLQMN |
| | | SLKPEDTAVYYCNTGRRLQTGSWGQGTQVTVSS |
| | | GGGGAQTLKFSVAGPSNMAVQGRNYIPGPSYRQ |
| | | QRVSTTVTQNNNSEFAWPGASSWALNGRNSLM |
| | | NPGPAMASHKEGEDRFFPLSGSLIFGKQGTGRDN |
| | | VDADKVMITNEEEIKTTNPVATESYGQVATNHQ |
| | | SAQAQAQTGWVQNQGILPGMVWQDRDVYLQG |
| | | PIWAKIPHTDGNFHPSPLMGGFGMKHPPPQILIK |
| | | NTPVPADPPTAFNKDKLNSFITQYSTGQVSVEIE |
| | | WELQKENSKRWNPEIQYTSNYYKSNNVEFAVN |
| | | TEGVYSEPRPIGTRYLTRNL |
| 54 | AAV-9- | TAPGKKRPVEQSPQEPDSSAGIGKSGAQPAKKRL |
| | VP2/3_v2 | NFGQTGDTESVPDPQPIGEPPAAPSGVGSLTMAS |
| | | GGGAPVADNNEGADGVGSSSGNWHCDSQWLG |
| | | DRVITTSTRTWALPTYNNHLYKQISNSTSGGSSN |
| | | DNAYFGYSTPWGYFDFNRFHCHFSPRDWQRLIN |
| | | NNWGFRPKRLNFKLFNIQVKEVTDNNGVKTIAN |
| | | NLTSTVQVFTDSDYQLPYVLGSAHEGCLPPFPAD |
| | | VFMIPQYGYLTLNDGSQAVGRSSFYCLEYFPSQ |
| | | MLRTGNNFQFSYEFENVPFHSSYAHSQSLDRLM |
| | | NPLIDQYLYYLSKTINGSGQNQQTLKFSVAGPSN |
| | | MAVQGRNYIPGPSYRQQRVSTTVTQNNNSEFA |
| | | WPGASSWALNGRNSLMNPGPAMASHKEGEDRF |
| | | FPLSGSLIFGKQGTGRDNVDADKVMITNEEEIKT |
| | | TNPVATESYGQVATNHQSAQAQAQTGWVQNQ |
| | | GILPGMVWQDRDVYLQGPIWAKIPHTDGNFHPS |
| | | PLMGGFGMKHPPPQILIKNTPVPADPPTAFNKDK |
| | | LNSFITQYSTGQVSVEIEWELQKENSKRWNPEIQ |
| | | YTSNYYKSNNVEFAVNTEGVYSEPRPIGTRYLTR |
| | | NL |
| 55 | AAV-5-VP1- | MSFVDHPPDWLEEVGEGLREFLGLEAGPPKPKP |
| | aTfR-Nb-1 | NQQHQDQARGLVLPGYNYLGPGNGLDRGEPVN |
| | | RADEVAREHDISYNEQLEAGDNPYLKYNHADA |
| | | EFQEKLADDTSFGGNLGKAVFQAKKRVLEPFGL |
| | | VEEGAKTAPTGKRIDDHFPKRKKARTEEDSKPST |
| | | SSDAEAGPSGSQQLQIPAQPASSLGADTMSAGG |
| | | GGPLGDNNQGADGVGNASGDWHCDSTWMGDR |
| | | VVTKSTRTWVLPSYNNHQYREIKSGSVDGSNAN |
| | | AYFGYSTPWGYFDFNRFHSHWSPRDWQRLINN |
| | | YWGFRPRSLRVKIFNIQVKEVTVQDSTTTIANNL |
| | | TSTVQVFTDDDYQLPYVVGNGTEGCLPAFPPQV |
| | | FTLPQYGYATLNRDNTENPTERSSFFCLEYFPSK |
| | | MLRTGNNFEFTYNFEEVPFHSSFAPSQNLFKLAN |
| | | PLVDQYLYRFVSTGGGGSGGGGSGGGGSGGGG |
| | | SGGGGSQVQLVESGGGLVQAGGSLRLSCAASGS |
| | | SISSIAMGWYRQAPGKERELVAAISSGGTINYAD |
| | | SVKGRFTISRDNAKNTVYLQMNSLKPEDTAVYY |
| | | CNTGRRLQTGSWGQGTQVTVSSGGGGAGGVQF |
| | | NKNLAGRYANTYKNWFPGPMGRTQGWNLGSG |
| | | VNRASVSAFATTNRMELEGASYQVPPQPNGMT |
| | | NNLQGSNTYALENTMIFNSQPANPGTTATYLEG |
| | | NMLITSESETQPVNRVAYNVGGQMATNNQSSTT |
| | | APATGTYNLQEIVPGSVWMERDVYLQGPIWAKI |
| | | PETGAHFHPSPAMGGFGLKHPPPMMLIKNTPVP |
| | | GNITSFSDVPVSSFITQYSTGQVTVEMEWELKKE |
| | | NSKRWNPEIQYTNNYNDPQFVDFAPDSTGEYRT |
| | | TRPIGTRYLTRPL |
| 56 | AAV-5-VP2/3 | KS*VDHPPDWLEEVGEGLREFLGLEAGPPKPKPN |
| | | QQHQDQARGLVLPGYNYLGPGNGLDRGEPVNR |
| | | ADEVAREHDISYNEQLEAGDNPYLKYNHADAEF |
| | | QEKLADDTSFGGNLGKAVFQAKKRVLEPFGLVE |
| | | EGAKTAPTGKRIDDHFPKRKKARTEEDSKPSTSS |
| | | DAEAGPSGSQQLQIPAQPASSLGADTMSAGGGG |
| | | PLGDNNQGADGVGNASGDWHCDSTWMGDRVV |
| | | TKSTRTWVLPSYNNHQYREIKSGSVDGSNANAY |
| | | FGYSTPWGYFDFNRFHSHWSPRDWQRLINNYW |
| | | GFRPRSLRVKIFNIQVKEVTVQDSTTTIANNLTST |
| | | VQVFTDDDYQLPYVVGNGTEGCLPAFPPQVFTL |
| | | PQYGYATLNRDNTENPTERSSFFCLEYFPSKMLR |
| | | TGNNFEFTYNFEEVPFHSSFAPSQNLFKLANPLV |
| | | DQYLYRFVSTNNTGGVQFNKNLAGRYANTYKN |
| | | WFPGPMGRTQGWNLGSGVNRASVSAFATTNRM |
| | | ELEGASYQVPPQPNGMTNNLQGSNTYALENTMI |
| | | FNSQPANPGTTATYLEGNMLITSESETQPVNRVA |
| | | YNVGGQMATNNQSSTTAPATGTYNLQEIVPGSV |
| | | WMERDVYLQGPIWAKIPETGAHFHPSPAMGGFG |
| | | LKHPPPMMLIKNTPVPGNITSFSDVPVSSFITQYS |
| | | TGQVTVEMEWELKKENSKRWNPEIQYTNNYND |
| | | PQFVDFAPDSTGEYRTTRPIGTRYLTRPL* |
| 57 | AAV-2-VP1^{RA}_ | MAADGYLPDWLEDTLSEGIRQWWKLKPGPPPP |
| | aTfR-Pept-1 | KPAERHKDDSRGLVLPGYKYLGPFNGLDKGEPV |
| | | NEADAAALEHDKAYDRQLDSGDNPYLKYNHAD |
| | | AEFQERLKEDTSFGGNLGRAVFQAKKRVLEPLG |
| | | LVEEPVKTAPGKKRPVEHSPVEPDSSSGTGKAG |
| | | QQPARKRLNFGQTGDADSVPDPQPLGQPPAAPS |
| | | GLGTNTMATGSGAPMADNNEGADGVGNSSGN |
| | | WHCDSTWMGDRVITTSTRTWALPTYNNHLYKQ |
| | | ISSQSGASNDNHYFGYSTPWGYFDFNRFHCHFSP |
| | | RDWQRLINNNWGFRPKRLNFKLFNIQVKEVTQN |
| | | DGTTTIANNLTSTVQVFTDSEYQLPYVLGSAHQ |
| | | GCLPPFPADVFMVPQYGYLTLNNGSQAVGRSSF |
| | | YCLEYFPSQMLRTGNNFTFSYTFEDVPFHSSYAH |
| | | SQSLDRLMNPLIDQYLYYLSRTNTPGGGGSGGG |
| | | GSGGGGSGGGGSGSREGCASRCTKYNAELEKCE |
| | | ARVMSMSNTEEDCEQELEDLLHCLDHCHSQGG |
| | | GGSGGGGSGGGGSGGGGSTQSRLQFSQAGASDI |
| | | RDQSRNWLPGPCYRQQRVSKTSADNNNSEYSW |
| | | TGATKYHLNGRDSLVNPGPAMASHKDDEEKFFP |
| | | QSGVLIFGKQGSEKTNVDIEKVMITDEEEIRTTNP |
| | | VATEQYGSVSTNLQAGNAQAATADVNTQGVLP |
| | | GMVWQDRDVYLQGPIWAKIPHTDGHFHPSPLM |
| | | GGFGLKHPPPQILIKNTPVPANPSTTFSAAKFASFI |
| | | TQYSTGQVSVEIEWELQKENSKRWNPEIQYTSN |
| | | YNKSVNVDFTVDTNGVYSEPRPIGTRYLTRNL |
| 58 | AAV-2-VP1^{RA}_ | MAADGYLPDWLEDTLSEGIRQWWKLKPGPPPP |
| | aTfR-Pept-2 | KPAERHKDDSRGLVLPGYKYLGPFNGLDKGEPV |
| | | NEADAAALEHDKAYDRQLDSGDNPYLKYNHAD |
| | | AEFQERLKEDTSFGGNLGRAVFQAKKRVLEPLG |
| | | LVEEPVKTAPGKKRPVEHSPVEPDSSSGTGKAG |
| | | QQPARKRLNFGQTGDADSVPDPQPLGQPPAAPS |
| | | GLGTNTMATGSGAPMADNNEGADGVGNSSGN |
| | | WHCDSTWMGDRVITTSTRTWALPTYNNHLYKQ |
| | | ISSQSGASNDNHYFGYSTPWGYFDFNRFHCHFSP |
| | | RDWQRLINNNWGFRPKRLNFKLFNIQVKEVTQN |
| | | DGTTTIANNLTSTVQVFTDSEYQLPYVLGSAHQ |
| | | GCLPPFPADVFMVPQYGYLTLNNGSQAVGRSSF |
| | | YCLEYFPSQMLRTGNNFTFSYTFEDVPFHSSYAH |
| | | SQSLDRLMNPLIDQYLYYLSRTNTPGGGGSGGG |
| | | GSGGGGSGGGGSGSREGCASRCMKYNDELEKC |
| | | EARMMSMSNTEEDCEQELEDLLYCLDHCHSQG |
| | | GGGSGGGGSGGGGSGGGGSTQSRLQFSQAGAS |
| | | DIRDQSRNWLPGPCYRQQRVSKTSADNNNSEYS |
| | | WTGATKYHLNGRDSLVNPGPAMASHKDDEEKF |
| | | FPQSGVLIFGKQGSEKTNVDIEKVMITDEEEIRTT |
| | | NPVATEQYGSVSTNLQAGNAQAATADVNTQGV |
| | | LPGMVWQDRDVYLQGPIWAKIPHTDGHFHPSPL |
| | | MGGFGLKHPPPQILIKNTPVPANPSTTFSAAKFAS |
| | | FITQYSTGQVSVEIEWELQKENSKRWNPEIQYTS |
| | | NYNKSVNVDFTVDTNGVYSEPRPIGTRYLTRNL |
| 59 | AAV-2-VP1^{RA}_ | MAADGYLPDWLEDTLSEGIRQWWKLKPGPPPP |
| | aCD98-Nb-1 | KPAERHKDDSRGLVLPGYKYLGPFNGLDKGEPV |
| | | NEADAAALEHDKAYDRQLDSGDNPYLKYNHAD |
| | | AEFQERLKEDTSFGGNLGRAVFQAKKRVLEPLG |
| | | LVEEPVKTAPGKKRPVEHSPVEPDSSSGTGKAG |
| | | QQPARKRLNFGQTGDADSVPDPQPLGQPPAAPS |
| | | GLGTNTMATGSGAPMADNNEGADGVGNSSGN |
| | | WHCDSTWMGDRVITTSTRTWALPTYNNHLYKQ |
| | | ISSQSGASNDNHYFGYSTPWGYFDFNRFHCHFSP |
| | | RDWQRLINNNWGFRPKRLNFKLFNIQVKEVTQN |
| | | DGTTTIANNLTSTVQVFTDSEYQLPYVLGSAHQ |
| | | GCLPPFPADVFMVPQYGYLTLNNGSQAVGRSSF |
| | | YCLEYFPSQMLRTGNNFTFSYTFEDVPFHSSYAH |
| | | SQSLDRLMNPLIDQYLYYLSRTNTPGGGGSGGG |
| | | GSGGGGSGGGGSGGGGSQVQLVESGGGLVQAG |
| | | GSLRLSCAASGRAFSSNGMGWFRQAPGKEREFV |
| | | ATISCSGTSTYYADSVKGRFTISRDNAKNTVYLQ |
| | | MNSLKPEDTAVYFCAADTRHCMGYTKDEGYAY |
| | | WGQGTQVTVSSGGGGATQSRLQFSQAGASDIRD |
| | | QSRNWLPGPCYRQQRVSKTSADNNNSEYSWTG |
| | | ATKYHLNGRDSLVNPGPAMASHKDDEEKFFPQS |
| | | GVLIFGKQGSEKTNVDIEKVMITDEEEIRTTNPV |
| | | ATEQYGSVSTNLQAGNAQAATADVNTQGVLPG |
| | | MVWQDRDVYLQGPIWAKIPHTDGHFHPSPLMG |
| | | GFGLKHPPPQILIKNTPVPANPSTTFSAAKFASFIT |
| | | QYSTGQVSVEIEWELQKENSKRWNPEIQYTSNY |
| | | NKSVNVDFTVDTNGVYSEPRPIGTRYLTRNL |
| 60 | AAV-2-VP1^{RA}_ | MAADGYLPDWLEDTLSEGIRQWWKLKPGPPPP |
| | aCD98-Nb-2 | KPAERHKDDSRGLVLPGYKYLGPFNGLDKGEPV |
| | | NEADAAALEHDKAYDRQLDSGDNPYLKYNHAD |
| | | AEFQERLKEDTSFGGNLGRAVFQAKKRVLEPLG |
| | | LVEEPVKTAPGKKRPVEHSPVEPDSSSGTGKAG |
| | | QQPARKRLNFGQTGDADSVPDPQPLGQPPAAPS |
| | | GLGTNTMATGSGAPMADNNEGADGVGNSSGN |
| | | WHCDSTWMGDRVITTSTRTWALPTYNNHLYKQ |
| | | ISSQSGASNDNHYFGYSTPWGYFDFNRFHCHFSP |
| | | RDWQRLINNNWGFRPKRLNFKLFNIQVKEVTQN |
| | | DGTTTIANNLTSTVQVFTDSEYQLPYVLGSAHQ |
| | | GCLPPFPADVFMVPQYGYLTLNNGSQAVGRSSF |
| | | YCLEYFPSQMLRTGNNFTFSYTFEDVPFHSSYAH |
| | | SQSLDRLMNPLIDQYLYYLSRTNTPGGGGSGGG |
| | | GSGGGGSGGGGSGGGGSQVHLVESGGGLVQAG |
| | | GSLRLSCAASGRTISTYGMGWFRQAPGKEREFV |
| | | GTISCSGTSTYYTDSVKGRFTISRDNAKNTVYLE |
| | | MNSLKPEDTAVYFCAADTRHCMGYTKHGGYD |
| | | YWGQGTQVTVSSGGGGATQSRLQFSQAGASDIR |
| | | DQSRNWLPGPCYRQQRVSKTSADNNNSEYSWT |
| | | GATKYHLNGRDSLVNPGPAMASHKDDEEKFFP |
| | | QSGVLIFGKQGSEKTNVDIEKVMITDEEEIRTTNP |
| | | VATEQYGSVSTNLQAGNAQAATADVNTQGVLP |
| | | GMVWQDRDVYLQGPIWAKIPHTDGHFHPSPLM |
| | | GGFGLKHPPPQILIKNTPVPANPSTTFSAAKFASFI |
| | | TQYSTGQVSVEIEWELQKENSKRWNPEIQYTSN |
| | | YNKSVNVDFTVDTNGVYSEPRPIGTRYLTRNL |
| 61 | peptidic linker | (GGGGS)5 |
| 62 | peptidic linker | (GGGGS)2 |
| 63 | peptidic linker | GGGGA |
| 64 | peptidic linker | (GGGGS)4 |
| 65 | AAV-2 VP1 | MAADGYLPDWLEDTLSEGIRQWWKLKPGPPPP |
| | wild-type | KPAERHKDDSRGLVLPGYKYLGPFNGLDKGEPV |
| | | NEADAAALEHDKAYDRQLDSGDNPYLKYNHAD |
| | | AEFQERLKEDTSFGGNLGRAVFQAKKRVLEPLG |
| | | LVEEPVKTAPGKKRPVEHSPVEPDSSSGTGKAG |
| | | QQPARKRLNFGQTGDADSVPDPQPLGQPPAAPS |
| | | GLGTNTMATGSGAPMADNNEGADGVGNSSGN |
| | | WHCDSTWMGDRVITTSTRTWALPTYNNHLYKQ |
| | | ISSQSGASNDNHYFGYSTPWGYFDFNRFHCHFSP |
| | | RDWQRLINNNWGFRPKRLNFKLFNIQVKEVTQN |
| | | DGTTTIANNLTSTVQVFTDSEYQLPYVLGSAHQ |
| | | GCLPPFPADVFMVPQYGYLTLNNGSQAVGRSSF |
| | | YCLEYFPSQMLRTGNNFTFSYTFEDVPFHSSYAH |
| | | SQSLDRLMNPLIDQYLYYLSRTNTPSGTTTQSRL |
| | | QFSQAGASDIRDQSRNWLPGPCYRQQRVSKTSA |
| | | DNNNSEYSWTGATKYHLNGRDSLVNPGPAMAS |
| | | HKDDEEKFFPQSGVLIFGKQGSEKTNVDIEKVMI |
| | | TDEEEIRTTNPVATEQYGSVSTNLQRGNRQAAT |
| | | ADVNTQGVLPGMVWQDRDVYLQGPIWAKIPHT |
| | | DGHFHPSPLMGGFGLKHPPPQILIKNTPVPANPST |
| | | TFSAAKFASFITQYSTGQVSVEIEWELQKENSKR |
| | | WNPEIQYTSNYNKSVNVDFTVDINGVYSEPRPI |
| | | GTRYLTRNL |
| 66 | microbial | MHKWFLRAAVVAAVGFGLPTLIATTAQAAAVA |
| | transglutaminase | APTPRAPLAPPLAEDRSYRTWRVEDYVEAWERY |
| | derived from | HGREMTEDERENLARGCIGVTVVNLNREDLSNP |
| | Kutzneria albida | PLNLSFGSLRTAEAVQAALNKIVDTHPSPAQYEA |
| | | AVAKDPILKRLKNVVKALPSWIDSAKLKASIFSK |
| | | RFYSWQNPDWSEERAHTTYRPDRETDQVDMST |
| | | YRYRARPGYVNFDYGWFDQDTNTWWHANHEE |
| | | PRMVVYQSTLRHYSRPLQDFDEQVFTVAFAKKD |
| 67 | aTfR-Nb-1 | QVQLVESGGGLVQAGGSLRLSCAASGSSISSIAM |
| | | GWYRQAPGKERELVAAISSGGTINYADSVKGRF |
| | | TISRDNAKNTVYLQMNSLKPEDTAVYYCNTGRR |
| | | LQTGSWGQGTQVTVSS |
| 68 | aTfR-Nb-2 | QVQLVESGGGLVQTGGSLRLSCAASGLTFSNYA |
| | | MGWFRQAPGKKREFVAHIGGSGGTWRYADSVK |
| | | GRFTISRDNAKNMVYLQMNSLNTEDTAIYYCAA |
| | | DQRAGSYSSGWYTRSSDSLYWGQGTQVTVSS |
| 69 | aTfR-Pept-1 | GSREGCASRCTKYNAELEKCEARVMSMSNTEED |
| | | CEQELEDLLHCLDHCHSQ |
| 70 | aTfR-Pept-2 | GSREGCASRCMKYNDELEKCEARMMSMSNTEE |
| | | DCEQELEDLLYCLDHCHSQ |
| 71 | aCD98-Nb-1 | QVQLVESGGGLVQAGGSLRLSCAASGRAFSSNG |
| | | MGWFRQAPGKEREFVATISCSGTSTYYADSVKG |
| | | RFTISRDNAKNTVYLQMNSLKPEDTAVYFCAAD |
| | | TRHCMGYTKDEGYAYWGQGTQVTVSS |
| 72 | aCD98-Nb-1 | QVHLVESGGGLVQAGGSLRLSCAASGRTISTYG |
| | | MGWFRQAPGKEREFVGTISCSGTSTYYTDSVKG |
| | | RFTISRDNAKNTVYLEMNSLKPEDTAVYFCAAD |
| | | TRHCMGYTKHGGYDYWGQGTQVTVSS |
| | AAV2-Qtag- | AAV2-Qtag SEQ ID NO: 1, 2, 3 |
| | aHER2-Fab- | aHER2-Fab-2G4S-Ktag SEQ ID NO: 25, 84 |
| | 2G4S-Ktag | |
| | AAV2-Qtag- | AAV2-Qtag SEQ ID NO: 1, 2, 3 |
| | aHER2-Fab- | aHER2-Fab-4G4S-Ktag SEQ ID NO: 26, 85 |
| | 4G4S-Ktag | |
| | AAV2-Qtag- | AAV2-Qtag SEQ ID NO: 1, 2, 3 |
| | aHER2-scFv- | aHER2-scFv-2G4S-Ktag SEQ ID NO: 27 |
| | 2G4S-Ktag | |
| | AAV2-Qtag- | AAV2-Qtag SEQ ID NO: 1, 2, 3 |
| | aHER2-scFv- | aHER2-scFv-4G4S-Ktag SEQ ID NO: 28 |
| | 4G4S-Ktag | |
| 94 | Recombinant | hERBB2_ECD: QVCTGTDMKLRLPASPETHLDML |
| | human, | RHLYQGCQVVQGNLELTYLPTNASLSFLQDIQE |
| | monovalent | VQGYVLIAHNQVRQVPLQRLRIVRGTQLFEDNY |
| | Her2_Fc | ALAVLDNGDPLNNTTPVTGASPGGLRELQLRSL |
| | | TEILKGGVLIQRNPQLCYQDTILWKDIFHKNNQL |
| | | ALTLIDTNRSRACHPCSPMCKGSRCWGESSEDC |
| | | QSLTRTVCAGGCARCKGPLPTDCCHEQCAAGCT |
| | | GPKHSDCLACLHFNHSGICELHCPALVTYNTDTF |
| | | ESMPNPEGRYTFGASCVTACPYNYLSTDVGSCT |
| | | LVCPLHNQEVTAEDGTQRCEKCSKPCARVCYGL |
| | | GMEHLREVRAVTSANIQEFAGCKKIFGSLAFLPE |
| | | SFDGDPASNTAPLQPEQLQVFETLEEITGYLYISA |
| | | WPDSLPDLSVFQNLQVIRGRILHNGAYSLTLQGL |
| | | GISWLGLRSLRELGSGLALIHHNTHLCFVHTVPW |
| | | DQLFRNPHQALLHTANRPEDECVGEGLACHQLC |
| | | ARGHCWGPGPTQCVNCSQFLRGQECVEECRVL |
| | | QGLPREYVNARHCLPCHPECQPQNGSVTCFGPE |
| | | ADQCVACAHYKDPPFCVARCPSGVKPDLSYMPI |
| | | WKFPDEEGACQPCPINCTHSCVDLDDKGCPAEA |
| | | AA |
| 122 | | Fc-Fragment Knob: |
| | | DKTHTCPPCP |
| | | APELLGGPSVFLFPPKPKDTLMISRTPEVTCVVV |
| | | DVSHEDPEVKFNWYVDGVEVHNAKTKPREEQY |
| | | NSTYRVVSVLTVLHQDWLNGKEYKCKVSNKAL |
| | | PAPIEKTISKAKGQPREPQVYTLPPCRDELTKNQ |
| | | VSLWCLVKGFYPSDIAVEWESNGQPENNYKTTP |
| | | PVLDSDGSFFLYSKLTVDKSRWQQGNVFSCSVM |
| | | HEALHNHYTQKSLSLSPGGGGSGLNDIFEAQKIE |
| | | WHE |
| 123 | | Fc Fragment (hole): |
| | | DKTHTCPPCPAPEAAGGPSVFLFPPKPKDTLMIS |
| | | RTPEVTCVVVDVSHEDPEVKFNWYVDGVEVHN |
| | | AKTKPREEQYNSTYRVVSVLTVLHQDWLNGKE |
| | | YKCKVSNKALGAPIEKTISKAKGQPREPQVCTLP |
| | | PSRDELTKNQVSLSCAVKGFYPSDIAVEWESNG |
| | | QPENNYKTTPPVLDSDGSFFLVSKLTVDKSRWQ |
| | | QGNVFSCSVMHEALHNRFTQKSLSLSPGK |
| 95 | Fc-only-K-WT | Fc Fragment (knob: |
| | | DKTHTCPPCPAPELLGGPSVFLFPPKPKDTLMISR |
| | | TPEVTCVVVDVSHEDPEVKFNWYVDGVEVHNA |
| | | KTKPREEQYNSTYRVVSVLTVLHQDWLNGKEY |
| | | KCKVSNKALPAPIEKTISKAKGQPREPQVYTLPP |
| | | CRDELTKNQVSLWCLVKGFYPSDIAVEWESNGQ |
| | | PENNYKTTPPVLDSDGSFFLYSKLTVDKSRWQQ |
| | | GNVFSCSVMHEALHNHYTQKSLSLSPG |
| 124 | | Fc Fragment (hole): |
| | | GGGRYESKGGGDKTHTCPPCPAPELLGGPSVFLF |
| | | PPKPKDTLMISRTPEVTCVVVDVSHEDPEVKEN |
| | | WYVDGVEVHNAKTKPREEQYNSTYRVVSVLTV |
| | | LHQDWLNGKEYKCKVSNKALPAPIEKTISKAKG |
| | | QPREPQVCTLPPSRDELTKNQVSLSCAVKGFYPS |
| | | DIAVEWESNGQPENNYKTTPPVLDSDGSFFLVSK |
| | | LTVDKSRWQQGNVFSCSVMHEALHNHYTQKSL |
| | | SLSPG |
| 96 | Fc-only-K-WT- | Fc Fragment (knob: |
| | 3A | DKTHTCPPCPAPELLGGPSVFLFPPKPKDTLMISR |
| | | TPEVTCVVVDVSHEDPEVKFNWYVDGVEVHNA |
| | | KTKPREEQYNSTYRVVSVLTVLHQDWLNGKEY |
| | | KCKVSNKALPAPIEKTISKAKGQPREPQVYTLPP |
| | | CRDELTKNQVSLWCLVKGFYPSDIAVEWESNGQ |
| | | PENNYKTTPPVLDSDGSFFLYSKLTVDKSRWQQ |
| | | GNVFSCSVMHEALHNHYTQKSLSLSPG |
| 125 | | Fc Fragment (hole): |
| | | GGGRYESKGGGDKTHTCPPCPAPELLGGPSVFLF |
| | | PPKPKDTLMISRTPEVTCVVVDVSHEDPEVKFN |
| | | WYVDGVEVHNAKTKPREEQYNSTYRVVSVLTV |
| | | LAQDWLNGKEYKCKVSNKALPAPIEKTISKAKG |
| | | QPREPQVCTLPPSRDELTKNQVSLSCAVKGFYPS |
| | | DIAVEWESNGQPENNYKTTPPVLDSDGSFFLVSK |
| | | LTVDKSRWQQGNVFSCSVMHEALANHATQKSL |
| | | SLSPG |
| 97 | Fc-only-K-3A | Fc Fragment (knob: |
| | | DKTHTCPPCPAPELLGGPSVFLFPPKPKDTLMISR |
| | | TPEVTCVVVDVSHEDPEVKFNWYVDGVEVHNA |
| | | KTKPREEQYNSTYRVVSVLTVLAQDWLNGKEY |
| | | KCKVSNKALPAPIEKTISKAKGQPREPQVYTLPP |
| | | CRDELTKNQVSLWCLVKGFYPSDIAVEWESNGQ |
| | | PENNYKTTPPVLDSDGSFFLYSKLTVDKSRWQQ |
| | | GNVFSCSVMHEALANHATQKSLSLSPGL |
| 126 | | Fc Fragment (hole): |
| | | GGGRYESKGGGDKTHTCPPCPAPELLGGPSVFLF |
| | | PPKPKDTLMISRTPEVTCVVVDVSHEDPEVKFN |
| | | WYVDGVEVHNAKTKPREEQYNSTYRVVSVLTV |
| | | LAQDWLNGKEYKCKVSNKALPAPIEKTISKAKG |
| | | QPREPQVCTLPPSRDELTKNQVSLSCAVKGFYPS |
| | | DIAVEWESNGQPENNYKTTPPVLDSDGSFFLVSK |
| | | LTVDKSRWQQGNVFSCSVMHEALANHATQKSL |
| | | SLSPG |
| 98 | FcRn, human | AIQRTPKIQVYSRHPAENGKSNFLNCYVSGFHPS |
| | single chain | DIEVDLLKNGERIEKVEHSDLSFSKDWSFYLLYY |
| | (hsc-FcRn) | TEFTPTEKDEYACRVNHVTLSQPKIVKWDRDMG |
| | | GGGSGGGGSGGGGSGGGGSAESHLSLLYHLTAV |
| | | SSPAPGTPAFWVSGWLGPQQYLSYNSLRGEAEP |
| | | CGAWVWENQVSWYWEKETTDLRIKEKLFLEAF |
| | | KALGGKGPYTLQGLLGCELGPDNTSVPTAKFAL |
| | | NGEEFMNFDLKQGTWGGDWPEALAISQRWQQQ |
| | | DKAANKELTFLLFSCPHRLREHLERGRGNLEWK |
| | | EPPSMRLKARPSSPGFSVLTCSAFSFYPPELQLRF |
| | | LRNGLAAGTGQGDFGPNSDGSFHASSSLTVKSG |
| | | DEHHYCCIVQHAGLAQPLRVELESPAKSSLEHH |
| | | HHHHHHHHIGLNDIFEAQKIEWHE |
| | AAV2-Qtag-Fc- | AAV2-Qtag SEQ ID NO: 1, 2, 3 |
| | only-Ktag-WT | Fc-only-K-WT SEQ ID NO: 95, 124 |
| | AAV2-Qtag-Fc- | AAV2-Qtag SEQ ID NO: 1, 2, 3 |
| | only-Ktag-WT- | Fc-only-K-WT-3A SEQ ID NO: 96, 125 |
| | 3A | |
| | AAV2-Qtag-Fc- | AAV2-Qtag SEQ ID NO: 1, 2, 3 |
| | only-Ktag-3A | Fc-only-K-3A SEQ ID NO: 97, 126 |
| 102 | aEGFR-aHer2- | aHER2scFv-Fc hole: |
| | scFv-Fc-Ktag | DIQMTQSPSSLSASVGDRVTITCRASQDVNTAVA |
| | | WYQQKPGKAPKLLIYSASFLYSGVPSRFSGSRSG |
| | | TDFTLTISSLQPEDFATYYCQQHYTTPPTFGCGT |
| | | KVEIKGGSGGGGSGGGGSGGGGSGGEVQLVESG |
| | | GGLVQPGGSLRLSCAASGFNIKDTYIHWVRQAP |
| | | GKCLEWVARIYPTNGYTRYADSVKGRFTISADT |
| | | SKNTAYLQMNSLRAEDTAVYYCSRWGGDGFYA |
| | | MDYWGQGTLVTVSSGGSGGGGSGGGGDKTHT |
| | | CPPCPAPEAAGGPSVFLFPPKPKDTLMISRTPEVT |
| | | CVVVDVSHEDPEVKFNWYVDGVEVHNAKTKPR |
| | | EEQYNSTYRVVSVLTVLHQDWLNGKEYKCKVS |
| | | NKALGAPIEKTISKAKGQPREPQVCTLPPSRDELT |
| | | KNQVSLSCAVKGFYPSDIAVEWESNGQPENNYK |
| | | TTPPVLDSDGSFFLVSKLTVDKSRWQQGNVFSC |
| | | SVMHEALHNRFTQKSLSLSPG |
| 127 | | aEGFRscFv-FC knob: |
| | | DILLTQSPVILSVSPGERVSFSCRASQSIGTNIHWY |
| | | QQRTNGSPRLLIKYASESISGIPSRFSGSGSGTDFT |
| | | LSINSVESEDIADYYCQQNNNWPTTFGCGTKLEL |
| | | KGGSGGGGSGGGGSGGGGSGGQVQLKQSGPGL |
| | | VQPSQSLSITCTVSGFSLTNYGVHWVRQSPGKCL |
| | | EWLGVIWSGGNTDYNTPFTSRLSINKDNSKSQVF |
| | | FKMNSLQSNDTAIYYCARALTYYDYEFAYWGQ |
| | | GTLVTVSAGGGGSGGGGSDKTHTCPPCPAPEAA |
| | | GGPSVFLFPPKPKDTLMISRTPEVTCVVVDVSHE |
| | | DPEVKFNWYVDGVEVHNAKTKPREEQYNSTYR |
| | | VVSVLTVLHQDWLNGKEYKCKVSNKALGAPIE |
| | | KTISKAKGQPREPQVYTLPPCRDELTKNQVSLW |
| | | CLVKGFYPSDIAVEWESNGQPENNYKTTPPVLD |
| | | SDGSFFLYSKLTVDKSRWQQGNVFSCSVMHEAL |
| | | HNHYTQKSLSLSPGGGGGSGGGGSGGGGSGGG |
| | | GSGGGRYESKGGG |
| | AAV2-Qtag- | AAV2-Qtag SEQ ID NO: 1, 2, 3 |
| | aEGFR-aHer2- | aEGFR-aHer2-scFv-Fc-Ktag SEQ ID NO: 102, 127 |
| | scFv-Fc-Ktag | |
| 104 | hu EGFRvIII | LEEKKGNYVVTDHGSCVRACGADSYEMEEDGV |
| | | RKCKKCEGPCRKVCNGIGIGEFKDSLSINATNIK |
| | | HFKNCTSISGDLHILPVAFRGDSFTHTPPLDPQEL |
| | | DILKTVKEITGFLLIQAWPENRTDLHAFENLEIIR |
| | | GRTKQHGQFSLAVVSLNITSLGLRSLKEISDGDVI |
| | | ISGNKNLCYANTINWKKLFGTSGQKTKIISNRGE |
| | | NSCKATGQVCHALCSPEGCWGPEPRDCVSCRNV |
| | | SRGRECVDKCNLLEGEPREFVENSECIQCHPECL |
| | | PQAMNITCTGRGPDNCIQCAHYIDGPHCVKTCP |
| | | AGVMGENNTLVWKYADAGHVCHLCHPNCTYG |
| | | CTGPGLEGCPTNGPKIPSVDGGSPTPPTPGGGSG |
| | | LNDIFEAQKIEWHEARAHHHHHH |
| 105 | aEGFR-Fab- | HC: |
| | Qtag-GSG | QVQLKQSGPGLVQPSQSLSITCTVSGFSLTNYGV |
| | | HWVRQSPGKGLEWLGVIWSGGNTDYNTPFTSR |
| | | LSINKDNSKSQVFFKMNSLQSNDTAIYYCARALT |
| | | YYDYEFAYWGQGTLVTVSAASTKGPSVFPLAPS |
| | | SKSTSGGTAALGCLVKDYFPEPVTVSWNSGALT |
| | | SGVHTFPAVLQSSGLYSLSSVVTVPSSSLGTQTYI |
| | | CNVNHKPSNTKVDKKVEPKSCGSGYRYRQEPEA |
| 128 | | LC (kappa): |
| | | DILLTQSPVILSVSPGERVSFSCRASQSIGTNIHWY |
| | | QQRTNGSPRLLIKYASESISGIPSRFSGSGSGTDFT |
| | | LSINSVESEDIADYYCQQNNNWPTTFGAGTKLEL |
| | | KRTVAAPSVFIFPPSDEQLKSGTASVVCLLNNFY |
| | | PREAKVQWKVDNALQSGNSQESVTEQDSKDST |
| | | YSLSSTLTLSKADYEKHKVYACEVTHQGLSSPV |
| | | TKSFNRGEC |
| | azide modified | AAV2-Qtag SEQ ID NO: 1, 2, 3 |
| | AAV-2-Q-tag | Ktag-PEG3-K-N3 SEQ ID NO: Formula 1 |
| | (AAV-2-N3) | |
| | AAV-2-aEGFR- | AAV2-Qtag SEQ ID NO: 1, 2, 3 |
| | Fab-GSG | aEGFR-Fab-GSG SEQ ID NO: 105, 128 |
| | Ktag-PEG3-K- | Formula 1 |
| | N3 | |
| | K-tag-PEG12- | Formula 2 |
| | DBCO | |
| 110 | AAV-5-VP1- | MSFVDHPPDWLEEVGEGLREFLGLEAGPPKPKP |
| | Qtag-VR1 | NQQHQDQARGLVLPGYNYLGPGNGLDRGEPVN |
| | | RADEVAREHDISYNEQLEAGDNPYLKYNHADA |
| | | EFQEKLADDTSFGGNLGKAVFQAKKRVLEPFGL |
| | | VEEGAKTAPTGKRIDDHFPKRKKARTEEDSKPST |
| | | SSDAEAGPSGSQQLQIPAQPASSLGADTMSAGG |
| | | GGPLGDNNQGADGVGNASGDWHCDSTWMGDR |
| | | VVTKSTRTWVLPSYNNHQYREIKSGSVGGGGSY |
| | | RYRQGGGGSNANAYFGYSTPWGYFDFNRFHSH |
| | | WSPRDWQRLINNYWGFRPRSLRVKIFNIQVKEV |
| | | TVQDSTTTIANNLTSTVQVFTDDDYQLPYVVGN |
| | | GTEGCLPAFPPQVFTLPQYGYATLNRDNTENPTE |
| | | RSSFFCLEYFPSKMLRTGNNFEFTYNFEEVPFHSS |
| | | FAPSQNLFKLANPLVDQYLYRFVSTNNTGGVQF |
| | | NKNLAGRYANTYKNWFPGPMGRTQGWNLGSG |
| | | VNRASVSAFATTNRMELEGASYQVPPQPNGMT |
| | | NNLQGSNTYALENTMIFNSQPANPGTTATYLEG |
| | | NMLITSESETQPVNRVAYNVGGQVATNNQSSTT |
| | | APATGTVNTQEIVPGSVWMERDVYLQGPIWAKI |
| | | PETGAHFHPSPAMGGFGLKHPPPMMLIKNTPVP |
| | | GNITSFSDVPVSSFITQYSTGQVTVEMEWELKKE |
| | | NSKRWNPEIQYTNNYNDPQFVDFAPDSTGEYRT |
| | | TRPIGTRYLTRPL |
| 111 | AAV-5-VP1- | MSFVDHPPDWLEEVGEGLREFLGLEAGPPKPKP |
| | Qtag-VR4 | NQQHQDQARGLVLPGYNYLGPGNGLDRGEPVN |
| | | RADEVAREHDISYNEQLEAGDNPYLKYNHADA |
| | | EFQEKLADDTSFGGNLGKAVFQAKKRVLEPFGL |
| | | VEEGAKTAPTGKRIDDHFPKRKKARTEEDSKPST |
| | | SSDAEAGPSGSQQLQIPAQPASSLGADTMSAGG |
| | | GGPLGDNNQGADGVGNASGDWHCDSTWMGDR |
| | | VVTKSTRTWVLPSYNNHQYREIKSGSVDGSNAN |
| | | AYFGYSTPWGYFDFNRFHSHWSPRDWQRLINN |
| | | YWGFRPRSLRVKIFNIQVKEVTVQDSTTTIANNL |
| | | TSTVQVFTDDDYQLPYVVGNGTEGCLPAFPPQV |
| | | FTLPQYGYATLNRDNTENPTERSSFFCLEYFPSK |
| | | MLRTGNNFEFTYNFEEVPFHSSFAPSQNLFKLAN |
| | | PLVDQYLYRFVSTNNGGGGSYRYRQGGGGSVQ |
| | | FNKNLAGRYANTYKNWFPGPMGRTQGWNLGS |
| | | GVNRASVSAFATTNRMELEGASYQVPPQPNGM |
| | | TNNLQGSNTYALENTMIFNSQPANPGTTATYLE |
| | | GNMLITSESETQPVNRVAYNVGGQVATNNQSST |
| | | TAPATGTVNTQEIVPGSVWMERDVYLQGPIWAK |
| | | IPETGAHFHPSPAMGGFGLKHPPPMMLIKNTPVP |
| | | GNITSFSDVPVSSFITQYSTGQVTVEMEWELKKE |
| | | NSKRWNPEIQYTNNYNDPQFVDFAPDSTGEYRT |
| | | TRPIGTRYLTRPL |
| 112 | AAV-5-VP1- | MSFVDHPPDWLEEVGEGLREFLGLEAGPPKPKP |
| | Qtag-VR8 | NQQHQDQARGLVLPGYNYLGPGNGLDRGEPVN |
| | | RADEVAREHDISYNEQLEAGDNPYLKYNHADA |
| | | EFQEKLADDTSFGGNLGKAVFQAKKRVLEPFGL |
| | | VEEGAKTAPTGKRIDDHFPKRKKARTEEDSKPST |
| | | SSDAEAGPSGSQQLQIPAQPASSLGADTMSAGG |
| | | GGPLGDNNQGADGVGNASGDWHCDSTWMGDR |
| | | VVTKSTRTWVLPSYNNHQYREIKSGSVDGSNAN |
| | | AYFGYSTPWGYFDFNRFHSHWSPRDWQRLINN |
| | | YWGFRPRSLRVKIFNIQVKEVTVQDSTTTIANNL |
| | | TSTVQVFTDDDYQLPYVVGNGTEGCLPAFPPQV |
| | | FTLPQYGYATLNRDNTENPTERSSFFCLEYFPSK |
| | | MLRTGNNFEFTYNFEEVPFHSSFAPSQNLFKLAN |
| | | PLVDQYLYRFVSTNNTGGVQFNKNLAGRYANT |
| | | YKNWFPGPMGRTQGWNLGSGVNRASVSAFATT |
| | | NRMELEGASYQVPPQPNGMTNNLQGSNTYALE |
| | | NTMIFNSQPANPGTTATYLEGNMLITSESETQPV |
| | | NRVAYNVGGQVATNNQSSGGGGSYRYRQGGG |
| | | GSPATGTVNTQEIVPGSVWMERDVYLQGPIWAK |
| | | IPETGAHFHPSPAMGGFGLKHPPPMMLIKNTPVP |
| | | GNITSFSDVPVSSFITQYSTGQVTVEMEWELKKE |
| | | NSKRWNPEIQYTNNYNDPQFVDFAPDSTGEYRT |
| | | TRPIGTRYLTRPL |
| 113 | AAV-5-VP2 | TAPTGKRIDDHFPKRKKARTEEDSKPSTSSDAEA |
| | | GPSGSQQLQIPAQPASSLGADTMSAGGGGPLGD |
| | | NNQGADGVGNASGDWHCDSTWMGDRVVTKST |
| | | RTWVLPSYNNHQYREIKSGSVDGSNANAYFGYS |
| | | TPWGYFDFNRFHSHWSPRDWQRLINNYWGFRP |
| | | RSLRVKIFNIQVKEVTVQDSTTTIANNLTSTVQVF |
| | | TDDDYQLPYVVGNGTEGCLPAFPPQVFTLPQYG |
| | | YATLNRDNTENPTERSSFFCLEYFPSKMLRTGNN |
| | | FEFTYNFEEVPFHSSFAPSQNLFKLANPLVDQYL |
| | | YRFVSTNNTGGVQFNKNLAGRYANTYKNWFPG |
| | | PMGRTQGWNLGSGVNRASVSAFATTNRMELEG |
| | | ASYQVPPQPNGMTNNLQGSNTYALENTMIFNSQ |
| | | PANPGTTATYLEGNMLITSESETQPVNRVAYNV |
| | | GGQVATNNQSSTTAPATGTVNTQEIVPGSVWME |
| | | RDVYLQGPIWAKIPETGAHFHPSPAMGGFGLKH |
| | | PPPMMLIKNTPVPGNITSFSDVPVSSFITQYSTGQ |
| | | VTVEMEWELKKENSKRWNPEIQYTNNYNDPQF |
| | | VDFAPDSTGEYRTTRPIGTRYLTRPL |
| 114 | AAV-5-VP3 | MSAGGGGPLGDNNQGADGVGNASGDWHCDST |
| | | WMGDRVVTKSTRTWVLPSYNNHQYREIKSGSV |
| | | DGSNANAYFGYSTPWGYFDFNRFHSHWSPRDW |
| | | QRLINNYWGFRPRSLRVKIFNIQVKEVTVQDSTT |
| | | TIANNLTSTVQVFTDDDYQLPYVVGNGTEGCLP |
| | | AFPPQVFTLPQYGYATLNRDNTENPTERSSFFCL |
| | | EYFPSKMLRTGNNFEFTYNFEEVPFHSSFAPSQN |
| | | LFKLANPLVDQYLYRFVSTNNTGGVQFNKNLAG |
| | | RYANTYKNWFPGPMGRTQGWNLGSGVNRASV |
| | | SAFATTNRMELEGASYQVPPQPNGMTNNLQGSN |
| | | TYALENTMIFNSQPANPGTTATYLEGNMLITSES |
| | | ETQPVNRVAYNVGGQVATNNQSSTTAPATGTV |
| | | NTQEIVPGSVWMERDVYLQGPIWAKIPETGAHF |
| | | HPSPAMGGFGLKHPPPMMLIKNTPVPGNITSFSD |
| | | VPVSSFITQYSTGQVTVEMEWELKKENSKRWNP |
| | | EIQYTNNYNDPQFVDFAPDSTGEYRTTRPIGTRY |
| | | LTRPL |
| | AAV-5-Qtag- | VP1-Qtag-VR1 SEQ ID NO: 110 |
| | VR1 | VP2 SEQ ID NO: 113 |
| | | VP3 SEQ ID NO: 114 |
| | AAV-5-Qtag- | VP1-Qtag-VR1 SEQ ID NO: 111 |
| | VR4 | VP2 SEQ ID NO: 113 |
| | | VP3 SEQ ID NO: 114 |
| | AAV-5-Qtag- | VP1-Qtag-VR1 SEQ ID NO: 112 |
| | VR8 | VP2 SEQ ID NO: 113 |
| | | VP3 SEQ ID NO: 114 |
| | AAV-5-aHER2- | AAV-5-Qtag-VR4 SEQ ID NO: 111, 113, 114 |
| | Fab-2G4S-Ktag | aHER2-Fab-2G4S-Ktag SEQ ID NO: 25, 84 |
| 119 | AAV-5-VP1 | MSFVDHPPDWLEEVGEGLREFLGLEAGPPKPKP |
| | | NQQHQDQARGLVLPGYNYLGPGNGLDRGEPVN |
| | | RADEVAREHDISYNEQLEAGDNPYLKYNHADA |
| | | EFQEKLADDTSFGGNLGKAVFQAKKRVLEPFGL |
| | | VEEGAKTAPTGKRIDDHFPKRKKARTEEDSKPST |
| | | SSDAEAGPSGSQQLQIPAQPASSLGADTMSAGG |
| | | GGPLGDNNQGADGVGNASGDWHCDSTWMGDR |
| | | VVTKSTRTWVLPSYNNHQYREIKSGSVDGSNAN |
| | | AYFGYSTPWGYFDFNRFHSHWSPRDWQRLINN |
| | | YWGFRPRSLRVKIFNIQVKEVTVQDSTTTIANNL |
| | | TSTVQVFTDDDYQLPYVVGNGTEGCLPAFPPQV |
| | | FTLPQYGYATLNRDNTENPTERSSFFCLEYFPSK |
| | | MLRTGNNFEFTYNFEEVPFHSSFAPSQNLFKLAN |
| | | PLVDQYLYRFVSTNNTGGVQFNKNLAGRYANT |
| | | YKNWFPGPMGRTQGWNLGSGVNRASVSAFATT |
| | | NRMELEGASYQVPPQPNGMTNNLQGSNTYALE |
| | | NTMIFNSQPANPGTTATYLEGNMLITSESETQPV |
| | | NRVAYNVGGQMATNNQSSTTAPATGTYNLQEI |
| | | VPGSVWMERDVYLQGPIWAKIPETGAHFHPSPA |
| | | MGGFGLKHPPPMMLIKNTPVPGNITSFSDVPVSS |
| | | FITQYSTGQVTVEMEWELKKENSKRWNPEIQYT |
| | | NNYNDPQFVDFAPDSTGEYRTTRPIGTRYLTRPL |
| 120 | AAV-2- | MAADGYLPDWLEDTLSEGIRQWWKLKPGPPPP |
| | VP1RA-aTfR- | KPAERHKDDSRGLVLPGYKYLGPFNGLDKGEPV |
| | Nb-1_T456 | NEADAAALEHDKAYDRQLDSGDNPYLKYNHAD |
| | | AEFQERLKEDTSFGGNLGRAVFQAKKRVLEPLG |
| | | LVEEPVKTAPGKKRPVEHSPVEPDSSSGTGKAG |
| | | QQPARKRLNFGQTGDADSVPDPQPLGQPPAAPS |
| | | GLGTNTMATGSGAPMADNNEGADGVGNSSGN |
| | | WHCDSTWMGDRVITTSTRTWALPTYNNHLYKQ |
| | | ISSQSGASNDNHYFGYSTPWGYFDFNRFHCHFSP |
| | | RDWQRLINNNWGFRPKRLNFKLFNIQVKEVTQN |
| | | DGTTTIANNLTSTVQVFTDSEYQLPYVLGSAHQ |
| | | GCLPPFPADVFMVPQYGYLTLNNGSQAVGRSSF |
| | | YCLEYFPSQMLRTGNNFTFSYTFEDVPFHSSYAH |
| | | SQSLDRLMNPLIDQYLYYLSRTNTPSGTTTGGGG |
| | | SGGGGSQVQLVESGGGLVQAGGSLRLSCAASGS |
| | | SISSIAMGWYRQAPGKERELVAAISSGGTINYAD |
| | | SVKGRFTISRDNAKNTVYLQMNSLKPEDTAVYY |
| | | CNTGRRLQTGSWGQGTQVTVSSGGGGSGGGGS |
| | | QSRLQFSQAGASDIRDQSRNWLPGPCYRQQRVS |
| | | KTSADNNNSEYSWTGATKYHLNGRDSLVNPGP |
| | | AMASHKDDEEKFFPQSGVLIFGKQGSEKTNVDIE |
| | | KVMITDEEEIRTTNPVATEQYGSVSTNLQAGNA |
| | | QAATADVNTQGVLPGMVWQDRDVYLQGPIWA |
| | | KIPHTDGHFHPSPLMGGFGLKHPPPQILIKNTPVP |
| | | ANPSTTFSAAKFASFITQYSTGQVSVEIEWELQK |
| | | ENSKRWNPEIQYTSNYNKSVNVDFTVDTNGVYS |
| | | EPRPIGTRYLTRNL |
| 121 | AAV-2- | MAADGYLPDWLEDTLSEGIRQWWKLKPGPPPP |
| | VPIRA-aTfR- | KPAERHKDDSRGLVLPGYKYLGPFNGLDKGEPV |
| | Nb-1_del453- | NEADAAALEHDKAYDRQLDSGDNPYLKYNHAD |
| | 459 | AEFQERLKEDTSFGGNLGRAVFQAKKRVLEPLG |
| | | LVEEPVKTAPGKKRPVEHSPVEPDSSSGTGKAG |
| | | QQPARKRLNFGQTGDADSVPDPQPLGQPPAAPS |
| | | GLGTNTMATGSGAPMADNNEGADGVGNSSGN |
| | | WHCDSTWMGDRVITTSTRTWALPTYNNHLYKQ |
| | | ISSQSGASNDNHYFGYSTPWGYFDFNRFHCHFSP |
| | | RDWQRLINNNWGFRPKRLNFKLFNIQVKEVTQN |
| | | DGTTTIANNLTSTVQVFTDSEYQLPYVLGSAHQ |
| | | GCLPPFPADVFMVPQYGYLTLNNGSQAVGRSSF |
| | | YCLEYFPSQMLRTGNNFTFSYTFEDVPFHSSYAH |
| | | SQSLDRLMNPLIDQYLYYLSRTNTPSGGGGSGG |
| | | GGSGGGGSGGGGSGGGGSQVQLVESGGGLVQA |
| | | GGSLRLSCAASGSSISSIAMGWYRQAPGKERELV |
| | | AAISSGGTINYADSVKGRFTISRDNAKNTVYLQM |
| | | NSLKPEDTAVYYCNTGRRLQTGSWGQGTQVTV |
| | | SSGGGGALQFSQAGASDIRDQSRNWLPGPCYRQ |
| | | QRVSKTSADNNNSEYSWTGATKYHLNGRDSLV |
| | | NPGPAMASHKDDEEKFFPQSGVLIFGKQGSEKT |
| | | NVDIEKVMITDEEEIRTTNPVATEQYGSVSTNLQ |
| | | AGNAQAATADVNTQGVLPGMVWQDRDVYLQG |
| | | PIWAKIPHTDGHFHPSPLMGGFGLKHPPPQILIKN |
| | | TPVPANPSTTFSAAKFASFITQYSTGQVSVEIEWE |
| | | LQKENSKRWNPEIQYTSNYNKSVNVDFTVDTNG |
| | | VYSEPRPIGTRYLTRNL |

### EXAMPLES

### Example 1

### Production of Modified AAV Particles

Viral capsid proteins VP1, VP2, and VP3 are encoded by one open reading frame (ORF) and assembled in a ratio of 1:1:10. In order to target only VP1 as a conjugation or genetic fusions site, two distinct expression plasmids for the viral capsid proteins were used. The first expression plasmid encoded the VP1 with an inserted Q-tag, nanobody or peptide and carried a mutated splice acceptor site (sa), which prevented expression of VP2 and VP3 with a Q-tag, nanobody or peptide. Non-modified VP2 and VP3 are encoded by a second expression plasmid, in which a mutated start codon (sc) for the VP1 coding region prevents expression of non-modified VP1.

To produce rAAVps a transfection protocol for HEK293 cells using polyethylenimine (PEI) as a transfection agent was used.

HEK293 cells were grown in a suitable growth medium (Xell AG; HEK VIP NB+8 mM Glutamine). One Day prior to transfection (Day 0), cells were seeded at a density of 1.8E+6 cells/mL.

rAAVps containing the modified VP1, i.e. either the VP1-Q-tag or the VP1 nanobody/peptide, were produced by quadruple transfection of HEK293 cells using two Rep/Cap plasmids for expression of modified VP1 and non-modified VP2 and VP3, a reporter-encoding transgene plasmid flanked by ITRs, and an adeno-helper plasmid. Rep/Cap plasmid, transgene plasmid and adeno-helper plasmids were used in a 1:1:1 molar ratio with a total DNA amount of 1.8 μg per mL cell suspension, whereby the amount for Rep/Cap-VP2/3 and Rep/Cap-modified VP1 was splitted equally. The DNA and PEI were diluted in culture medium. Total volume of the transfection mixture was 4% of the total cell culture volume. The DNA and PEI solutions were combined with a ratio of PEI to DNA of 2.5:1 (w/w), mixed gently, and allowed to incubate at room temperature for 5 minutes to facilitate transfection complex formation. The formed transfection complex was thereafter added dropwise to the cells, which were subsequently returned to the incubator for a period of 72 hours to allow the transfection to take place.

Three days post-transfection the medium, which now contained the modified AAV particles, was harvested along with the cells. The cells were lysed by adding a lysis buffer (volume: 5% of the total culture volume, 20× lysis buffer: 10% Triton CG110, 40 mM MgCl₂) and DNA degrading agent (Denarase, final concentration: 50 Units Denarase/mL) to cell culture broth, incubated for 60 min. at 37° C. with agitation. Thereafter, salt and diatomaceous earth were added to the lysed cell suspension and shook gently for 30 sec. The total volume was then directly applied to a depth filter (see, e.g., Meierriecks, F., et al. J. Biotechnol. 10 (2023) 31-41).

The rAAVps were purified using affinity chromatography (POROS™ GoPure™ AAVX) and anion-exchange chromatography. Following purification, the AAV preparation was desalted and concentrated using appropriate methods, such as diafiltration, and stored in PBS, pH 7.4. To ensure the quality of the rAAVps, the titer was determined using quantitative PCR (qPCR) or digital PCR (dPCR) and enzyme-linked immunosorbent assay (ELISA) for viral genome and viral particle titers, respectively. Finally, the purified AAV particles were aliquoted and stored at −80° C.

| | | | Capsid | |
|---|---|---|---|---|
| | | Titer | Titer | Full/Empty |
| SEQ ID NO: | Sample | [vg/mL] | [vp/mL] | Ratio |
| 1, 2, | AAV-2-Q-tag | 2.20E+14 | 5.29E+14 | 42% |
| 3, 4 | variant particle | | | |
| | (HSPG silenced), | | | |
| | mGL transgene | | | |

For recombinant (genetic) fusions the yields are shown in FIGS. 28 and 29.

AAV Particles with Genetically Inserted Nanobody and Peptide Sequences

Each AAV-nanobody and -peptide modified particle was produced as described above by transfecting 400 ml HEK293 cells (3E+06 cells/mL).

AAV-2^{RA}-aTfR-Nb were produced as described above with the following plasmids in a 1:1:1:1 molar ratio with a total DNA amount of 1.8 μg/mL:
pAAV-2-Rep/Cap-VP1^{RA}-aTfR-Nb
pAAV-2-Rep/Cap-VP2^{RA}/VP3^{RA}
or
pAAV-2-Rep/Cap-VP2^{RA-YF}/VP3^{RA-YF}
pTransgene-ITR-CAG-mGreenLantern-ITR
pAdHelper

AAV-2^{RA}-aCD98-Nb were produced as described above with the following plasmids in a 1:1:1:1 molar ratio with a total DNA amount of 1.8 μg/mL:
pAAV-2-Rep/Cap-VP1^{RA}-aCD98-Nb
pAAV-2-Rep/Cap-VP2^{RA}/VP3^{RA}
pTransgene-ITR-CAG-mGreenLantern-ITR
pAdHelper

chAAV-9/2^{RA}-aTfR-Nb were produced as described above with the following plasmids in a 1:1:1:1 molar ratio with a total DNA amount of 1.8 μg/mL:
pAAV-2-Rep/Cap-VP1^{RA}-aTfR-Nb
pAAV-9-Rep/Cap-VP2/VP3
pTransgene-ITR-CAG-mGreenLantern-ITR
pAdHelper

AAV-5-aTfR-Nb and AAV-9-TfR-Nb fusions were produced as described above with the following plasmids in a 1:1:1:1 molar ratio with a total DNA amount of 1.8 μg/mL:
pAAV-5-Rep/Cap-VP1-aTfR-Nb
or
pAAV-9-Rep/Cap-VP1-aTfR-Nb
pAAV-5-Rep/Cap-VP2/VP3
or
pAAV-9-Rep/Cap-VP2/VP3
pTransgene-ITR-CAG-mGreenLantern-ITR
pAdHelper

AAV-2^{RA}-aTfR-Pept and chimeric AAV-9/2^{RA}-aTfR-Pept variant particles were produced as described above using the following plasmids in a 1:1:1:1 molar ratio with a total DNA amount of 1.8 μg/mL:
pAAV-2-Rep/Cap-VP1^{RA}-aTfR-Pept
pAAV-2-Rep/Cap-VP2^{RA}/VP3^{RA}
OR
pAAV-9-Rep/Cap-VP2/VP3
pTransgene-ITR-CAG-mGreenLantern-ITR
pAdHelper

See FIGS. 50 and 51 as well as Table 7.

### Example 2

### Production of K-Tag Modified Binding Sites

### K-Tag Modified Nanobodies

All nanobody based constructs were produced as Fc-region fusions with a protease cleavage site (SEQ ID NO: 44) in HEK293f cells using the ExpiFectamine™ 293 transfection kit (Gibco™), following the supplier's protocol. Seven days after transfection, the cells were centrifuged and the supernatant containing the fusion protein was collected. The fusion proteins were purified by affinity chromatography (HiTrap MabSelect SuRe, Cytiva). Elution fractions were pooled and dialyzed against PBS pH 7.4 at 4° C. overnight. The next day, protease mediated cleavage of the Fc-region was carried out by co-incubation with PreScission Protease (Cytiva) at 4° C. overnight, according to the supplier's protocol. Free nanobody-K-tag was purified by applying the cleavage mixture on to HiTrap MabSelect SuRe (Cytiva) and the flow-through was collected.

### K-Tag Modified Fabs

All Fab based constructs were produced in HEK293f cells using the ExpiFectamine™ 293 transfection kit (Gibco™), following the supplier's protocol. Seven days after transfection, the cells were centrifuged and the supernatant containing the protein of interest was collected and purified by affinity chromatography (KappaSelect, Cytiva). Elution fractions were pooled and dialyzed against PBS pH 7.4 at 4° C. overnight and stored thereafter at 4° C.

K-Tag Modified scFvs:
All scFv based constructs were produced as Fc-region fusions with a protease cleavage site (SEQ ID NO: 44). Fab and scFv constructs were produced in HEK293f cells using the ExpiFectamine™ 293 transfection kit (Gibco™), following the supplier's protocol. Seven days after transfection, the cells were centrifuged and the supernatant containing the protein of interest was collected and purified by affinity chromatography (Fab: KappaSelect, Cytiva, scFv: MabSelect Sure, Cytiva). Elution fractions were pooled and dialyzed against PBS pH 7.4 at 4° C. overnight and stored at 4° C. or in case of Fc-region fusion proteins incubated with protease for the cleavage of the Fc-region with PreScission Protease (Cytiva) at 4° C. overnight, according to the supplier's protocol. Free scFv-K-tag was purified by applying the cleavage mixture on to HiTrap MabSelect SuRe (Cytiva) and collecting the flow-through.

### Example 3

Conjugation of K-Tag Modified Binding Sites with Q-Tag Modified AAV Variant Particles

### General Method:

A protein lo-bind tube (0.5 mL) was used to prepare the reaction mixture. First, the AAV-Q-tag variant particle was added to the tube to achieve a final concentration of 300 nM (approx. 1.8E+14 vg/mL), within a reaction volume in the range of from 20 μL to 100 μL of PBS at pH 7.4. Second, the K-tag containing binding site was introduced to reach a final concentration between 4,500 and 15,000 nM (4.5-15 μM), ensuring that the ratio of AAV-Q-tag variant particle to K-tag binding site is in the range of 1:15 and 1:50. Finally, KTG was added to the mixture to obtain a concentration of 50 nM, which corresponds to an AAV-Q-tag variant particle to KTG ratio of 1:1/6.

After the sequential addition of all components, the tube was placed in a Thermoblock or Thermocycler preset to 37° C. to allow the conjugation reaction to proceed overnight, for approximately 15 hours.

Upon completion of the incubation period, the reaction mixture optionally can be subjected to further processing steps such as purification or concentration, which are contingent upon the intended downstream applications of the conjugated AAV particles.

The constructs have conjugation efficiencies of up to more than 40%, depending on the conditions and the molecules.

### Nanobodies:

The conjugation of the AAV-2-Q-tag variant particles with nanobody-K-tag conjugates was performed with a concentration of about 300 nM of the AAV-2-Q-tag variant particle (corresponding to 1.8E+14 vp/mL), a concentration of about 50 nM of KTG and a concentration of about 15 μM of the respective nanobody-K-tag at a temperature of 37° C. for about 15 hours. The conjugates were analyzed via Western Blot (Simple Western™ Automated Western Blot Systems, Biotechne).

| | variant | | Coupling |
|---|---|---|---|
| SEQ ID NO: | particle | Target | efficiency [%] |
| 1, 2, | AAV-2-Q-tag-MH12 | TfR | 42 |
| 3, 14 | (C-terminal K-tag) | | |

### Fabs:

The reactions were carried with 300 nM AAV-2-Q-tag variant particle (1.8E+14 vg/mL), 50 nM KTG, and 15 μM Fab-K-tag, at 37° C. overnight. The conjugates were analyzed via Western Blot (Simple Western™ Automated Western Blot Systems, Biotechne).

The constructs are shown in Table 3 and the results in FIG. 12.

| | variant | | Coupling |
|---|---|---|---|
| SEQ ID NO: | particle | Target | efficiency [%] |
| 1, 2, 3, | AAV-2-Q-tag-aTfR_Fab- | TfR | 20 |
| 20, 79 | 2G4S-K-tag | | |
| | (low affinity) | | |
| 1, 2, | AAV-2-Q-tag-aTfR_Fab- | TfR | 15 |
| 3, 18 | 2G4S-K-tag | | |
| | (high affinity) | | |
| 1, 2, 3, | AAV-2-Q-tag-aTfR_Fab- | TfR | 16 |
| 22, 81 | 4G4S-K-tag | | |
| | (low affinity) | | |
| 1, 2, 3, | AAV-2-Q-tag-aTfR_Fab- | TfR | 19 |
| 21, 80 | 4G4S-K-tag | | |
| | (high affinity) | | |
| 1, 2, 3, | AAV-2-Q-tag-aEGFR_Fab- | EGFR | 10 |
| 15, 48 | 2G4S-K-tag | | |
| 1, 2, 3, | AAV-2-Q-tag-aEGFR_Fab- | EGFR | 19 |
| 16, 77 | 4G4S-K-tag | | |

scFv:

The reactions were carried out with 300 nM AAV-2-Q-tag variant particle (1.8E+14 vp/mL), 50 nM KTG, and 15 μM scFv-K-tag, at 37° C. overnight. Samples were analyzed via Western Blot (Simple Western™ Automated Western Blot Systems, Biotechne).

The constructs are shown in Table 3 and the results in FIG. 12.

| | variant | | Coupling |
|---|---|---|---|
| SEQ ID NO: | particle | Target | efficiency [%] |
| 1, 2, | AAV-2-Q-tag-aEGFR_scFv- | EGFR | 33 |
| 3, 17 | 2G4S-K-tag | | |
| 1, 2, | AAV-2-Q-tag-aEGFR_scFv- | EGFR | 30 |
| 3, 18 | 4G4S-K-tag | | |

### Example 4

### Western Blot Analysis

The analyses were performed with a commercially available method (Simple Western™ Automated Western Blot Systems, Biotechne). Capillaries with the range of 66-440 kDa were used to separate the proteins and bands were visualized with an anti-AAV VP1/VP2/VP3 rabbit polyclonal primary antibody (VP51; 61084, Progen) and an anti-Rabbit Detection Kit (Biotechne). Samples were diluted to a titer of 2E+10 vp/mL and 4 μL was applied with a loading time of 6 sec, and separation time was set to 34 min. Blocking was carried out for 30 min., followed by wash steps and 60 min. incubation with the primary antibody, followed by wash steps and secondary antibody incubation for 30 min. Finally, a detection solution, which was a mixture of Luminol-S and Peroxide, was applied.

The results are shown in FIGS. 3, 4, 5, 6, 12 and FIGS. 31-34.

### Example 5

### Surface Plasmon Resonance (SPR) Binding Kinetics Assay

### General Method:

Recombinant target of the binding site conjugated or fused to the rAAVps was used as analyte for the binding kinetics assay. Analyte concentrations were prepared with a maximum concentration of 1,000 nM to 1,500 nM, with serial dilutions (2-3-fold) to generate five different concentrations for the kinetic analysis.

The respective AAV-binding site conjugate was used as a capture ligand. Immobilization of the capture ligand was conducted on a CM5 sensor chip using a T200 device. The chip surface was prepared with CaptureSelect™ Biotin Anti-AAVX Conjugate (Thermo Fisher, 7103522500) at a concentration of 1 mg/mL, diluted 1:20. The ligand was immobilized at a flow rate of 10 μL/min for 420 seconds. The immobilization buffer and sample diluent comprised 10 mM sodium acetate (pH 4.5). The immobilization running buffer was HBS-N. During the experiment, HBS-EP+buffer containing 0.05% BSA and 150 mM NaCl was used to minimize non-specific binding. AAV-binding site conjugates were captured for 420 seconds at 5 μL/min, followed by a 120 second stabilization period, with concentrations ranging from 1E+12 to 1E+13 vp/mL (diluted in PBS). Thereafter, five different concentrations of the respective analyte were injected in a single cycle modus. The association phase was monitored at a flow rate of 50 μL/min for 180 seconds, followed by the dissociation phase at the same flow rate for 360 or 600 seconds. Three regeneration conditions were used sequentially to ensure the complete removal of bound analyte and to restore the sensor chip to baseline conditions. The first and second regeneration steps involved 10 mM Glycine-HCl (pH 1.5) at a flow rate of 30 μL/min for 90 seconds, with a 60 second stabilization time. The third regeneration used 25 mM NaOH at a flow rate of 30 μL/min for 60 seconds, also followed by a 60 second stabilization time.

The temperature of the chip and sample was maintained at 25° C. The sample tray was cooled to 10° C. to preserve analyte integrity.

### AAV-2-Nanobody Conjugates:

For the AAV-2-Q-tag-MH12-K-tag variant particle (AAV-2-MH12) recombinant human transferrin receptor (human (dimeric) TfR ectodomain (SEQ ID NO: 06)) was used as an analyte for the binding kinetics assay. The results are shown in FIGS. 7, 8 and 9 as well as Table 2.

### AAV-2-Fab (TfR) Conjugates:

For the AAV-2-Q-tag-anti-TfR Fab-K-tag variant particle (AAV-2-aTfR Fab) recombinant human transferrin receptor (human (dimeric) TfR ectodomain (SEQ ID NO: 06)) was used as an analyte for the binding kinetics assay. The results are shown in FIGS. 13 to 17 as well as Table 4.

### AAV-2-Fab/scFv (EGFR) Conjugates:

For the AAV-2-Q-tag-anti-EGFR Fab/scFv-K-tag variant particles (AAV-2-aEGFR Fab/scFv) recombinant human EGFR (SEQ ID NO: 07) was used as an analyte for the binding kinetics assay. The results are shown in FIGS. 21 to 25 as well as Table 6.

### AAV-Nb and -Peptide Variant Particles:

For the AAV-Nb and -peptide variant particles recombinant human transferrin receptor (human (dimeric) TfR ectodomain (SEQ ID NO: 06)) or recombinant human CD98 (SEQ ID NO: 45) was used as an analyte for the binding kinetics assay. The results are shown in FIGS. 35-40, 41-47, 48-51 as well as Table 8.

### AAV-2-Fab/scFv (Her2) Conjugates:

For the AAV-2-Q-tag-anti-Her2 Fab/scFv-K-tag variant particles (AAV-2-aHer2-Fab/scFv) recombinant Her2 (SEQ ID NO: 94) at maximum concentration of 100 nM, with 2-fold serial dilution to generate five different concentrations, was used as an analyte for the binding kinetics assay. The results are shown in FIGS. 29 to 32 as well as Table 8.

### AAV-2-Fc Fragment Conjugates:

For the AAV-2-Q-tag-anti-Fc-K-tag variant particles recombinant target, here hsc-FcRn (SEQ ID NO: 98) at maximum concentration of 3000 nM, 2-fold serial dilution to generate five different concentrations, was used for the kinetic analysis. Running Buffer was PBS at pH 6.0. The results are shown in FIGS. 36 to 41 as well as Table 8.

AAV-2-Bispecific scFv (EGFR and Her2) Conjugates:

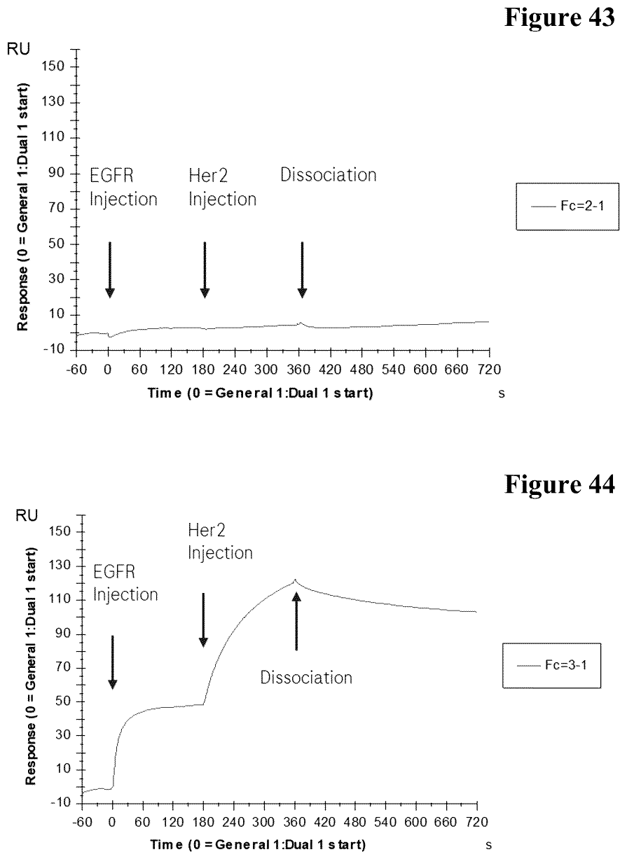

For the AAV-2-Q-tag-anti-EGFR/anti-Her2-Fc-K-tag variant particles recombinant target, here human EGFR (SEQ ID NO: 104) at maximum concentration of 100 nM and human Her2 (SEQ ID NO: 94) at maximum concentration of 300 nM were used for the kinetic analysis. The results are shown in FIGS. 43 and 44.

### Example 6

### Transduction Assay

### General Method:

Target cells were first cultivated in a suitable medium and then seeded in a 96-well plate at a density of 5E+3 cells per well in a volume of 100 μL. After allowing for a 5-hour adhesion period, the cells were transduced with a series of the respective AAV particles at varying multiplicities of infection (MOI), which were prepared in phosphate-buffered saline (PBS). For each well, 25 μL of the AAV MOI series solution was added directly to the cell culture volume, bringing the final volume in the well to 125 μL. The cells were then incubated for 72 hours to facilitate viral transduction and subsequent transgene expression.

For the assessment of transgene expression, the following readout strategy was employed:
To assess transduction with AAV particles comprising a transgene encoding a fluorescent protein, fluorescence intensity was measured as a readout of transgene expression using an Incucyte live-cell imaging or mGL analysis, which enabled the quantification of fluorescence intensity in the transduced cells.

The data from these assays were analyzed to determine the efficiency of AAV-mediated gene delivery across the different MOIs tested. The assay provided a comprehensive evaluation of the transduction capabilities of the AAV vectors and was designed to be adaptable for high-throughput applications, suitable for a variety of cell types and AAV constructs.

### AAV-2-MH12 (SEQ ID NO: 1, 2, 3, 14);

To demonstrate the biological activity of the AAV-2-MH12 conjugate (SEQ ID NO: 1, 2, 3, 14), cell transduction assays were performed. Therefore, conjugated and non-conjugated AAV-2-Q-tag variant particles carrying mGL as transgene were incubated on TfR displaying HeLa (WT) cells and HeLa TfR knockout (KO) cells.

Cells were seeded at a density of 5E+3 cell/well in 100 μL appropriate medium in 96-well format. After 5 h, cells were transduced with MOI series (5E+5-7.81E+3) of AAV-2-MH12 variant particles (SEQ ID NO: 1, 2, 3, 14)/AAV-2-Q-tag variant particles (SEQ ID NO: 1, 2, 3, all three proteins with R585A and R587A) at 37° C. After 72 hours, the cells were analyzed via FACS.

As the AAV particle comprised a transgene encoding a fluorescent protein (mGL), a FACS-based readout was conducted. The data was analyzed to determine the efficiency of AAV-mediated gene delivery across the different MOIs tested. The results are shown in FIGS. 10 and 11.

### AAV-2-aTfR-Fab (SEQ ID NO: 1, 2, 3, 19, 78, SEQ ID NO: 1, 2, 3, 20, 79, SEQ ID NO: 1, 2, 3, 21, 80, SEQ ID NO: 1, 2, 3, 22, 81);

To demonstrate the biological activity of the AAV-Fab conjugates, cell transduction assays were performed. Therefore, conjugated and non-conjugated AAV-2 particles carrying mGL as transgene were incubated with TfR displaying HeLa (WT) cells and HeLa TfR knockout (KO) cells.

Cells were seeded at a density of 5E+3 cell/well in 100 μL appropriate medium in 96-well format. After 5 h, cells were transduced with MOI series (5E+5-7.81E+3) of AAV-2-Fab with HSPG-binding silencing mutations. After 72 hours, the cells were analyzed using FACS.

As the AAV particle comprised a transgene encoding a fluorescent protein (mGL), a FACS-based readout was conducted. The data was analyzed to determine the efficiency of AAV-mediated gene delivery across the different MOIs tested. The results are shown in FIGS. 18 and 19.

### AAV-2-aEGFR-Fab/scFv (SEQ ID NO: 1, 2, 3, 15, 48, SEQ ID NO: 1, 2, 3, 16, 77, SEQ ID NO: 1, 2, 3, 17, SEQ ID NO: 1, 2, 3, 18):

To demonstrate the biological activity of the generated AAV-2-aEGFR conjugates, cell transduction assays were performed. Therefore, conjugated and non-conjugated AAV-2-Q-tag variant particles carrying mGL as transgene were incubated on EGFR displaying HCC827 cells and the negative cell line NCL-H446 (ATCC, HTB-171).

Cells were seeded at a density of 5E+3 cell/well in 100 μL appropriate medium in 96-well format. After 5 h, cells were transduced with MOI series (5E+5-7.81E+3) of AAV-2-Fab/scFv with HSPG-binding silencing mutations. After 72 hours, the cells were analyzed using FACS.

As the AAV particle comprised a transgene encoding a fluorescent protein (mGL), a FACS-based readout was conducted. The data was analyzed to determine the efficiency of AAV-mediated gene delivery across the different MOIs tested. The results are shown in FIGS. 26 and 27.

### AAV-2-aHer2-Fab/scFv (SEQ ID NO: 1, 2, 3, 73, SEQ ID NO: 1, 2, 3, 74, SEQ ID NO: 1, 2, 3, 75, SEQ ID NO: 1, 2, 3, 76):

To demonstrate the biological activity of the generated AAV-2-aHer2 conjugates, cell transduction assays were performed. Therefore, conjugated and non-conjugated AAV-2-Q-tag variant particles carrying mGL as transgene were incubated on Her2 displaying SK-BR3 cells and the Her2 negative cell line NCL-H446

Cells were seeded at a density of 5E+3 cell/well in 100 μL appropriate medium in 96-well format. After 5 h, cells were transduced with MOI series (5E+5-7.81E+3) of AAV-2-Fab/scFv with HSPG-binding silencing mutations. After 72 hours, the cells were analyzed using FACS.

As the AAV particle comprised a transgene encoding a fluorescent protein (mGL), a FACS-based readout was conducted. The data was analyzed to determine the efficiency of AAV-mediated gene delivery across the different MOIs tested. The results are shown in FIGS. 33 and 34.

### Example 7

Analysis of Binding Specificity of AAV-aTfR-Nb and -Peptide Variant Particles to huTfR and Transferrin Competition Assay

To determine the binding specificity of AAV-aTfR-Nb and -peptide variant particles, CHO cells expressing a recombinant human transferrin receptor were used. 80,000 cells were plated in U-bottom 96-well plates coated with 1% BSA and incubated in serum-free DMEM medium. To assess whether AAV-Nb and -peptide variant particles and transferrin bind competitively to huTfR, cells were pre-incubated with 2.7 mg/mL holo-transferrin for 30 minutes. Afterwards, AAV-Nb and -Pept variant particles were added at three different MOIs (1E+3 to 1E+5) to the cells and incubated with or without 2.7 mg/mL holo-transferrin for 60 minutes on ice. After incubation, cells were fixed with 4% PFA in PBS−/− for 15 minutes and blocked overnight with 1% BSA/10% FBS in PBS−/− at 4° C. Cells were stained using monoclonal mouse anti-AAV-2 antibody (2.5 μg/mL, Progen 61055) or anti-AAV-9 antibody (0.5 μg/mL, Progen 690162) for 60 minutes at room temperature. After staining, cells were washed three times with 0.05% Tween in PBS−/− and then incubated with an anti-mouse Alexa647 (1:500, Invitrogen) secondary antibody for 30 minutes at room temperature. Afterwards, cells were washed twice with 0.05% Tween in PBS−/− and the percentage of AAV-2- or AAV-9-positive cells was determined using a BD FACSCanto flow cytometer and analyzed by FlowJo v10.8.1 software.

Results are shown in FIGS. 48-55.

### Example 8

Analysis of Binding of AAV-aTfR-Nb and -aTfR-Pept Variant Particles to Chimeric Human/Mouse TfR and Transferrin Competition Assay

To determine the binding specificity of AAV-aTfR-Nb and -peptide variant particles, MDCK cells expressing a chimeric human/mouse transferrin receptor (TfR^{hu/mu}) were used. 80,000 cells were plated in U-bottom 96-well plates coated with 1% BSA and incubated in serum-free DMEM medium. To assess whether AAV-Nb and -peptide variant particles and transferrin bind competitively to TfR^{hu/mu}, cells were pre-incubated with 2.7 mg/mL holo-transferrin for 30 minutes. Afterwards, AAV-Nb and -peptide variant particles were added at three different MOIs (1E+3 to 1E+5) to the cells and incubated with or without 2.7 mg/mL holo-transferrin for 60 minutes on ice. After incubation, cells were fixed with 4% PFA in PBS−/− for 15 minutes and blocked overnight with 1% BSA/10% FBS in PBS−/− at 4° C. Cells were stained using monoclonal mouse anti-AAV-2 antibody (2.5 μg/mL, Progen 61055) or anti-AAV-9 antibody (0.5 μg/mL, Progen 690162) for 60 minutes at room temperature. After staining, cells were washed three times with 0.05% Tween in PBS−/− and then incubated with an anti-mouse secondary antibody conjugated to Alexa647 (1:500, Invitrogen) for 30 minutes at room temperature. Afterwards, cells were washed twice with 0.05% Tween in PBS−/− and the percentage of AAV-2- or AAV-9-positive cells was determined using a BD FACSCanto flow cytometer and analyzed by FlowJo v10.8.1 software.

The results are shown in FIGS. 68-74 and FIGS. 75-80.

### Example 9

Binding of AAV-2-aCD98-Nb Variant Particles to huCD98 Expressing Cells

To determine the binding specificity of AAV-2-aCD98-Nb fusions, MDCK cells expressing the human CD98 receptor were used. 80,000 cells were plated in U-bottom 96-well plates coated with 1% BSA and incubated in serum-free DMEM medium. Afterwards, AAV-2-aCD98-Nb variant particles were added at three different MOIs (1E+3 to 1E+5) to the cells and incubated for 60 minutes on ice. After incubation, cells were fixed with 4% PFA in PBS−/− for 15 minutes and blocked overnight with 1% BSA/10% FBS in PBS−/− at 4° C. Cells were stained using monoclonal mouse anti-AAV-2 antibody (2.5 μg/mL, Progen 61055) for 60 minutes at room temperature. After staining, cells were washed three times with 0.05% Tween in PBS−/− and then incubated with an anti-mouse secondary antibody conjugated to Alexa647 (1:500, Invitrogen) for 30 minutes at room temperature. Afterwards, cells were washed twice with 0.05% Tween in PBS−/− and the percentage of AAV-2-positive cells was determined using a BD FACSCanto flow cytometer and analyzed by FlowJo v10.8.1 software.

The results are shown in FIGS. 81-83.

### Example 10

### Transduction Assay for Genetic AAV-X-aTfR-Nb Variant Particles

### HeLa Cells

To infect cells, AAV-2-aTfR-Nb variant particles or AAV-9-aTfR-Nb variant particles or chimeric AAV-9/2-aTfR variant particles packaged with a mGL reporter-encoding transgene were added to 5,000 wild-type HeLa or HeLa TfR knock-out cells at MOIs ranging from 1E+3 to 1E+5. Transduction kinetics were followed by live imaging every six hours using an Incucyte® Live-Cell Instrument. Transduction efficiency was determined by quantifying the percentage of cells expressing the mGL reporter using the Incucyte® Live-Cell Analysis software.

The results are shown in FIG. 105 for the AAV-2-aTfR-Nb variant particles and in FIG. 114 for the AAV-9-aTfR-Nb and AAV-9/2-aTfR variant particles.

Three days after infection, wild-type HeLa and HeLa TfR KO cells were trypsinized and resuspended in 10% FBS in PBS−/−. The percentage of mGL-positive cells was determined using a BD FACSCanto flow cytometer and analyzed by FlowJo v10.8.1 software.

The results are shown in FIGS. 106-113 and 157-158 for the AAV-2-aTfR-Nb variant particles and in FIGS. 114-120 and 159-160 for the AAV-9-aTfR-Nb and AAV-9/2-aTfR variant particles

Glutamatergic Neurons Derived from Human iPSC

Transduction efficiencies of AAV-2-Nb and AAV-9-Nb variant particles as well as non-modified AAV-2 and AAV-9 wild-type particles with glutamatergic neurons differentiated from human iPSCs were determined (iCell GlutaNeurons, FUJIFILM Cellular Dynamics Inc.). To infect cells, particles packaged with an mGreenLantern reporter encoding transgene were added to 65,000 iCell GlutaNeurons at MOIs ranging from 1E+3 to 1E+5. Transduction kinetics were followed by live imaging every six hours for one week using an Incucyte® Live-Cell Instrument. Seven days post-infection, cell were stained with the nuclear dye Draq5 and transduction efficiencies were determined by quantifying the percentage of mGL-/Draq5-positive cells using the Incucyte® Live-Cell Analysis software.

The results are shown in FIGS. 121-131.

NGN2-Positive Neurons Derived from Human iPSC

Transduction efficiencies of AAV-9-Nb variant particles and non-modified AAV-9 wild-type particles with NGN2-positive neurons differentiated from human iPSCs were determined (hiPSC line BIONi010-C-13). To infect cells, particles packaged with an mGreenLantern reporter encoding transgene were added to 75,000 iCell NGN2-positive neurons at MOIs ranging from 1E+3 to 1E+5. Transduction kinetics were followed by live imaging every six hours for one week using an Incucyte® Live-Cell Instrument. Seven days post-infection, transduction efficiencies were determined by quantifying mGL intensity per image using the Incucyte® Live-Cell Analysis software.

The results are shown in FIGS. 132-147.

### Example 11

### Mouse In Vivo Study and Assessment of Brain Delivery

The ability of genetically engineered AAV capsids for transport across the blood-brain barrier and enhanced brain delivery, AAV-aTfR-nanobody variant particles were assessed in an in vivo mouse study. Biodistribution of chAAV-9/2^{RA}-aTfR-Nb-1 particles (group 1), AAV-2^{RA}_aTfR-Nb-1 particles (group 2), AAV-2^{RA} particles (group 3), AAV-9 particles (group 4) and AAV-9-PHP.eB particles (group 5) was assessed after a single intravenous injection to male mice expressing chimeric human/mouse TfR (TfR^{hu/mu} knock-in mice). Twenty-eight days after application tissue samples were dissected for DNA and RNA isolation. Both viral genomes (vg) and RNA expression of the transgene were determined in different regions of the brain (on-target) as well as in liver, lung and spleen (off-target).

The results are shown in FIGS. 148-156.

1. A variant adeno-associated virus (AAV) capsid protein comprising a heterologous peptide or polypeptide covalently inserted into or replacing a part of the GH-loop of the capsid protein relative to a corresponding parental AAV capsid protein, wherein the heterologous peptide or polypeptide comprises a recognition sequence of a transglutaminase.

2. A variant adeno-associated virus (AAV) capsid protein comprising a heterologous peptide or polypeptide covalently inserted into or replacing a part of loop 4 or loop 8 of the capsid protein relative to a corresponding parental AAV capsid protein, wherein the heterologous peptide or polypeptide comprises a recognition sequence of a transglutaminase.

3. The AAV capsid protein of claim 1, wherein
a) the amino acids residues at positions 452-455 or 453-459 of VP1 of AAV-2 (SEQ ID NO: 65) or at the corresponding positions in the capsid protein of another AAV serotype are replaced by the heterologous peptide or polypeptide; or
b) the heterologous peptide or polypeptide is inserted at amino acid position 456 of VP1 of AAV-2 (SEQ ID NO: 65) or at the corresponding positions in the capsid protein of another AAV serotype; or
c) the amino acids residues at positions 577-579 of VP1 of AAV-5 (SEQ ID NO: 119) or at the corresponding positions in the capsid protein of another AAV serotype are replaced by the heterologous peptide or polypeptide.

4. A variant adeno-associated virus (AAV) capsid protein comprising a heterologous peptide or polypeptide covalently inserted relative to a corresponding parental AAV capsid protein, wherein
a) the amino acids residues at positions 452-455 or 453-459 of VP1 of AAV-2 (SEQ ID NO: 65) or at the corresponding positions in the capsid protein of another AAV serotype are replaced by the heterologous peptide or polypeptide;
b) the heterologous peptide or polypeptide is inserted at amino acid position 456 of VP1 of AAV-2 (SEQ ID NO: 65) or at the corresponding positions in the capsid protein of another AAV serotype; or
c) the amino acids residues at positions 577-579 of VP1 of AAV-5 (SEQ ID NO: 119) or at the corresponding positions in the capsid protein of another AAV serotype are replaced by the heterologous peptide or polypeptide.

5. The variant AAV capsid protein according to claim 4, wherein the heterologous peptide or polypeptide comprises a recognition sequence of a transglutaminase.

6.-9. (canceled)

10. The variant AAV capsid protein according to claim 1, wherein the heterologous peptide or polypeptide comprises a recognition sequence of the microbial transglutaminase derived from Kutzneria albida (SEQ ID NO: 66).

11. The variant AAV capsid protein according to claim 1, wherein the heterologous peptide or polypeptide comprises the amino acid sequence YRYRQ (SEQ ID NO: 73).

12. The variant AAV capsid protein according to claim 1, wherein the heterologous peptide or polypeptide comprises
i) the amino acid sequence YRYRQ (SEQ ID NO: 73), and
ii) at its N-terminus and at its C-terminus independently of each other a peptidic linker of at most 10 amino acid residues.

13. The variant AAV capsid protein according to claim 1, wherein the heterologous peptide or polypeptide comprises the amino acid sequence GGGGSYRYRQGGGGS (SEQ ID NO: 74).

14-16. (canceled)

17. The variant AAV capsid protein according to claim 1, wherein the heterologous peptide or polypeptide is covalently conjugated to an antigen binding site or an Fc fragment which comprises the amino acid sequence RYESK (SEQ ID NO: 75).

18. The variant AAV capsid protein according to claim 1, wherein the heterologous peptide or polypeptide is covalently conjugated to an antigen binding site or Fc fragment which comprises the amino acid sequence GGGRYESKGGG (SEQ ID NO: 76).

19. The variant AAV capsid protein according to claim 1, wherein the heterologous peptide or polypeptide is covalently conjugated to an antigen binding site or an Fc fragment which comprises the amino acid sequence RYESK (SEQ ID NO: 75) at its N-terminus or at its C-terminus.

20. The variant AAV capsid protein according to claim 1, wherein the heterologous peptide or polypeptide is covalently conjugated to an antigen binding site which comprises the amino acid sequence GGGRYESKGGG (SEQ ID NO: 76) at its C-terminus.

21. The variant AAV capsid protein according to claim 4, wherein the heterologous peptide or polypeptide is an antigen binding site.

22-30. (canceled)

31. The variant AAV capsid protein according to claim 17, wherein the heterologous peptide or polypeptide is covalently conjugated to a Fc fragment wherein
a) both Fc heavy chains comprise a binding sites for human single chain FcRn;
b) one Fc heavy chain comprise a binding site for human single chain FcRn and the other Fc heavy chain demonstrates abolished binding to human single chain FcRn; or
c) both Fc heavy chains demonstrates abolished binding to human single chain FcRn.

32. (canceled)

33. The variant AAV capsid protein according to claim 17, wherein the heterologous peptide or polypeptide is covalently conjugated to a Fc fragment wherein
a) the Fc fragment comprises the sequences of SEQ ID NO: 95 and SEQ ID NO: 124;
b) the Fc fragment comprises the sequences of SEQ ID NO: 96 and SEQ ID NO: 125; or
c) the Fc fragment comprises the sequences of SEQ ID NO: 97 and SEQ ID NO: 126.

34-46. (canceled)

47. The AAV capsid protein of claim 17, wherein the antigen binding site is
a) a Fab specifically binding to huTfR and is conjugated to the heterologous peptide or polypeptide by the amino acid sequence (GGGGS) 4 (SEQ ID NO: 64),
b) a Fab specifically binding to EGFR and is conjugated to the heterologous peptide or polypeptide by the amino acid sequence (GGGGS) 4 (SEQ ID NO: 64),
c) a Fab specifically binding to EGFR and is conjugated to the heterologous peptide or polypeptide by the amino acid sequence (GGGGS) 2 (SEQ ID NO: 62),
d) a scFv specifically binding to EGFR and is conjugated to the heterologous peptide or polypeptide by the amino acid sequence (GGGGS) 4 (SEQ ID NO: 64),
e) a scFv specifically binding to EGFR and is conjugated to the heterologous peptide or polypeptide by the amino acid sequence (GGGGS) 2 (SEQ ID NO: 62),
f) a Fab specifically binding to Her2 and is conjugated to the heterologous peptide or polypeptide by the amino acid sequence (GGGGS) 4 (SEQ ID NO: 64),
g) a scFv specifically binding to Her2 and is conjugated to the heterologous peptide or polypeptide by the amino acid sequence (GGGGS) 4 (SEQ ID NO: 64), or
h) a scFv specifically binding to Her2 and a scFv specifically binding to EGFR and is conjugated to the heterologous peptide or polypeptide by the amino acid sequence (GGGGS) 2 (SEQ ID NO: 62).

48-57. (canceled)

58. A recombinant adeno-associated virus particle (rAAVp) comprising the variant AAV capsid protein according to claim 1.

59-60. (canceled)

61. A method for producing a rAAVp according claim 58, comprising the step of
incubating a mixture comprising the rAAVp according to claim 59 with an antigen binding site or a Fc fragment in the presence of the microbial transglutaminase derived from Kutzneria albida (KTG) (SEQ ID NO: 66) at pH 7.4 and about 37° C. for about 15 hours,
whereby the rAAVp is present in the mixture at a concentration of about 150 nM to 300 nM, the transglutaminase is present in the mixture at a concentration of about 50 nM to 200 nM and the concentration of the binding site or Fc fragment is about 15 μM.

62-93. (canceled)

94. A nucleic acid encoding the variant AAV capsid protein according to claim 1.

95. A cell comprising the nucleic acid according to claim 94.

96-97. (canceled)

98. A method for producing a rAAVp comprising the variant AAV capsid protein according to claim 1 comprising the steps of
a) cultivating a cell comprising a nucleic acid encoding said variant AAV capsid protein in a medium suitable for the production of the rAAVp and under conditions suitable for the production of the rAAVp,
b) recovering the rAAVp from the cell and the cultivation medium, and
c) optionally purifying the rAAVp obtained in step b) with one or more chromatography steps,
and thereby producing the rAAVp.

99-100. (canceled)

101. A variant adeno-associated virus (AAV) capsid protein comprising the amino acid sequence of SEQ ID NO: 1.

102. A variant adeno-associated virus (AAV) capsid protein comprising the amino acid sequence of SEQ ID NO: 1 and comprising the amino acid sequence of SEQ ID NO: 19.
